(19) United States
(12) Patent Application Publication
(10) Pub. No.: US 2026/0297627 A1
(43) Pub. Date: Oct. 1, 2026

(54) MODULATION OF GLOBAL REGULATORY MECHANISMS AND BIOSYNTHETIC GENE CLUSTERS FOR SIMPLIFIED PRODUCTION OF NATURAL PRODUCTS

(71) Applicant: Max-Planck-Gesellschaft zur Förderung der Wissenschaften e.V., Munich (DE)

(72) Inventors: Helge Bode, Marburg (DE); Paushali Chaudhury, Marburg (DE); Edna Bode, Marburg (DE); Timo Obermeier, Marburg (DE); Alexander Rill, Vienna (DE)

(21) Appl. No.: 19/490,961

(22) PCT Filed: Jun. 7, 2024
(86) PCT No.: PCT/EP2024/065835

§ 371 Date: Dec. 8, 2025

(30) Foreign Application Priority Data
Jun. 8, 2023 (EP) 23 178208.7

(51) Int. Cl. C12N 15/90; C07D 277/14; C12N 9/22; C12N 15/11; C12Q 1/689; C12Q 1/6897

(52) U.S. Cl. CPC C12N 15/902; C07D 277/14; C12N 9/226; C12N 15/11; C12Q 1/689; C12Q 1/6897

(57) ABSTRACT

The present invention relates to a method for finding global regulators in order to induce or increase production of target secondary metabolites. The method comprises providing a bacterium indicator strain producing two fluorescent reporter signals under the same control of two different biosynthetic gene clusters (BGC), preferably highly expressed BGC, performing random mutagenesis in said bacterium indicator strain, and selecting mutant bacteria not producing said reporter signals. The invention also relates to a method to activate the production of target secondary metabolites, and to a CRISPR/Cas based single plasmid to delete or inactivate a global regulator and/or activate a BGC. Finally, the inventive method can be performed in high-throughput format allowing to speed-up the procedure.

The present invention relates to a method for finding global regulators in order to induce or increase production of target secondary metabolites. The method comprises providing a bacterium indicator strain producing two fluorescent reporter signals under the same control of two different biosynthetic gene clusters (BGC), preferably highly expressed BGC, performing random mutagenesis in said bacterium indicator strain, and selecting mutant bacteria not producing said reporter signals. The invention also relates to a method to activate the production of target secondary metabolites, and to a CRISPR/Cas based single plasmid to delete or inactivate a global regulator and/or to activate a BGC. Finally, the inventive method can be performed in high-throughput format allowing to speed-up the procedure.

### BACKGROUND OF THE INVENTION

Numerous medicines are directly originated or are inspired from bacterial natural products and their secondary metabolites. These secondary metabolites are known to be produced by bacteria in response to environmental stress or interaction with host, providing competitive advantages. Proteins essential for the production of the bioactive compound are usually encoded by large cryptic gene clusters that remain silent under normal laboratory conditions, which hinders the discovery of new secondary metabolites. The corresponding biosynthetic gene cluster (BGC) can be identified biometric and act as an indicator or marker of bacterial capacity for the production of secondary metabolites. In general, cryptic BGCs are essentially present 5-10 times more than expressed BGCs, so that all bacterial genomes contain many more BGCs than there are secondary metabolites known from that particular strain.

Understanding the modality of expression of these BGCs will not only allow discovery of new beneficial compounds but also revealing pathogenic mechanisms.

The recognition of the global regulators controlling silent biosynthetic gene clusters can help to achieve these goals. In contrast to pathway-specific regulators which control the transcription of a small number of genes, global regulators control hundreds of genes.

Activation/inactivation of global regulators is related to significant changes in the production of secondary metabolites and to the induction of corresponding biosynthetic gene clusters. For example, global regulatory mechanisms dramatically affect the production of almost all secondary metabolites (SM, also called natural products (NPs)), in bacteria such as those of the genera Photorhabdus and Xenorhabdus.

Several BGCs are silent under the conditions used in laboratories for growing bacterial or fungi strain, or the secondary metabolites are only produced in very minute amounts. Since these secondary metabolites might have interesting biological activities acting as antibiotics, anti-cancer or immune suppressive drugs with applications in medicine, biotechnology and agriculture, it would be desirable to speed-up access to these secondary metabolites.

International patent application WO 2019 192 281 refers to a method for mining secondary metabolism strong promoter based on Streptomyces transcriptome, and application thereof. Taking the strong promoter ermE*p as a reference, using the egfp reporter gene to characterize the activity of the ultimate promoters, and confirm the relatively high activity of the promoter in secondary metabolism. Using high-expression Streptomyces natamycin pathway specific positive regulation factors of the promoters obtained by screening increases the natamycin yield.

U.S. patent application U.S. Pat. No. 2,016,348 097 discloses compositions and methods for activating a silent gene or gene cluster with a bacteriophage and/or Streptomyces Antibiotic Regulatory Protein (SARP) transcription factor.

Park Jimin et al. (ACS Synthetic Biology, 2021, 10 (8), pp. 1859-73) report on a high-throughput transcriptional characterization of regulatory sequences from bacterial biosynthetic gene clusters (BGCs). In the publication a cell-free expression platform for rapid characterization of regulatory sequence activities in S. albidoflavus is described.

Xiang et al. (Communications Biology, 2022, 5 (1), p. 901) report on a visualization reporter system for characterizing antibiotic biosynthetic gene clusters expression with high-sensitivity. A visualization reporter system based on Gram-negative bacterial acyl-homoserine lactone quorum-sensing (VRS-bAHL) is also disclosed. The authors showed that VRS-bAHL can be widely used for characterizing gene expression in Streptomyces. With the guidance of VRS-bAHL, a novel oxazolomycin derivatives is discovered.

Jingjing et al. (Journal of Biological Chemistry, 2017, 292 (48), pp. 19708-20) report on a double-reporter-guided targeted activation of the oxytetracycline silent gene cluster in Streptomyces rimosus M527. In Streptomyces rimosus M527, the oxytetracycline (OTC) biosynthetic gene cluster is not expressed under laboratory conditions. The authors used a reported-guided mutant selection (RGMS) procedure to activate the cluster and to obtain a working mutant M527-PAGT-R7. The authors could show that OTC gene cluster was successfully activated using the RGMS method.

At the present a robust and efficient method to find global regulators in bacteria such as bacteria and fungi, and for producing secondary metabolites at high levels is missing. This is particular relevant for the bacteria Photorhabdus and Xenorhabdus.

It is the objective of the present invention to provide a method for screening for global regulator genes that can be manipulated to activate biosynthetic gene clusters (BGC) for producing target secondary metabolites at high level and/or inhibit background production of other not relevant metabolites. Thus, the present invention further comprises a method to elicit production of a secondary metabolite from a BGC based on deleting a global regulator and/or activating a BGC or based on activating a BGC and/or deleting a global regulator. Importantly, the present invention provides a recombination single plasmid for CRISPR/Cas based gene editing that allows efficient gene deletion and replacement.

The objective of the present invention is solved by the teaching of the independent claims. Further advantageous features, aspects and details of the invention are evident from the dependent claims, the description, the figures, and the examples of the present application.

### BRIEF DESCRIPTION OF THE INVENTION

The present invention relates to a method for finding global regulators in order to induce or increase production of target secondary metabolites. The method comprises providing a bacterium indicator strain producing two fluorescent reporter signals under the same control of two different biosynthetic gene clusters (BGC), preferably highly expressed BGC; performing random mutagenesis in said bacterium indicator strain, and selecting mutant bacteria not producing said reporter signals. The invention also relates to a method to activate the production of target secondary metabolites, and to a CRISPR/Cas based single plasmid for gene editing to delete or inactivate a global regulator, to activate one or more BGC, to substitute a BGC with a fluorescent reporter, or for refactoring. Finally, the method can be performed in high-throughput format allowing to speed-up the procedure.

Therefore, the present invention provides a method for screening for a global regulator (GR) gene in a bacterium, the method comprising:
a) replacing a gene sequence of a first biosynthetic gene cluster (BGC) with a first fluorescent reporter gene;
b) replacing a gene sequence of a second BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first BGC and of the second BGC in said bacterium;
wherein the replacing of a gene sequence of a BGC at step a) and b) does not disrupt a regulatory element of said BGC;
wherein the replacing of step a) and b) is in frame;
   wherein said GR gene regulates production of a target secondary metabolite.

In a preferred embodiment, the method for screening for a global regulator (GR) gene in a bacterium, the method comprises:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite.

In a preferred embodiment, the method for screening for a global regulator (GR) gene in a bacterium, the method comprises:
a) replacing a gene sequence of a first highly expressed biosynthetic gene cluster (BGC) with a first fluorescent reporter gene;
b) replacing a gene sequence of a second highly expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first highly expressed BGC and of the second highly expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite.

Reworded, the inventice method for screening for a global regulator (GR) gene in a bacterium, the method comprises:
a) replacing a gene sequence of a first biosynthetic gene cluster (BGC) with a first fluorescent reporter gene;
b) replacing a gene sequence of a second BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first BGC and of the second BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite, and
   wherein the first BGC and the second BGC are no cryptic BGCs.

In a preferred embodiment of the above inventive method, the step e) is performed by fluorescence activated cell sorting, fluorescence spectroscopy, stereo microscopy, or fluorescence imaging.

In a more preferred embodiment of the invention, the method further comprises after step h), the following step i):
i) screening for production of secondary metabolites by HPLC/mass spectrometry analysis.

In a more preferred embodiment of the invention, the method further comprises after step h), the following step i):
i) screening for production of secondary metabolites regulated by said GR gene by HPLC/mass spectrometry analysis.

In a further preferred embodiment of the above inventive method, performing random mutagenesis of step d) comprises chemical random mutagenesis, or UV-mediated random mutagenesis, or error-prone PCR or transposon mutagenesis.

In a further more preferred embodiment of the above inventive method, said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP).

In a still more preferred embodiment of the above inventive method, the secondary metabolite is an antibiotic, or an anti-cancer drug, or an immune suppressive drug, and is preferably selected from the group comprising Puromycin, Madumycin II, Xenoamicin, rupshomycin, rupshomycin derivatives, safracin, safracin derivatives.

In a preferred embodiment of the above inventive method, the bacterium is selected from the group comprising Xenorhabdus, Photorhabdus, Pseudomonas, Serratia, Vibrio, myxobacteria, cyanobacteria, Bacillus, Paenibacillus and Streptomyces.

In a preferred embodiment of the above inventive method, the bacterium is selected from the group comprising Xenorhabdus, Photorhabdus, Pseudomonas, Serratia, Vibrio, myxobacteria, cyanobacteria, Bacillus, Paenibacillus.

In a preferred embodiment of the above inventive method, the bacterium is not Streptomyces.

In a preferred embodiment of the above inventive method, the steps a) and b) are performed introducing in said bacterium a Cas gene and a first and second BGC specific crRNA array, a first and second fluorescent reporter gene, a first and second pair of BGC specific homology regions left and right.

In a preferred embodiment of the above inventive method, steps a) to e) are performed culturing and handling the bacteria in microtiter plates.

In a preferred embodiment of the above inventive method, steps a) to e) are performed using an automated liquid handling robotics, and wherein said robotics enables high-throughput manipulation of liquid added to or removed from cultures comprising the bacteria.

The invention also relates to a method to elicit production of a secondary metabolite from a BGC in a bacterium, wherein the method comprises the following steps:
i) deleting a GR gene in said bacterium, and
ii) optionally activating a BGC in said bacterium;
   or wherein the method comprises the following steps:
i′) essentially activating a BGC in said bacterium, and
ii′) optionally deleting a GR gene in said bacterium;
   and the method further comprises:
iii) expressing in said bacterium at least one positive selectable marker gene and at least one negative selectable marker gene;
wherein deleting a GR gene of steps i) or ii′) comprises introducing in said bacterium at least one GR specific siRNA, or a group of sequences comprising a Cas gene, at least one GR specific crRNA array, a pair of GR specific homology regions left and right; and/or
   wherein activating a BGC of steps ii) or i′) comprises introducing in said bacterium a group of sequences comprising a Cas gene, at least one BGC specific crRNA array, a pair of BGC specific homology regions left and right, a promoter system, and optionally an enhancer sequence.

In other words, the invention also relates to a method to elicit production of a secondary metabolite by a bacterium, wherein the method comprises the following steps:
i) deleting a global regulator (GR) gene in said bacterium, and
ii) optionally activating a biosynthetic gene cluster (BGC) involved in production of said secondary metabolite in said bacterium;
or wherein the method comprises the following steps:
i′) essentially activating a biosynthetic gene cluster (BGC) involved in production of said secondary metabolite in said bacterium, and
ii′) optionally deleting a GR gene in said bacterium;
   and the method further comprises:
iii) expressing in said bacterium at least one positive selectable marker gene and at least one negative selectable marker gene;
   wherein deleting a GR gene of steps i) or ii′) comprises introducing in said bacterium at least one GR specific siRNA, or a group of sequences comprising a Cas gene, at least one GR specific crRNA array, a pair of GR specific homology regions left and right; and/or
   wherein activating a BGC of steps ii) or i′) comprises introducing in said bacterium a group of sequences comprising a Cas gene, at least one BGC specific crRNA array, a pair of BGC specific homology regions left and right, a promoter system, and optionally an enhancer sequence.

In a preferred embodiment of the above method to elicit production of a secondary metabolite from a BGC in a bacterium, the GR gene is cyaA gene.

In a further preferred embodiment of the above method to elicit production of a secondary metabolite from a BGC in a bacterium, the BGC is xenoamicin.

In a preferred embodiment of the above method to elicit production of a secondary metabolite from a BGC in a bacterium, deleting a GR gene of steps i) or ii′) comprises introducing in said bacterium at least one group of sequences comprising a Cas gene, at least one GR specific crRNA array, a pair of GR specific homology regions left and right.

In a further preferred embodiment of the method to elicit production of a secondary metabolite from a BGC in a bacterium, the Cas is selected from the group comprising Cas9, Cas12 and Cas13.

In a more preferred embodiment of the method to elicit production of a secondary metabolite from a BGC in a bacterium, step ii) or step i′) comprises activating at least 2 BGCs in said bacterium.

In a still more preferred embodiment of the method to elicit production of a secondary metabolite from a BGC in a bacterium, said BGC is selected from the group comprising puromycin, madumycin II, xenoamicin, rupshomycin, rupshomycin derivatives, safracin, safracin derivatives.

In other words, in a still more preferred embodiment of the method to elicit production of a secondary metabolite from a BGC in a bacterium, said secondary metabolite is selected from the group comprising puromycin, madumycin II, xenoamicin, rupshomycin, rupshomycin derivatives, safracin, safracin derivatives.

In a preferred embodiment, the method further comprises culturing and handling the bacteria in microtiter plates.

In a further preferred embodiment, the step i)-iii) are performed using an automated liquid handling robotics, and wherein said robotics enables high-throughput manipulation of liquid added to or removed from cultures comprising the bacteria.

Finally, the present invention provides a recombination single-plasmid to activate or silence a BGC in a bacterium by CRISPR/Cas-mediated homology-directed repair, the plasmid comprising:
an origin of transfer sequence (oriT),
a cas gene under control of a first inducible promoter,
genes for a suitable recombinase system under control of a second inducible promoter,
at least one positive selectable marker gene,
at least one negative selectable marker gene,
a crRNA system selected from the group consisting of:
I) a crRNA system to activate a BGC comprising:
   at least one crRNA framework,
   at least one target spacer specific for a genome sequence positioned upstream to a BGC gene,
   homology region left complementary to a genome sequence positioned upstream to said BGC gene,
   homology region right complementary to a genome sequence positioned inside said BGC gene, and
   a promoter system positioned between said homology region left and said homology region right, and optionally an enhancer sequence;
II) a crRNA system to silence a BGC or to delete or inactivate a GR comprising:
   at least one crRNA framework,
   at least one target spacer specific for a genome sequence positioned inside a BGC gene or inside said GR,
   homology region left complementary to a genome sequence positioned upstream said BGC gene or said GR,
   homology region right complementary to a genome sequence positioned downstream said BGC gene or said GR, and
   optionally a fluorescent reporter gene positioned between said homology region left and said homology region right.

In a particular embodiment of the recombinant plasmid, the Cas is selected from the group comprising Cas9, Cas12, Cas13, dCas9.

### DESCRIPTION OF THE INVENTION

The inventors have here developed a method to finding global regulators to increase secondary metabolite production. The method is based on replacement of at least two constitutively expressed biosynthetic gene clusters by two different fluorescent reporters, followed by random mutagenesis and analysis for loss of fluorescence for both reporters (FIG. 1).

BGC expression can be analysed by using fluorescence microscopy or FACS.

Thus, the disclosed method comprises random mutagenesis, e.g. transposon mutagenesis, screening for loss of fluorescence and mass spectrometry analysis to detect all secondary metabolites.

The disclosed method can also be performed in high-throughput in 96-well format or higher (FIG. 16A-C), allowing replacement of up to 96 BGCs or more in parallel.

The disclosed method allows direct bioactivity testing from crude extracts or simplified isolation of the secondary metabolites.

The inventors have also developed a single and easy to assemble vector to apply CRISPR/Cas (FIGS. 1B, 3A-B, 6A-C) to generate bacterial indicator strains that express a fluorescent protein, for example mNeonGreen, in replacement for a biosynthetic gene cluster (FIG. 17A-B) under the same control of said BGC.

When global regulatory mechanisms are modified to shut-off global SM production, and then a biosynthetic gene clusters (BGC) for the production of a desired SM is selectively activated, only a single SM class is produced at high level, as showed by the activation of rupshomicin BGC in mutants with deletion of the hfq gene encoding a global regulator (strains V-IX in FIG. 12). This is of particular advantage to elicit or increase production of a SM from a silent or lowly-expressed BGC.

The developed CRISPR/Cas single-plasmid can also be used to delete or inactivate a global regulator or part of it (FIG. 1D, 3B), or to activate BGC expression by promoter exchange (FIGS. 1C, 1D, 3B, 6A-C, 7, 8, 9). Genome editing can be checked by PCR on isolated colonies (FIG. 5).

Therefore, the present invention also provides a method to elicit production of a secondary metabolite from a BGC based on deleting a global regulator and/or activating a BGC or based on activating a BGC and/or deleting a global regulator, by using the described CRISPR/Cas single-plasmid (FIG. 3A-B).

Refactoring of mxn BGC (Example 5) led to a substantial increase in the production titer of madumycin to approximately 250 mg/l (FIGS. 8 B, C and D, FIG. 9).

Refactoring of xsc BGC (Example 6, FIG. 10) allowed reaching safracin B production titers above 150 mg/l. Remarkably, the safracin B amount of the Xenorhadus sp. TS4 strain harboring a single-plasmid for xsc BGC activation was 14 times higher than that of E. coli strain LZ84 harboring the xsc Cluster encoded on three expression plasmids (FIG. 11 B).

Importantly, the inventive method for refactoring allowed discovering a novel BGC named rpmA-O, producing the compounds 19-26, wherein 26 is the primary product named rupshomycin. Compounds 19-26 (FIGS. 12, 13, 14) are not described in the prior art.

Therefore, the inventive method has the advantage over the prior art to speed-up access to SM normally produced in very minute amounts, and/or from silent BCG, which is particularly relevant for SMs acting as antibiotics, anti-cancer or immune suppressive drugs.

Moreover, the inventive method allows production of several secondary metabolites by a single bacterial multi-producer strain wherein at least 2 BGC are activated by promoter exchange and/or refactoring (Example 13). Such multi-producer strains (or extracts derived thereof) have the advantage of being easier to handle compared to culture of several mono-producing strains or handling of many extracts of individual compounds to be combined.

The inventive method also allows direct bioactivity testing from crude extracts or simplified isolation of SMs that can also be achieved in high-throughput in 96-well format or higher.

Deletion of crucial global regulators allows a much “cleaner” production of desired SM due to the lack of production of interfering SMs.

The inventive CRISPR/Cas single plasmid allows multiple rounds of gene cluster optimization, that is especially important for multiple transcriptional units, such as for mxn (FIGS. 8, 9), xsc (FIG. 10) or rpm (FIG. 12, 13) BGCs.

The inventive CRISPR/Cas single plasmid also allows direct conjugation of the plasmid from E. coli to the recipient strain without any integration of the plasmid or its parts into the genome.

Importantly, the developed inventive CRISPR/Cpf1 single plasmid allows transformation of Photorhabdus and Xenorhabdus, wherein Photorhabdus had poor transformation efficiency and Xenorhabdus was not transformable at all with the prior art methods (Example 3).

Global regulators and global regulatory mechanisms that affect natural product biosynthesis can be transcription factors (TF), chaperones, metabolic switches, signalling compounds binding to TFs. Exemplary global regulator are Hfq, which is a RNA chaperone mediating interaction of mRNA and sRNA (Example 7); ArcZ, a sRNA partner; DNA methyltransferase Dam1; the CAMP synthase CyaA (Example 12).

Exemplary global regulators are transcription factors that bind to the regulatory element of DNA of a BGC and help to coordinate the responses of several genes to direct the production of biosynthesis of secondary metabolites.

Only non-essential regulators can be addressed with the methodology disclosed herein, as individual clones need to be grown in production cultures to test and verify the effect on natural product production. On the other side, most regulators involved in natural product biosynthesis are non-essential even if they are quite global since the natural products are often not essential. Here might be a slight “Bacterial indicator strain” refers to a strain wherein a first BGC of interest is replaced by a first fluorescent reporter and the second BGC of interest is replaced by a second fluorescent reporter. “Bacterial indicator strain” is used interchangeably with “bacterial reporter strain”.

Thus, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite.

The screening of fluorescent reporter strains is preferably performed by fluorescence activated cell sorting, fluorescence spectroscopy, stereo microscopy, or fluorescence imaging.

Table 11 reports some fluorescent proteins that can be inserted in a BGC according to the disclosed method.

Therefore, the present invention also relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the step e) is performed by fluorescence activated cell sorting, fluorescence spectroscopy, stereo microscopy, or fluorescence imaging.

An embodiment of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite.

A particular embodiment of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the step e) is performed by fluorescence activated cell sorting, fluorescence spectroscopy, stereo microscopy, or fluorescence imaging.

Random mutagenesis can be performed by a method selected from chemical random mutagenesis, UV mediated random mutagenesis, transposon mutagenesis, or error prone PCR.

Transposon mutagenesis allows isolating mutants easily with antibiotic resistance caused by transposon insertion successfully.

Accordingly, a particular embodiment of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein performing random mutagenesis of step d) comprises chemical random mutagenesis, or UV-mediated random mutagenesis, or error-prone PCR or transposon mutagenesis.

A still more particular embodiment of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the step e) is performed by fluorescence activated cell sorting, fluorescence spectroscopy, stereo microscopy, or fluorescence imaging,
   wherein performing random mutagenesis of step d) comprises chemical random mutagenesis, or UV-mediated random mutagenesis, or error-prone PCR or transposon mutagenesis.

A further more particular embodiment of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
   wherein the replacing of a gene sequence of a BGC at step a) and b) does not disrupt a regulatory element of said BGC;
   wherein in steps a) and b) the reading frame is conserved;
   wherein said GR gene regulates production of a target secondary metabolite;
   wherein performing random mutagenesis of step d) comprises chemical random mutagenesis, or UV-mediated random mutagenesis, or error-prone PCR or transposon mutagenesis.

A still more particular embodiment of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
   wherein the replacing of a gene sequence of a BGC at step a) and b) does not disrupt a regulatory element of said BGC;
   wherein in steps a) and b) the reading frame is conserved;
   wherein said GR gene regulates production of a target secondary metabolite,
   wherein the step e) is performed by fluorescence activated cell sorting, fluorescence spectroscopy, stereo microscopy, or fluorescence imaging;
   wherein performing random mutagenesis of step d) comprises chemical random mutagenesis, or UV-mediated random mutagenesis, or error-prone PCR or transposon mutagenesis.

### “Biosynthetic Gene Cluster”

A “biosynthetic gene cluster” (BGC) can be defined as a physically clustered group of two or more genes in a particular genome that together encode a biosynthetic pathway for the production of a specialized metabolite.

A “silent BCG” refers to silent or cryptic BGC under standard laboratory growth conditions, so the SM for which are responsible are not produced.

Selective activation of a BGC refers to the selective stimulation of the expression of a particular BGC in order to obtain production of the SM for which is responsible at high level.

Examples of BGC that can be modulated with the present invention:
encoding enzymes responsible for production of secondary metabolytes, wherein the enzyme is selected from the group comprising non-ribosomal peptide synthetase, terpene synthase/cyclase, polyketide synthase, ribosomally produced peptide (RiPP).
producing secondary metabolites selected from the group comprising an antibiotic, or an anti-cancer drug, or an immune suppressive drug.
producing a secondary metabolite selected from the group comprising Puromycin, Madumycin II, Xenoamicin, Rupshomycin, safracin.

Non-ribosomal peptides (NRP) are not directly encoded in the genome like typical proteins or peptides but are produced by metabolic pathways encoded by BGCs. NRPs are a large family of structurally diverse and pharmacologically useful natural products with broad biological activities. Prominent examples are the antibiotic daptomycin or the immunosuppressant cyclosporine A2. They are assembled by multifunctional enzyme complexes called non-ribosomal peptide synthetases (NRPSs) that are organized in a modular fashion. Each module activates and modifies a specific amino acid (aa) that is then subsequently elongated with an aa activated and modified by the next module thereby generating peptides with their length depending on the number of modules used

Therefore, a particular embodiment of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP).

An alternative embodiment of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the step e) is performed by fluorescence activated cell sorting, fluorescence spectroscopy, stereo microscopy, or fluorescence imaging;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP).

A preferred alternative embodiment of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP).

A further preferred alternative embodiment of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the step e) is performed by fluorescence activated cell sorting, fluorescence spectroscopy, stereo microscopy, or fluorescence imaging;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP).

A further more preferred alternative embodiment of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein performing random mutagenesis of step d) comprises chemical random mutagenesis, or UV-mediated random mutagenesis, or error-prone PCR or transposon mutagenesis;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP).

An aspect of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the step e) is performed by fluorescence activated cell sorting, fluorescence spectroscopy, stereo microscopy, or fluorescence imaging,
   wherein performing random mutagenesis of step d) comprises chemical random mutagenesis, or UV-mediated random mutagenesis, or error-prone PCR or transposon mutagenesis;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP).

A further aspect of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
   wherein the replacing of a gene sequence of a BGC at step a) and b) does not disrupt a regulatory element of said BGC;
   wherein in steps a) and b) the reading frame is conserved;
   wherein said GR gene regulates production of a target secondary metabolite;
   wherein performing random mutagenesis of step d) comprises chemical random mutagenesis, or UV-mediated random mutagenesis, or error-prone PCR or transposon mutagenesis;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP).

Another aspect of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
   wherein the replacing of a gene sequence of a BGC at step a) and b) does not disrupt a regulatory element of said BGC;
   wherein in steps a) and b) the reading frame is conserved;
   wherein said GR gene regulates production of a target secondary metabolite,
   wherein the step e) is performed by fluorescence activated cell sorting, fluorescence spectroscopy, stereo microscopy, or fluorescence imaging;
   wherein performing random mutagenesis of step d) comprises chemical random mutagenesis, or UV-mediated random mutagenesis, or error-prone PCR or transposon mutagenesis;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP).

### Secondary Metabolites (Natural Products)

Secondary metabolites (SM) are natural products (NP) synthesized mainly by bacteria, fungi and plants. They are molecules of low molecular weight with diverse chemical structures and biological activities. Exemplary SMs are pigments, antibiotics, anti-cancer or immune suppressive drugs with applications in medicine, biotechnology and agriculture. Secondary metabolites play important roles in cellular growth and signaling, nutrient acquisition, intra- and interspecies communication, and virulence. A subset of natural products is produced by nonribosomal peptide synthetases (NRPSs).

Exemplary SMs are: Safracin compounds, comprising Safracin B that can be used for the semisynthesis of the two chemoterapeutics Ecteinascidin 743 and (−)-Jorumycin (FIGS. 10, 11), rupshomycin and derivatives produced from rpm BGC (FIG. 12-14), Madumycin II (FIG. 8, 9), Xenoamicine (FIG. 22), Puromycin, Stilbenes (FIG. 2, 4), GameXPeptides (gxpS, FIG. 7A, Table 10), glidobactin (glbA, FIG. 7A), Xenocoumacins (xcnA, FIG. 7B, Table 10), Rhabdopeptides (rxpA, FIG. 7B), Indigoidine, (indC, FIG. 6, Table 10).

Genetic loci involved in the biosynthesis of indigoidine have been found in E. chrysanthemi (indA~indC), V. indigofera (igiA~igiE) (GenBank™ accession number AF088856), and Photorhabdus luminescens (plu2182, plu2186, and plu2187). indC, igiD, and plu2186 genes in these loci seem to encode NRPS.

The glidobactin-like natural products (GLNPs) glidobactin A and cepafungin I have been reported to be potent proteasome inhibitors and are regarded as promising candidates for anticancer drug development. Their biosynthetic gene cluster (BGC) plu1881-1877 is present in entomopathogenic Photorhabdus laumondii but silent under standard laboratory conditions. The plu1881 has the same function as the homologue glbB, i.e. catalysis of the 4-hydroxylation reaction of L-lysine.

The biosynthesis of xenortides A-D consists of two NRPS coded by genes XndA and XndB. The XndA consists of a condensation, adenylation, methylation, and thiolation domain, and has been implicated for the loading of N-methylleucine (xenortides A-B) or N-methylvaline (xenortides C-D). The XndB consists of a condensation, adenylation, methylation, thiolation, and terminal condensation domains. XndB has been implicated in elongation with N-methylphenylalanine, as well as the final condensation of the enzyme-bound peptide with either decarboxylated phenylalanine (phenylethylamine in xenortides A and C) or decarboxylated tryptophan (tryptamine in xenortides B and D), ending the biosynthesis.

In Xenorhabdus and Photorhabdus (XP), GxpS, an NRPS with five modules, is responsible for the biosynthesis of GameXPeptides (Table 10), which are a class of cyclic pentapeptides composed of valine, leucine, and phenylalanine.

RXP are rhabdopeptide/xenortide-like peptides (Table 10).

Other natural products produced by XP: silathride, xenoautoxin, phenylethylamide, tryptamide, rhabdopeptide, and PAX.

Thus, a preferred aspect of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives.

A more preferred aspect of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the step e) is performed by fluorescence activated cell sorting, fluorescence spectroscopy, stereo microscopy, or fluorescence imaging.

A still more preferred aspect of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives.

A further preferred aspect of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein the step e) is performed by fluorescence activated cell sorting, fluorescence spectroscopy, stereo microscopy, or fluorescence imaging.

A further more preferred aspect of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein the secondary metabolite is an antibiotic, or an anti-cancer drug, or an immune suppressive drug and is preferably selected from the group comprising
   Puromycin, Madumycin II, Xenoamicin, rupshomycin, rupshomycin derivatives, safracin, safracin derivatives;
   wherein performing random mutagenesis of step d) comprises chemical random mutagenesis, or UV-mediated random mutagenesis, or error-prone PCR or transposon mutagenesis.

A further still more preferred aspect of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein the step e) is performed by fluorescence activated cell sorting, fluorescence spectroscopy, stereo microscopy, or fluorescence imaging,
   wherein performing random mutagenesis of step d) comprises chemical random mutagenesis, or UV-mediated random mutagenesis, or error-prone PCR or transposon mutagenesis.

The present invention also relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
   wherein the replacing of a gene sequence of a BGC at step a) and b) does not disrupt a regulatory element of said BGC;
   wherein in steps a) and b) the reading frame is conserved;
   wherein said GR gene regulates production of a target secondary metabolite;
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein performing random mutagenesis of step d) comprises chemical random mutagenesis, or UV-mediated random mutagenesis, or error-prone PCR or transposon mutagenesis.

The present invention further relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
   wherein the replacing of a gene sequence of a BGC at step a) and b) does not disrupt a regulatory element of said BGC;
   wherein in steps a) and b) the reading frame is conserved;
   wherein said GR gene regulates production of a target secondary metabolite,
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein the step e) is performed by fluorescence activated cell sorting, fluorescence spectroscopy, stereo microscopy, or fluorescence imaging; wherein performing random mutagenesis of step d) comprises chemical random mutagenesis, or UV-mediated random mutagenesis, or error-prone PCR or transposon mutagenesis.

The present invention alternatively relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP).

The present invention preferably relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   wherein the step e) is performed by fluorescence activated cell sorting, fluorescence spectroscopy, stereo microscopy, or fluorescence imaging.

An embodiment of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP).

A particular embodiment of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   wherein the step e) is performed by fluorescence activated cell sorting, fluorescence spectroscopy, stereo microscopy, or fluorescence imaging.

A more particular embodiment of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   wherein performing random mutagenesis of step d) comprises chemical random mutagenesis, or UV-mediated random mutagenesis, or error-prone PCR or transposon mutagenesis.

A still more particular embodiment of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   wherein the step e) is performed by fluorescence activated cell sorting, fluorescence spectroscopy, stereo microscopy, or fluorescence imaging;
   wherein performing random mutagenesis of step d) comprises chemical random mutagenesis, or UV-mediated random mutagenesis, or error-prone PCR or transposon mutagenesis.

A further more particular embodiment of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
   wherein the replacing of a gene sequence of a BGC at step a) and b) does not disrupt a regulatory element of said BGC;
   wherein in steps a) and b) the reading frame is conserved;
   wherein said GR gene regulates production of a target secondary metabolite;
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   wherein performing random mutagenesis of step d) comprises chemical random mutagenesis, or UV-mediated random mutagenesis, or error-prone PCR or transposon mutagenesis.

A further still more particular embodiment of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
   wherein the replacing of a gene sequence of a BGC at step a) and b) does not disrupt a regulatory element of said BGC;
   wherein in steps a) and b) the reading frame is conserved;
   wherein said GR gene regulates production of a target secondary metabolite,
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   wherein the step e) is performed by fluorescence activated cell sorting, fluorescence spectroscopy, stereo microscopy, or fluorescence imaging;
   wherein performing random mutagenesis of step d) comprises chemical random mutagenesis, or UV-mediated random mutagenesis, or error-prone PCR or transposon mutagenesis.

### Contemplated Bacteria:

Preferred bacteria for the present invention are Gram negative bacteria (Pseudomonas, Xenorhabdus, Photorhabdus, Serratia, Vibrio etc.) and other strains that produce natural products, such as myxobacteria, cyanobacteria, Pseudomonades, Bacillus, Paenibacillus or Streptomyces.

Therefore, an aspect of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein the bacterium is selected from the group comprising Xenorhabdus, Photorhabdus, Pseudomonas, Serratia, Vibrio, myxobacteria, cyanobacteria, Bacillus, Paenibacillus and Streptomyces.

A further aspect of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein the bacterium is selected from the group comprising Xenorhabdus, Photorhabdus, Pseudomonas, Serratia, Vibrio, myxobacteria, cyanobacteria, Bacillus, and Paenibacillus.

A further aspect of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein the bacterium is not Streptomyces.

A further aspect of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the step e) is performed by fluorescence activated cell sorting, fluorescence spectroscopy, stereo microscopy, or fluorescence imaging;
   wherein the bacterium is selected from the group comprising Xenorhabdus, Photorhabdus, Pseudomonas, Serratia, Vibrio, myxobacteria, cyanobacteria, Bacillus, Paenibacillus and Streptomyces.

Preferably, the bacterium is selected from the group comprising Xenorhabdus, Photorhabdus, Pseudomonas, Serratia, Vibrio, myxobacteria, cyanobacteria, Bacillus, and Paenibacillus.

A particular aspect of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein the bacterium is selected from the group comprising Xenorhabdus, Photorhabdus, Pseudomonas, Serratia, Vibrio, myxobacteria, cyanobacteria, Bacillus, Paenibacillus and Streptomyces.

Preferably, the bacterium is selected from the group comprising Xenorhabdus, Photorhabdus, Pseudomonas, Serratia, Vibrio, myxobacteria, cyanobacteria, Bacillus, and Paenibacillus.

A preferable aspect of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the step e) is performed by fluorescence activated cell sorting, fluorescence spectroscopy, stereo microscopy, or fluorescence imaging;
   wherein the bacterium is selected from the group comprising Xenorhabdus, Photorhabdus, Pseudomonas, Serratia, Vibrio, myxobacteria, cyanobacteria, Bacillus, Paenibacillus and Streptomyces.

Preferably, the bacterium is selected from the group comprising Xenorhabdus, Photorhabdus, Pseudomonas, Serratia, Vibrio, myxobacteria, cyanobacteria, Bacillus, and Paenibacillus.

An alternative aspect of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein performing random mutagenesis of step d) comprises chemical random mutagenesis, or UV-mediated random mutagenesis, or error-prone PCR or transposon mutagenesis;
   wherein the bacterium is selected from the group comprising Xenorhabdus, Photorhabdus, Pseudomonas, Serratia, Vibrio, myxobacteria, cyanobacteria, Bacillus, Paenibacillus and Streptomyces.

Preferably, the bacterium is selected from the group comprising Xenorhabdus, Photorhabdus, Pseudomonas, Serratia, Vibrio, myxobacteria, cyanobacteria, Bacillus, and Paenibacillus.

A preferred aspect of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the step e) is performed by fluorescence activated cell sorting, fluorescence spectroscopy, stereo microscopy, or fluorescence imaging, wherein performing random mutagenesis of step d) comprises chemical random mutagenesis, or UV-mediated random mutagenesis, or error-prone PCR or transposon mutagenesis;
   wherein the bacterium is selected from the group comprising Xenorhabdus, Photorhabdus, Pseudomonas, Serratia, Vibrio, myxobacteria, cyanobacteria, Bacillus, Paenibacillus and Streptomyces.

Preferably, the bacterium is selected from the group comprising Xenorhabdus, Photorhabdus, Pseudomonas, Serratia, Vibrio, myxobacteria, cyanobacteria, Bacillus, and Paenibacillus.

A further preferred aspect of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
   wherein the replacing of a gene sequence of a BGC at step a) and b) does not disrupt a regulatory element of said BGC;
   wherein in steps a) and b) the reading frame is conserved;
   wherein said GR gene regulates production of a target secondary metabolite;
   wherein performing random mutagenesis of step d) comprises chemical random mutagenesis, or UV-mediated random mutagenesis, or error-prone PCR or transposon mutagenesis;
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces.

Preferably, the bacterium is selected from the group comprising Xenorhabdus, Photorhabdus, Pseudomonas, Serratia, Vibrio, myxobacteria, cyanobacteria, Bacillus, and Paenibacillus.

A more preferred aspect the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
   wherein the replacing of a gene sequence of a BGC at step a) and b) does not disrupt a regulatory element of said BGC;
   wherein in steps a) and b) the reading frame is conserved;
   wherein said GR gene regulates production of a target secondary metabolite,
   wherein the step e) is performed by fluorescence activated cell sorting, fluorescence spectroscopy, stereo microscopy, or fluorescence imaging;
   wherein performing random mutagenesis of step d) comprises chemical random mutagenesis, or UV-mediated random mutagenesis, or error-prone PCR or transposon mutagenesis;
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces.

Preferably, the bacterium is selected from the group comprising Xenorhabdus, Photorhabdus, Pseudomonas, Serratia, Vibrio, myxobacteria, cyanobacteria, Bacillus, and Paenibacillus.

A still more preferred aspect the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces.

Preferably, the bacterium is selected from the group comprising Xenorhabdus, Photorhabdus, Pseudomonas, Serratia, Vibrio, myxobacteria, cyanobacteria, Bacillus, and Paenibacillus.

A particular aspect of present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the step e) is performed by fluorescence activated cell sorting, fluorescence spectroscopy, stereo microscopy, or fluorescence imaging;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces.

Preferably, the bacterium is selected from the group comprising Xenorhabdus, Photorhabdus, Pseudomonas, Serratia, Vibrio, myxobacteria, cyanobacteria, Bacillus, and Paenibacillus.

A more particular aspect of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces.

Preferably, the bacterium is selected from the group comprising Xenorhabdus, Photorhabdus, Pseudomonas, Serratia, Vibrio, myxobacteria, cyanobacteria, Bacillus, and Paenibacillus.

A still more particular aspect of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the step e) is performed by fluorescence activated cell sorting, fluorescence spectroscopy, stereo microscopy, or fluorescence imaging;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP).

A further more particular aspect of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein performing random mutagenesis of step d) comprises chemical random mutagenesis, or UV-mediated random mutagenesis, or error-prone PCR or transposon mutagenesis;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces.

Preferably, the bacterium is selected from the group comprising Xenorhabdus, Photorhabdus, Pseudomonas, Serratia, Vibrio, myxobacteria, cyanobacteria, Bacillus, and Paenibacillus.

An alternative embodiment of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the step e) is performed by fluorescence activated cell sorting, fluorescence spectroscopy, stereo microscopy, or fluorescence imaging,
   wherein performing random mutagenesis of step d) comprises chemical random mutagenesis, or UV-mediated random mutagenesis, or error-prone PCR or transposon mutagenesis;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces.

Preferably, the bacterium is selected from the group comprising Xenorhabdus, Photorhabdus, Pseudomonas, Serratia, Vibrio, myxobacteria, cyanobacteria, Bacillus, and Paenibacillus.

A preferred embodiment of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
   wherein the replacing of a gene sequence of a BGC at step a) and b) does not disrupt a regulatory element of said BGC;
   wherein in steps a) and b) the reading frame is conserved;
   wherein said GR gene regulates production of a target secondary metabolite;
   wherein performing random mutagenesis of step d) comprises chemical random mutagenesis, or UV-mediated random mutagenesis, or error-prone PCR or transposon mutagenesis;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces.

Preferably, the bacterium is selected from the group comprising Xenorhabdus, Photorhabdus, Pseudomonas, Serratia, Vibrio, myxobacteria, cyanobacteria, Bacillus, and Paenibacillus.

A more preferred embodiment of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
   wherein the replacing of a gene sequence of a BGC at step a) and b) does not disrupt a regulatory element of said BGC;
   wherein in steps a) and b) the reading frame is conserved;
   wherein said GR gene regulates production of a target secondary metabolite,
   wherein the step e) is performed by fluorescence activated cell sorting, fluorescence spectroscopy, stereo microscopy, or fluorescence imaging;
   wherein performing random mutagenesis of step d) comprises chemical random mutagenesis, or UV-mediated random mutagenesis, or error-prone PCR or transposon mutagenesis;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces.

Preferably, the bacterium is selected from the group comprising Xenorhabdus, Photorhabdus, Pseudomonas, Serratia, Vibrio, myxobacteria, cyanobacteria, Bacillus, and Paenibacillus.

A still more preferred embodiment of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives,
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces.

Preferably, the bacterium is selected from the group comprising Xenorhabdus, Photorhabdus, Pseudomonas, Serratia, Vibrio, myxobacteria, cyanobacteria, Bacillus, and Paenibacillus.

A further more preferred embodiment of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the step e) is performed by fluorescence activated cell sorting, fluorescence spectroscopy, stereo microscopy, or fluorescence imaging,
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces.

Preferably, the bacterium is selected from the group comprising Xenorhabdus, Photorhabdus, Pseudomonas, Serratia, Vibrio, myxobacteria, cyanobacteria, Bacillus, and Paenibacillus.

In particular, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives,
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces.

Preferably, the bacterium is selected from the group comprising Xenorhabdus, Photorhabdus, Pseudomonas, Serratia, Vibrio, myxobacteria, cyanobacteria, Bacillus, and Paenibacillus.

More in particular, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein the step e) is performed by fluorescence activated cell sorting, fluorescence spectroscopy, stereo microscopy, or fluorescence imaging;
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces.

Preferably, the bacterium is selected from the group comprising Xenorhabdus, Photorhabdus, Pseudomonas, Serratia, Vibrio, myxobacteria, cyanobacteria, Bacillus, and Paenibacillus.

Also more in particular, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein performing random mutagenesis of step d) comprises chemical random mutagenesis, or UV-mediated random mutagenesis, or error-prone PCR or transposon mutagenesis;
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces.

Preferably, the bacterium is selected from the group comprising Xenorhabdus, Photorhabdus, Pseudomonas, Serratia, Vibrio, myxobacteria, cyanobacteria, Bacillus, and Paenibacillus.

Still more in particular, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein the step e) is performed by fluorescence activated cell sorting, fluorescence spectroscopy, stereo microscopy, or fluorescence imaging,
   wherein performing random mutagenesis of step d) comprises chemical random mutagenesis, or UV-mediated random mutagenesis, or error-prone PCR or transposon mutagenesis;
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces.

Preferably, the bacterium is selected from the group comprising Xenorhabdus, Photorhabdus, Pseudomonas, Serratia, Vibrio, myxobacteria, cyanobacteria, Bacillus, and Paenibacillus.

A particular embodiment of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
   wherein the replacing of a gene sequence of a BGC at step a) and b) does not disrupt a regulatory element of said BGC;
   wherein in steps a) and b) the reading frame is conserved;
   wherein said GR gene regulates production of a target secondary metabolite;
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein performing random mutagenesis of step d) comprises chemical random mutagenesis, or UV-mediated random mutagenesis, or error-prone PCR or transposon mutagenesis;
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces.

Preferably, the bacterium is selected from the group comprising Xenorhabdus, Photorhabdus, Pseudomonas, Serratia, Vibrio, myxobacteria, cyanobacteria, Bacillus, and Paenibacillus.

A more particular embodiment of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
   wherein the replacing of a gene sequence of a BGC at step a) and b) does not disrupt a regulatory element of said BGC;
   wherein in steps a) and b) the reading frame is conserved;
   wherein said GR gene regulates production of a target secondary metabolite,
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein the step e) is performed by fluorescence activated cell sorting, fluorescence spectroscopy, stereo microscopy, or fluorescence imaging;
wherein performing random mutagenesis of step d) comprises chemical random mutagenesis, or UV-mediated random mutagenesis, or error-prone PCR or transposon mutagenesis;
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces.

Preferably, the bacterium is selected from the group comprising Xenorhabdus, Photorhabdus, Pseudomonas, Serratia, Vibrio, myxobacteria, cyanobacteria, Bacillus, and Paenibacillus.

A further more particular embodiment of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces.

Preferably, the bacterium is selected from the group comprising Xenorhabdus, Photorhabdus, Pseudomonas, Serratia, Vibrio, myxobacteria, cyanobacteria, Bacillus, and Paenibacillus.

A further still more particular embodiment of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   wherein the step e) is performed by fluorescence activated cell sorting, fluorescence spectroscopy, stereo microscopy, or fluorescence imaging; and wherein the bacterium is selected from the group comprising Xenorhabdus, Photorhabdus, Pseudomonas, Serratia, Vibrio, myxobacteria, cyanobacteria, Bacillus, Paenibacillus and Streptomyces.

Preferably, the bacterium is selected from the group comprising Xenorhabdus, Photorhabdus, Pseudomonas, Serratia, Vibrio, myxobacteria, cyanobacteria, Bacillus, and Paenibacillus.

Preferably, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces.

Preferably, the bacterium is selected from the group comprising Xenorhabdus, Photorhabdus, Pseudomonas, Serratia, Vibrio, myxobacteria, cyanobacteria, Bacillus, and Paenibacillus.

More preferably, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   wherein the step e) is performed by fluorescence activated cell sorting, fluorescence spectroscopy, stereo microscopy, or fluorescence imaging;
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces.

Preferably, the bacterium is selected from the group comprising Xenorhabdus, Photorhabdus, Pseudomonas, Serratia, Vibrio, myxobacteria, cyanobacteria, Bacillus, and Paenibacillus.

Further preferably, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   wherein performing random mutagenesis of step d) comprises chemical random mutagenesis, or UV-mediated random mutagenesis, or error-prone PCR or transposon mutagenesis;
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces.

Preferably, the bacterium is selected from the group comprising Xenorhabdus, Photorhabdus, Pseudomonas, Serratia, Vibrio, myxobacteria, cyanobacteria, Bacillus, and Paenibacillus.

Further more preferably, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   wherein the step e) is performed by fluorescence activated cell sorting, fluorescence spectroscopy, stereo microscopy, or fluorescence imaging;
   wherein performing random mutagenesis of step d) comprises chemical random mutagenesis, or UV-mediated random mutagenesis, or error-prone PCR or transposon mutagenesis;
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces.

Preferably, the bacterium is selected from the group comprising Xenorhabdus, Photorhabdus, Pseudomonas, Serratia, Vibrio, myxobacteria, cyanobacteria, Bacillus, and Paenibacillus.

The present invention also relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
   wherein the replacing of a gene sequence of a BGC at step a) and b) does not disrupt a regulatory element of said BGC;
   wherein in steps a) and b) the reading frame is conserved;
   wherein said GR gene regulates production of a target secondary metabolite;
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   wherein performing random mutagenesis of step d) comprises chemical random mutagenesis, or UV-mediated random mutagenesis, or error-prone PCR or transposon mutagenesis;
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces.

Preferably, the bacterium is selected from the group comprising Xenorhabdus, Photorhabdus, Pseudomonas, Serratia, Vibrio, myxobacteria, cyanobacteria, Bacillus, and Paenibacillus.

The present invention particularly relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
   wherein the replacing of a gene sequence of a BGC at step a) and b) does not disrupt a regulatory element of said BGC;
   wherein in steps a) and b) the reading frame is conserved;
   wherein said GR gene regulates production of a target secondary metabolite,
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   wherein the step e) is performed by fluorescence activated cell sorting, fluorescence spectroscopy, stereo microscopy, or fluorescence imaging; wherein performing random mutagenesis of step d) comprises chemical random mutagenesis, or UV-mediated random mutagenesis, or error-prone PCR or transposon mutagenesis,
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces.

Preferably, the bacterium is selected from the group comprising Xenorhabdus, Photorhabdus, Pseudomonas, Serratia, Vibrio, myxobacteria, cyanobacteria, Bacillus, and Paenibacillus.

### CRISPR/Cas Based Gene Editing

CRISPR-Cas systems are native to bacteria and Archaea and provide adaptive immunity against viruses and plasmids. The CRISPR-Cas endonuclease system is utilized in genomic engineering as follows: the gRNA complex (either a crRNA:tracrRNA complex or an sgRNA) binds to Cas9, inducing a conformational change that activates Cas9 and opens the DNA binding cleft, the protospacer domain of the crRNA (or sgRNA) aligns with the complementary target DNA and Cas9 binds the PAM sequence, initiating unwinding of the target DNA followed by annealing of the protospacer domain to the target, after which cleavage of the target DNA occurs. The Cas9 contains two domains, homologous to endonucleases HNH and RuvC respectively, wherein the HNH domain cleaves the DNA strand complementary to the crRNA and the RuvC-like domain cleaves the non-complementary strand. This results in a double-stranded break in the genomic DNA. When repaired by non-homologous end joining (NHEJ) the break is typically repaired in an imprecise fashion, resulting in the DNA sequence being shifted by 1 or more bases, leading to disruption of the natural DNA sequence and, in many cases, leading to a frameshift mutation if the event occurs in a coding exon of a protein-encoding gene. The break may also be repaired by homology directed recombination (HDR), which permits insertion of new genetic material based upon exogenous DNA introduced into the cell with the Cas9/gRNA complex, which is introduced into the cut site created by Cas9 cleavage

A second class 2 CRISPR system, assigned to type V, has been identified. This type V CRISPR-associated system contains Cpf1, which is a ^{~}1300 amino acid protein—slightly smaller than Cas9 from S. pyogenes. The PAM recognition sequence of Cpf1 is TTTN, in contrast to the NGG PAM recognition domain of S. pyogenes Cas9. Having the ability to target AT-rich areas of the genome will be greatly beneficial to manipulate and study gene targets in regions that are lacking GG dinucleotide motifs. The Cpf1 system is also remarkably simple in that it does not utilize a separate tracrRNA, and only requires a single short crRNA of 40-45 base length that both specifies target DNA sequence and directs binding of the RNA to the Cpf1 nuclease.

In contrast to Cas9 which produces blunt-ended cleavage products, Cpf1 facilitates double stranded breaks with 4-5 nucleotide overhangs. The advantage of this is that it may ensure proper orientation as well as providing microhomology during non-homologous end joining (NHEJ). This could also be advantageous in non-dividing cell types that tend to be resistant to homology-directed repair (HDR). Furthermore, when Cpf1 cleaves, it does so further away from PAM than Cas9, which is also further away from the target site. As a result, the protospacer, and especially the seed sequence of the protospacer, are less likely to be edited, thereby leaving open the potential for a second round of cleavage if the desired repair event doesn't happen the first time.

The Cpf1 protein forms a complex with a single stranded RNA oligonucleotide to mediate targeted DNA cleavage. The single strand guide RNA oligonucleotide consists of a constant region of 20 nt and a target region of 21-24 nt for an overall length of 41-44 nt.

A further suitable Cas for the present invention is Cas13.

The term “crRNA framework” (crRNA FW) refers to a nucleotide sequence comprising a constitutive or inducible promoter, a crRNA leader, direct repeat, a spacer dummy (comprising a Bsal or Bsmbl or any other type II restriction sites for insertion of target spacers A and B), direct repeat, a terminator. Suitable crRNA frameworks for the present invention are shown in FIG. 19.

The term “crRNA array” refers to a group of nucleotide sequences comprising:
a crRNA framework;
“target” specific target spacers A and/or B inserted inside said crRNA framework;
optionally further elements such as promoter system as defined herein, translational enhancer, fluorescent reporter gene.

Thus, a crRNA array is target specific. The target specific crRNA array is formed in the plasmid for gene editing from the elements of the homology arms left and right. The “target” can be for example a gene of a BGC for BGC activation, or a gene of a BGC for silencing or replacement with a FR gene, or a GR for GR deletion.

The term “crRNA system” refers to a group of dsDNA fragments (dsDNA nucleotide sequences) comprising:
at least one crRNA framework,
at least one target spacer A or B,
target specific homology region left,
target specific homology region right, and
optionally a fluorescent reporter gene, or a promoter system or enhancer sequence positioned between said homology region left and said homology region right.

A “crRNA leader” sequence can be an AT-rich sequence, but can also be part of the UTR of the promoter, so it is not necessary as an independent component.

A “direct repeat” refers to a 36 bp long direct repeat that is an essential part of the crRNA framework and must be encoded upstream of any target specific spacer to be recognized by the Cas protein, such as Cpf1. A repeat after the spacer is not necessary if the spacer has already been shortened to the mature length of 23 bp.

The term “target spacer” refers to a nucleic acid sequence having the function of “target specific crRNA”. A “target spacer” is selected from the genome of the target organism and is located distally after a PAM sequence (in the best-case TTTV).

Best editing results can be achieved selecting one spacer for each leading and lagging strand, i.e. target spacer A and target spacer B. It should be avoided to have three or more “T” in the sequence. A GC content of 50% should be aimed at. moreover, a “target spacer” has a nucleic acid sequence with a length between 23 and 31 bp depending on whether there is a direct repeat after the target spacer.

A “spacer dummy” refers to a polynucleotide sequence which should not have homology to the target host; and must be accessible for any kind of cloning (Gibson, Golden Gate, Gateway, Restriction cloning).

A “spacer dummy” can comprise a reporter gene (e.g. mCherry) or a toxin (e.g. ccdB) to allow identification of the cloning success;

A “terminator” or “transcriptional terminator” refers to a target host adapted terminator or standard terminator from the iGEM library (http://parts.igem.org/Terminators/Catalog).

The term “homology arm left” (HA-L) refers to a synthetic dsDNA fragment comprising the following elements in this order: spacer with restriction site (e.g. Bsal site), target spacer A or B (TS-A or TS-B), direct repeat (DR), optionally a terminator, homology region left (HR-L), spacer with restriction site (e.g. Bsal site). The target spacer A or B and DR form the “target specific crRNA array” in the assembled single plasmid, e.g. pAR20 in the Examples of this invention.

The term “homology arm right” (HA-R) refers to a synthetic dsDNA fragment comprising the following elements in this order: spacer with restriction site (e.g. Bsal site), homology region right (HR-R), a constitutive promoter (e.g. J23119), direct repeat (DR), target spacer A or B (TS-A or TS-B), spacer with restriction site (e.g. Bsal site). The constitutive promoter, crRNA leader, DR, and target spacer form the “target specific crRNA array” in the assembled single plasmid, e.g. pAR20 in the Examples of this invention.

The term “homology region” left or right refers to a nucleic acid sequence which is contained in the single plasmid for CRISPR/Cas gene editing and is complementary to the target BGC or global regulatory gene. It is used for repair after CRISPR induced double strand break. A nucleic acid sequence for “homology region” left or right depends on the recombination genes and the used strains. A nucleic acid sequence for “homology region” left or right can comprise at least 50, 100, 200, 300, 400, 500, 600, 700, 800, 900, 1000 bp (basepair). A nucleic acid sequence for “homology region” left or right can comprise at most 100, 200, 300, 400, 500, 600, 700, 800, 900, 1000, 1200, 1400, 1600, 1700, 1800, 1900, 2000 bp. A nucleic acid sequence for “homology region” left or right preferably comprises between 100 and 500 bp, 100 and 600 bp, 100 and 700 bp, 100 and 800 bp, 100 and 900 bp, 100 and 1000 bp, 100 and 1100 bp, 100 and 1200 bp, 100 and 1300 bp, 100 and 1400 bp, 100 and 1500 bp, 100 and 1600 bp, 100 and 1700 bp, 100 and 1800 bp, 100 and 1900 bp, 100 and 2000 bp, 50 and 500 bp, 50 and 600 bp, 50 and 700 bp, 50 and 800 bp, 50 and 900 bp, 50 and 1000 bp, 50 and 1100 bp, 50 and 1200 bp, 50 and 1300 bp, 50 and 1400 bp, 50 and 1500 bp, 50 and 1600 bp, 50 and 1700 bp, 50 and 1800 bp, 50 and 1900 bp, 50 and 2000 bp,

The present invention alternatively relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
   wherein the replacing of a gene sequence of a BGC at step a) and b) does not disrupt a regulatory element of said BGC;
   wherein in steps a) and b) the reading frame is conserved;
   wherein said GR gene regulates production of a target secondary metabolite;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

More in particular, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
   wherein the replacing of a gene sequence of a BGC at step a) and b) does not disrupt a regulatory element of said BGC;
   wherein in steps a) and b) the reading frame is conserved;
   wherein said GR gene regulates production of a target secondary metabolite,
   wherein the step e) is performed by fluorescence activated cell sorting, fluorescence spectroscopy, stereo microscopy, or fluorescence imaging;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

In other embodiments, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

In particular embodiments, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
   wherein the replacing of a gene sequence of a BGC at step a) and b) does not disrupt a regulatory element of said BGC;
   wherein in steps a) and b) the reading frame is conserved;
   wherein said GR gene regulates production of a target secondary metabolite,
   wherein the step e) is performed by fluorescence activated cell sorting, fluorescence spectroscopy, stereo microscopy, or fluorescence imaging;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

In particular embodiments, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
   wherein the replacing of a gene sequence of a BGC at step a) and b) does not disrupt a regulatory element of said BGC;
   wherein in steps a) and b) the reading frame is conserved;
   wherein said GR gene regulates production of a target secondary metabolite;
   wherein performing random mutagenesis of step d) comprises chemical random mutagenesis, or UV-mediated random mutagenesis, or error-prone PCR or transposon mutagenesis;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

In preferred embodiments, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
   wherein the replacing of a gene sequence of a BGC at step a) and b) does not disrupt a regulatory element of said BGC;
   wherein in steps a) and b) the reading frame is conserved;
   wherein said GR gene regulates production of a target secondary metabolite,
   wherein the step e) is performed by fluorescence activated cell sorting, fluorescence spectroscopy, stereo microscopy, or fluorescence imaging,
   wherein performing random mutagenesis of step d) comprises chemical random mutagenesis, or UV-mediated random mutagenesis, or error-prone PCR or transposon mutagenesis,
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

In more preferred embodiments, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
   wherein the replacing of a gene sequence of a BGC at step a) and b) does not disrupt a regulatory element of said BGC;
   wherein in steps a) and b) the reading frame is conserved;
   wherein said GR gene regulates production of a target secondary metabolite;
   wherein performing random mutagenesis of step d) comprises chemical random mutagenesis, or UV-mediated random mutagenesis, or error-prone PCR or transposon mutagenesis;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

In alternative embodiments, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
   wherein the replacing of a gene sequence of a BGC at step a) and b) does not disrupt a regulatory element of said BGC;
   wherein in steps a) and b) the reading frame is conserved;
   wherein said GR gene regulates production of a target secondary metabolite,
   wherein the step e) is performed by fluorescence activated cell sorting, fluorescence spectroscopy, stereo microscopy, or fluorescence imaging;
   wherein performing random mutagenesis of step d) comprises chemical random mutagenesis, or UV-mediated random mutagenesis, or error-prone PCR or transposon mutagenesis;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

In further embodiments, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

More in particular, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the step e) is performed by fluorescence activated cell sorting, fluorescence spectroscopy, stereo microscopy, or fluorescence imaging;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

The present invention further relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

The present invention also relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the step e) is performed by fluorescence activated cell sorting, fluorescence spectroscopy, stereo microscopy, or fluorescence imaging;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

The present invention is also directed to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein performing random mutagenesis of step d) comprises chemical random mutagenesis, or UV-mediated random mutagenesis, or error-prone PCR or transposon mutagenesis;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

More in particular, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the step e) is performed by fluorescence activated cell sorting, fluorescence spectroscopy, stereo microscopy, or fluorescence imaging,
   wherein performing random mutagenesis of step d) comprises chemical random mutagenesis, or UV-mediated random mutagenesis, or error-prone PCR or transposon mutagenesis;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

The present invention preferably relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
   wherein the replacing of a gene sequence of a BGC at step a) and b) does not disrupt a regulatory element of said BGC;
   wherein in steps a) and b) the reading frame is conserved;
   wherein said GR gene regulates production of a target secondary metabolite;
   wherein performing random mutagenesis of step d) comprises chemical random mutagenesis, or UV-mediated random mutagenesis, or error-prone PCR or transposon mutagenesis;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

The present invention more preferably relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
   wherein the replacing of a gene sequence of a BGC at step a) and b) does not disrupt a regulatory element of said BGC;
   wherein in steps a) and b) the reading frame is conserved;
   wherein said GR gene regulates production of a target secondary metabolite,
   wherein the step e) is performed by fluorescence activated cell sorting, fluorescence spectroscopy, stereo microscopy, or fluorescence imaging;
   wherein performing random mutagenesis of step d) comprises chemical random mutagenesis, or UV-mediated random mutagenesis, or error-prone PCR or transposon mutagenesis;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

The present invention still more preferably relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

The present invention further more preferably relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the step e) is performed by fluorescence activated cell sorting, fluorescence spectroscopy, stereo microscopy, or fluorescence imaging;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

The present invention also preferentially relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

The present invention more preferentially relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein the step e) is performed by fluorescence activated cell sorting, fluorescence spectroscopy, stereo microscopy, or fluorescence imaging;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

The present invention more preferentially relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein performing random mutagenesis of step d) comprises chemical random mutagenesis, or UV-mediated random mutagenesis, or error-prone PCR or transposon mutagenesis;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

The present invention still more preferentially relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein the step e) is performed by fluorescence activated cell sorting, fluorescence spectroscopy, stereo microscopy, or fluorescence imaging,
   wherein performing random mutagenesis of step d) comprises chemical random mutagenesis, or UV-mediated random mutagenesis, or error-prone PCR or transposon mutagenesis;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

Thus, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
   wherein the replacing of a gene sequence of a BGC at step a) and b) does not disrupt a regulatory element of said BGC;
   wherein in steps a) and b) the reading frame is conserved;
   wherein said GR gene regulates production of a target secondary metabolite;
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein performing random mutagenesis of step d) comprises chemical random mutagenesis, or UV-mediated random mutagenesis, or error-prone PCR or transposon mutagenesis;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

In a particular aspect, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
   wherein the replacing of a gene sequence of a BGC at step a) and b) does not disrupt a regulatory element of said BGC;
   wherein in steps a) and b) the reading frame is conserved;
   wherein said GR gene regulates production of a target secondary metabolite,
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein the step e) is performed by fluorescence activated cell sorting, fluorescence spectroscopy, stereo microscopy, or fluorescence imaging;
   wherein performing random mutagenesis of step d) comprises chemical random mutagenesis, or UV-mediated random mutagenesis, or error-prone PCR or transposon mutagenesis;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

Thus, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

More in particular, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   wherein the step e) is performed by fluorescence activated cell sorting, fluorescence spectroscopy, stereo microscopy, or fluorescence imaging;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

Thus, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

More in particular, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   wherein the step e) is performed by fluorescence activated cell sorting, fluorescence spectroscopy, stereo microscopy, or fluorescence imaging;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

In an aspect, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   wherein performing random mutagenesis of step d) comprises chemical random mutagenesis, or UV-mediated random mutagenesis, or error-prone PCR or transposon mutagenesis;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

More in particular, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
   wherein the replacing of a gene sequence of a BGC at step a) and b) does not disrupt a regulatory element of said BGC;
   wherein in steps a) and b) the reading frame is conserved;
   wherein said GR gene regulates production of a target secondary metabolite,
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   wherein the step e) is performed by fluorescence activated cell sorting, fluorescence spectroscopy, stereo microscopy, or fluorescence imaging;
   wherein performing random mutagenesis of step d) comprises chemical random mutagenesis, or UV-mediated random mutagenesis, or error-prone PCR or transposon mutagenesis;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

Moreover, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
   wherein the replacing of a gene sequence of a BGC at step a) and b) does not disrupt a regulatory element of said BGC;
   wherein in steps a) and b) the reading frame is conserved;
   wherein said GR gene regulates production of a target secondary metabolite;
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   wherein performing random mutagenesis of step d) comprises chemical random mutagenesis, or UV-mediated random mutagenesis, or error-prone PCR or transposon mutagenesis;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

More in particular, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
   wherein the replacing of a gene sequence of a BGC at step a) and b) does not disrupt a regulatory element of said BGC;
   wherein in steps a) and b) the reading frame is conserved;
   wherein said GR gene regulates production of a target secondary metabolite,
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   wherein the step e) is performed by fluorescence activated cell sorting, fluorescence spectroscopy, stereo microscopy, or fluorescence imaging;
   wherein performing random mutagenesis of step d) comprises chemical random mutagenesis, or UV-mediated random mutagenesis, or error-prone PCR or transposon mutagenesis;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

Thus, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein the bacterium is selected from the group comprising Xenorhabdus, Photorhabdus, Pseudomonas, Serratia, Vibrio, myxobacteria, cyanobacteria, Bacillus, Paenibacillus and Streptomyces;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

More in particular, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the step e) is performed by fluorescence activated cell sorting, fluorescence spectroscopy, stereo microscopy, or fluorescence imaging;
   wherein the bacterium is selected from the group comprising Xenorhabdus, Photorhabdus, Pseudomonas, Serratia, Vibrio, myxobacteria, cyanobacteria, Bacillus, Paenibacillus and Streptomyces;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

Thus, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein the bacterium is selected from the group comprising Xenorhabdus, Photorhabdus, Pseudomonas, Serratia, Vibrio, myxobacteria, cyanobacteria, Bacillus, Paenibacillus and Streptomyces;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

More in particular, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the step e) is performed by fluorescence activated cell sorting, fluorescence spectroscopy, stereo microscopy, or fluorescence imaging;
   wherein the bacterium is selected from the group comprising Xenorhabdus, Photorhabdus, Pseudomonas, Serratia, Vibrio, myxobacteria, cyanobacteria, Bacillus, Paenibacillus and Streptomyces;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

Thus, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein performing random mutagenesis of step d) comprises chemical random mutagenesis, or UV-mediated random mutagenesis, or error-prone PCR or transposon mutagenesis;
   wherein the bacterium is selected from the group comprising Xenorhabdus, Photorhabdus, Pseudomonas, Serratia, Vibrio, myxobacteria, cyanobacteria, Bacillus, Paenibacillus and Streptomyces;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

More in particular, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the step e) is performed by fluorescence activated cell sorting, fluorescence spectroscopy, stereo microscopy, or fluorescence imaging, wherein performing random mutagenesis of step d) comprises chemical random mutagenesis, or UV-mediated random mutagenesis, or error-prone PCR or transposon mutagenesis;
   wherein the bacterium is selected from the group comprising Xenorhabdus, Photorhabdus, Pseudomonas, Serratia, Vibrio, myxobacteria, cyanobacteria, Bacillus, Paenibacillus and Streptomyces;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

In a further more particular embodiment, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
   wherein the replacing of a gene sequence of a BGC at step a) and b) does not disrupt a regulatory element of said BGC;
   wherein in steps a) and b) the reading frame is conserved;
   wherein said GR gene regulates production of a target secondary metabolite;
   wherein performing random mutagenesis of step d) comprises chemical random mutagenesis, or UV-mediated random mutagenesis, or error-prone PCR or transposon mutagenesis;
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

In a still more particular embodiment, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
   wherein the replacing of a gene sequence of a BGC at step a) and b) does not disrupt a regulatory element of said BGC;
   wherein in steps a) and b) the reading frame is conserved;
   wherein said GR gene regulates production of a target secondary metabolite,
   wherein the step e) is performed by fluorescence activated cell sorting, fluorescence spectroscopy, stereo microscopy, or fluorescence imaging;
   wherein performing random mutagenesis of step d) comprises chemical random mutagenesis, or UV-mediated random mutagenesis, or error-prone PCR or transposon mutagenesis;
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

In a more particular embodiment, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

In a more particular embodiment, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the step e) is performed by fluorescence activated cell sorting, fluorescence spectroscopy, stereo microscopy, or fluorescence imaging;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

In a particular embodiment, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

In a particular aspect, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the step e) is performed by fluorescence activated cell sorting, fluorescence spectroscopy, stereo microscopy, or fluorescence imaging;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

Moreover, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein performing random mutagenesis of step d) comprises chemical random mutagenesis, or UV-mediated random mutagenesis, or error-prone PCR or transposon mutagenesis;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

In other particular embodiments, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the step e) is performed by fluorescence activated cell sorting, fluorescence spectroscopy, stereo microscopy, or fluorescence imaging,
   wherein performing random mutagenesis of step d) comprises chemical random mutagenesis, or UV-mediated random mutagenesis, or error-prone PCR or transposon mutagenesis;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

In other alternative embodiments, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
   wherein the replacing of a gene sequence of a BGC at step a) and b) does not disrupt a regulatory element of said BGC;
   wherein in steps a) and b) the reading frame is conserved;
   wherein said GR gene regulates production of a target secondary metabolite;
   wherein performing random mutagenesis of step d) comprises chemical random mutagenesis, or UV-mediated random mutagenesis, or error-prone PCR or transposon mutagenesis;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

In other particular embodiments, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
   wherein the replacing of a gene sequence of a BGC at step a) and b) does not disrupt a regulatory element of said BGC;
   wherein in steps a) and b) the reading frame is conserved;
   wherein said GR gene regulates production of a target secondary metabolite,
   wherein the step e) is performed by fluorescence activated cell sorting, fluorescence spectroscopy, stereo microscopy, or fluorescence imaging;
   wherein performing random mutagenesis of step d) comprises chemical random mutagenesis, or UV-mediated random mutagenesis, or error-prone PCR or transposon mutagenesis;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

In other preferred embodiments, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives,
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

In other embodiments, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the step e) is performed by fluorescence activated cell sorting, fluorescence spectroscopy, stereo microscopy, or fluorescence imaging, and wherein the bacterium is selected from the group comprising Xenorhabdus, Photorhabdus, Pseudomonas, Serratia, Vibrio, myxobacteria, cyanobacteria, Bacillus, Paenibacillus and Streptomyces;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

A further aspect of present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives,
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

A still more particular aspect of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein the step e) is performed by fluorescence activated cell sorting, fluorescence spectroscopy, stereo microscopy, or fluorescence imaging;
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

A more particular aspect of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein performing random mutagenesis of step d) comprises chemical random mutagenesis, or UV-mediated random mutagenesis, or error-prone PCR or transposon mutagenesis;
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

A particular aspect of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein the step e) is performed by fluorescence activated cell sorting, fluorescence spectroscopy, stereo microscopy, or fluorescence imaging,
   wherein performing random mutagenesis of step d) comprises chemical random mutagenesis, or UV-mediated random mutagenesis, or error-prone PCR or transposon mutagenesis;
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

A particular embodiment of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
   wherein the replacing of a gene sequence of a BGC at step a) and b) does not disrupt a regulatory element of said BGC;
   wherein in steps a) and b) the reading frame is conserved;
   wherein said GR gene regulates production of a target secondary metabolite;
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein performing random mutagenesis of step d) comprises chemical random mutagenesis, or UV-mediated random mutagenesis, or error-prone PCR or transposon mutagenesis;
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

An alternative embodiment of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
   wherein the replacing of a gene sequence of a BGC at step a) and b) does not disrupt a regulatory element of said BGC;
   wherein in steps a) and b) the reading frame is conserved;
   wherein said GR gene regulates production of a target secondary metabolite,
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein the step e) is performed by fluorescence activated cell sorting, fluorescence spectroscopy, stereo microscopy, or fluorescence imaging;
   wherein performing random mutagenesis of step d) comprises chemical random mutagenesis, or UV-mediated random mutagenesis, or error-prone PCR or transposon mutagenesis;
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

A preferred embodiment of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

A particular embodiment of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   wherein the step e) is performed by fluorescence activated cell sorting, fluorescence spectroscopy, stereo microscopy, or fluorescence imaging;
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

An embodiment of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

More in particular, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   wherein the step e) is performed by fluorescence activated cell sorting, fluorescence spectroscopy, stereo microscopy, or fluorescence imaging; and wherein the bacterium is selected from the group comprising Xenorhabdus, Photorhabdus, Pseudomonas, Serratia, Vibrio, myxobacteria, cyanobacteria, Bacillus, Paenibacillus and Streptomyces;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

Thus, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite;
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   wherein performing random mutagenesis of step d) comprises chemical random mutagenesis, or UV-mediated random mutagenesis, or error-prone PCR or transposon mutagenesis;
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

More in particular, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in steps a) and b) the reading frame is conserved;
   wherein said GR gene regulates production of a target secondary metabolite,
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   wherein the step e) is performed by fluorescence activated cell sorting, fluorescence spectroscopy, stereo microscopy, or fluorescence imaging; wherein performing random mutagenesis of step d) comprises chemical random mutagenesis, or UV-mediated random mutagenesis, or error-prone PCR or transposon mutagenesis;
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

A particular embodiment of the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
   wherein the replacing of a gene sequence of a BGC at step a) and b) does not disrupt a regulatory element of said BGC;
   wherein in steps a) and b) the reading frame is conserved;
   wherein said GR gene regulates production of a target secondary metabolite;
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   wherein performing random mutagenesis of step d) comprises chemical random mutagenesis, or UV-mediated random mutagenesis, or error-prone PCR or transposon mutagenesis;
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

More in particular, the present invention relates to a method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
i) screening for production of secondary metabolites regulated by said GR gene by HPLC/mass spectrometry analysis;
   wherein the replacing of a gene sequence of a BGC at step a) and b) does not disrupt a regulatory element of said BGC;
   wherein in steps a) and b) the reading frame is conserved;
   wherein said GR gene regulates production of a target secondary metabolite,
   wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives;
   wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP);
   wherein the step e) is performed by fluorescence activated cell sorting, fluorescence spectroscopy, stereo microscopy, or fluorescence imaging;
   wherein performing random mutagenesis of step d) comprises chemical random mutagenesis, or UV-mediated random mutagenesis, or error-prone PCR or transposon mutagenesis,
   and wherein the bacterium is selected from the group comprising Xenorhabdus, Photorhabdus, Pseudomonas, Serratia, Vibrio, myxobacteria, cyanobacteria, Bacillus, Paenibacillus and Streptomyces;
   wherein the steps a) and b) are performed introducing in said bacterium a Cas gene, a first and second constitutively expressed BGC specific crRNA array, a first and second fluorescent reporter gene, and a first and second pair of BGC specific homology regions left and right.

### “Plasmids”

A “helper plasmid” is a plasmid for a bacterium that provides functions required for efficient recombination. For CRISPR/Cas system, an helper plasmid contains the gene encoding the endonuclease Cas12a (Cpf1) under an inducible promoter, and a lambda red (gam, exo, bet) recombination system under an inducible promoter.

A “donor plasmid” is a plasmid carrying a crRNA sequence for a specific target gene under a constitutive promoter or a strong constitutive promoter or an inducible promoter, as well as homology regions left and right to the target gene serving as repair templates after double strand break.

“Promoter” refers to genetic elements controlling the binding of RNA polymerase and transcription factors. Since the promoter region drives transcription of a target gene, it therefore determines the timing of gene expression and largely defines the amount of recombinant protein that will be produced.

A promoter can be specific for a species or genus, but often a promoter from a bacterium can function well in a distantly related bacterium. For example, a promoter from Bacillus subtilis or a phage that normally grows on B. subtilis can function well in E. coli.

“Constitutive Promoter” Refers to Promoters that are Always Active. Suitable Examples are rpsL (30S Ribosomal Subunit Protein S12 (V00355.1)), Amp (bla, BBa_I14018) promoter

Suitable promoters to carry out the methods described herein are disclosed in Shimada T. et al., The Whole Set of Constitutive Promoters Recognized by RNA Polymerase RpoD Holoenzyme of Escherichia coli. PLOS ONE 2014, 9 (3): e90447.

Further constitutive artificial promoter can be found in Anderson library: ‘Promoters/Catalog/Anderson’, http://parts.igem.org/Promoters/Catalog/Anderson. Promoters from primary metabolism or from ribosomal proteins can be used herein, as for example: glyceraldehyde-3-phosphate dehydrogenase promoter (GenBank: CP097883.1) or ribosomal protein S12 promoter (GenBank: CP097884.1).

“Strong constitutive promoter” are for examples the strong constitutive from Bacillus subtilis phage SP01, and coliphage lambda PR, and PJ23119 from the Anderson library.

“Inducible promoters” refer to promoters that are only active under specific circumstances and which can be switched from an OFF to an ON state, or from an ON to an OFF state.

Inducible promoters suitable for expression of cpf1 and recombinases in the present invention are AraC-P_{BAD} (Meyer A J 2019, Nature Chemical Biology 15, 196-204), Lacl-P_{tac} (Meyer A J 2019), the xylose-inducible expression system: xutR-Pxut, Genbank MT344942, and MN857504, and the L-rhamnose-inducible promoter from Burkholderia, PrhaBAD, controlled by AraC/XyIS-family transcriptional regulators rhaS and rhaR (Hogan A M et al., 2021. Appl Environ Microbiol 87: e00647-21).

Inducible promoter systems have been described in the prior art (e.g. in Meyer A J 2019), and comprise the following elements: a 3′-Terminator, a repressor/regulator gene (Table 6), a RBS specific for the regulator (Table 6), a strong constitutive promotor for expression of the regulator (Table 6), a terminator to separate regulator regulation from target gene regulation: (ex.: L3S2P21), a Insulator element (examples listed below), a strong RBS for target gene, such as BBa_B0064.

The vanillic Acid promoter system (SEQ ID NO 131, 132) used in the examples of the present invention comprises the following elements: PvanCC modified (Promoter), BBa_B0064 (RBS), RiboJ (Insulator), RiboJ51 (Insulator).

The expression of a gene cluster can be increased by additionally introducing “enhancer sequences” such as a T7g10 transcriptional enhancer, or a “translation enhancement by a Dictyostelium gene sequence”, TED sequence. The transcriptional enhancer T7g10 (also abbreviated as g10T7 UTR), (SEQ ID NO: 135, comprising BBa_K1758100, sequence available online) comprises a 5′ untranslated region (5′-UTR) and a strong ribosomal binding site from bacteriophage T7 gene 10 (g10-L).

The TED sequence derived by a Dictyostelium has to be placed upstream of the Shine-Dalgarno sequence located between the promoter and the initiation codon of a target gene in order to achieve an enhancement in gene expression. Suitable TED sequences are mlcR sequences, such as mlcR10, mlcR20, mlcR25, mlcR30, mlcR40, mlcR45, mlcR60, and mlcR74, previously described by Kondo and Yumura 2019, Applied Microbiology and Biotechnology 103:3501-3510.

Further enhancement sequences and structures has been described in Xiao et al., ACS Synth. Biol., 9, 1051-1058

The wording “refactoring” refers to the manipulation of a BGC cluster comprising: replacing natural promoters with constitutive or readily inducible promoters and heterologous expression in strains optimized for heterologous expression. BGC refactoring has thus the potential to provide access to the chemical diversity encoded by silent BGCs.

TABLE 1
Elements of the suitable promoter systems for promoter exchange

| | Regulator | Output | Promoter | | | |
|---|---|---|---|---|---|---|
| Inducer | (R) | Promoter | for R | RBS | Notes | Ref. |
| DAPG, | PhlF^{AM} | P_{PhlF} | P_{J23119} | phl2 | PhlF is a | Meyer |
| 2,4- | | | | | TetR family | A J |
| (diacetylpho- | | | | | repressor | 2019 |
| phloroglucinol) | | | | | from | |
| | | | | | P. fluorescens | |
| Cumic acid | CymR^{AM} | P_{CymRC} | P_{J23119} | cym1 | CymR is a | Meyer |
| | | | | | TetR family | A J |
| | | | | | repressor | 2019 |
| | | | | | from P. putida | |
| OC6 | luxR | P_{luxB} | P_{J23119} | lux1 | LuxR is an | Meyer |
| | | | | | activator from | A J |
| | | | | | Vibrio fischeri | 2019 |
| Van | VanR^{AM} | P_{VanCC} | P_{J23119} | van2 | VanR is a | Meyer |
| (vanillic | | | | | GntR family | A J |
| acid) | | | | | repressor | 2019 |
| | | | | | from | |
| | | | | | Caulobacter | |
| | | | | | crescentus | |
| IPTG | LacI^{AM} | P_{Tac} | P_{LacIR} | lac1 | LaI is a | Meyer |
| | | | | | repressor | A J |
| | | | | | from E. coli. | 2019 |
| aTc | tetR | Pₜₑₜ | P_{LacIR} | tet1 | TetR is a | Meyer |
| | | | | | repressor | A J |
| | | | | | from E. coli | 2019 |
| Ara | AraC^{AM}, | P_{BAD} | P_{LacIR} | ara1, | AraC is | Meyer |
| (L- | araE | | | e1 | activator/repressor | A J |
| arabinose) | | | | | from E. coli. | 2019 |
| Cho | BetI^{AM} | P_{BetI} | P_{J23100} | bet2 | BetI is a TetR | Meyer |
| (choline | | | | | family | A J |
| chloride) | | | | | repressor | 2019 |
| | | | | | from E. coli. | |
| Nar | TtgR^{AM} | P_{Ttg} | P_{J23100} | ttg2 | TtgR is a | Meyer |
| (naringenin) | | | | | TetR family | A J |
| | | | | | repressor | 2019 |
| | | | | | from P. | |
| | | | | | putida | |
| DHBA | PcaU^{AM} | P_{3B5B} | P_{J23100} | pca2 | PcaU is a | Meyer |
| (3,4- | | | | | IclR family | A J |
| dihydroxybenzoic | | | | | repressor from | 2019 |
| acid) | | | | | Acinetobacter sp. | |
| Sal | NahR^{AM} | P_{SaITTC} | P_{J23100} | nah2 | NahR is a | Meyer |
| (sodium | | | | | LysR family | A J |
| salicylate) | | | | | activator from | 2019 |
| | | | | | P. putida | |
| OHC14 | CinR^{AM} | P_{Cin} | P_{J23100} | cln1 | CinR is a | Meyer |
| (3- | | | | | LuxR family | A J |
| Hydroxytetr | | | | | activator from | 2019 |
| adecanoyl- | | | | | Rhizobium | |
| homoserine | | | | | leguminosarum | |
| lactone) | | | | | | |

Suitable Insulators are RiboJ, RiboJ10, RiboJ51, RiboJ53, BydvJ, ElvJ. (Meyer A J, 2019), LtsvJ, SccJ, SarJ, PlmJ, and VtmoJ (Lou et al., Nat Biotechnol 2012, 30 (11), 1137-42).
Suitable Terminators are B0053, ECK120017009, ECK120033736, ECK120033737, IOT, L3S2P21, L3S3P21, L3S2P56, L3S3P41, L3S1P52, L3S2P41. (Meyer A J, 2019).
The system of the Xylose inducible promotor PxylA comprises a modified 5-UTR containing g10-L RBS (RBS from gene 10 of the T7 phage 5), a strong ribosomal binding site from bacteriophage T7. This sequence greatly enhances translation of a following gene. The sequence can be found at https://parts.igem.org/Part:BBa_K1758100.

### Suitable Recombinase Systems:

Recombineering system based on three Rac bacteriophage RecET-like operons: RecEThe_{BDU8}, RecETh_{TJI49} and RecETh1h2e_{YI23}, optionally combined with exonuclease inhibitors Pluγ or Redγ. RecEThe_{BDU8} from Burkholderia sp. BDU8 harbours a four-gene operon predicted to encode: the YqaJ viral recombinase family protein RecE_{BDU8} (protein ID: KVE53656.1; locus tag: WS71_06320); the recombinase RecT_{BDU8} (protein ID: KVE53655.1; locus tag: WS71_06315); and two hypothetical proteins h_{BDU8} (protein ID: KVE53654.1; locus tag: WS71_06310) and e_{BDU8} (protein ID: KVE53653.1; locus tag: WS71_06305). The operon, RecETh_{TJI49} from Burkholderia sp. TJI49, is predicted to encode the following three proteins: the hypothetical protein RecE_{TJI49} (protein ID: EGD06616.1; locus tag: B1M_00520),; the phage-related DNA recombination protein RecT_{TJI49} (protein ID: EGD06615.1; locus tag: B1M_00515); and the hypothetical protein h_{TJI49} (protein ID: EGD06614.1; locus tag: B1M_00510). The third operon, RecETh1h2e_{YI23} from Burkholderia cordobensis YI23, contains genes encoding the putative 5′-3′ specific dsDNA RecE_{YI23} exonuclease (protein ID: AET91062.1; locus tag: BYI23_B004550); the putative recombinase protein RecT_{Y123} (protein ID: AET91060.1; locus tag: BYI23_B004530); three hypothetical proteins h1_{YI23} (protein ID: AET91061.1; locus tag: BYI23_B004540), h2_{YI23} (protein ID: AET91063.1; locus tag: BYI23_B004560) and e_{Y123} (protein ID: AET91059.1; locus tag: BYI23_B004520). Ruijuan Li et al., Microb. Biotechnol. (2021) 14 (4), 1809-1826.

Pseudomonas endogenous phage recombinant systems BAS and RecET_{psy}, (Yin et al., iScience 14, 1-14, Apr. 26, 2019), E. coli Red and Plu Red recombinant proteins Redγβα and Pluγβα (Yin et al., 2019) Photorhabdus luminescens lambda Red-like operon, Plu2934/Plu2935/Plu2936, Jia Yin, et al., Nucleic Acids Research, 43 (6), 2015, e36.

A number of selectable marker genes are known in the art and several antibiotic resistance markers satisfy these criteria, including those conferring resistance to hygromycin, kanamycin, bleomycin, G418, streptomycin or spectinomycin, ampicillin, tetracycline, neomycin, Zeocin™, and the like.

A preferred negative selectable marker gene is SacB. SacB (Bacillus subtilis levansucrase) is a counter selectable marker which is lethal to E. coli cells in the presence of sucrose. SacB encoded levansucrase converts sucrose to levans, which is harmful to the bacteria, and allows plasmid selection on sucrose. The sacB gene including its native promoter can be amplified with AR432/433 from pEP17-KM. A further preferred negative selectable marker gene is mutated PheS, which encodes α-subunit of phenylalanyl tRNA synthase. Counterselection is with 0.1% p-chlorophenylalanine), GenBank EU329004.1.

### Single Plasmid for Gene Editing

A “single-plasmid” refers to a plasmid comprising elements of helper plasmid, donor plasmid, optionally repair template, and positive and negative selectable markers.

In detail, the present invention relates to the following types of plasmids for CRISPR/Cas based gene editing:

A single plasmid for replacement of a BGC with a fluorescent reporter (FIG. 1B) comprises the following sequences:
first crRNA array comprising a crRNA FW (J23119, DR, DR, Term) with a BGC specific TS-A inserted in the spacer dummy of the crRNA FW.
BGC specific HR-L complementary to a genome sequence positioned upstream said BGC gene;
Fluorescent Reporter gene (for example a gene encoding for a protein of Table 11)
BGC specific HR-R complementary to a genome sequence positioned downstream said BGC gene;
second crRNA array comprising a crRNA FW (J23119, DR, DR, Term) with a BGC specific TS-B inserted in the spacer dummy of the crRNA FW.

TS-A and TS-B are selected inside the target gene (GOI=gene of interest) of the BGC, at a variable distance between them.

A single plasmid for activation of a BGC by promoter exchange (FIG. 1C, 6A-C) comprises the following sequences:
first crRNA array comprising a crRNA FW (J23119, DR, DR, Term) with a BGC specific TS-A inserted in the spacer dummy of the crRNA FW.
BGC specific HR-L complementary to a genome sequence positioned upstream to said BGC gene;
Promoter system, e.g.: repressor with constitutive promoter and Terminator all in reverse direction; an Inducible Promoter;
BGC specific HR-R complementary to a genome sequence positioned inside said BGC gene;
second crRNA array comprising a crRNA FW (J23119, DR, DR, Term) with a BGC specific TS-B inserted in the spacer dummy of the crRNA FW.

Both TS-A and TS-B are situated in the genome before the target gene (GOI=gene of interest) of the BGC, at a variable distance between them.

A single plasmid for inactivation or deletion of a GR (Example 12; FIG. 2 shows the optimization process to obtain such a single plasmid) comprises the following sequences:
first crRNA array comprising a crRNA FW (J23119, DR, DR, Term) with a GR specific TS-A inserted in the spacer dummy of the crRNA FW.
GR specific HR-L complementary to a genome sequence positioned upstream the GR;
GR specific HR-R complementary to a genome sequence positioned downstream the GR;
second crRNA array comprising a crRNA FW (J23119, DR, DR, Term) with a GR specific TS-B inserted in the spacer dummy of the crRNA FW.

TA-A and TS-B are selected inside the target gene (GOI=gene of interest) of the GR, at a variable distance between them.

A preferred embodiment of the present invention is directed to a recombination single-plasmid to activate or silence a BGC in a bacterium by CRISPR/Cas-mediated homology-directed repair, the plasmid comprising:
an origin of transfer sequence (oriT),
a cas gene under control of a first inducible promoter,
genes for a suitable recombinase system under control of a second inducible promoter,
at least one positive selectable marker gene,
at least one negative selectable marker gene,
a crRNA system selected from the group consisting of:
I) a crRNA system to activate a BGC comprising:
   at least one crRNA framework,
   at least one target spacer specific for a genome sequence positioned upstream to a BGC gene,
   homology region left complementary to a genome sequence positioned upstream to said BGC gene,
   homology region right complementary to a genome sequence positioned inside said BGC gene, and
   a promoter system positioned between said homology region left and said homology region right, and optionally an enhancer sequence;
II) a crRNA system to silence a BGC or to delete or inactivate a GR comprising:
   at least one crRNA framework,
   at least one target spacer specific for a genome sequence positioned inside a BGC gene or inside said GR,
   homology region left complementary to a genome sequence positioned upstream said BGC gene or said GR,
   homology region right complementary to a genome sequence positioned downstream said BGC gene or said GR, and
   optionally a fluorescent reporter gene positioned between said homology region left and said homology region right.

A more preferred embodiment of the present invention is directed to a recombination single-plasmid to activate or silence a BGC in a bacterium by CRISPR/Cas-mediated homology-directed repair, the plasmid comprising:
an origin of transfer sequence (oriT),
a cas gene under control of a first inducible promoter,
genes for a suitable recombinase system under control of a second inducible promoter,
at least one positive selectable marker gene,
at least one negative selectable marker gene,
a crRNA system selected from the group consisting of:
I) a crRNA system to activate a BGC comprising:
   at least one crRNA framework,
   at least one target spacer specific for a genome sequence positioned upstream to a BGC gene,
   homology region left complementary to a genome sequence positioned upstream to said BGC gene,
   homology region right complementary to a genome sequence positioned inside said BGC gene, and
   a promoter system positioned between said homology region left and said homology region right, and optionally an enhancer sequence;
II) a crRNA system to silence a BGC or to delete or inactivate a GR comprising:
   at least one crRNA framework,
   at least one target spacer specific for a genome sequence positioned inside a BGC gene or inside said GR,
   homology region left complementary to a genome sequence positioned upstream said BGC gene or said GR,
   homology region right complementary to a genome sequence positioned downstream said BGC gene or said GR, and
   optionally a fluorescent reporter gene positioned between said homology region left and said homology region right; and
      wherein the Cas is selected from the group comprising Cas9, Cas12, and Cas13.

### Method to Elicit Production of a Secondary Metabolite

An embodiment of the present invention relates to a method to elicit production of a secondary metabolite from a BGC in a bacterium, wherein the method comprises the following steps:
i) deleting a GR gene in said bacterium, and
ii) optionally activating a BGC in said bacterium;
or wherein the method comprises the following steps:
i′) essentially activating a BGC in said bacterium, and
ii′) optionally deleting a GR gene in said bacterium;
   and the method further comprises:
iii) expressing in said bacterium at least one positive selectable marker
   gene and at least one negative selectable marker gene;
      wherein deleting a GR gene of steps i) or ii′) comprises introducing in said bacterium at least one GR specific siRNA, or a group of sequences comprising a Cas gene, at least one GR specific crRNA array, a pair of GR specific homology regions left and right; and/or
wherein activating a BGC of steps ii) or i′) comprises introducing in said bacterium a group of sequences comprising a Cas gene, at least one BGC specific crRNA array, a pair of BGC specific homology regions left and right, a promoter system, and optionally an enhancer sequence.

Reworded, the method to elicit production of a secondary metabolite by a bacterium, wherein the method comprises the following steps:
i) deleting a global regulator (GR) gene in said bacterium, and
ii) optionally activating a biosynthetic gene cluster (BGC) involved in production of said secondary metabolite in said bacterium;
   or wherein the method comprises the following steps:
i′) essentially activating a biosynthetic gene cluster (BGC) involved in production of said secondary metabolite in said bacterium, and
ii′) optionally deleting a GR gene in said bacterium;
   and the method further comprises:
iii) expressing in said bacterium at least one positive selectable marker gene and at least one negative selectable marker gene;
   wherein deleting a GR gene of steps i) or ii′) comprises introducing in said bacterium at least one GR specific siRNA, or a group of sequences comprising a Cas gene, at least one GR specific crRNA array, a pair of GR specific homology regions left and right; and/or
   wherein activating a BGC of steps ii) or i′) comprises introducing in said bacterium a group of sequences comprising a Cas gene, at least one BGC specific crRNA array, a pair of BGC specific homology regions left and right, a promoter system, and optionally an enhancer sequence.

A preferred embodiment of the present invention relates to a method to elicit production of a secondary metabolite from a BGC in a bacterium, wherein the method comprises the following steps:
i) deleting a GR gene in said bacterium, and
ii) optionally activating a BGC in said bacterium;
or wherein the method comprises the following steps:
i′) essentially activating a BGC in said bacterium, and
ii′) optionally deleting a GR gene in said bacterium;
and the method further comprises:
iii) expressing in said bacterium at least one positive selectable marker gene and at least one negative selectable marker gene;
wherein deleting a GR gene of steps i) or ii′) comprises introducing in said bacterium a group of sequences comprising a Cas gene, at least one GR specific crRNA array, a pair of GR specific homology regions left and right; and/or
wherein activating a BGC of steps ii) or i′) comprises introducing in said bacterium a group of sequences comprising a Cas gene, at least one BGC specific crRNA array, a pair of BGC specific homology regions left and right, a promoter system, and optionally an enhancer sequence.

A more preferred embodiment of the present invention relates to a method to elicit production of a secondary metabolite from a BGC in a bacterium, wherein the method comprises the following steps:
i) deleting a GR gene in said bacterium, and
ii) optionally activating a BGC in said bacterium;
or wherein the method comprises the following steps:
i′) essentially activating a BGC in said bacterium, and
ii′) optionally deleting a GR gene in said bacterium;
and the method further comprises:
iii) expressing in said bacterium at least one positive selectable marker gene and at least one negative selectable marker gene;
wherein deleting a GR gene of steps i) or ii′) comprises introducing in said bacterium at least one GR specific siRNA, or a group of sequences comprising a Cas gene, at least one GR specific crRNA array, a pair of GR specific homology regions left and right; and/or
wherein activating a BGC of steps ii) or i′) comprises introducing in said bacterium a group of sequences comprising a Cas gene, at least one BGC specific crRNA array, a pair of BGC specific homology regions left and right, a promoter system, and optionally an enhancer sequence;
wherein the Cas is selected from the group comprising Cas9, Cas12 and Cas13.

A further more preferred embodiment of the present invention relates to a method to elicit production of a secondary metabolite from a BGC in a bacterium, wherein the method comprises the following steps:
i) deleting a GR gene in said bacterium, and
ii) optionally activating at least 2 BGCs in said bacterium;
or wherein the method comprises the following steps:
i′) essentially activating at least 2 BGCs in said bacterium, and
ii′) optionally deleting a GR gene in said bacterium;
and the method further comprises:
iii) expressing in said bacterium at least one positive selectable marker gene and at least one negative selectable marker gene;
wherein deleting a GR gene of steps i) or ii′) comprises introducing in said bacterium at least one GR specific siRNA, or a group of sequences comprising a Cas gene, at least one GR specific crRNA array, a pair of GR specific homology regions left and right; and/or
wherein activating a BGC of steps ii) or i′) comprises introducing in said bacterium a group of sequences comprising a Cas gene, at least one BGC specific crRNA array, a pair of BGC specific homology regions left and right, a promoter system, and optionally an enhancer sequence.

A more preferred alternative embodiment of the present invention relates to a method to elicit production of a secondary metabolite from a BGC in a bacterium, wherein the method comprises the following steps:
i) deleting a GR gene in said bacterium, and
ii) optionally activating a BGC in said bacterium;
or wherein the method comprises the following steps:
i′) essentially activating a BGC in said bacterium, and
ii′) optionally deleting a GR gene in said bacterium;
and the method further comprises:
iii) expressing in said bacterium at least one positive selectable marker gene and at least one negative selectable marker gene;
wherein deleting a GR gene of steps i) or ii′) comprises introducing in said bacterium at least one GR specific siRNA, or a group of sequences comprising a Cas gene, at least one GR specific crRNA array, a pair of GR specific homology regions left and right; and/or
wherein activating a BGC of steps ii) or i′) comprises introducing in said bacterium a group of sequences comprising a Cas gene, at least one BGC specific crRNA array, a pair of BGC specific homology regions left and right, a promoter system, and optionally an enhancer sequence;
   wherein said BGC is selected from the group comprising puromycin, madumycin II, xenoamicin, rupshomycin, rupshomycin derivatives, safracin, safracin derivatives.

An embodiment of the present invention relates to a method to elicit production of a secondary metabolite from a BGC in a bacterium, wherein the method comprises the following steps:
i) deleting a GR gene in said bacterium, and
ii) optionally activating at least 2 BGCs in said bacterium;
or wherein the method comprises the following steps:
i′) essentially activating at least 2 BGCs in said bacterium, and
ii′) optionally deleting a GR gene in said bacterium;
and the method further comprises:
iii) expressing in said bacterium at least one positive selectable marker gene and at least one negative selectable marker gene;
wherein deleting a GR gene of steps i) or ii′) comprises introducing in said bacterium at least one GR specific siRNA, or a group of sequences comprising a Cas gene, at least one GR specific crRNA array, a pair of GR specific homology regions left and right; and/or
wherein activating a BGC of steps ii) or i′) comprises introducing in said bacterium a group of sequences comprising a Cas gene, at least one BGC specific crRNA array, a pair of BGC specific homology regions left and right, a promoter system, and optionally an enhancer sequence;
   wherein said BGC is selected from the group comprising puromycin, madumycin II, xenoamicin, rupshomycin, rupshomycin derivatives, safracin, safracin derivatives.

A particularly more preferred embodiment of the present invention relates to a method to elicit production of a secondary metabolite from a BGC in a bacterium, wherein the method comprises the following steps:
i) deleting a GR gene in said bacterium, and
ii) optionally activating a BGC in said bacterium;
or wherein the method comprises the following steps:
i′) essentially activating a BGC in said bacterium, and
ii′) optionally deleting a GR gene in said bacterium;
and the method further comprises:
iii) expressing in said bacterium at least one positive selectable marker gene and at least one negative selectable marker gene;
wherein deleting a GR gene of steps i) or ii′) comprises introducing in said bacterium at least one GR specific siRNA, or a group of sequences comprising a Cas gene, at least one GR specific crRNA array, a pair of GR specific homology regions left and right; and/or
wherein activating a BGC of steps ii) or i′) comprises introducing in said bacterium a group of sequences comprising a Cas gene, at least one BGC specific crRNA array, a pair of BGC specific homology regions left and right, a promoter system, and optionally an enhancer sequence;
   wherein the step i)-iii) are performed using an automated liquid handling robotics, and wherein said robotics enables high-throughput manipulation of liquid added to or removed from cultures comprising the bacteria.

A preferable embodiment of the present invention relates to a method to elicit production of a secondary metabolite from a BGC in a bacterium, wherein the method comprises the following steps:
i) deleting a GR gene in said bacterium, and
ii) optionally activating at least 2 BGCs in said bacterium;
or wherein the method comprises the following steps:
i′) essentially activating at least 2 BGCs in said bacterium, and
ii′) optionally deleting a GR gene in said bacterium;
and the method further comprises:
iii) expressing in said bacterium at least one positive selectable marker gene and at least one negative selectable marker gene;
wherein deleting a GR gene of steps i) or ii′) comprises introducing in said bacterium at least one GR specific siRNA, or a group of comprising a Cas gene, at least one GR specific crRNA array, a pair of GR specific homology regions left and right; and/or
wherein activating a BGC of steps ii) or i′) comprises introducing in said bacterium a group of sequences comprising a Cas gene, at least one BGC specific crRNA array, a pair of BGC specific homology regions left and right, a promoter system, and optionally an enhancer sequence;
   wherein the step i)-iii) are performed using an automated liquid handling robotics, and wherein said robotics enables high-throughput manipulation of liquid added to or removed from cultures comprising the bacteria.

A further more preferable embodiment of the present invention relates to a method to elicit production of a secondary metabolite from a BGC in a bacterium, wherein the method comprises the following steps:
i) deleting a GR gene in said bacterium, and
ii) optionally activating a BGC in said bacterium;
or wherein the method comprises the following steps:
i′) essentially activating a BGC in said bacterium, and
ii′) optionally deleting a GR gene in said bacterium;
and the method further comprises:
iii) expressing in said bacterium at least one positive selectable marker gene and at least one negative selectable marker gene;
wherein deleting a GR gene of steps i) or ii′) comprises introducing in said bacterium at least one GR specific siRNA, or a group of sequences comprising a Cas gene, at least one GR specific crRNA array, a pair of GR specific homology regions left and right; and/or
wherein activating a BGC of steps ii) or i′) comprises introducing in said bacterium a group of sequences comprising a Cas gene, at least one BGC specific crRNA array, a pair of BGC specific homology regions left and right, a promoter system, and optionally an enhancer sequence;
   wherein said BGC is selected from the group comprising puromycin, madumycin II, xenoamicin, rupshomycin, rupshomycin derivatives, safracin, safracin derivatives;
   wherein the step i)-iii) are performed using an automated liquid handling robotics, and wherein said robotics enables high-throughput manipulation of liquid added to or removed from cultures comprising the bacteria.

A more preferred aspect of the present invention relates to a method to elicit production of a secondary metabolite from a BGC in a bacterium, wherein the method comprises the following steps:
i) deleting a GR gene in said bacterium, and
ii) optionally activating at least 2 BGCs in said bacterium;
or wherein the method comprises the following steps:
i′) essentially activating at least 2 BGCs in said bacterium, and
ii′) optionally deleting a GR gene in said bacterium;
and the method further comprises:
iii) expressing in said bacterium at least one positive selectable marker gene and at least one negative selectable marker gene;
wherein deleting a GR gene of steps i) or ii′) comprises introducing in said bacterium at least one GR specific siRNA, or a group of sequences comprising a Cas gene, at least one GR specific crRNA array, a pair of GR specific homology regions left and right; and/or
wherein activating a BGC of steps ii) or i′) comprises introducing in said bacterium a group of sequences comprising a Cas gene, at least one BGC specific crRNA array, a pair of BGC specific homology regions left and right, a promoter system, and optionally an enhancer sequence;
   wherein said BGC is selected from the group comprising puromycin, madumycin II, xenoamicin, rupshomycin, rupshomycin derivatives, safracin, safracin derivatives; wherein the step i)-iii) are performed using an automated liquid handling robotics, and wherein said robotics enables high-throughput manipulation of liquid added to or removed from cultures comprising the bacteria.

A particularly preferred embodiment of the present invention is a method to elicit production of a secondary metabolite from a BGC in a bacterium, wherein the method comprises the following steps:
i) deleting CyaA gene in said bacterium, and
ii) optionally activating a BGC in said bacterium;
or wherein the method comprises the following steps:
i′) essentially activating a BGC in said bacterium, and
ii′) optionally deleting a CyaA gene in said bacterium;
and the method further comprises:
iii) expressing in said bacterium at least one positive selectable marker gene and at least one negative selectable marker gene;
wherein deleting a CyaA gene of steps i) or i′) comprises introducing in said bacterium at least one CyaA specific siRNA, or a group of sequences comprising a Cas gene, at least one CyaA specific crRNA array, a pair of CyaA specific homology regions left and right; and/or
wherein activating a BGC of steps ii) or i′) comprises introducing in said bacterium a group of sequences comprising a Cas gene, at least one BGC specific crRNA array, a pair of BGC specific homology regions left and right; a promoter system sequence, and optionally an enhancer sequence.

A particularly more preferred embodiment of the present invention is a method to elicit production of a secondary metabolite from a BGC in a bacterium, wherein the method comprises the following steps:
i) deleting CyaA gene in said bacterium, and
ii) optionally activating a BGC in said bacterium;
or wherein the method comprises the following steps:
i′) essentially activating a BGC in said bacterium, and
ii′) optionally deleting a CyaA gene in said bacterium;
and the method further comprises:
iii) expressing in said bacterium at least one positive selectable marker gene and at least one negative selectable marker gene;
wherein deleting a CyaA gene of steps i) or i′) comprises introducing in said bacterium at least one CyaA specific siRNA, or a group of sequences comprising a Cas gene, at least one CyaA specific crRNA array, a pair of CyaA specific homology regions left and right; and/or
wherein activating a BGC of steps ii) or i′) comprises introducing in said bacterium a group of sequences comprising a Cas gene, at least one BGC specific crRNA array, a pair of BGC specific homology regions left and right, a promoter system sequence, and optionally an enhancer sequence, and
   wherein said BGC is selected from the group comprising puromycin, madumycin II, xenoamicin, rupshomycin, rupshomycin derivatives, safracin, safracin derivatives.

A further more preferred embodiment of the present invention is a method to elicit production of a secondary metabolite from a BGC in a bacterium, wherein the method comprises the following steps:
i) deleting CyaA gene in said bacterium, and
ii) optionally activating xenoamicin BGC in said bacterium;
or wherein the method comprises the following steps:
i′) essentially activating xenoamicin BGC in said bacterium, and
ii′) optionally deleting a CyaA gene in said bacterium;
and the method further comprises:
iii) expressing in said bacterium at least one positive selectable marker iii) gene and at least one negative selectable marker gene;
wherein deleting a CyaA gene of steps i) or i′) comprises introducing in said bacterium at least one CyaA specific siRNA, or a group of comprising a Cas gene, at least one CyaA specific crRNA array, a pair of GR specific homology regions left and right; and/or
wherein activating xenoamicin BGC of steps i) or i′) comprises introducing in said bacterium a group of sequences comprising a Cas gene, at least one xenoamicin BGC specific crRNA array, a pair of xenoamicin BGC specific homology regions left and right, a promoter system sequence, and optionally an enhancer sequence.

### Rupshomycin Derivatives

Another aspect of the present invention is directed to rupshomycin and its derivatives discovered and obtained by the inventive methods described herein. Said compounds (see FIGS. 12-14, and 23-25) are the product of a novel BGC named rpmA-O and exhibit interesting biological activities. Therefore, thee compounds are particularly useful as antibiotic, or anti-drug, or immune suppressive drug.

Thus, the present invention is also directed to a compound of general formula (I)

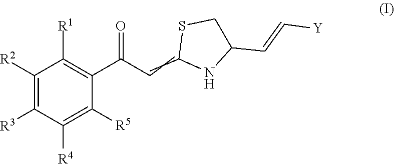

wherein
Y represents —CH═CH₂ or

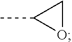

R¹, R², R³, R⁴, and R⁵ are independently of each other selected from —H, —NH₂, —NHR⁶, —F, —OH, —OCH₃, —OCH₂CH₃, and —OCH₂CH₂CH₃;
or R² and R³ or R³ and R⁴ together with the carbon atom to which they are bound to form one of the following rings:

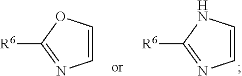

wherein R⁶ represents —H, —CH₃, —CH₂CH₂CH₃, —CH(CH₃)₂, —CH₂ (CH₂)₂CH₃, —CH₂ (CH₂)₃CH₃, —CH₂ (CH₂)₄CH₃, —CH₂ (CH₂)₅CH₃, —CH₂ (CH₂)₆CH₃, —CH₂ (CH₂)₇CH₃, —CH₂ (CH₂)₈CH₃, —CH₂ (CH₂)₉CH₃, —CH₂ (CH₂)₁₀CH₃, —CH₂ (CH₂)₁₁CH₃, —CH₂ (CH₂)₁₂CH₃, —CH₂CH(CH₃)₂, —C(CH₃)₃, —CH₂CH═CH₂, —CH₂CH═CH(CH₃), —CH₂CH═C(CH₃)₂, or —CH₂CH═CHCH₂CH₃;
with the proviso that when Y represents —CH═CH₂, R¹, R², R⁴, and R⁵ cannot be —H, and R³ cannot be —OH simultaneously.

The structural fragment [symbol] indicates a mixture of cis and trans isomers.

In a preferred embodiment, the rupshomycin compounds are in a (2Z)-thiazolidine isomer form of formula (IA)

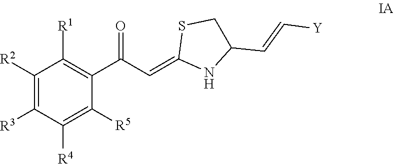

wherein
Y represents —CH═CH₂ or

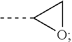

R¹, R², R³, R⁴, and R⁵ are independently of each other selected from —H, —NH₂, —NHR⁶, —F, —OH, —OCH₃, —OCH₂CH₃, and —OCH₂CH₂CH₃;
or R² and R³ or R³ and R⁴ together with the carbon atom to which they are bound to form one of the following rings:

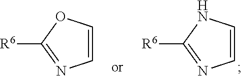

wherein R⁶ represents-H, —CH₃, —CH₂CH₂CH₃, —CH(CH₃)₂, —CH₂ (CH₂)₂CH₃, —CH₂ (CH₂)₃CH₃, —CH₂ (CH₂)₄CH₃, —CH₂ (CH₂)₅CH₃, —CH₂ (CH₂)₆CH₃, —CH₂ (CH₂)₇CH₃, —CH₂ (CH₂)₈CH₃, —CH₂ (CH₂)₉CH₃, —CH₂ (CH₂)₁₀CH₃, —CH₂ (CH₂)₁₁CH₃, —CH₂ (CH₂)₁₂CH₃, —CH₂CH(CH₃)₂, —C(CH₃)₃, —CH₂CH═CH₂, —CH₂CH═CH(CH₃), —CH₂CH═C(CH₃)₂, or —CH₂CH═CHCH₂CH₃;
with the proviso that when Y represents-CH═CH₂, R¹, R², R⁴, and R⁵ cannot be —H, and R³ cannot be —OH simultaneously.

In a preferred embodiment, the rupshomycin compounds are in a (2E)-thiazolidine isomer form of formula (IB)

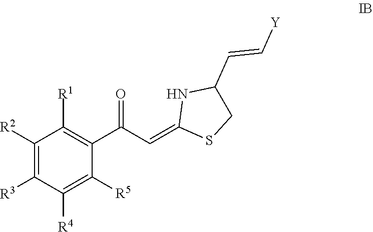

wherein
Y represents-CH═CH₂ or

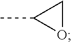

R¹, R², R³, R₄, and R⁵ are independently of each other selected from —H, —NH₂, —NHR⁶, —F, —OH, —OCH₃, —OCH₂CH₃, and —OCH₂CH₂CH₃;
or R² and R³ or R³ and R⁴ together with the carbon atom to which they are bound to form one of the following rings:

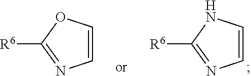

wherein R⁶ represents-H, —CH₃, —CH₂CH₂CH₃, —CH(CH₃)₂, —CH₂ (CH₂)₂CH₃, —CH₂ (CH₂)₃CH₃, —CH₂ (CH₂)₄CH₃, —CH₂ (CH₂)₅CH₃, —CH₂ (CH₂)₆CH₃, —CH₂ (CH₂)₇CH₃, —CH₂ (CH₂)₈CH₃, —CH₂ (CH₂)₉CH₃, —CH₂ (CH₂)₁₀CH₃, —CH₂ (CH₂)₁₁CH₃, —CH₂ (CH₂)₁₂CH₃, —CH₂CH(CH₃)₂, —C(CH₃)₃, —CH₂CH═CH₂, —CH₂CH═CH(CH₃), —CH₂CH═C(CH₃)₂, or —CH₂CH═CHCH₂CH₃;
with the proviso that when Y represents-CH═CH₂, R¹, R², R⁴, and R⁵ cannot be —H, and R³ cannot be —OH simultaneously.

Particularly preferred is a compound selected from the group consisting of:

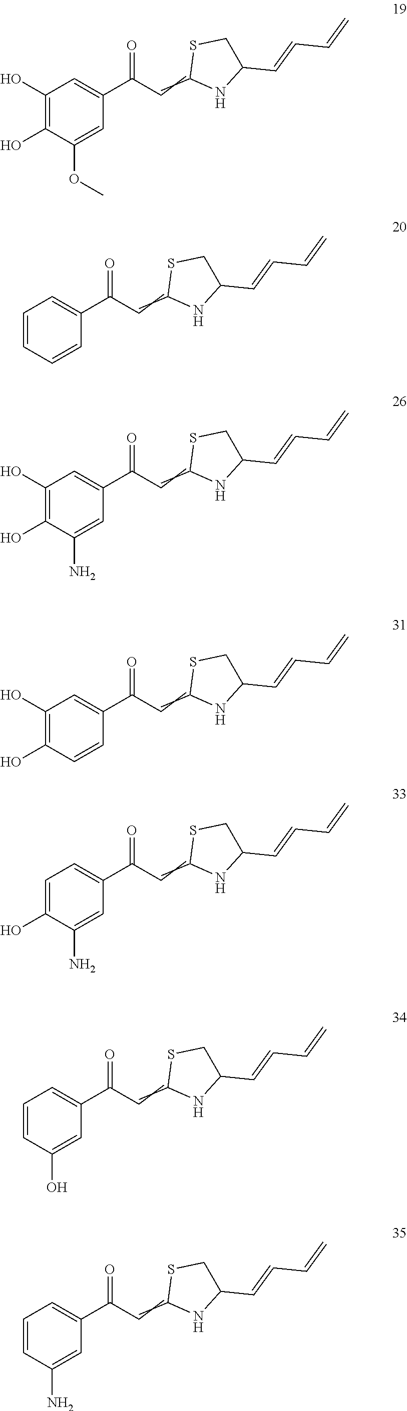

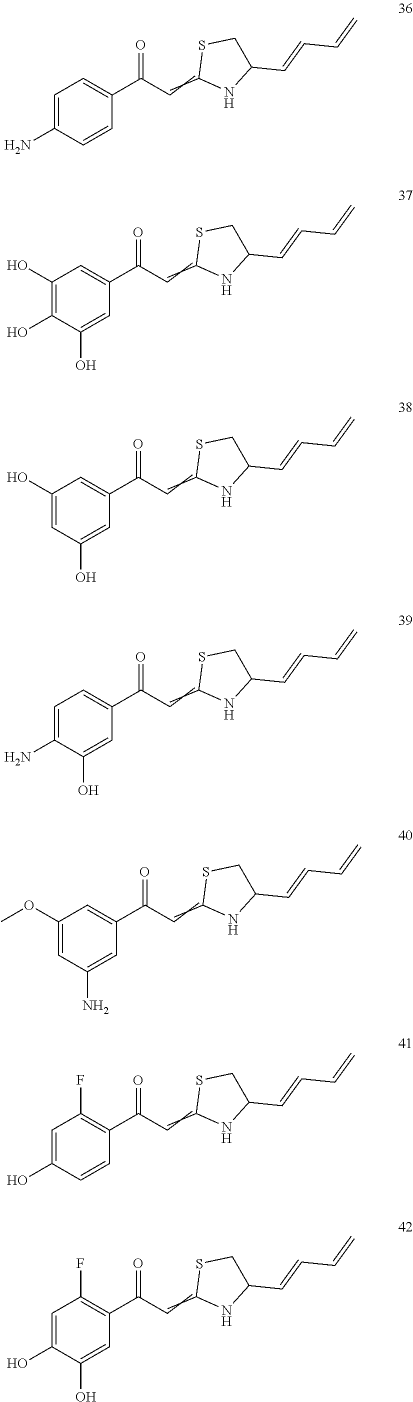

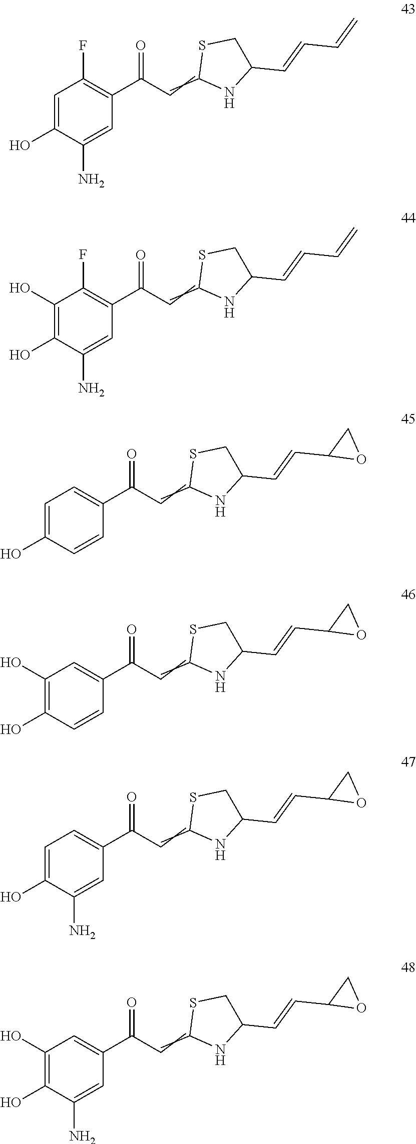 [SMILES: C=C/C=C/C1CSC(=CC(=O)c2cc(N)c(O)cc2F)N1 ; C=C/C=C/C1CSC(=CC(=O)c2cc(N)c(O)c(O)c2F)N1 ; O=C(C=C1NC(/C=C/C2CO2)CS1)c1ccc(O)cc1 ; O=C(C=C1NC(/C=C/C2CO2)CS1)c1ccc(O)c(O)c1 ; Nc1cc(C(=O)C=C2NC(/C=C/C3CO3)CS2)ccc1O ; Nc1cc(C(=O)C=C2NC(/C=C/C3CO3)CS2)cc(O)c1O]

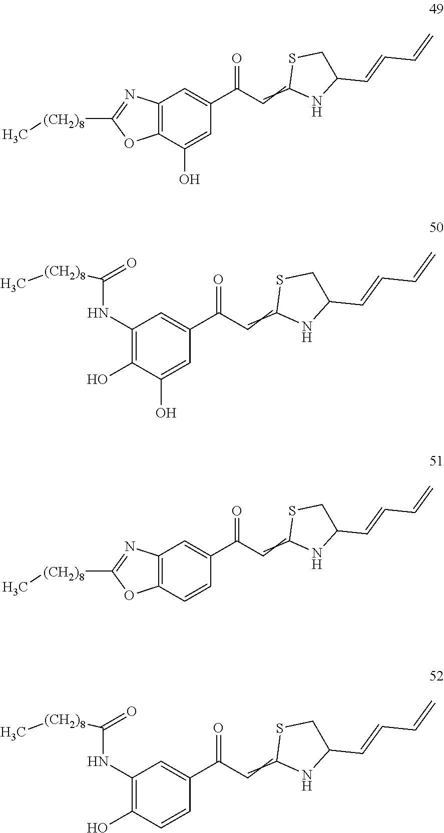

Particularly preferred is when the compound of the above shown group is a (2E)-thiazolidine isomer.

Particularly preferred is when the compound of the above shown group is a (2Z)-thiazolidine isomer.

TABLE 2
Strains and plasmids used in this study.

| | Genotype | Source/Reference |
|---|---|---|
| Strains | | |
| E. coli ST18 | pro thi hsdR⁺ Tp^{r} Sm^{r}; chromosome::RP4-2 | DSMZ, DSM No.: 22074 |
| | Tc::Mu-Kan::Tn7/λpir; ΔhemA | |
| E. coli DH10B | F⁻mcrA Δ(mrr-hsdRMS-mcrBC) φ80lacZΔM15 | Invitrogen |
| | ΔlacX74 recA1 endA1 araD139 Δ(ara-leu)7697 | |
| | galU galK λ⁻rpsL(Str^{R}) nupG | |
| P. laumondii subsp. | Wild type | DSMZ, DSM No.: 15139 |
| laumondii TTO1 | | |
| X. nematophila ATCC | Wild type | ATCC 19061 |
| 19061 | | |
| X. doucetiae FRM16 | Wild type | DSMZ, DSM No.: 17909 |
| X. indica DSM 17382 | Wild type | |
| Xenorhabdus sp. TS4 | Wild type | |
| Plasmid | | |
| pEB17 | pDS132 based, R6K ori, oriT, kanR, cipB, sacB | |
| pCEP_kan | pDS132 based, R6K ori, oriT, kanR, araC, P_{BAD} | |
| pTargetF | pMB1 Spec^{R} sgRNA-cadA | Addgene plasmid # 62226 |
| p46Cpf1-OP2 | repA101(Ts) cat tetR Pₜₑₜ-Red araC P_{BAD} codon | Addgene # 98592 |
| | optimized FnCas12a | |
| pAR14 | pMB1 Spec^{R} sacB | This study |
| pAR15 | pMB1 Gm^{R} sacB | This study |
| pAR16 | p15A cat tetR Pₜₑₜ-Red araC P_{BAD} codon | This study |
| | optimized FnCas12a | |
| pAR18 | pMB1 Gm^{R} sacB crRNA framework | This study |
| pAR20 | pSeva231 tetR Pₜₑₜ-Red araC P_{BAD} codon | This study |
| | optimized FnCas12a sacB crRNA framework | |
| pDS132 | R6K ori, mob RP4, cat, sacB, sacB promoter, | Genbank AY489048 |
| | lac operator | |
| pMB1 | — | GenBank: M77789.2 |
| repA101(Ts) | — | GenBank: K00042.1 |
| p15A | — | GenBank: V00309.1 |
| pSeva231 | pBBR1 oriV, RK2 oriT, kanR | |
| pAR29bad | p15A oriV, RK2 oriT, kanR, araC^{AM}, P_{BAD} | |
| pAR30bad | pBBR1 oriV, RK2 oriT, GmR | |
| pCola_PBad_ xscA-H | P_{BAD} promoter, pCOLA ori, xscA-H | This work |
| pACYC_PBad_xscIJ | P_{BAD} promoter, pACYC ori , xscIJ | This work |
| pCDF_PBad_xscK | P_{BAD} promoter, pCloDF13 ori, xscK | This work |
| pCola | pCOLA ori, KmR | Novagen |
| pACYC | pACYC ori, CmR | Novagen |
| pCDF-1b | pCloDF13 ori, SmR | Novagen |

Legend: KanR: kanamicin resistance, Spec^{R}: Spectinomycin resistance; Gm^{R}: gentamicin resistance, cipB promoter pEB17, pCEP_kan disclosed by Nat. Chem. 14, 701-712 (2022).

TABLE 3
Overview of performed experiments of genome editing in P. laumondii TTO1 and X. nematophila

| | | Associated | Locus | Gene | | | Efficiency |
|---|---|---|---|---|---|---|---|
| Gene | Organism | SM | Tag | Editing | Type | Size | (%) |
| StlA | P. laumondii TTO1 | Isopropylstilbene (1) | plu2234 | Deletion | BGC | 1.9 | 100 |
| | BX571866.1 | | ΔP [symbol] ::P [symbol] | promoter | | Δ3.4::1.3* | 100 |
| | | | | exchange | | | |
| gxpS | P. laumondii TTO1 | GameXPeptide A (3) | plu3263 | Deletion | NRPS | 19.4 | 100 |
| | BX571870.1 | | ΔP [symbol] ::P [symbol] | promoter | BGC | Δ0.1::1.3* | 74 |
| | | | | exchange | | | |
| xcnA | X. nematophila | Xenocoumacin I & | XNC1_1711 | Deletion | BGC | 7.5 | 100 |
| | FN667742.1 | II (7-8) | ΔP [symbol] ::P [symbol] | promoter | BGC | Δ0.3::1.3* | 100 |
| | | | | exchange | | | |
| rxpA-C | X. nematophila | Rhabdopeptides | XNC1_2228-2230 | Deletion | BGC | 15.7 | 100 |
| | | (9-12) | rxpA-C (XNC1_2228- | Reporter | | n.d. | n.d. |
| | | | 2230)::mNeonGreen | replacement | | | |
| rxpA | | | ΔP [symbol] ::P [symbol] | promoter | | Δ0.5::1.3* | 100 |
| | | | | exchange | | | |
| indC | P. laumondii TTO1 | Indigoidine (2) | ΔP [symbol] ::P [symbol] | promoter | NRPS | Δ0.2::1.3* | 100 |
| | | | | exchange | | | |
| glbA | P. laumondii TTO1 | Glidobactins (4-5) | ΔP [symbol] ::P [symbol] | promoter | BGC | Δ0.08::1.3* | 68 |
| | | | | exchange | | | |
| stlCDE | P. laumondii TTO1 | Isopropylstilbene | plu2163-plu2165 | Reporter | BGC | n.d. | n.d. |
| | | | | replacement | | | |
| ppyS | X. nematophila | Unknown pyrone | XNC1_3338 | Reporter | BGC | n.d. | |
| | | | | replacement | | | |
| ppyS | P. laumondii TTO1 | Unknown pyrone | PluTTO1m_24855 | Reporter | BGC | n.d. | n.d. |
| | | | | replacement | | | |
| AQ MT4 | P. laumondii TTO1 | Anthraquinone | plu4895 | Reporter | BGC | n.d. | n.d. |
| | | | | replacement | | | |
| odlC | X. nematophila | Odilorhabdins | XNC1_2467 | Reporter | BGC | n.d. | n.d. |
| | | | | replacement | | | |
| mxn | X. nematophila | madumycin II | see Table 4 | refactoring | BGC | n.d. | n.d. |
| rpm | X. indica | rupshomycin | see Table 5 | refactoring + | BGC | n.d. | n.d. |
| | DSM 17382 | | | Hfq GR deletion | | | |
| xsc | Xenorhabdus | safracin | see Table 6 | refactoring | BGC | n.d. | n.d. |
| | sp. TS4 | | | | | | |
| cyaA | P. laumondii TTO1 | — | TTO1_PLUMV2_22995, | deletion | GR | n.d. | n.d. |
| | Xenorhabdas | | XDOUV2_01760 | | | | |
| | doucetiae | | | | | | |
| hfq | X. indica | — | | deletion | GR | n.d. | n.d. |
| xobA | | xenoamicin | | | | n.d. | n.d. |

*indicates the replaced sequence between homology arms by the 1.3 kb promoter sequence;
X. nematophila ATCC 19061;
n.d.: not determined [symbol]
indicates data missing or illegible when filed

TABLE 4
Proteins of the mxn cluster in X. nematophila ATCC 19061,
their proposed function, protein size and closest homologues.

| | | Size | |
|---|---|---|---|
| Protein | Locus | (aa) | Proposed function |
| MxnA | XNC1_RS07585 | 81 | acyl carrier protein |
| MxnB | XNC1_RS07580 | 408 | beta-ketoacyl-ACP synthase |
| MxnC | XNC1_RS07575 | 411 | HMG-CoA synthase-like protein |
| MxnD | XNC1_RS20555 | 262 | enoyl-CoA hydratase |
| MxnE | XNC1_RS07565 | 250 | enoyl-CoA hydratase |
| MxnF | XNC1_RS07560 | 1980 | PKS |
| MxnG | XNC1_RS07555 | 2407 | Mixed trans-AT type I PKS/NRPS |
| MxnH | XNC1_RS07550 | 2302 | NRPS |
| MxnI | fabD | 286 | Acyltransferase |
| MxnJ | XNC1_RS07540 | 400 | major facilitator superfamily |
| | | | transporter |
| MxnK | XNC1_RS07535 | 229 | thioesterase |
| MxnL | XNC1_RS07530 | 664 | BkdA |
| MxnM | XNC1_RS22760 | 4823 | hybrid PKS/NRPS |
| MxnN | XNC1_RS07520 | 2344 | PKS type I |
| MxnO | XNC1_RS07515 | 529 | ATPase component of ABC |
| | | | transporters |

TABLE 5
Proteins of the rpm cluster in X. indica DSM 17382, their
proposed function, protein size and closest homologues.

| | Gene NCBI | Gene | Protein NCBI | Proposed function of corresponding |
|---|---|---|---|---|
| Gene | Accession No. | size (bp) | Accession No. | protein |
| rpmA | Xind_RS13800 | 795 | WP_047680461.1 | Aldolase |
| rpmB | Xind_RS13805 | 1659 | WP_187650276.1 | Acyl-CoA ligase |
| rpmC | Xind_RS13810 | 429 | WP_246432556.1 | OsmC family protein |
| rpmD | Xind_RS13815 | 7839 | WP_187650277.1 | PKS-NRPS hybrid |
| rpmE | Xind_RS13820 | 4305 | WP_047680469.1 | NAD(P)-dependent oxireductase (PKS) |
| rpmF | Xind_RS13825 | 6024 | WP_187650278.1 | Alpha/beta fold hydrolase (PKS) |
| rpmG | Xind_RS13830 | 1485 | WP_052189457.1 | 4-hydroxyphenylacetate 3-hydroxlyase |
| rpmH | Xind_RS13835 | 1344 | WP_047680471.1 | MATE family efflux transporter |
| rpmI | Xind_RS13840 | 366 | WP_047680473.1 | Hypothetical protein |
| rpmJ | Xind_RS13845 | 1224 | WP_187650279.1 | 3-dehydroquinate synthase |
| rpmK | Xind_RS13850 | 768 | WP_187650280.1 | Alpha/beta fold hydrolase |
| rpmL | Xind_RS13855 | 861 | WP_052189461.1 | ACP S-malonyltransferase |
| rpmM | Xind_RS13860 | 1035 | WP_047680476.1 | Cupin domain-containing protein |
| rpmN | Xind_RS13865 | 612 | WP_222941817.1 | SAP domain-containg protein |
| rpmO | Xind_RS13870 | 459 | WP_047680486.1 | Gyrl-like domain-containing protein |

TABLE 6
Proteins of the xsc cluster (safracin) in Xenorhabdus sp. TS4,
their proposed function, protein size and closest homologues.

| | NCBI Reference | Size | |
|---|---|---|---|
| Protein | Sequence | (aa) | Proposed function |
| XscA | Xets_RS15840 | 1055 | non-ribosomal peptide synthetase |
| XscB | Xets_RS15835 | 1073 | non-ribosomal peptide synthetase |
| XscC | Xets_RS15830 | 1444 | non-ribosomal peptide synthetase |
| XscD | Xets_RS15825 | 352 | putative hydroxylase |
| XscE | Xets_RS15820 | 66 | MbtH family NRPS accessory protein |
| XscF | Xets_RS15815 | 355 | O-methyltransferase |
| XscG | Xets_RS15810 | 343 | O-methyltransferase |
| XscH | Xets_RS15805 | 180 | dihydrofolate reductase/dehydratase |
| XscI | Xets_RS15845 | 220 | SAM-dependent methyltransferase |
| XscJ | Xets_RS15850 | 501 | FAD-dependent monooxygenase |
| XscK | Xets_RS24810 | 512 | metallopeptidase |

TABLE 7
Sequences used in this study (sequences
present in the Sequence Listing file).
Table 7a Primers

| | | | Direc- |
|---|---|---|---|
| ID | Name | Target gene/template | tion |
| NO 1 | AR533 | pAR20 or pCpf1-XP | fw |
| NO 2 | AR534 | pAR20 or pCpf1-XP | rev |
| NO 3 | PC O45 | XDOUV2_01760, CyaA | fw |
| NO 4 | PC O46 | XDOUV2_01760, CyaA | rv |
| NO 5 | PC O47 | XDOUV2_01760, CyaA | fw |
| NO 6 | PC O48 | XDOUV2_01760, CyaA | rv |
| NO 7 | PC O49 | XDOUV2_01760, CyaA | fw |
| NO 8 | PC O50 | XDOUV2_01760, CyaA | rw |
| NO 9 | PC O69 | TT01_PLUMV2_22995 | fw |
| | | (CyaA) | |
| NO 10 | PC O70 | TT01_PLUMV2_22995 | rw |
| | | (CyaA) | |
| NO 11 | PC O71 | TT01_PLUMV2_22995 | fw |
| | | (CyaA) | |
| NO 12 | PC O72 | TT01_PLUMV2_22995 | rw |
| | | (CyaA) | |
| NO 13 | PC O73 | TT01_PLUMV2_22995 | fw |
| | | (CyaA) | |
| NO 14 | PC O74 | TT01_PLUMV2_22995 | rw |
| | | (CyaA) | |
| NO 15 | ODA1 | XDOUV2_10775(XabA) | fw |
| NO 16 | ODA2 | XDOUV2_10775 (XabA) | rv |
| NO 17 | ODA3 | XDOUV2_10775 (XabA) | rv |
| NO 18 | VPpCEP-fw | pCEP_Kan | fw |
| NO 19 | VPpDS132-rv | pCEP_kan | rv |
| NO 20 | AR426 | p46Cpf1-OP2 | fw |
| NO 21 | AR427 | p46Cpf1-OP2 | rev |
| NO 22 | AR428 | p15A vector | fw |
| NO 23 | AR429 | p15A vector | rev |
| NO 24 | AR430 | pTargetF | fw |
| NO 25 | AR431 | pTargetF | rev |
| NO 26 | AR432 | sacB (pEB17_KM) | fw |
| NO 27 | AR433 | sacB (pEB17_KM) | rev |
| NO 28 | AR434 | pSEVA631 | fw |
| NO 29 | AR435 | pSEVA631 | rev |
| NO 30 | AR436 | pAR15 | fw |
| NO 31 | AR437 | pAR15 | rev |
| NO 32 | AR_seq_07 | Pex_gxpS_TTO1 | fw |
| NO 33 | AR_seq_08 | Pex_gxpS_TTO1 | rev |
| NO 34 | AR_seq_11 | Pex_indC | fw |
| NO 35 | AR_seq_12 | Pex_indC | rev |
| NO 36 | AR_seq_13 | dgxpS_TTO1 | fw |
| NO 37 | AR_seq_14 | dgxpS_TTO1 | rev |
| NO 38 | AR_seq_17 | dstlA | fw |
| NO 39 | AR_seq_18 | dstlA | rev |
| NO 40 | AR_seq_21 | Pex_glb | fw |
| NO 41 | AR_seq_22 | Pex_glb | rev |
| NO 42 | AR_seq_29 | drxp | fw |
| NO 43 | AR_seq_30 | drxp | rev |
| NO 44 | AR_seq_31 | dxcnA | fw |
| NO 45 | AR_seq_32 | dxcnA | rev |
| NO 46 | AR_seq_33 | Pex_rxpA | fw |
| NO 47 | AR_seq_34 | Pex_rxpA | rev |
| NO 48 | AR_seq_35 | Pex_xcnA | fw |
| NO 49 | AR_seq_36 | Pex_xcnA | rev |
| NO 50 | AR_seq_37 | Pex_mxnA_XNEM | fw |
| NO 51 | AR_seq_38 | Pex_mxnA_XNEM | rev |
| NO 52 | AR_seq_76 | Pex_stlA | fw |
| NO 53 | AR_seq_77 | Pex_stlA | rev |
| NO 54 | AR_seq_84 | Xnem_C7.2.Pex | fw |
| NO 55 | AR_seq_85 | Xnem_C7.2.Pex | rev |
| NO 56 | AR577 | PvanCC promoter | fw |
| NO 57 | AR606 | rpmA | fw |
| NO 58 | AR607 | rpmA | rev |
| NO 59 | AR608 | rpmJ | fw |
| NO 60 | AR609 | rpmJ | rev |
| NO 61 | AR620 | rpmG | fw |
| NO 62 | AR621 | rpmG | rev |
| NO 63 | AR616 | rpmMN | fw |
| NO 64 | AR617 | rpmMN | rev |
| NO 65 | AR604 | rpmO | fw |
| NO 66 | AR605 | rpmO | rev |
| NO 201 | AR_seq_86 | XNEM_hfq | fw |
| NO 202 | AR_seq_87 | XNEM_hfq | rev |
| NO 203 | AR_seq_88 | XNEM_cyaA | fw |
| NO 204 | AR_seq_89 | XNEM_cyaA | rev |
| NO 205 | AR_seq_90 | Pex_dpr | fw |
| NO 206 | AR_seq_91 | Pex_dpr | rev |
| NO 207 | | XDUO_cyaA | fw |
| NO 208 | | XDUO_cyaA | rev |
| NO 209 | | XDUO_Pex_xab | fw |
| NO 210 | | XDUO_Pex_xab | rev |
| NO 211 | clxD (codon optimized) | | |
| NO 212 | styA (Bacillus) | | |
| | *T1-→A | | |

TABLE 7b
Gene specific target spacers used for CRISPR/Cas12 mediated genome
editing (the PAM sequence is present in the genome closed to TS)

| SEQ | Target | | | PAM |
|---|---|---|---|---|
| ID | spacer | Gene | Type of editing | sequence |
| NO 67 | TS-A | stlA | deletion | TTTG |
| NO 68 | TS-B | | | TTTA |
| NO 69 | TS-A | stlA | promoter exchange | TTTA |
| NO 70 | TS-B | | | TTTG |
| NO 71 | TS-A | gxpS | deletion | TITG |
| NO 72 | TS-B | | | TTTG |
| NO 73 | TS-A | gxpS | promoter exchange | TTTA |
| NO 74 | TS-B | | | TTTG |
| NO 75 | TS-A | xcnA | deletion | TTTG |
| NO 76 | TS-B | | | TTTC |
| NO 77 | TS-A | xcnA | promoter exchange | TTTC |
| NO 78 | TS-B | | | TTTG |
| NO 79 | TS-A | rxpA-C | deletion | TTTA |
| NO 80 | TS-B | | | TTTA |
| NO 81 | TS-A | rxpA | promoter exchange | TTTC |
| NO 82 | TS-B | | | TTTA |
| NO 83 | TS-A | rxpA-C | Fluorescent reporter | TTTC |
| NO 84 | TS-B | | replacement | TTTC |
| NO 85 | TS-A | indC | promoter exchange | TTTA |
| NO 86 | TS-B | | | TTTC |
| NO 87 | TS-A | glbA | promoter exchange | TTTA |
| NO 88 | TS-B | | | ATTA |
| NO 89 | TS-A | mxnA | refactoring | TTTA |
| NO 90 | TS-B | | | TTTC |
| NO 91 | TS-A | mxnB | promoter exchange | TTTA |
| NO 92 | TS-B | | | TTTG |
| NO 93 | TS-A | mxnL | promoter exchange | TTTC |
| NO 94 | TS-B | | | TTTG |
| NO 95 | TS-A | stlCDE | Fluorescent reporter | TTTG |
| NO 96 | TS-B | | replacement | TTTA |
| NO 97 | TS-A | ppyS | Fluorescent reporter | TTTA |
| NO 98 | TS-B | PluTT01m_24855 | replacement | TTTC |
| NO 99 | TS-A | AQ MT4 | Fluorescent reporter | TTTA |
| NO 100 | TS-B | | replacement | TTTG |
| NO 101 | TS-A | ppys | Fluorescent reporter | TTTA |
| NO 102 | TS-B | | replacement | TTTC |
| NO 103 | TS-A | odlC | Fluorescent reporter | TTTA |
| NO 104 | TS-B | | replacement | TTTC |
| NO 105 | TS-A | xscA/I | Bidirectional promoter | TTTC |
| NO 106 | TS-B | | exchange | TTTC |
| NO 107 | TS-A | xscK | Deletion/promoter | TTTC |
| NO 108 | TS-B | | exchange | TTTG |
| NO 109 | TS-A | rpmA | promoter exchange | TTTG |
| NO 110 | TS-B | | | TTTC |
| NO 111 | TS-A | rpmD | promoter exchange | TTTG |
| NO 112 | TS-B | | | TTTC |
| NO 113 | TS-A | rpmG | Deletion | TTTC |
| NO 114 | TS-B | | | TTTC |
| NO 115 | TS-A | rpmM-O | Deletion | TTTC |
| NO 116 | TS-B | | | TTTA |
| NO 117 | TS-A | hfq X. | Deletion | TTTC |
| NO 118 | TS-B | indica | | TTTA |
| NO 191 | TS-A | hfq X. | Deletion | TTTG |
| NO 192 | TS-B | nematophila | | TTTC |
| NO 193 | TS-A | cyaA X. | Deletion | TTTA |
| NO 194 | TS-B | nematophila | | TTTA |
| NO 195 | TS-A | dprA | promoter exchange | TTTG |
| NO 196 | TS-B | | | TTTC |
| NO 197 | TS-A | cyaA_XDUO | Deletion | TTTA |
| NO 198 | TS-B | | | TTTG |
| NO 199 | TS-A | xabA | promoter exchange | TTTC |
| NO 200 | TS-B | | | TTTA |

TABLE 7c
Sequence list of synthetic dsDNA fragments used for CRISPR/Cas12 mediated genome editing

| SEQ ID | Name | Type of Editing/Description | Organism/note |
|---|---|---|---|
| NO 119 | crRNA | | P_{J23119}, crRNA leader, direct repeat, spacer- |
| | framework | | dummy (Bsal sites), direct repeat, terminator |
| NO 120 | HA-L | Fluorescent reporter | Adapter with Bsal site, [selected target spacer |
| | dummy | | (23-31 bp)], direct repeat, TERMINATOR, |
| | | | placeholder (lowercase), [homologues region |
| | | | (up to 500 bp)], GS-linker sequence I, Adapter |
| | | | with Bsal site |
| NO 121 | HA-L | Deletions | Adapter with Bsal site, [selected target spacer |
| | dummy | | (23-31 bp)], direct repeat, TERMINATOR, |
| | | | placeholder (lowercase), [homologues region |
| | | | (up to 500 bp)], Adapter with Bsal site |
| NO 122 | HA-L | Promoter exchange | Adapter with Bsal site, [selected target spacer |
| | dummy | | (23-31 bp)], direct repeat, TERMINATOR, |
| | | | placeholder (lowercase), [homologues region |
| | | | (up to 500 bp)], Adapter with Bsal site |
| NO 123 | HA-L | Promoter exchange with | Adapter with Bsal site, [selected target spacer |
| | dummy | Bidirectional Promoter (proD) | (23-31 bp)], direct repeat, TERMINATOR, |
| | | | placeholder (lowercase), [homologues region |
| | | | (up to 500 bp)], proD-UTR(reversed), Adapter |
| | | | with Bsal site |
| NO 124 | dsDNA | dsDNA Fragment for Fluorescent | Bsal site, GS-linker sequence II, [Insertion site |
| | Fragment | reporter editing | for Fluorescent Reporter Sequence] Bsal site |
| NO 125 | HA-R | Fluorescent reporter | Adapter with Bsal site, [homologues region (up |
| | Dummy | | to 500 bp)], placeholder (lowercase), P_{J23119}, |
| | | | crRNA-leader, direct repeat, [selected target |
| | | | spacer (23-31 bp)], Adapter with Bsal site |
| NO 126 | HA-R | deletion | Adapter with Bsal site, [homologues region (up |
| | Dummy | | to 500 bp)], placeholder (lowercase), P_{J23119}, |
| | | | crRNA-leader, direct repeat, [selected target |
| | | | spacer (23-31 bp)], Adapter with Bsal site |
| NO 127 | HA-R | promotor exchange | Adapter with Bsal site, [Start-codon (ATG) with |
| | Dummy | | homologues region (up to 500 bp)], placeholder |
| | | | (lowercase), P_{J23119}, crRNA-leader, direct repeat, |
| | | | [selected target spacer (23-31 bp)], Adapter |
| | | | with Bsal site |
| NO 128 | HA-R | promotor exchange with | Adapter with Bsal site, [Start-codon (ATG) with |
| | Dummy | translational enhancer | homologues region (up to 500 bp)], placeholder |
| | | | (lowercase), P_{J23119}, crRNA-leader, direct repeat, |
| | | | [selected target spacer (23-31 bp)], Adapter |
| | | | with Bsal site |
| NO 129 | FnCas12a | Codon Optimized | |
| NO 130 | mNeonGreen | Fluorescen Reporter Sequence | |
| NO 131 | PvanCC | VA- Inducible Promoter | Bsal site, terminator (reverse), vanR-AM CDS |
| | modified | | (reverse), van2 RBS and P_{J23100}, terminator |
| | | | (reverse), P_{vanCC}, RiboJ, B0064 RBS, Bsal site |
| NO 132 | P_{vanCC}_{_{—}}T7 | P_{vanCC} modified for T7 | Adapter with Bsal site, terminator (reverse), |
| | | Translational enhancer | spacing bases, |
| | | | vanR-AM CDS (reverse), van2 RBS and P_{J23100}, |
| | | | spacing bases, terminator (reverse), spacing |
| | | | bases, P_{vanCC}, RiboJ, T7-g10 translational |
| | | | enhancer part, Adapter with Bsal site |
| NO 133 | ProD* | Bidirectional Promoter | C106G, Davis et. al 2011 |
| NO 134 | Ptac | Ptac_Promoter alone | Bsal site, terminator (reverse, spacing bases, |
| | | | P_{tac}, RiboJ, spacing bases, B0064 RBS, spacing |
| | | | bases, Bsal site |
| NO 135 | T7g10 & RBS | T7g10 = Translational Enhancer | Enhancer, Poly-A, RBS |
| | | | Olins P O, Rangwala S H.; A novel sequence |
| | | | element derived from bacteriophage T7 mRNA |
| | | | acts as an enhancer of translation of the lacZ |
| | | | gene in Escherichia coli. J Biol Chem. 1989 Oct. |
| | | | 15; 264(29): 16973-6. |
| NO 136 | Ptac_mod | modified Ptac Promoter | Bsal site, terminator (reverse), spacing bases, |
| | | | lacI-AM CDS (reverse), ara1 RBS and _{PJ23100}, |
| | | | spacing bases, terminator (reverse), spacing |
| | | | bases, P_{tac}, RiboJ, spacing bases, B0064 RBS, Bsal |
| | | | site |
| NO 137 | ΔstlA_L | Homology arm left for ΔstlA | |
| | | deletion | |
| NO 138 | ΔstlA_R | Homology arm right for ΔstlA | |
| | | deletion | |
| NO 139 | stlA_L | Homology arm left for stlA- | |
| | | Promoter exchange | |
| NO 140 | stlA_R | Homology arm right for stlA- | |
| | | Promoter exchange | |
| NO 141 | ΔgxpS_L | Homology arm left for ΔgxpS | |
| | | deletion | |
| NO 142 | ΔgxpS_R | Homology arm right for ΔgxpS | |
| | | deletion | |
| NO 143 | gxpS_L | Homology arm left for gxpS- | |
| | | Promoter exchange | |
| NO 144 | gxpS_R | Homology arm right for gxpS- | |
| | | Promoter exchange | |
| NO 145 | ΔxcnA_L | Homology arm left for ΔxcnA | Xenorhabdus nematophila |
| | | deletion | |
| NO 146 | ΔxcnA_R | Homology arm right for ΔxcnA | |
| | | deletion | |
| NO 147 | xcnA_L | Homology arm left for xcnA | |
| | | Promoter exchange | |
| NO 148 | xcnA_R | Homology arm right for xcnA | |
| | | Promoter exchange | |
| NO 149 | ΔrxpA-C_L | Homology arm left for ΔrxpA-C | |
| | | deletion | |
| NO 150 | ΔrxpA-C_R | Homology arm right for ΔrxpA-C | |
| | | deletion | |
| NO 151 | rxpA-C_L | Homology arm left for ΔrxpA-C | |
| | | Promoter exchange | |
| NO 152 | rxpA-C_R | Homology arm right for ΔrxpA-C | |
| | | Promoter exchange | |
| NO 153 | rxpA-C_L | Homology arm left for | |
| | | Fluorescent reporter | |
| | | replacement of rxpA-C | |
| NO 154 | rxpA-C_R | Homology arm right for | |
| | | Fluorescent reporter | |
| | | replacement of rxpA-C | |
| NO 155 | indC_L | Homology arm left for indC | |
| | | promoter exchange | |
| NO 156 | indC_R | Homology arm right for indC | |
| | | promoter exchange | |
| NO 157 | glbA_L | Homology arm left for promoter | |
| | | exchange of glbA | |
| NO 158 | glbA_R | Homology arm right for promoter | |
| | | exchange of glbA | |
| NO 159 | mxnB_L | Homology arm left for mxnB | |
| | | Promoter exchange | |
| NO 160 | mxnB_R | Homology arm right for mxnB | |
| | | Promoter exchange | |
| NO 161 | mxnK_L | Homology arm left mxnK | |
| | | Promoter exchange | |
| NO 162 | mxnK_R | Homology arm right for mxnK | |
| | | Promoter exchange | |
| NO 163 | stlCDE_L | Homology arm left for stlCDE | |
| | | Fluorescent reporter | |
| | | replacement | |
| NO 164 | stlCDE_R | Homology arm right for stlCDE | |
| | | Fluorescent reporter | |
| | | replacement | |
| NO 165 | ppyS_L | Homology arm left for ppyS | PluTT01m_24855 |
| | | Fluorescent reporter | |
| | | replacement | |
| NO 166 | ppyS_R | Homology arm right for ppyS | PluTT01m_24855 |
| | | Fluorescent reporter | |
| | | replacement | |
| NO 167 | xscA/xscI_L | Homology arm left for xscA/xscI | Xenorhabdus sp. TS4_R |
| | | Promoter exchance/Refactoring | |
| NO 168 | xscA/xscI_R | Homology arm right for xscA/xscI | Xenorhabdus sp. TS4 |
| | | Promoter exchance/Refactoring | |
| NO 169 | xscKΔxscH_L | Homology arm left for xscKΔxscH | Xenorhabdus sp. TS4 |
| | | Promoter exchange/Refactoring | |
| NO 170 | xscKΔxscH_R | Homology arm right for | Xenorhabdus sp. TS4 |
| | | xsCKΔxscH Promoter | |
| | | exchance/Refactoring | |
| NO 171 | rpmA_L | Homology arm left for rpmA | Xenorhabdus indica |
| | | Promoter exchange | |
| NO 172 | rpmA_R | Homology arm right for rpmA | Xenorhabdus indica |
| | | Promoter exchange | |
| NO 173 | rpmD_L | Homology arm left for rpmD | Xenorhabdus indica |
| | | Promoter exchange | |
| NO 174 | rpmD_R | Homology arm right for rpmD | Xenorhabdus indica |
| | | Promoter exchange | |
| NO 175 | ΔrpmG_L | Homology arm left for ΔrpmG | Xenorhabdus indica |
| | | deletion | |
| NO 176 | ΔrpmG_R | Homology arm right for ΔrpmG | Xenorhabdus indica |
| | | deletion | |
| NO 177 | ΔrpmM-O_L | Homology arm left for ΔrpmM-O | Xenorhabdus indica |
| | | deletion | |
| NO 178 | ΔrpmM-O_R | Homology arm right for ΔrpmM- | Xenorhabdus indica |
| | | O deletion | |
| NO 179 | Δhfq_L | Homology arm left for Δhfq | Xenorhabdus indica |
| | | deletion | |
| NO 180 | Δhfq_R | Homology arm right for Δhfqv | Xenorhabdus indica |
| NO 181 | Δhfq_L | Homology arm left for Δhfq | Xenorhabdus nematophila |
| | | deletion | |
| NO 182 | Δhfq_R | Homology arm right for Δhfq | Xenorhabdus nematophila |
| | | deletion | |
| NO 183 | ΔcyaA_L | Homology arm left for ΔcyaA | Xenorhabdus nematophila |
| | | deletion | |
| NO 184 | ΔcyaA_R | Homology arm right for ΔcyaA | Xenorhabdus nematophila |
| | | deletion | |
| NO 185 | dprA_L | Homology arm left for activating | Xenorhabdus nematophila |
| | | dprA by promoter exchange | |
| NO 186 | dprA_R | Homology arm right for | Xenorhabdus nematophila |
| | | activating dprA by promoter | |
| | | exchange | |
| NO 187 | ΔcyaA_L | Homology arm left for ΔcyaA | Xenorhabdus doucetiae |
| | | deletion | |
| NO 188 | ΔcyaA_R | Homology arm right for ΔcyaA | Xenorhabdus doucetiae |
| | | deletion | |
| NO 189 | xabA_L | Homology arm left for activating | Xenorhabdus doucetiae |
| | | xabA by promoter exchange | |
| NO 190 | xabA_R | Homology arm right for | Xenorhabdus doucetiae |
| | | activating xabA by promoter | |
| | | exchange | |

TABLE 9
Chemical structures of benzoic acid (BA) derivatives and
rupshomycin compounds 19-25 (FIG. 12)

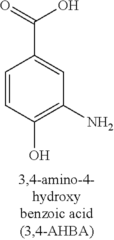 [SMILES: Nc1cc(C(=O)O)ccc1O]

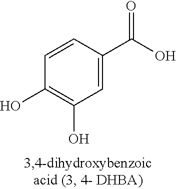 [SMILES: O=C(O)c1ccc(O)c(O)c1]

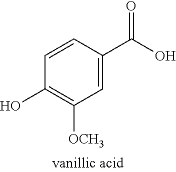 [SMILES: COc1cc(C(=O)O)ccc1O]

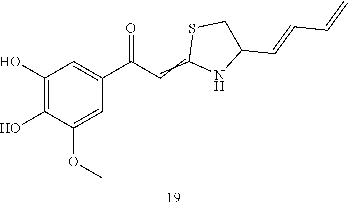 [SMILES: C=C/C=C/C1CSC(=CC(=O)c2cc(O)c(O)c(OC)c2)N1]

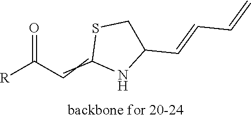

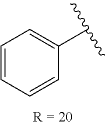 [SMILES: *c1ccccc1]

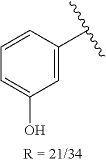 [SMILES: *c1cccc(O)c1]

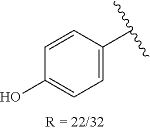 [SMILES: *c1ccc(O)cc1]

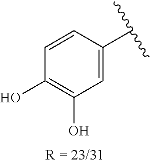 [SMILES: *c1ccc(O)c(O)c1]

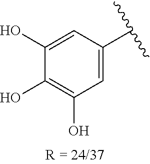 [SMILES: *c1cc(O)c(O)c(O)c1]

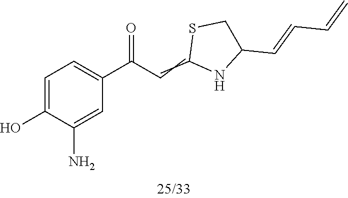 [SMILES: C=C/C=C/C1CSC(=CC(=O)c2ccc(O)c(N)c2)N1]

TABLE 10
structures of described compounds

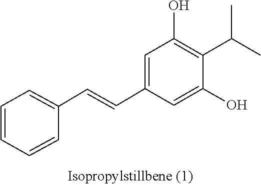 [SMILES: CC(C)c1c(O)cc(/C=C/c2ccccc2)cc1O]

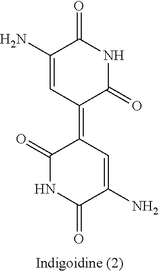 [SMILES: NC1=C/C(=C2/C=C(N)C(=O)NC2=O)C(=O)NC1=O]

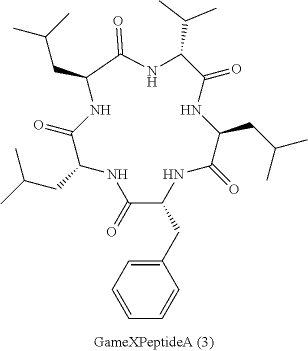 [SMILES: CC(C)C[C@@H]1NC(=O)[C@@H](CC(C)C)NC(=O)[C@@H](Cc2ccccc2)NC(=O)[C@H](CC(C)C)NC(=O)[C@@H](C(C)C)NC1=O]

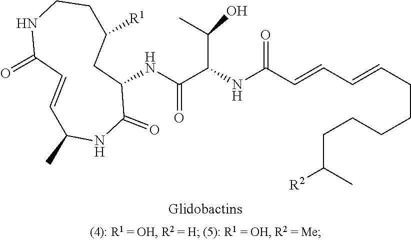

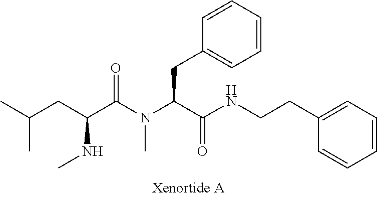 [SMILES: CN[C@@H](CC(C)C)C(=O)N(C)[C@@H](Cc1ccccc1)C(=O)NCCc1ccccc1]

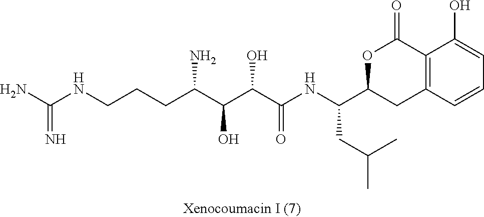 [SMILES: CC(C)C[C@H](NC(=O)[C@@H](O)[C@@H](O)[C@@H](N)CCCNC(=N)N)[C@@H]1Cc2cccc(O)c2C(=O)O1]

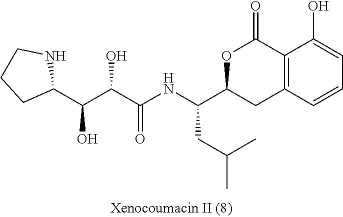 [SMILES: CC(C)C[C@H](NC(=O)[C@@H](O)[C@@H](O)[C@@H]1CCCN1)[C@@H]1Cc2cccc(O)c2C(=O)O1]

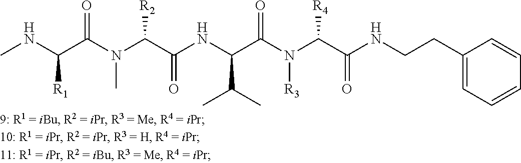

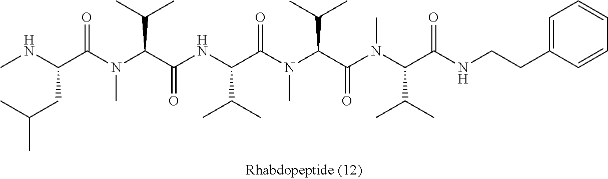 [SMILES: CN[C@@H](CC(C)C)C(=O)N(C)[C@H](C(=O)N[C@H](C(=O)N(C)[C@H](C(=O)N(C)[C@H](C(=O)NCCc1ccccc1)C(C)C)C(C)C)C(C)C)C(C)C]

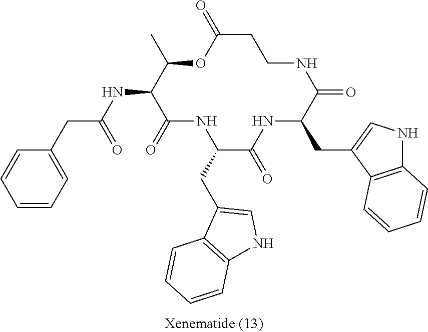 [SMILES: C[C@H]1OC(=O)CCNC(=O)[C@@H](Cc2c[nH]c3ccccc23)NC(=O)[C@H](Cc2c[nH]c3ccccc23)NC(=O)[C@H]1NC(=O)Cc1ccccc1]

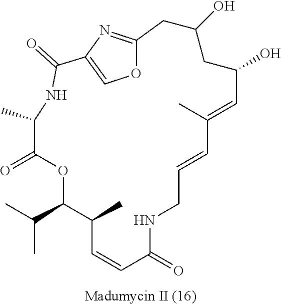 [SMILES: CC1=C\[C@@H](O)CC(O)Cc2nc(co2)C(=O)N[C@@H](C)C(=O)O[C@H](C(C)C)[C@@H](C)/C=C\C(=O)NC\C=C\1]

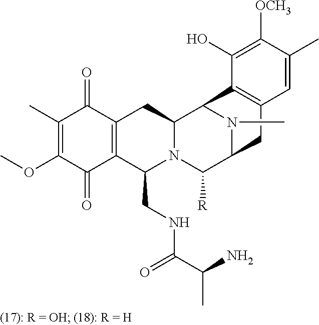

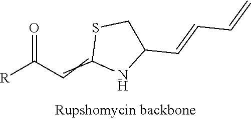

R for Rupshomycin compounds 19-26:

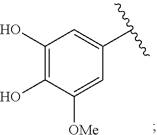 [SMILES: *c1cc(O)c(O)c(OC)c1]

19:

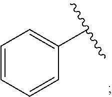 [SMILES: *c1ccccc1]

20:

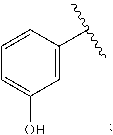 [SMILES: *c1cccc(O)c1]

21:

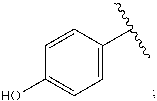 [SMILES: *c1ccc(O)cc1]

22:

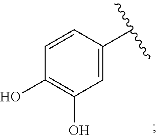 [SMILES: *c1ccc(O)c(O)c1]

23:

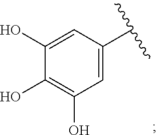 [SMILES: *c1cc(O)c(O)c(O)c1]

24:

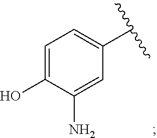 [SMILES: *c1ccc(O)c(N)c1]

25:

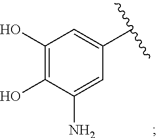 [SMILES: *c1cc(N)c(O)c(O)c1]

26:

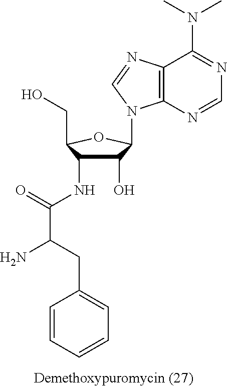

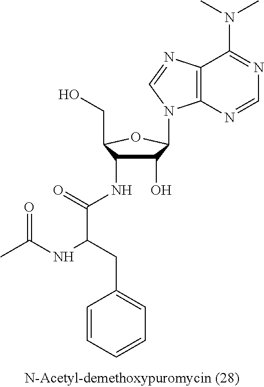

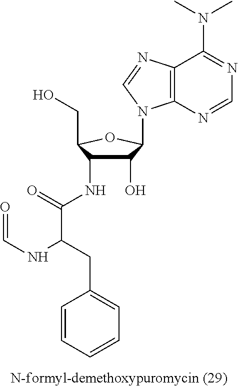

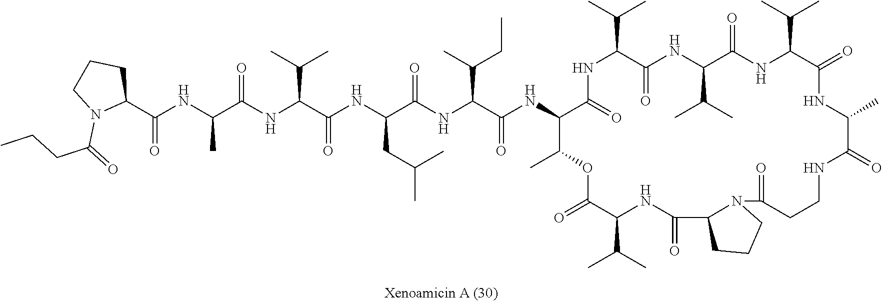 [SMILES: CCCC(=O)N1CCC[C@H]1C(=O)N[C@H](C)C(=O)N[C@H](C(=O)N[C@H](CC(C)C)C(=O)N[C@H](C(=O)N[C@H]1C(=O)N[C@@H](C(C)C)C(=O)N[C@H](C(C)C)C(=O)N[C@@H](C(C)C)C(=O)N[C@H](C)C(=O)NCCC(=O)N2CCC[C@H]2C(=O)N[C@@H](C(C)C)C(=O)O[C@@H]1C)C(C)CC)C(C)C]

TABLE 11
List of fluorescent proteins with key parameters.
Values for excitation/emission maxima (λₑₓ,
λₑₘ) were taken from www.fpbase.org

| | λₑₓ | λₑₘ | | Fbbase ID |
|---|---|---|---|---|
| Protein | [nm] | [nm] | Characteristics | (www.fpbase.org) |
| mTagBFP2 | 399 | 454 | monomeric | ZO7NN |
| sfGFP | 485 | 510 | monomeric, fast | B4SOW |
| (superfolded GFP) | | | maturation | |
| mNeonGreen | 506 | 517 | monomeric, | ZRKRV |
| | | | superior | |
| | | | brightness | |
| mGold | 515 | 531 | monomeric, | UNETX |
| | | | bright, suitable | |
| | | | for FACS | |
| mScarlet-I | 569 | 593 | monomeric, high | 6VVTK |
| | | | brightness | |
| mCherry | 587 | 610 | faster maturation | ZERB6 |
| | | | than mScarlet-I, | |
| | | | but less bright | |

### DESCRIPTION OF THE FIGURES

FIG. 1 shows A) an overview of the method described herein to identify global regulators comprising replacement of at least two constitutively expressed BGCs by two different fluorescent reporters, followed by transposon mutagenesis and analysis for loss of fluorescence for both reporters. Non-fluorescent mutants are identified, isolated and then analysed by whole-genome sequencing to identify global regulators. Non-fluorescent mutants are also screened for the production of Natural Products by HPLC/MS analysis. B) Schematic of pAR20 plasmid with crRNA frameworks and elements for fluorescent reporter replacement, and of cloning of synthetic dsDNA fragments harboring the repair template and target spacer together with a fluorescent reporter sequence into the synthetic crRNA framework encoded on pAR20. C) Schematic of pAR20 plasmid with crRNA frameworks, synthetic dsDNA fragments, and elements for cluster activation, DR: direct repeat. D) Schematic representation of an mass spectroscopy (MS) chromatograms according to expected process: deletion of a global regulator with subsequent activation of a selected secondary metabolite. 1) Wild type (WT) strain with complex metabolite profile; 2) after deletion of a global regulator the metabolite profile becomes clearer; 3) Exchange/replacement of native secondary metabolite promotor by an inducible promotor in the global regulator deletion background; 4) targeted induction of the desired secondary metabolite.

FIG. 2 shows design of genome editing based on a two plasmid system in Photorhabdus using stlA deletion as an example. A) Phenotypic comparison of wild type (WT) and ΔstlA deletion mutant; B) Role of StlA in the biosynthesis of isopropylstilbene (1). C) Schematic representation of the selection of target spacer and homology arms. D) schematic cloning of synthetic dsDNA fragments harboring the repair template and target spacer into synthetic crRNA framework encoded on pAR18; detailed genotype of pAR18 is shown in Table 1. The figure was created with BioRender.com.

FIG. 3 shows A) schematic representation of pAR20 plasmid that can be used for genome editing with CRISPR/Cpf1 approach digested with Bsal. B) Overview of the developed single plasmid CRISPR-Cpf1 genome editing workflow, e.g. for BGC activation and/or global regulator deletion. The approach starts with conjugation of the target specific pAR20 into the strain to be edited (with or without previous global regulator (GR) deletion, day 1); selection of conjugates and induction of CRISPR/Cpf1 components followed by selection of correct edited strains (day 3); plasmid curing with sucrose (day 5); screening for new or known compounds (day 6); entering a new editing cycle (day 7). Figure created with BioRender.com.

FIG. 4 shows overview of MS spectra of deletion mutants. A) Base peak chromatograms (BPC) of P. laumondii TTO1 wild type and deletion mutants. B) BPCs of X. nematophila wild type and deletion mutants.

FIG. 5 shows gel electrophoresis images of deletion and promoter exchange mutants after colony PCR.

FIG. 6 shows single plasmid CRISPR/Cpf1 genome editing method for promoter exchange upstream of indC. A) Schematic representation of target spacer and homology arms selection; B) schematic cloning of synthetic dsDNA fragments harboring the repair template and target spacer and the vanillic acid promoter sequence into synthetic crRNA framework encoded on pAR20. C) Schematic representation of the genome editing approach mediated by pAR20; the sequence between the homology arms are replaced by the promoter sequence; detailed genotype of pAR20 can be found in Table 1. D) Phenotypic comparison of induced and non-induced mutants (VA=vanillic acid). E) Structure of indigoidine (2). The figure was created with BioRender.com.

FIG. 7 shows overview of MS spectra of induced (solid line) and non-induced (dashed line) cultures after successful promoter exchanges. A) Chromatograms of P. laumondii TTO1 and edited mutants; wild type (WT) BPC and for better visualization extracted ion chromatograms (EICs) representing major derivatives of associated NPs. B) Chromatograms of Xenorhabdus nematophila wild type and genome edited mutants. Structures are shown in Tables 9 and 10.

FIG. 8 shows strain engineering towards madumycin production in X. nematophila. A) Putative madumycin BGC mxn; B) Cluster refactoring; replacing of native promoters PmxnA and PmxnB by insertion by the PvanCC promoter at different positions and insertion of genetic parts like insulators riboJ51 and ribosomal binding sites. C) Putative biosynthesis of 16 by the mxn encoded assembly line. A: adenylation domain, C: condensation domain, CY: cyclization domain, E: epimerization domain, MT: methylation domain, PCP: peptidylcarrier protein domain, ACP (small black circle): acyl carrier protein domain, KS: β-ketoacyl carrier protein synthase domain, KR: ketoreductase domain, DH: dehydratase domain, OX: oxidation domain, TE: thioesterase domain. D) HPLC/MS chromatograms of “re”-engineered mxn cluster strains; BPC (black), EIC for 16 (504.2 m/z [M+H]+, red), induced (solid line) and non-induced (dashed line).

FIG. 9 shows structure elucidation and proposed biosynthesis pathway of the formation of madumycin II (16) in X. nematophila. A) Three different promotor exchange constructs and EICs for 16 (504.2 m/z [M+H]⁺) of induced cultures. B) HR-MS spectrum for 1; HR-MS spectrum for 1 after addition of L-alanine-D₄; HR-MS spectrum for 1 after addition of D₈-L-valine. C) MS/MS fragmentation comparison of produced compound 16 and its chemical standards.

FIG. 10 shows A) the xsc BGC responsible for the production of safracin A (SAC-A, 2) and safracin B (SAC-B, 1) in Xenorhabdus sp. TS4. B) shows strains I-III and corresponding EICs for SAC-A and SAC-B production; I: wild type stain with silent xsc BGC; II: Bidirectional promoter exchange between xscA and xscJ, production of SAC-A and B; III: Additional promoter exchange upstream of xscK while deleting xscH; producing SAC-B exclusively. C) Biosynthetic pathway of SAC-A and B.

FIG. 11 shows A) semisynthesis overview to produce ecteinascidin 743 and (−)-Jorumycin starting from safracin B; B) comparison of SAC-B production in Xenorhabdus sp. TS4 and E. coli LZ84 harboring different plasmids encoding the xsc BGC.

FIG. 12 shows stepwise activation and characterization of the rupshomycin biosynthetic gene cluster (rpm). I: native and silent BGC rpm under laboratory conditions. II: P_{vanCC} promoter exchange upstream of rpmA, no production observed after induction. III: P_{vanCC} promoter exchange upstream of rpmD, no production observed after induction. IV: promoter exchange upstream of rpmA with exchange of the intergenic region between rpmC and rpmD by RiboJ51 and T7g10 UTR, production of 19 observed after induction. V: P_{tac} promoter exchange upstream of rpmA with exchange of the intergenic region between rpmC and rpmD by RiboJ51 and T7g10 UTR; feeding of several benzoic acid derivatives (all 1 mM) leading to the production of compounds 20-24 after induction. VI: strain V with deletion of rpmG, conjugated with expression vector expressing rpmA and rpmJ; detection of compound 25 after induction. VII: strain V with deletion of rpmMNO and feeding of 3,4-AHBA; no production observed, culture dies after induction. VIII: strain VII conjugated with expression vector expressing rpmM and rpmN. IX: strain VII conjugated with expression vector expressing rpmO. In strains V-IX, in addition to promoter exchange, the global regulator Hfq was also deleted.

FIG. 13A) Overexpression of rpmA and rpmG encoded on pAR29badad reveled 3-amino-4-hydroxy benzoic acid (3,4-AHBA) as natural starting unit for rpm BGC. B) Biosynthetic gene cluster for the formation of rupshomycin (26) and derivatives thereof. C) Putative biosynthetic pathway for rupshomycin (26) and derivatives 31 and 33. Hydroxylation in 5-position is catalyzed by rpmG (shaded); Removal of 3 amino group is catalyzed by RpmB and RpmM in the presence of Trp; C: condensation domain, CY: cyclization domain, PCP: peptidylcarrier protein domain, ACP (small black circle): acyl carrier protein domain, KS: β-ketoacyl carrier protein synthase domain, KR: ketoreductase domain, DH: dehydratase domain, TE: thioesterase domain.

FIG. 14 Growth curves recorded with the Tecan Sparc microtiter plate reader, 100 μl culture in a 96 well microtiter plate over 24 hours for X. indica strains producing rupshomycin derivatives. Addition of the inducer and benzoic acid was added from the start. The numbering I-IX corresponds to the strains in FIG. 12. A: growth of strain I culture with and without induction (1 mM IPTG), with and without addition of 3,4-AHBA. B: growth of strain V culture with and without induction, with and without addition of 3,4-AHBA. C: growth of strain I culture with and without induction, with and without addition of 3,4-DHBA. D: growth of strain V culture with and without induction, with and without addition of 3,4-DHBA. E: growth of induced and 3,4-AHBA supplemented strain VIII culture with vector expressing rpmMN or empty expression vector. F: growth of induced and 3,4-AHBA supplemented strain IX culture with vector expressing rpmO or empty expression vector.

FIG. 15A) shows schematic representation of the high-throughput genome editing approach using microtiter plates. Overnight cultures grown in a deepwell plate are diluted 1:25 in 100 μl and transferred to a 96 well microtiter plate. Cultures are incubated in a microplate reader at 30° C. while growth is recorded. After the CRISPR procedure, two dilutions are prepared and transferred to selection plates using a multichannel pipette. Single colonies can be tested for successful genome editing after two days. B) shows an example of a 7×7 patches spotted with the ROTOR⁺ from Singer Instruments.

FIG. 16 shows spotting and plating of CRISPR cultures. Plate with edited colonies after 2 days of incubation. A) Place the square agar plate at an angle of about 45°. B) Using a 12 channel pipette, add 20 μl to the upper end of the plate and allow the drops to run to the lower end without running into each other. C) Plates with cultures of different dilutions after two days of regeneration.

FIG. 17 A) shows fluorescence imaging of pigmented Photorhabdus luminescens TTO1 indicator strains. Strains were constructed using the CRISPR-Cas12 approach described in this patent. In these strains, BGCs stlCDE, ppyS, AQ MT were replaced by an mNeonGreen gene. Strains were spotted onto LB agar plates and imaged at different days using an automated Leica Thunder Imager stereo microscope. Selected indicator strains develop fluorescence over time and are thus suited for screening. Intensities were manually adjusted for good contrast for each strain, while the intensity range was kept identical over the time course. B) shows imaging of non-pigmented Xenorhabdus nematophila indicator strains. Strains were constructed using the CRISPR-Cas12 approach described in this patent. In these strains, BGCs rxp, ppyS, Odilorhabdin (odIC) were replaced by a mNeonGreen gene. Strains were spotted onto LB agar plates and imaged at different days using an automated Leica Thunder Imager stereo microscope. Selected indicator strains develop fluorescence over time and are thus suited for screening. Intensities were manually adjusted for good contrast for each strain, while the intensity range was kept identical over the time course.

FIG. 18 shows the method to obtain multi-producer strains. First strains with single BGC activation in clean background (without other active BGCs) producing single secondary metabolites are identified and analysed for the individual bioactivities of the metabolites. These experiments allow the correlation between BGC and SM with interesting bioactivities and which can be combined. Thus, the so individuated BGC are inserted together into a single strain to generate a multi-producer strain, which can be used for applications in animal health, agriculture and medicine.

FIG. 19 shows suitable crRNA frameworks: A) A crRNA framework consisting of 5 components: 1. host specific promoter, constitutive or inducible, or derived from a housekeeping gene; 2. A leader sequence; 3. cas12 specific direct repeat; 4. A dummy sequence carrying two opposite type II restriction enzyme recognition sites separated by at least 4 basepairs for scarless insertion of target specific spacer sequence; 5. a transcription terminator. After assembly a target specific crRNA array is obtained. Since the spacer sequence is not framed by two direct repeats, it is not fully processed by the cas12 enzyme and the length of the spacer should be 20-23 bp; B) crRNA framework consisting of the same components as in A only with further direct repeat behind the dummy spacer sequence. This provides for independent processing of the pre-crRNA. Here, in addition, with simultaneous fusion with homologous regions, two distinct crRNA arrays can be created (to target leading and lagging strand). R repair templates for the double-strand break is located in between. C) Structure as in B crRNA array assembled by short DNA fragments carrying a target-specific spacer sequence and a cas12 direct repeat. Depending on the strength of the promoter, up to 10 spacers can be transcribed. D) Opposed crRNA framework. Assembly introduces target-specific spacers, direct repeats, terminators, and repair templates.

FIG. 20 shows A) Investigated biosynthetic gene cluster dpr (deoxy-puromycin) of X. nematophila ATCC 19061, graphical representation via antiSMASH; the red arrow marks the position of the promoter exchange. B) Base peak chromatograms (BPC) of the induced production cultures (top=Sf900 medium, bottom=XPP medium) of X. nematophila ΔhfqΔP_{dprA}::vanR_P_{vanCC} in the retention time range between 2 and 7 min; induced in blue, non-induced in red, wild type in green; with the mass to charge ratios (m/z) of the strongest signals; measured by RP-UPLC-ESI-qTOF-MS (Impact). C) MS/MS spectra of induced production cultures (Sf900, ¹³C, ¹⁵N medium); showing the mass shifts as well as the postulated structural and molecular formulae and molecular masses in Dalton (Da); the MS spectrum corresponds to the retention time between 4.3-4.6 min; measured by RP-UPLC-ESI-qTOF-MS (Impact).

FIG. 21 shows spectroscopic data from NMR measurements (¹H (500 MHz) and ¹³C (125 MHz)); HMBC correlations as arrows, COSY correlations as bold bonds; sample in DMSO-d6, δ in ppm, J in Hz. A) Structure of demethoxypuromycin (M1, 27) (441 Da). B) Structure of N-acetyl-demethoxypuromycin (M2, 28) (483 Da). C) Structure of N-formyl-demethoxypuromycin (M3, 29) (469 Da).

FIG. 22 shows A) Investigated biosynthetic gene cluster xab of Xenorhabdus doucetiae, graphical representation via antiSMASH; the red arrow marks the position of the promoter exchange. B) Promoter exchange in ΔcyaA results in specific production of Xenoamicine in the “cleaner” background as comparison to Wiltype in Xenorhabdus doucetiae. HPLC/MS analysis of targeted Xenoamicine A (30) production in X. doucetiae WT and ΔcyaA are shown as base peak chromatograms (BPC) where WT, ΔcyaA, induced are represented with black line and WT and ΔcyaA non-induced with gray line.

FIG. 23 shows chemical structures of further rupshomycin derivatives 31-44 prepared according to the methods described herein.

FIG. 24A) shows extracted-ion chromatograms (EIC) of rupshomycin derivatives 26, 31, and epoxy-rupshomycin derivatives 45-48; B) chemical structures of epoxy-rupshomycin derivatives 45-48; C) mass spectra of compounds 26, 31, 46 and 47.

FIG. 25 upper part shows extracted-ion chromatograms (EIC) of rupshomycin derivatives 49-52; lower part shows chemical structures of rupshomycin derivatives 49-52.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the scope of the invention.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the scope of the invention as described in the following claims.

### EXAMPLES

### Material and Methods

### Bacterial Culture Conditions

Photorhabdus luminescence laumondii subsp. TTO1 and Xenorhabdus doucetiae were grown in LB with shaking for at least 16 hours at 30° C. E. coli strains were grown in LB with shaking for at least 16 hours at 37° C. The medium was supplemented with kanamycin (50 μg/ml), ALA (5-Aminolevulinic acid) (50 μg/ml), DAP (Diaminopimelic acid) (300 μM) or 0.2% L-arabinose when appropriate. Promoter exchange mutants were induced by adding L-arabinose (0.2%, v/v) to the cultures. All plasmids and strains used in this study are listed in Table 2.

Production of selected metabolites took place in XPP3 medium or Bacto™ CD Supreme Fermentation Production Medium (SFPM) for 72 h at 28° C.

### Transposon Mutants Construction

Transposon mutagenesis was performed by conjugating Xenorhabdus doucetiae and Photorhabdus laumondii with a pool of donor E. coli APA752 (harboring the pKMW3 (mariner transposon vector library with ~3 million unique 20mer DNA barcodes flanked by common PCR priming sites) in WM3064 (conjugation donor strain)). APA752 was a gift of Prof. Adam Deutschbauer (Berkeley, California, USA). The donor strain APA752 and the recipient strains X. doucetiae and P. laumondii were grown to mid-log-phase (OD 0.6 to 0.8) then mixed at a donor/recipient ratio of 1:3 for 24 h at 30° C. on LB agar plates. The conjugation reaction mixtures were scraped into LB and the cells plated on LB agar plates supplemented with 100 ug/ml Kanamycin, and incubated the plates at 30° C. After 2 days of growth, non-fluorescent colonies were selected and those were grown in 10 ml LB with 100 ug/ml Kanamycin at 30° C. to a final OD of 1.5. Final volume of 20% glycerol was added, 2 ml−80° C. freezer stocks were made, and simultaneously cell pellets were collected for genomic DNA extraction for Whole genome sequencing.

### Genomic DNA Extraction

For sequencing of transposon-insertion mutants, genomic DNA was extracted using the DNeasy Blood & Tissue Kit (Qiagen) following the manufacturer's instructions.

### Whole Genome Sequencing and Identification of Transposon Insertion Sites

Genomic DNA mutant libraries were constructed using NEBNext Ultra II FS DNA Library preparation Kit and Whole genome sequencing was performed using 2×150 bp paired-end chemistry on Illumina MiniSeq platform. Transposon insertion sites were identified by mapping the IR and IL (Right and Left Inverted Repeat sequence of the mariner Transposon) against each sequenced fastaQ file of individual mutants and viewed in Geneious Prime (https://www.geneious.com).

### Metabolite Extraction and HPLC-MS/MS Analysis

Fresh LB medium (10 ml) was inoculated with an overnight culture to an OD₆₀₀ of 0.1. After 72 h of cultivation at 30° C. with shaking, 500 μl of the culture was mixed with equal volume of Methanol and vortexed at full speed for 1 min. The Mixture was then diluted to 1:5 with Methanol and centrifuged for 20 min at 13,000 r.p.m. and the supernatant was directly subjected for analysis by HPLC-MS/MS using a Dionex Ultimate 3000 system with a Bruker AmaZon Speed mass spectrometer. The peak areas of the compounds were quantified using DataAnalysis (Bruker).

Metabolite Production, Extraction and HPLC-MS/MS with Absorbing Amberlite XAD-16 Resin

For in situ extraction of metabolites 10 ml production medium containing appropriate antibiotics and 2% (v/v) of XAD-16 slurry, was inoculated to an OD₆₀₀ of 0.5 and incubated at 30° C. until OD600 of ~1 was reached. Afterwards the appropriate inducers were added and the culture was set to 28° C. After 72 h incubation under shaking conditions (180 r.p.m.), the culture supernatant was removed while the XAD-16 resin remained in the flask. 10 ml of methanol was added to the resin and the metabolites were extracted during an incubation time of 1 h at room temperature with continuous shaking. 1 ml of the extraction was then diluted to 1:5 with Methanol and centrifuged for 20 min at 13,000 r.p.m. and the supernatant was directly subjected for analysis by HPLC-MS/MS using a Dionex Ultimate 3000 system with a Bruker AmaZon Speed mass spectrometer. DataAnalysis (Bruker) was used to analyse and quantify the chromatograms and peak areas of the compounds.

### Flow Cytometry for Characterization of Expression Dynamics of the Reporter Strains.

Flow cytometry measurements were performed on a BD Fortessa Flow Cytometer (BD Bioscience) to select reporter strains and optimize growth condition, e.g. time point for maximal expression of reporter genes and media with highest expression level. For this, cells were grown for 1-3 days in low-salt LB medium. Other media, such as minimal media (M9 and derivatives), insect cell culture media (Sf900 II) or defined production media (XPPM or XPP3) can also be used, depending on the expression profile of the reporter genes. The samples were washed once with PBS (pelleting at 5000 rcf for one minute) and diluted 1:300 with PBS before measuring. Reporter strains carrying for example mNeonGreen and mScarlet were measured using the 488 nm and 561 nm laser lines (100 mW) and the bandpass filter 510/20 and 586/15 respectively. Gating was set according to forward scatter, sideward scatter and fluorescence signal. 30,000 events were acquired. The data were recorded using the BD FACS DIVA software (BD Bioscience). This kind of experiment is suitable for high throughput approaches (see M4).

FACS for Collecting of Possible Candidates with Deletion of Global Regulators.

Fluorescence activated cell sorting measurements were performed on a BD FACSAria™ Fusion Flow Cytometer (BD Biosciences). Reporter strains carrying mNeonGreen and mScarlet were measured after transposon mutagenesis. Cells were excited with 488 nm and 561 nm lasers (100 mW) and fluorescence was recorded using appropriate filter sets. Gates for sorting were set to sort for cells without any fluorescent signal and double fluorescence (negative control).

### Screening of Indicator Mutants Having Loss Fluorescence Emission

Identification of potential global regulator mutants can be performed by different methods at varying throughput (Table 1).

TABLE 1
Methods for screening of fluorescent reporter strains

| | | | Time |
|---|---|---|---|
| Method | Sensitivity | Throughput | effort |
| (M1) Fluorescence imaging of | High | Low | High |
| colony smear | | | |
| (M2) Stereo microscopy of | Medium | Medium | Medium |
| colonies on solid agar | | | |
| (M3) Fluorescence spectroscopy | Medium- | Medium- | Medium |
| using a microplate reader | High | High | |
| (M4) FACS | High | High | Low |

### M1) Fluorescence Imaging of Colony Smear

This screening method is based on manual fluorescence microscopy imaging using an upright or inverted fluorescence microscope with a high magnification objective and a sensitive detector (preferably cooled EMCCD or sCMOS camera). For example it can be made by using a Zeiss AxioPlan 2 upright wide field microscope with a 100× oil NeoFluor phase contrast objective and a halogen lamp.

Colonies are picked with a sterile tip or spatula and smeared onto a standard microscopy glass slide. The smear is covered with a 170 μm thick cover glass (optional: add a drop of buffered solution, e.g. PBS), mounted onto the microscope and imaged in phase contrast and fluorescence mode. Fluorescence signal of bacterial cells is recorded using appropriate filter sets (here: Zeiss GFP filter cube for mNeonGreen or Zeiss Texas Red filter cube for mScarlet-I). This approach has a low throughput, but equipment is available in many laboratories.

### M2) Stereo Microscopy of Colonies on Solid Agar

For faster screening of transposon mutants, colonies on solid agar (typically grown for 2-5 days) can be imaged using a stereo microscope with a white light and fluorescence module. Systems with automated stages increase the throughput as they facilitate screening of entire plates. Here, a Leica Thunderlmager M205FC with an LED illumination system can be used. Non-fluorescent colonies are identified in overlays of fluorescence and white light images and can be subsequently picked and cultured. Sequencing and HPLC-MS is used to identify global regulator mutants.

### M3) Fluorescence Spectroscopy Using a Microplate Reader

Transposon mutants can also be screened using microplate readers. Microtiter plates (96-well or 384-well format) are inoculated with single colonies from selection agar plates. Plates are incubated at optimized conditions for 1-3 days and subsequently measured using a microplate reader with absorption and fluorescence modules. For this, a Tecan Spark system with a fluorescence module can be used. Measuring the OD₆₀₀ for each well reports on the biomass, while the fluorescence signal in two spectral windows is recorded to screen for loss of reporter protein fluorescence. Automatic colony picking using robotic systems increase the throughput, as shown here using a Singer PIXL colony picking robot.

### M4) FACS

FACS allows for sorting of non-fluorescent bacteria from liquid cultures at high throughput. After transposon mutagenesis, bacteria are grown in liquid medium including appropriate selection markers for 1-3 days. As FACS can separate individual cells with specific properties, culturing on solid agar is not required. Non-fluorescent cells can be sorted either directly into well plates or into reaction tubes followed by streaking onto solid agar to isolate individual mutant clones. Sorting into well plates can be combined with M3 to verify the loss of fluorescence. In the examples described herein was used a BD FACSAria™ Fusion Flow Cytometer.

### Strains, Plasmids, and Growth Conditions for BGC/Fluorescence Reporter Replacement and BGC Activation/Refactoring

All bacterial strains and plasmids used are listed in Table 1. E. coli DH10B were used as cloning for plasmid construction E. coli ST18 was used as donor strain for conjugation to transfer plasmids into Photorhabdus laumondii subsp. laumondii TTO1 and Xenorhabdus nematophila ATCC 19061. E. coli was cultured in LB medium at 37° C., and Photorhabdus/Xenorhabdus was cultured in LB medium at 30° C. All liquid cultures were incubated at 200 rpm and antibiotics and other supplements were added as follows: 50 μg/ml kanamycin (Km) and 50 μg/ml ALA for E. coli. 25 μg/ml kanamycin for Photorhabdus/Xenorhabdus. 1.5 g/L agar was added to LB for solid plates. In addition, the concentration of L-arabinose (Ara) and anhydrotetracycline (AHT) to induce expression of the Cpf1 protein and lambda red proteins was 0.4% (w/v) and 200 nM, respectively.

All strains were kept as glycerol stocks prepared in LB containing 25% glycerol at −80° C.

Plasmid Construction for BGC/Fluorescence Reporter Replacement Based on pAR20 Series

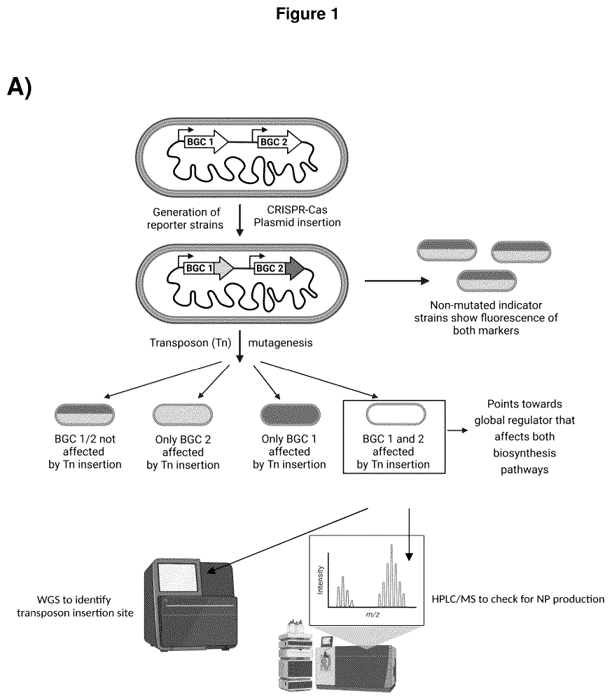

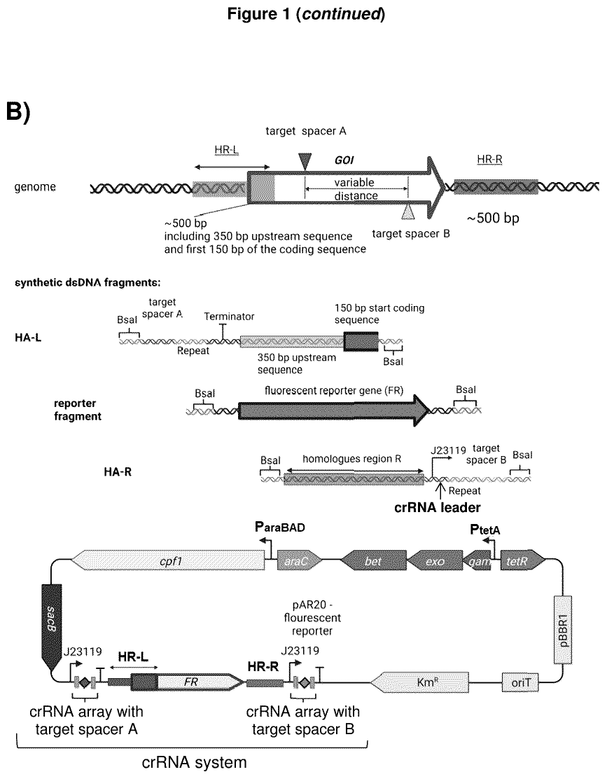

The empty pAR20 was constructed in silico and synthesized as whole plasmid by Double Helix Technologies (DOULIX™). To overcome multiple assembly rounds, the repair template containing homologues regions left and right (HR-L, HR-R) was fused together with target specific crRNA sequence on a synthetic dsDNA fragment (FIG. 1B). The position of the left/upstream homologues region was selected appropriately to conserve the native regulation of the gene and generate a fusion protein from partial origin gene and the fluorescent reporter. For this purpose, 350 bp of the UTR and 150 bp of the coding gene, including the start codon, were selected. The right homologues region was selected depending on the desired deletion size. The sequence does not necessarily have to be deleted as long as the PAM sequences of the selected spacers are mutated and another double strand break is prevented. The selection of the target spacer (TS), wherein TS-B is located shortly before HR-R, and TS-A is located shortly after the HR-L, was made by using the “CRISPR Sites” prediction function of the cloning software Geneious Prime. The selection criteria were target spacer with 31 bp length (23 bp minimum length) and TTTY as PAM sequence. At least one spacer sequence is selected per editing procedure. Selecting two spacer sequences, targeting each leading and lagging strand, the best results are achieved. Target spacer, homology repair templates and fluorescent reporter gene were ordered as synthetic dsDNA gene fragments, from Twist Bioscience and Integrated DNA Technologies, respectively (FIG. 1B). The NEB Golden Gate assembly mix containing pAR20 and dsDNA inserts was prepared according to the manufacturer's instructions resulting in a final editing plasmid (FIG. 1B). After the reaction, 1-2 μl of the mix was transformed into chemically or electro competent E. coli cells. Correct assembly was checked by colony PCR using primer pair AR533/534. Regulatory seuqences and homology repair templates are listed in Table 7c. Selected target spacer can be found in Table 7b.

Plasmid Construction for BGC Activation/Refactoring Based on pAR20 Series

For all cluster activations and refactoring, the above described pAR20-empty was used. The TS sequence was selected as follows: A region 50-1000 bp upstream of the cluster of interest were screened and annotated using the “CRISPR Sites” prediction (FIG. 1C). Two sequences were selected, oriented in opposite directions to target the leading and lagging strand respectively. The average GC content was between 30-60%, sequences with more than two Ts in a row were excluded. The left homologous region were then identified upstream of the most left selected target spacers with a minimal distance of 50 bp to the PAM sequence. The length was 500 (+/−100) bp and the average GC content was between 30 and 60% (FIG. 1C). The right homologues region is located downstream of the most right target spacer, but it must initiate with the start codon of the gene to be activated (FIG. 1C). In cases where the described distances could not be maintained, e.g. due to a limited number of suitable target spacer sequences, target spacer sequences located in the later homologous regions were also selected. Here artificial silent mutations were introduced into the PAM sequence to prevent the CRISPR complex from ‘attacking’ the repair template. Target spacer, homology repair templates and regulatory sequence (inducible promoter and regulator) ordered as synthetic dsDNA gene fragments, were obtained from Twist Bioscience and Integrated DNA Technologies, respectively (FIG. 1C). The assembly is carried out with the NEB Golden Gate assembly mix containing pAR20 and dsDNA inserts was prepared according to the manufacturer's instructions resulting in a final editing plasmid (FIG. 1C). After the reaction, 1-2 μl of the mix was transformed into chemically or electro competent E. coli cells. Correct assembly was checked by colony PCR using primer pair AR533/534. Regulatory seuqences and homology repair templates are listed in Table 7c. Selected target spacer can be found in Table 7b.

Plasmid Construction for BGC or Single Gene Deletion Based on pAR20 Series

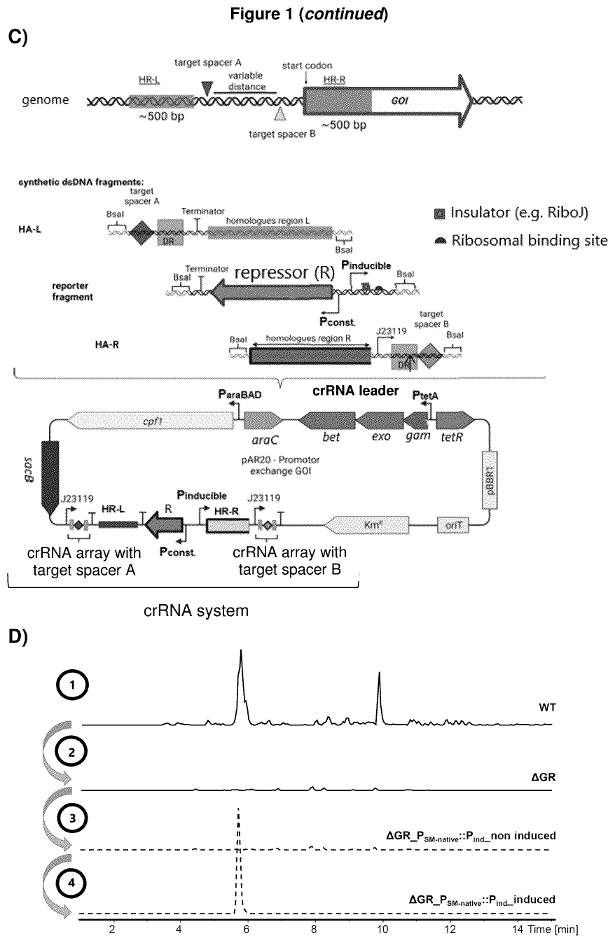

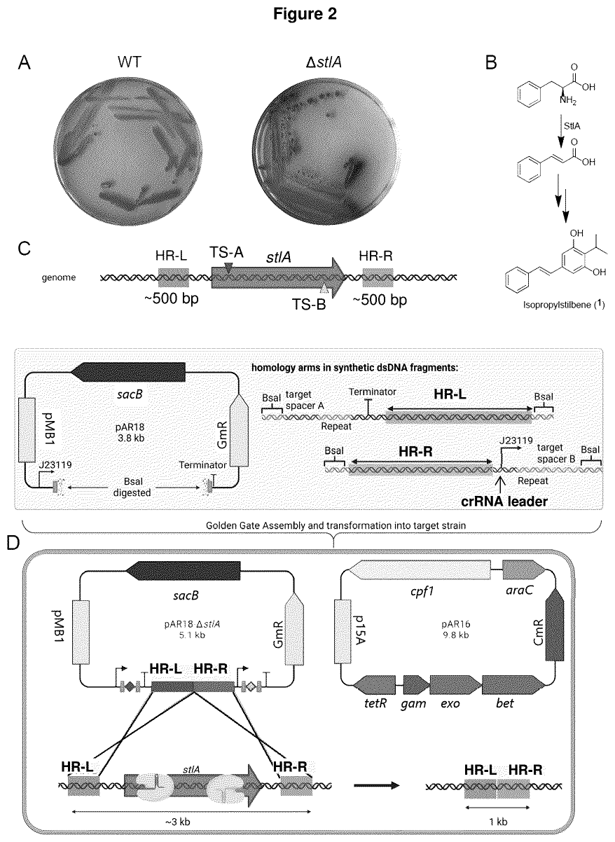

For all cluster activations and refactoring, the above described pAR20-empty was used. The TS sequence was selected as follows: within the coding sequence of the BGC/gene of interest were screened and annotated using the “CRISPR Sites” prediction (FIG. 2C). Two sequences were selected, oriented in opposite directions to target the leading and lagging strand respectively (FIG. 2C). The average GC content was between 30-60%, sequences with more than two “T”s in a row were excluded. The left homologous region were then identified upstream of the most left selected target spacers with a minimal distance of 50 bp to the PAM sequence, the region can be located within the coding sequence for a partial deletion or before the start codon for a complete deletion. The length was 500 bp (+/−50 bp) and the average GC content was between 30 and 60% (complete deletion, FIG. 2C). The right homologues region is located downstream of the most right target spacer, the region can be located within the coding sequence for a partial deletion or behind the stopp codon for a complete deletion (complete deletion, FIG. 2C). In cases where the described distances could not be maintained, e.g. due to a limited number of suitable target spacer sequences, target spacer sequences located in the later homologous regions were also selected. Here artificial silent mutations were introduced into the PAM sequence to prevent the CRISPR complex from ‘attacking’ the repair template. Target spacer, homology repair ordered as synthetic dsDNA gene fragments, were obtained from Twist Bioscience and Integrated DNA Technologies, respectively (FIG. 2C). The assembly is carried out with the NEB Golden Gate assembly mix containing pAR20 and dsDNA inserts was prepared according to the manufacturer's instructions resulting in a final editing plasmid. After the reaction, 1-2 μl of the mix was transformed into chemically or electro competent E. coli cells. Correct assembly was checked by colony PCR using primer pair AR533/534. Regulatory sequences and homology repair templates are listed in Table 7c. Selected target spacer can be found in Table 7b.

### Electro Transformation and Biparental Conjugation

pAR20 plasmids could be introduced into the target bacterial strains either by electro transformation (BTX™ ECM™ 630 Exponential Decay Wave Electroporator) or by conjugation. Electro competent Photorhabdus and Xenorhabdus cells were prepared following standard protocols.

For conjugation of the pAR20 derivatives overnight cultures of E. coli ST18, having a hemA deletion mutation, and Photorhabdus/Xenorhabdus were used, respectively. The optical density of the two cultures was measured and set to a ratio of 4:1 Recipient/Donor in a total volume of 1 ml. The cell mixture was centrifuged at 5000×g and washed twice with 1 ml LB. Finally, the cell pellet was resuspended in 100 μl LB and spotted onto a LB agar plate containing 50 g/ml 5-aminolevulinic acid, ALA. The next day, the cell mass was scraped off and resuspended in 1 ml LB. A dilution of 1:50 was plated on a selection plate with 25 μg/ml kanamycin (Km) and without ALA in order to select only for Photorhabdus/Xenorhabdus mutants and counter selecting for E. coli, due to the hemA deletion causing a defect in tetrapyrrole biosynthesis.

Cpf1 (Cas12a) Assisted Gene Editing (BGC Activation, BGC/FR Replacement, BGC Deletion) with pAR20 Series and Plasmid Curing

For general gene editing, a strain carrying a gene-specific pAR20 vector was inoculated as a 5 ml overnight culture in a 50 ml Erlenmeyer flask. The next day, this culture was diluted to a final OD of 0.5 in 10 ml LB containing 25 μg/ml kanamycin. The culture was then incubated at 30° C. until an OD of 0.8-1.0 was reached. After addition of AHT, the culture was further incubated at 25° C. for 1h. Subsequently, L-arabinose was added and incubated for another 3 h at the same temperature. Finally, 50 μl of the cell suspension was plated on a selection plate with kanamycin and arabinose. Successful editing approaches were confirmed by colony PCR.

To generate a plasmid free stain a 1:500 dilution of the overnight culture was inoculated into fresh LB culture containing 6% sucrose and incubated for further 20 h. This culture could be used directly for further rounds of genome editing.

### Example 1: Transposon Mutagenesis and Mutant Screening Based on Loss-of-fluorescence

Many bacterial strains potentially producing interesting secondary metabolites form non-pigmented colonies, rendering visual identification by color unsuitable. Replacing sequences of BGCs, such as NPRS, in such non-pigmented strains by fluorescent reporters, i.e. fluorescent proteins, allows for identification of global regulator mutants using fluorescence microscopy, spectroscopy or fluorescence-assisted cell sorting (FACS).

For the loss-of-fluorescence based screening, genetic replacement was performed in frame after the first 9-150 amino acids of the target enzyme of Xenorhabdus nematophila to avoid disturbance of native gene regulation, as regulatory elements are often positioned within the coding region. Notably, these mutants can be constructed using classical methods, e.g. suicide plasmids, or the CRISPR/Cas-based method presented here to increase the throughput. To screen for global regulator mutants the inventors created indicator strains in which highly expressed NRPS encoding genes are replaced by fluorescent reporters, so that the fluorescent reporter is under control of the same regulatory elements as the original NPRS encoding gene. Suitable indicator strains can also be created by replacing another gene being part of the BGC instead of NPRS.

BGCs responsible for Rhabdopeptide production was chosen as model BGC due to their high expression level. Growth conditions were optimized in 96-well format using a microplate reader Tecan Spark, to ensure that reporters were expressed at detectable levels.

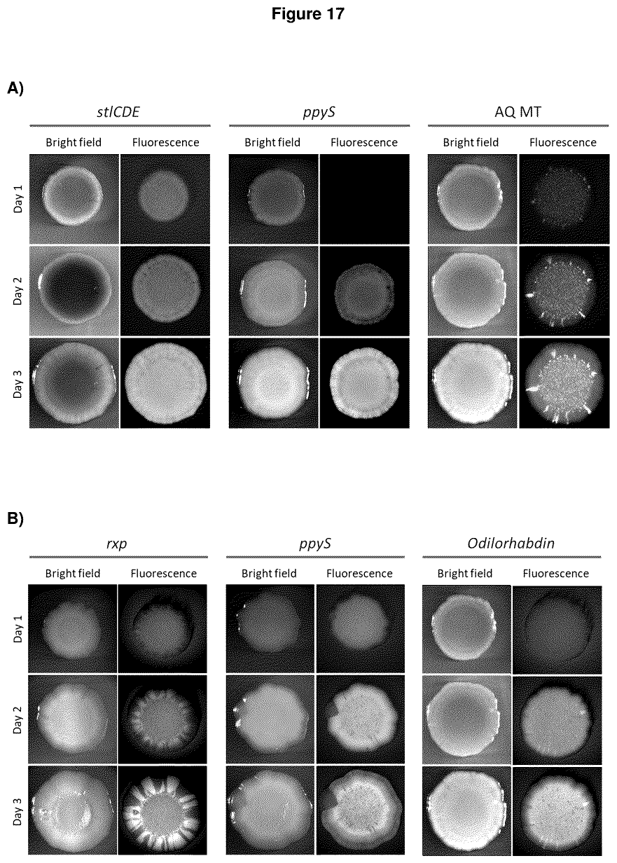

FIG. 17A shows Photorhabdus luminescens TTO1 indicator strains where BGCs stlCDE (Isopropylstilbene), ppyS (pyrones), and AQMT4 (anthraquinone) were replaced by a mNeonGreen gene. FIG. 17B shows Xenorhabdus nematophila indicator strains, where BGCs rxpA-C(XNC1_2228-2230) (Rhabdopeptides (9-12)), ppyS, and odlC (Odilorhabdins) were replaced by a mNeonGreen gene.

Afterwards, the constructed indicator strains were subjected to transposon mutagenesis (Method section).

Following transposon mutagenesis, cultures were plated out on LB agar plates including appropriate selection markers, incubated at suitable conditions (see Methods) and colonies were screened for the absence of fluorescence signal as described in the Method section above.

Absence of fluorescence signal of one reporter might be the result of transposon integration into the fluorescent reporter itself or the promoter region of the respective NRPS. Absence of both reporter signals, however, might indicate transposon integration into the genomic locus of a global regulator.

Candidate mutant bacteria colonies having lost fluorescence emission were verified by sequencing and HPLC-MS in order to identify global regulator gene: If no production of secondary metabolites is found in the mutants by HPLC/MS analysis, suggesting that the transposon might have hit the global regulators involved in the global regulatory pathways, WGS was performed in those mutants in order to identify the transposon insertion sites and thus the global regulator gene affecting the global regulation of secondary metabolite biosynthesis.

More than two fluorescent reporters could be used, but this might affect the strain fitness and additionally increases the complexity of the screening procedure.

Only non-essential global regulators can be identified with this method, as mutants have to be isolated and cultured for strain verification and characterization.

### Example 2: Optimization of the Two-Plasmid-Based CRISPR/Cpf1 Method for Photorhabdus

The present method represents an improvement of the CRISPR/Cpf1 editing method described by Ao X et al. 2018 (Front. Microbiol. 9:2307). A helper and a donor plasmid were used to perform genome editing supported by the lambda recombination system. The helper plasmid p46Cpf1-OP2 encodes an E. coli codon-optimized variant of the cpf1 gene under the P_{BAD} promoter, which is inducible with L-arabinose (Ara), and the lambda red (gam, exo, bet) recombination system controlled by a Pₜₑₜ promoter, inducible with AHT. The donor plasmid is based on a high copy pUC vector carrying the crRNA sequence for a specific target gene, as well as homology regions to the target serving as repair templates after double strand break.

Cas12a processes the transcript from the donor plasmid to generate mature crRNAs. Guided by the crRNA, Cas12a finds the genomic target and induces a double-strand break. Recombination occurs between the genomic target and the donor DNA mediated by I-Red.

In order to validate this method in Photorhabdus laumondii TTO1, the stlA gene was selected as the first target for genome editing because of an easily recognizable phenotype (FIG. 2A). StIA is a phenylalanine ammonia lyase involved in the biosynthesis of isopropylstilbene (IPS; 1) produced by all Photorhabdus strains (FIG. 2B) and its deletion results in overproduction of the orange anthraquinone pigment. The stilbene biosynthetic gene “cluster” in P. luminescens comprises a stilbene epoxidase gene (plu2236), which is adjacent to stlA (plu2234), a phenylalanine ammonia lyase that converts phenylalanine to cinnamic acid, initiating phenylpropanoid/stilbene biosynthesis.

A donor plasmid (pAR18, FIG. 2C) was designed based on the pTargetF (Addgene #62226) vector for constitutive expression of crRNA (specific for a target DNA), with gentamycin resistance as well as the sacB gene as counter-selection marker enabling rapid loss of the donor plasmid allowing for several rounds of genome editing. Moreover, the donor plasmid comprises homology regions left and right (HR-L and HR-R) to the target serving as repair templates after double strand break. Two crRNA sequences were used for each deletion to enable targeting of the leading and lagging strand, respectively. For this purpose was constructed a synthetic framework consisting of the constitutive promoter J23119, a transcriptional terminator and the direct repeats of the crRNA sequence.

The inventors modified a standard CRISPR array replacing the spacer sequence between the direct repeats by two Bsal restriction sites containing spacer sequence, (also named spacer dummy, SD) (FIG. 2C, 2D). The stlA specific target spacers were determined by “Annotate & Predict” function of Geneious Prime, “TTTN” was set as PAM site and a target spacer length of 23-31 bp. Furthermore, to facilitate plasmid assembly, the homology repair arms (HA-L/HA-R; ~500 bp) were each coupled with one of the crRNAs and synthesized as a dsDNA fragment (FIG. 2C).

Since the original p46Cpf1-OP2 of Ao et al., 2018 was not transformable into Photorhabdus, the inventors first exchanged the origin of replication from pSC101 to p15A resulting in helper plasmid pAR16, assuming it would increase transformation efficiency.

The stlA-specific donor pAR18, assembled by the Golden Gate reaction, was transformed, together with helper pAR16 into Photorhabdus laumondii TTO1 by electroporation (FIG. 2D).

To determine the optimal starting OD₆₀₀ of the liquid CRISPR culture, OD=0.1, 0.2 and 0.5 after inoculation from an overnight culture were selected. Since Photorhabdus has a much slower doubling time compared to E. coli, the required OD₆₀₀ of 0.8-1.0 could not be achieved within a single working day when the starting OD₆₀₀ was below 0.5. Therefore, 0.5 was chosen as the starting OD₆₀₀ of the liquid culture for the following experiments. When the OD₆₀₀ of 0.8-1.0 was reached, the inducers for cpf1 (Ara) and lambda red (AHT) were added. As the system was previously tested (Ao et al., 2018) only the overall method instead of the individual components of the system, was checked. To reduce cell growth and promote protein expression, the inventors tested if temperature had an effect on the editing after induction of all components. Briefly, constant incubation at 30° C. was compared with a culture that was incubated at 25° C. after induction. An editing efficiency of 100% in the low-temperature culture was observed whereas only half of the tested colonies previously grown at 30° C. were positive.

### Example 3: Construction of the Single-Plasmid CRISPR-Cpf1 Approach

Since it was observed Photorhabdus had poor transformation efficiency and Xenorhabdus was not transformable at all with the p46Cpf1-OP2 and its derivative pAR16, the inventors decided to develop a single plasmid solution. Based on the broad-host vector pSEVA231 (GenBank: JX560328.1), a plasmid pAR20 combining the components from the donor plasmid pAR18 and helper plasmid p46Cpf1-OP2 was constructed. A major advantage of the method is the possibility to conjugate the plasmid directly from E. coli to the recipient strain without any integration of the plasmid or its parts into the genome.

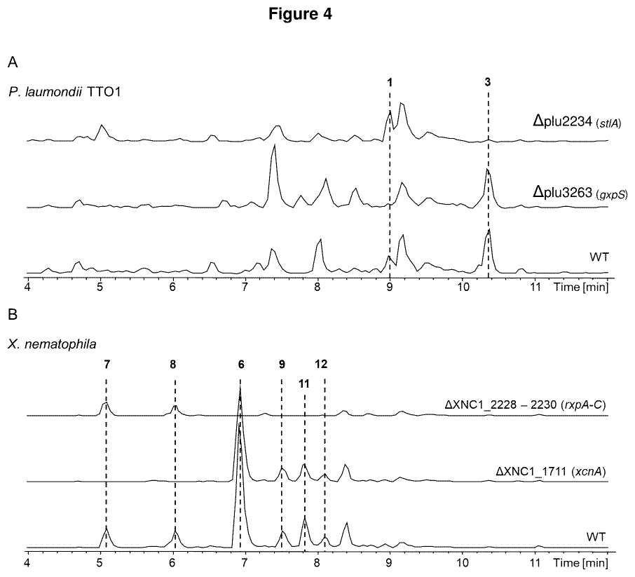

To test its efficiency, stlA (plu2234) gene was deleted. The results showed that stlA was deleted in all colonies (Efficiency=100%) that had the expected pigmentation phenotype (FIG. 2A, FIG. 4, Table 3).

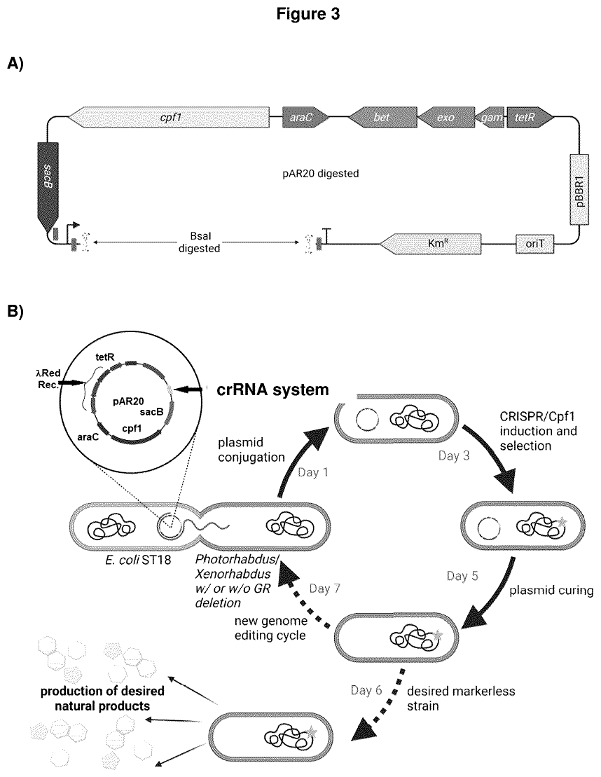

The Bsal digested pAR20 (FIG. 3) can be used for CRISPR/Cpf1 mediated genome editing such as deletions, promoter exchange and replacement of sequences with fluorescent reporters.

In addition to the deletion of small gene sequences such as stlA (plu2234, 1.9 kb deletion), the inventors wanted to evaluate the potential of the method for medium and large deletions and whether the editing process can be speed up. The inventors focused on genes whose manipulation has visible phenotypes or results in metabolite changes easily detectable by mass spectrometry. The selected and successfully deleted genes/genome segments include (Table 3, FIG. 4):
gxpS (plu3263, 15.4 kb deletion) in TT01 and
xcnA (XNC1_1711, 7.5 kb deletion) in Xenorhabdus nematophila,
rxpABC (XNC1 2228-2230, 15.7 kb deletion) in Xenorhabdus nematophila

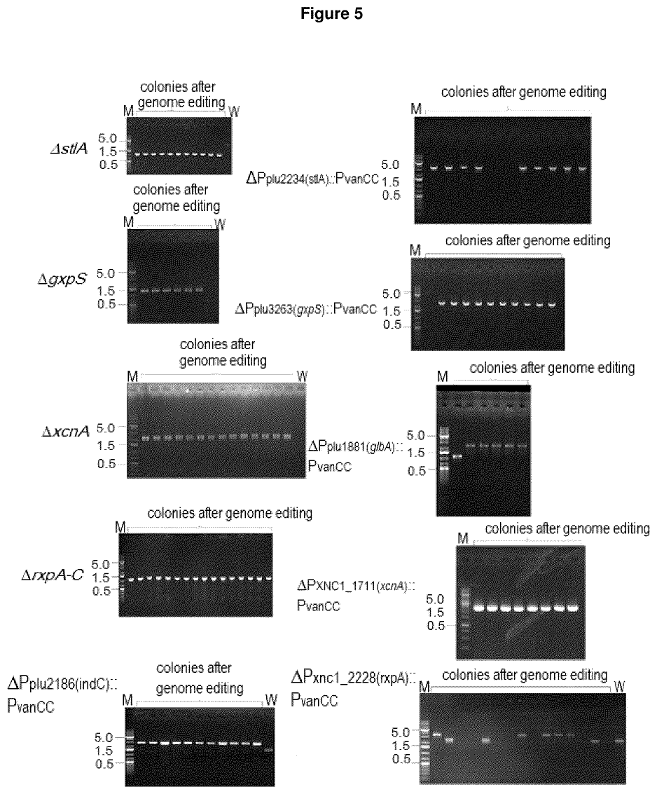

Efficiency was determined by dividing the number of positive edited colonies by the total number of colonies tested. The corresponding agarose gel electrophoresis pictures are shown in FIG. 5. Target spacers used for each approach are listed in Table 7b.

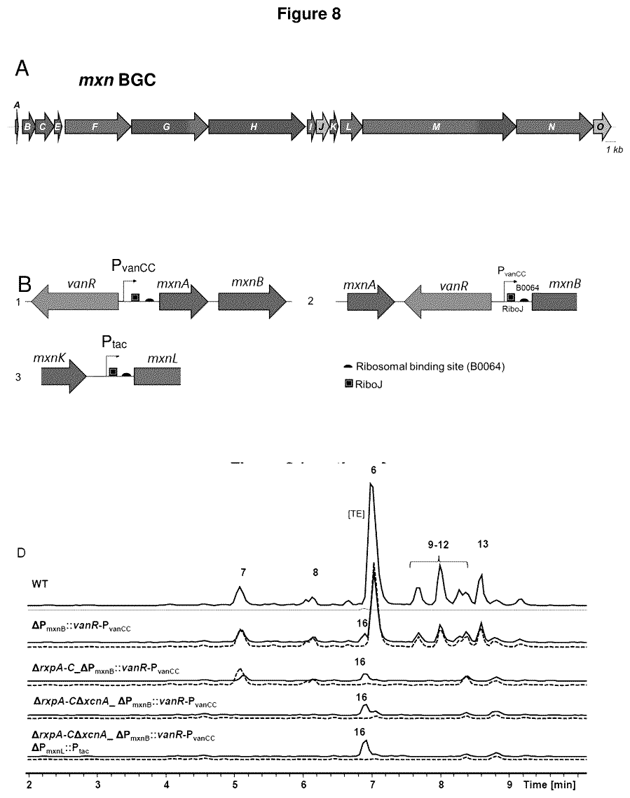

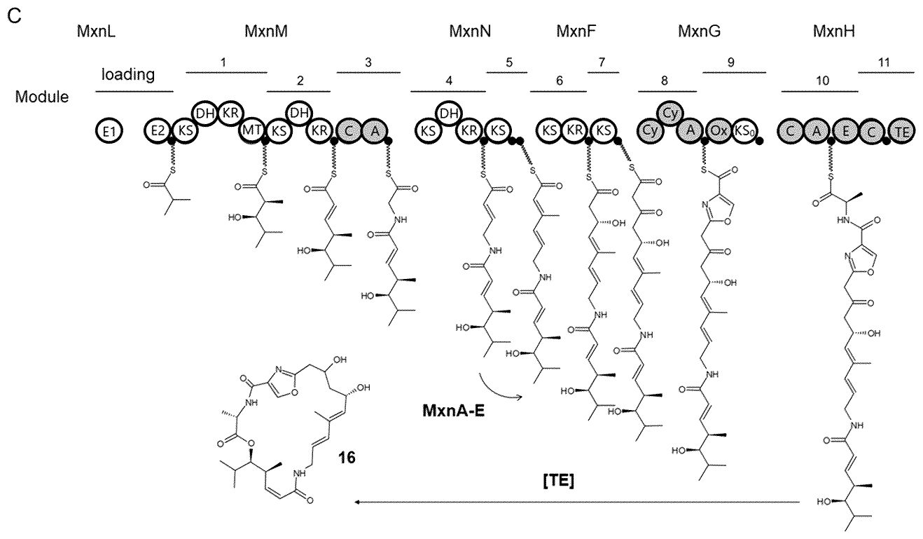

Large gene deletions up to 15 kb in P. laumondii TTO1 and X. nematophila were easily accessed with high efficiency (Table 3). Furthermore, there was no major impact on the metabolic profile after deletion of gxpS in P. laumondii TTO1 or xcnA in X. nematophila except the expected loss of GameXPeptides and xenocoumacins, respectively. Deletion of the rxpABC genes responsible for the production of rhabdopeptide (9-12) in X. nematophila however also leads to an additional loss of xenortide A (6) as well as an increase of xenocoumacin II (8) (FIG. 8D, third line). These findings indicate a regulatory or metabolic network of natural products in X. nematophila associated with rhabdopeptide biosynthesis that will be studied in the future.

### Example 4: Induction of Secondary Metabolites by Insertion of Regulatory Sequences in Target BGC

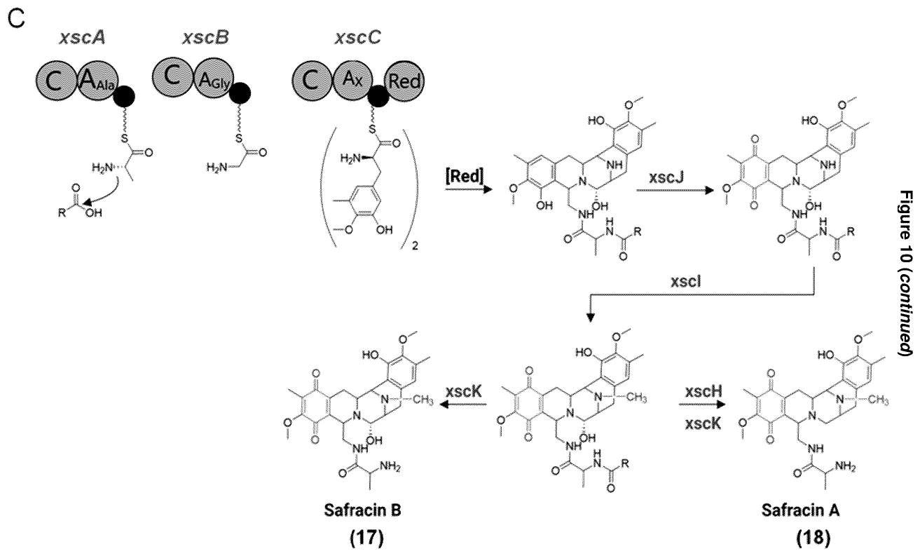

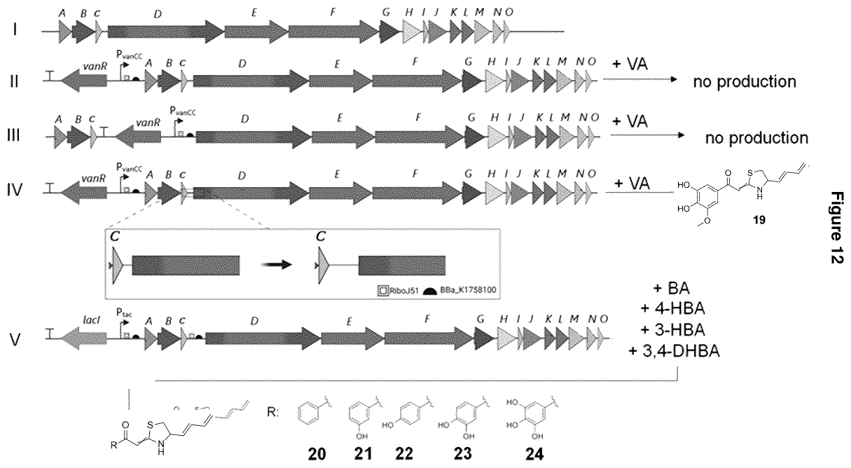

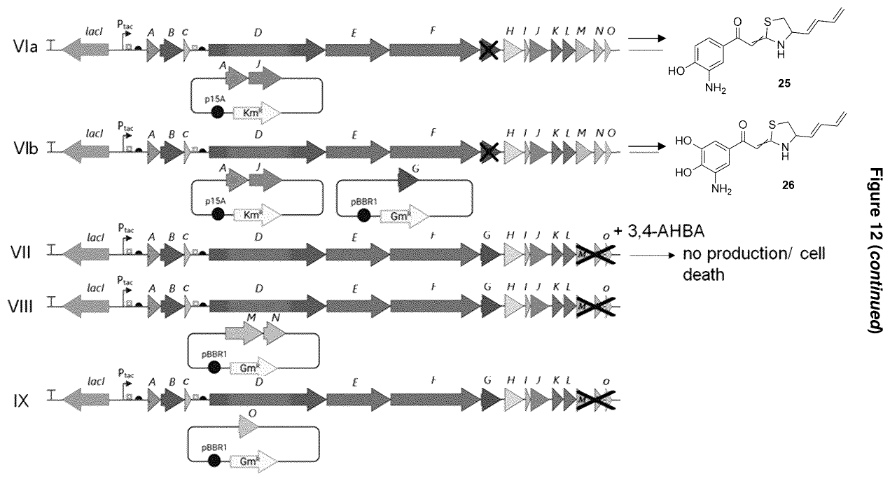

In addition to deletions, the use of small regulatory sequences like inducible promoters is particularly important in natural product research and for biosynthesis elucidation. For this purpose, various inducible promoters described in Table 1 (Nat Chem Biol, 2019. 15 (2): p. 196-204) were tested, in particular those featuring an insulator (riboJ) and a strong ribosomal binding site (RBS) to switch on and off different genes and gene clusters. As shown in FIGS. 10 and 12, activation of Safranin biosynthetic gene cluster and of rupshomycin biosynthetic gene cluster requires also the presence of such additional regulatory elements.

As example, indC was selected, which encodes a NRPS involved in the biosynthesis of the blue pigment indigoidine. This gene is silent under laboratory conditions but can be activated by the introduction of a foreign promoter.

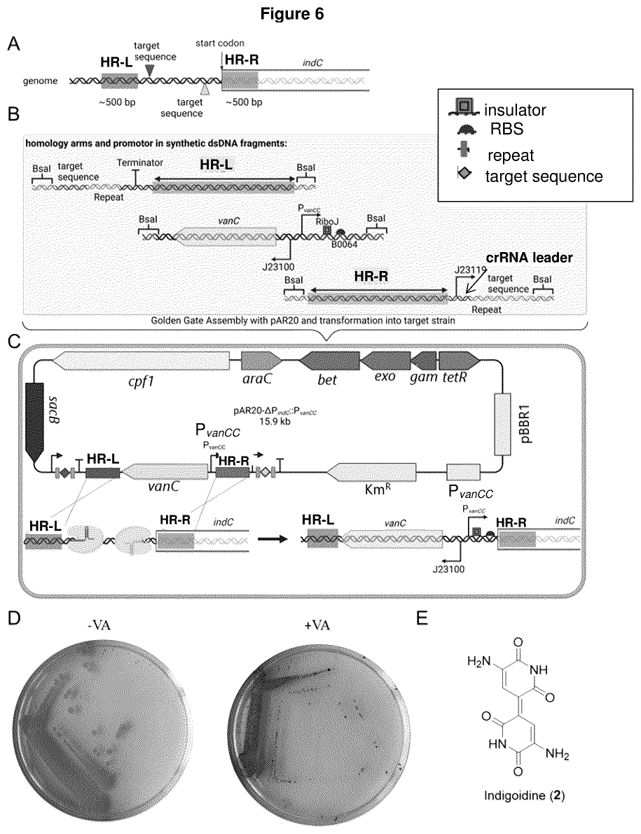

For the CRISPR/Cpf1 approach, the homology regions “selected” are positioned 500 bp upstream of the indC coding sequence (HR-L) and 500 bp downstream of the start codon (HR-R), while the distance between both can be variable (FIG. 6A). In contrast to a deletion, a sequence of the VA inducible promoter system (SEQ ID NO 131) was assembled between the homology arms during golden gate assembly (FIG. 6B-C). After the editing approach, the sequence between the homology regions was replaced by the promoter sequence comprising the repressor vanC in reverse direction with the related elements: PJ23100, RBS, insulator and a terminator, and the inducible PvanCC with an insulator and RBS element “in frame” with indC.

The colonies were tested by PCR and showed 100% editing (FIG. 5). After induction of the promoter with vanillic acid, the typical blue pigment was observed (FIG. 6D).

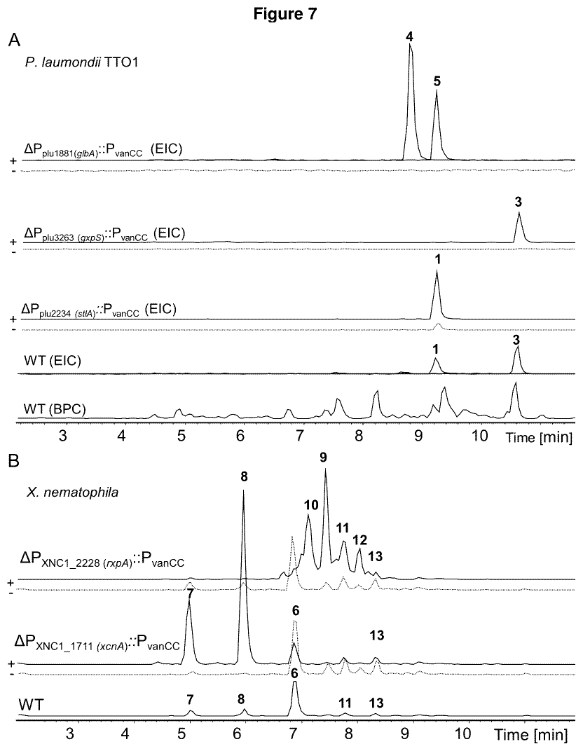

This approach was also applied to other genes of known BGCs facilitating overexpression of previously moderate produced metabolites as well as production of previously “silent” BGCs. The successfully edited targets are listed in Table 3 and include the different compound classes: Glidobactins (4-5) (glbA), GameXPeptide A (3) (gxpS), Isopropylstilbene (1) (stlA), Rhabdopeptides (9-12) (rxpA), and Xenocoumacin I & II (7-8) (xcnA). MS spectra of induced and non-induced cultures after promoter insertion are shown in FIG. 7. In general, a significantly higher production of natural products was observed through compared to the wild type level. As previously observed during rxpABC deletion, the promoter exchange upstream of rxpA had an impact on xenortide A (6) production. While 6 was still detectable in the non-induced culture, it was significantly reduced after induction of the rxp BGC, which might be due to the cross talk between RxpABC and XrdAB as described above.

### Example 5: Activation and Refactoring of the Silent BGC Mxn (Madumycin II)

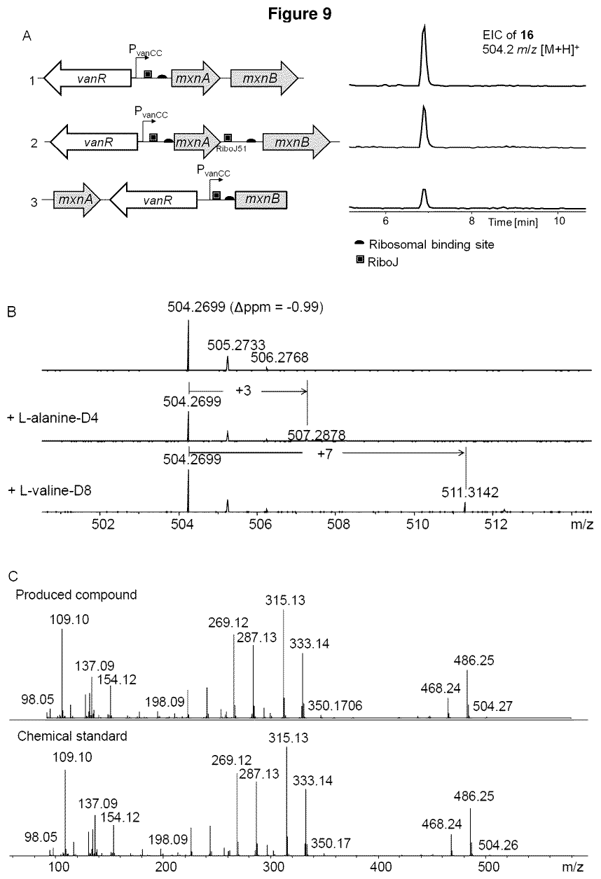

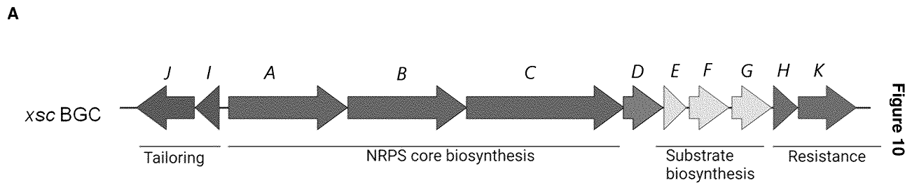

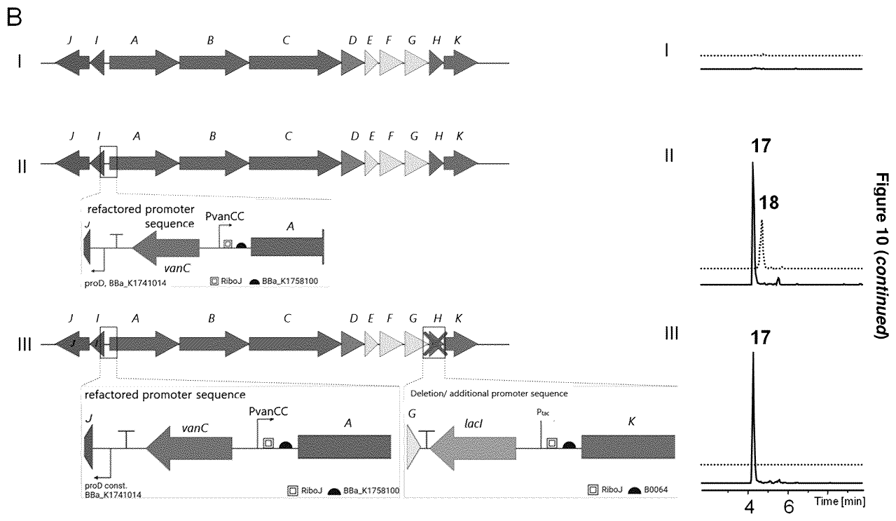

Like indC or glbA the putative pristinamycin II BGC (FIG. 8A) is not active under laboratory conditions. Previous findings showed activation of the BGC during infection of Galleria mellonella larvae and a mass of 526.2 m/z [M+H]⁺ was detected, which is consistent with pristinamycin II. Feeding experiments with isotopically labeled amino acids in infected insects and highly similar BGC architecture further confirmed this. In order to activate this BGC with the above described pAR20 based approach, the region for promoter insertions were selected upstream of the gene encoding a beta-ketoacyl-ACP synthase (Table 4). For this the inventors used the VA inducible promoter system (SEQ ID NO 131) assembled with HA-L and HA-R mxnB_L/R: SEQ ID NO 159-160 on pAR20 backbone. The inventors revealed the previously identified mass of 526.2 m/z [M+H]⁺ as the sodium adduct of the mass of 504.2 m/z [M+H]⁺. HR-MS studies allowed the prediction of a sum formula (C₂₆H₃₇N₃O₇, Δppm=−0.99) consistent with madumycin II, which also belongs to the streprogramin class A as pristinamycin II. Feeding experiments with deuterated valine and alanine confirmed their incorporation and MS/MS analyses of the fragmentation pattern, what was compared with a chemical standard, confirmed that the compounds indeed is madumycin II (FIGS. 9 B and C). Therefore the inventors updated the nomenclature of the BGC from pxn to mxn and adapted the biosynthetic pathway according to the new findings (FIG. 8A). Since the retention times of madumycin II (16) and xenortide A (6) are almost identical, strain engineering was necessary. From the previous observations, it was known that deletion of the rxp cluster also leads to loss of both rhabdopeptides and xenortides. Therefore, it was used the same construct as described above for the rxp deletion (FIG. 8D) to prevent production of both compound classes. Since xenocoumacin is also becoming more dominant after rxp deletion xcnA was deleted as well, resulting in a strain with a much cleaner production of madumycin II (16). The introduction of another strong constitutive promoter (P_{tac} without the lacl repressor, SEQ ID NO 134) upstream of mxnL led to a substantial increase in the production titer of approximately 250 mg (FIGS. 8 B, C and D). The according editing plasmid was based on pAR20 with mxnK specific HA-L and HA-R (SEQ ID NOS 161-162). Production of madumycin II was performed in XPP3 medium with 2% (v/v) XAD-16 for in situ extraction.

### Example 6: Activation and Refactoring of the Silent BGC Xsc (Safracin)

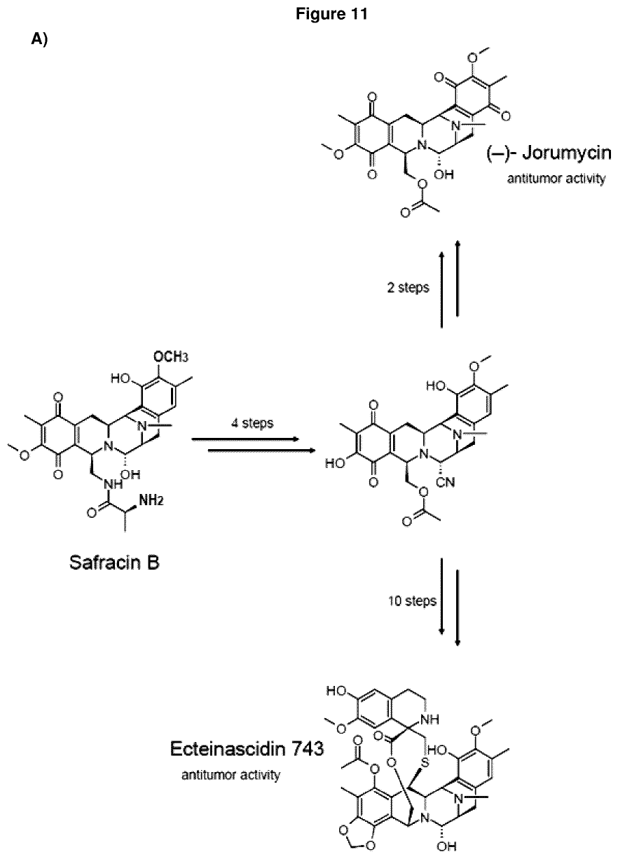

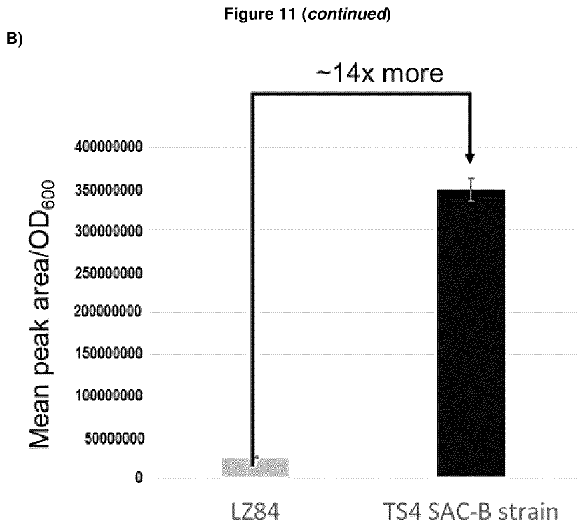

In addition to “simple” and multiple promoter exchanges, complex BGC activations are also possible with the method developed herein. The activation of the putative safracin-associated BGC, named xsc, in Xenorhabdus sp. TS4 is such an example due to its bidirectional architecture (FIGS. 10A and 10B I). Safracins have been previously described and characterized in Pseudomonas fluorescens. Like the mxn BGC above, this xsc (Xenorhabdus safracin cluster) BGC is not active under laboratory conditions (FIG. 10B I). For activation, a strong inducible P_{vanCC} promoter was introduced upstream of xscA and a strong constitutive promoter (proD) in the opposite direction upstream of xsc/(FIG. 10B II) in Xenorhabdus sp. TS4. In addition, the 5′-UTR between the RiboJ and the start codon was replaced with a translation enhancer sequence (g10T7 UTR (also abbreviated as T7g10), BBa_K1758100, sequence available online) comprising a 5′ untranslated region (5′-UTR) and a strong ribosomal binding site from bacteriophage T7 gene 10 (g10-L). (FIG. 10B II box). The approach is based on pAR20 editing plasmid with synthetic dsDNA fragments used for CRISPR/Cas12 mediated genome editing: xscA/xscl_specific HA-L and HA-R (SEQ ID NOS 167-168) and SEQ ID NO 132 as promoter. To obtain SAC-B as the exclusive main product, xscH was deleted with simultaneous promoter exchange before xsck (FIG. 10 B III). Herefore the following synthetic dsDNA fragments used for CRISPR/Cas12 mediated genome editing for pAR20 based assembly were used: xscKΔxscH specific HA-L and HA-R (SEQ ID NOs 169-170) and SEQ ID NO 136 as promoter sequence. High SAC-B production is very valuable as SAC-B can be used as starting material for semisynthetic approaches to produce potent antitumor drugs (ET-743 and (−)-jorumycin, FIG. 11A). With the strain III production titers for SAC-B 150 mg/ml can be reached, which is higher than production reached in the prior art CN103074395B, and CA2510069A1. Production of SAC-B was performed in XPP3 medium with 2% (v/v) XAD-16 for in situ extraction. The production of SAC-B by the constructed Xenorhadus sp. TS4 SAC-B producer strain (shown in FIG. 10 B III) was compared to E. coli strain LZ84 harboring the xsc Cluster encoded on three expression plasmids (pCola_PBad_xscA-H, PACYC_PBad_xsclJ, pCDF_PBad_xscK, Table 2). Triplicates were made from both strains and cultivated in XPP3 production medium according to the previously described “Metabolite production, extraction and HPLC-MS/MS with absorbing Amberlite XAD-16 resin” method. TS4 strains were cultivated at 28° C. and LZ84 strains at 22° C. After cultivation, the resins were harvested, eluted with methanol and extracts were analyzed for their SAC-B production via LC-MS. With respect to the final OD600, the SAC-B amount of the constructed TS4 strain was 14 times higher than that of LZ84 (FIG. 11B).

### Example 7: Activation and Refactoring of the Silent Novel Rpm BGC (Rupshomycin)

A further example of complex and cryptic cluster activation is a trans-AT NRPS/PKS hybrid cluster (rpm BGC, genes rpmA-rpmO) from Xenorhabdus indica (FIG. 12 I). When testing different insertion points for single promoter exchange based on the developed pAR20 approach, new products were not detectable (FIG. 12 II and III).

First, an inducible promoter (Pvan modified, SEQ ID NO 131, SEQ ID NO 171-172) was inserted upstream of rpmA (FIG. 12 II), not resulting in production of a new compound. Thereafter, a promoter exchange with Pvan modified (SEQ ID NO 132, NO 173-174) was performed before rpmD (FIG. 12 III), also without success.

As it was presumed the presence of another regulatory sequence between rpmC and rpmD, this sequence was replaced by ribozyme RiboJ51 and the T7g10 translational enhancer in the PvanCC_rpmA strain, generating mutant strain “IV” (FIG. 12 IV). For this purpose, the synthetic dsDNA fragments rpmD specific HA-L and R with SEQ ID NOs 173-174 and Translational Enhancer SEQ ID NO 135 inserted between them were used. This leads to a new compound 19 with a mass of 320 Da. Compound 19 was then isolated and structurally confirmed by NMR.

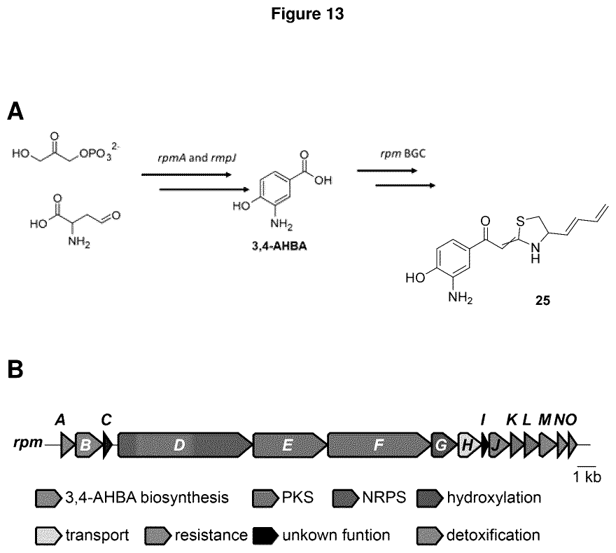

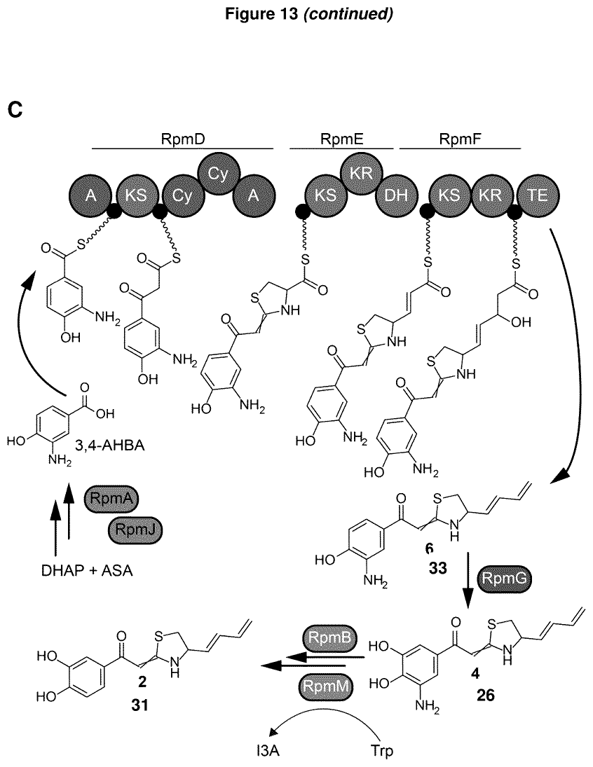

However, Compound 19 is an artificially created product, as the BGC uses the inducer for the P_{vanCC} promoter, vanillic acid, as a building block for the biosynthesis. To obtain the true natural product, the P_{vanCC} was replaced by the IPTG inducible P_{tac} promoter (SEQ ID NO 136) and the gene encoding the global regulator Hfq was deleted in this strain (FIG. 12 V). Both based on pAR20 gene editing with the synthetic dsDNA fragments rpmA specific HA-L and R SEQ ID NOs 171-172 for Ptac introduction and SEQ ID NOs 179-180 for hfq gene deletion. Again, a natural product could not be detected. Therefore different benzoic acid (BA) derivatives were added to the culture medium at 1 mM. Results showed that all BA derivatives were accepted and incorporated by the BGC in the produced SMs (FIG. 12 V). Single and double hydroxylated BAs were found to undergo further hydroxylation, putatively catalyzed by rpmG. This assumption was confirmed by deleting rpmG. In order to find the component acting as starter BA, the genes rmpA and rpmJ, encoding enzymes for starter unit biosynthesis, were overexpressed together on plasmid pAR29bad in the ΔrpmG mutant. Thereby, it could be detected compound 25, which incorporated 3-amino-4-hydroxy BA (3,4-AHBA) as a starter unit. By supplementary feeding of 3,4-AHBA this observation was confirmed (FIG. 12 VIa). Subsequently, strain I (FIG. 12) was fed with 3,4-AHBA in excess for three days leading to the detection of the primary product, compound 26, of the rpm BGC. The product found was named by the inventors rupshomycin (26). The inventor were also able to predict the putative biosynthesis (FIG. 13). Compound 26 was obtained also after overexpression of rpmAJ encoded on pAR29bad together with rpmG encoded on pAR30bad (FIG. 12 VIb). According primers are listed in Table 7a.

To elucidate the influence of rpmM-O on biosynthesis, they were deleted together bases on pAR20 with the synthetic dsDNA fragments used for CRISPR/Cas12 mediated genome editing specific HA-L and R SEQ ID NOs 177-178 The resulting mutant ΔrpmM-O was then analysed for production and growth. Also with the addition of 3,4-AHBA, no growth and thus no production could be observed (FIG. 12 VII).

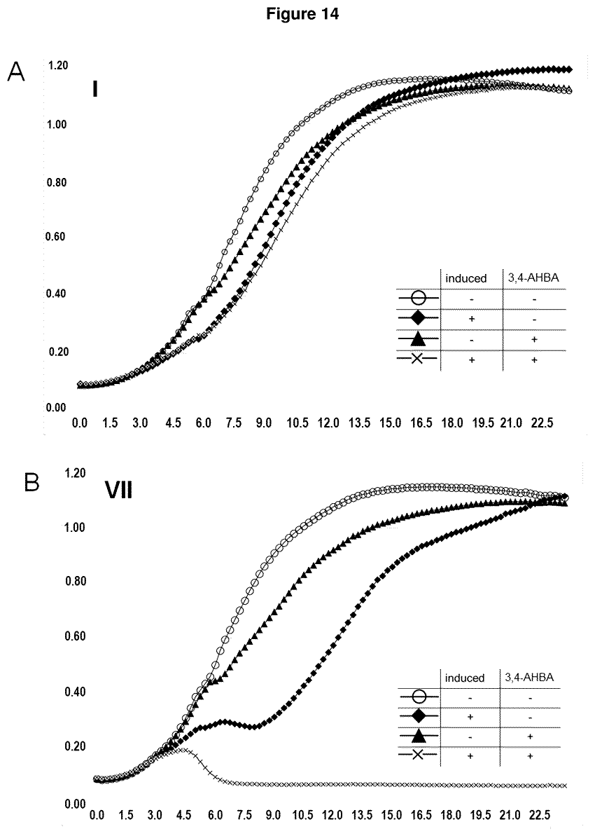

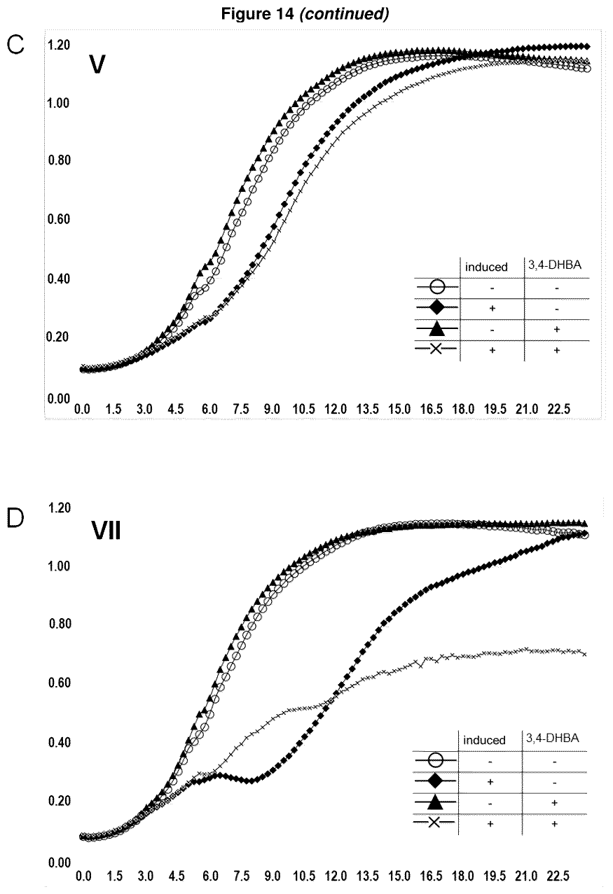

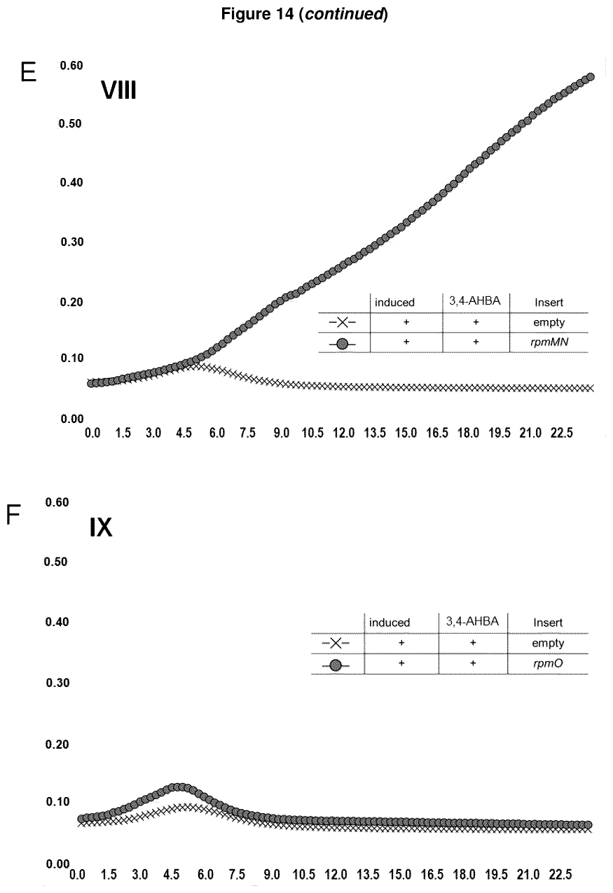

In order to analyze the growth curve, the strains were cultured in microtiter plates in a Tecan Spark microplate reader (FIG. 14). A comparison was initially made between strain V and strain VII with and without the addition of 3,4-AHBA (FIGS. 14 A and B). Furthermore, to investigate the sole effect of 3,4-AHBA on the cells, 3,4-AHBA was added to the cells in presence or not of the rpm BGC inducer IPTG (FIGS. 14A and B). In strain V, no particular growth differences were observed under the above mentioned conditions. In contrast, the ΔrpmM-O mutant showed a saddle point after 6-7 hours in growth in an induction background without the addition of 3,4-AHBA. When 3,4-AHBA was added to induced cells, a weak growth was observed in the first 3 hours, which then quickly collapsed and fell below the optical density of the starting point of the experiment. Subsequently, in order to investigate the effect of the amino group on the benzoic acid, 3,4-DHBA was fed to both strains (FIGS. 14 C and D). The addition of 3,4-DHBA did not lead to the complete death of the ΔrpmM-O culture, but to a growth deficiency compared to the non-induced culture (FIG. 14 D). It can therefore be concluded that the amino group is crucial for the bioactivity of rupshomycin.

Which of the rpmM-O genes are responsible for resistance to the biosynthesis product of the rpm BGC was the question addressed in the following studies. To this aim, rpmMN and rpmO were cloned, individually into pAR30bad expression vectors (Primer sequenced can be found in Table 7a), followed by their conjugation into the ΔrpmM-O mutant, and analysis of the growth of the resulting strains “VIII” and “IX” in presence of 3,4-AHBA and/or IPTG, to induce rpm BGC (FIG. 12 VIII and IX, FIGS. 14 C and D). The influence of the vector was excluded by conjugating an empty vector into the strains as a control. It was observed that the genes rpmM and rpmN expressed on a vector allowed significant growth of the culture compared to the empty vector control (FIG. 14 E) that resulted in a severe growth defect of the culture similar to that in FIG. 14 B. For rmpO expressed on a vector, no significant growth of the culture was observed compared to the empty vector control (FIG. 14 F). Therefore, it could be concluded that rpmMN together or separately are mainly responsible for the resistance to the biosynthetic products of rpm BGC. However, it might be possible that all three genes together rpmM-O are required for full resistance. Production of rupshomycins were performed in XPP3 medium with 2% (v/v) XAD-16 for in situ extraction.

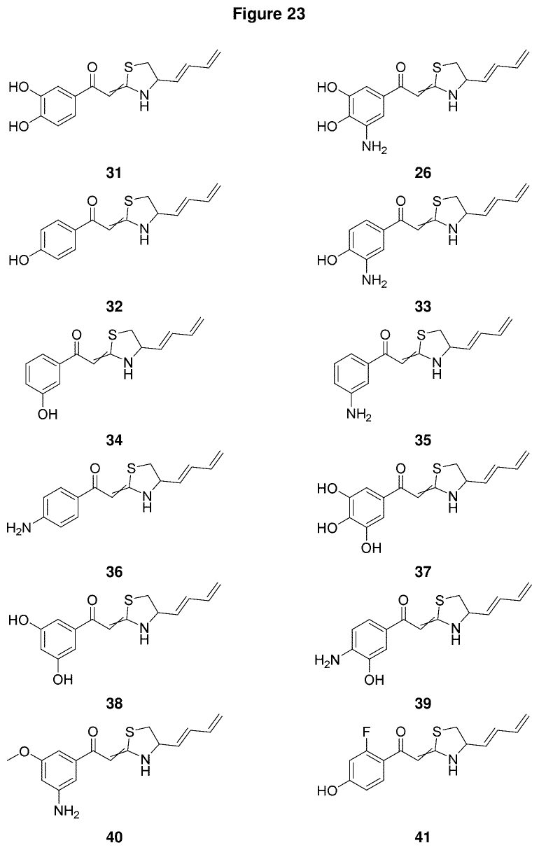

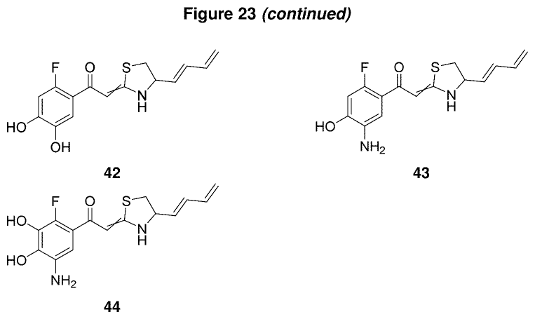

This approach was applied to further benzoic acid derivatives, thereby the rupshomycin derivatives 31 to 44 were obtained. The structures of compounds 31 to 44 are shown in FIG. 23.

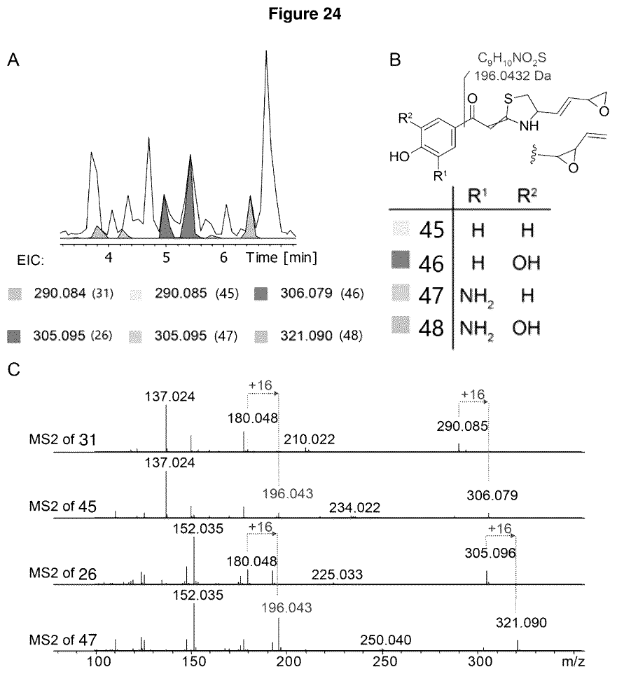

By insertion of additional gene encoding styrene monooxygenase enzyme (StyA, SEQ ID NO 212) epoxy functionalized rupshomycin derivatives 45 to 48 were obtained (see FIG. 24).

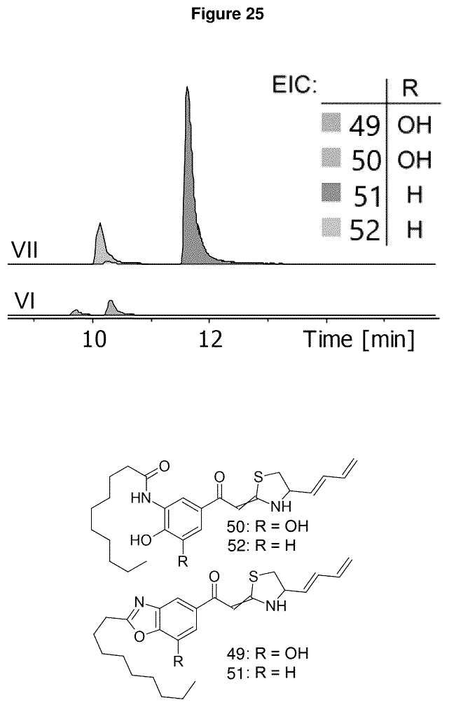

By insertion of additional clxD gene encoding an enzyme catalyzing benzoxazole cyclisation (ClxD, SEQ ID NO 211) benzoxazole-containing rupshomycin derivatives 49 and 51 were obtained from compounds 50 and 52, respectively (see FIG. 25).

Extraction and Isolation of these compounds were carried out as follows exemplarily described for compounds 32 and 33:

XAD beads were extracted with twice with MeOH+ 1% formic acid, and dried under reduced pressure to give a crude extract of 1.23 g. The crude extract was purified by semi-preparative HPLC (20 mL/min, A: water+0.1% formic acid, B: acetonitrile+0.1% formic acid, gradient: 5% of B for 2 min, 5% to 40% of B in 25 min). The fraction F2 (2.8 mg) was obtained, containing an E/Z-isomer mixture of 32 in ratio of 1:1. The fraction F1 (10.8 mg) was further purified with analytical HPLC (3 mL/min, A: water+0.1% formic acid, B: acetonitrile+0.1% formic acid, gradient: 25% of B for 2 min then 25% to 55% of B in 12 min) leading to the isolation of fraction F1F1 (0.6 mg) containing an E/Z-isomer mixture of 33 in ratio of 1:1.

32: white amorphous solid, ESI-MS m/z: 274.0894 [M+H]⁺ corresponding to C₁₅H₁₆NO₂S.

33: white amorphous solid, ESI-MS m/z: 289.1007 [M+H]⁺ corresponding to C₁₅H₁₇N₂O₂S.

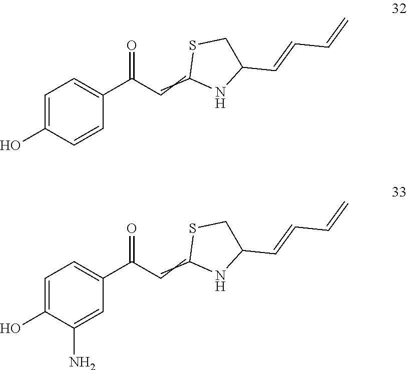 [SMILES: C=C/C=C/C1CSC(=CC(=O)c2ccc(O)cc2)N1 ; C=C/C=C/C1CSC(=CC(=O)c2ccc(O)c(N)c2)N1]

TABLE 12
NMR data of compound 32

| (Z)-32 | | | | (E)-32 | | | |
|---|---|---|---|---|---|---|---|
| | | | HMBC (¹H | | | | HMBC (¹H |
| δ_{C} | δ_{H}(J in Hz) | COSY | to ¹³C) | δ_{C} | δ_{H}(J in Hz) | COSY | to ¹³C) |
| 118.7 | a. 5.29 dd | 1b, 2 | 2, 3 | 118.7 | a. 5.29 dd | 1b, 2 | 2, 3 |
| | (1.2, 16.5) | | | | (1.3, 16.5) | | |
| | b. 5.16 dd | 1a, 2 | 3 | | b. 5.16 dd(1.0, | 1a, 2 | 3 |
| | (1.2, 9.0) | | | | 9.1) | | |
| 136.3 | 6.38 td | 1a, 1b | 3 | 136.3 | 6.38 td | 1a, 1b | 3 |
| | (10.3, 4.1) | | | | (10.5, 4.2) | | |
| 132.6 | 6.28 m | 4 | 1, 5 | 132.6 | 6.28 m | 4 | 1, 5 |
| 132.0 | 5.85 dd | 3, 5 | 2 | 133.0 | 5.82 dd | 3, 5 | 2, 5 |
| | (6.8, 15.3) | | | | (6.9, 15.5) | | |
| 62.9 | 4.80 q (7.0) | 6a, 6b | 4, 6, 7 | 60.1 | 4.46 q (7.0) | 6a, 6b | 4, 6, 7 |
| 33.6 | 3.50 m | 6b, 5 | 4, 5 | 34.8 | a. 3.26 m | 6b, 5 | 4, 5, 7 |
| | 3.10 dd | 6a, 5 | 4, 5, 7 | | b. 2.86 dd | 6a, 5 | 4, 5, 7 |
| | (6.1, 11.0) | | | | (6.9, 11.0) | | |
| 168.0 | — | | | 168.3 | — | | |
| 86.2 | 5.98 s | | 7, 9 | 87.5 | 6.31 s | | 7, 9 |
| 184.0 | — | | | 183.6 | — | | |
| 131.2 | — | | | 131.2 | — | | |
| 129.4 | 7.74 d (8.7) | 12 | 9, 13, 15 | 129.0 | 7.64 d (8.7) | 12 | 9, 13, 15 |
| 115.1 | 6.80 m | 11 | 10, 13, 14 | 115.1 | 6.80 m | 11 | 10, 13, 14 |
| 160.0 | — | | | 160.0 | — | | |
| 115.1 | 6.80 m | 15 | 12, 13 | 115.1 | 6.80 m | 15 | |
| 128.9 | 7.74 d (8.7) | 14 | 9, 11, 13 | 128.9 | 7.64 d (8.7) | 14 | 11 |
| — | — | — | — | — | 8.45 s | | 5, 6, 7 |

TABLE 13
NMR data of compound 33

| (Z)-33 | | | | (E)-33 | | | |
|---|---|---|---|---|---|---|---|
| | δH (J | | HMBC (1H | | δH (J | | HMBC (1H |
| δC | in Hz) | COSY | to 13C) | δC | in Hz) | COSY | to 13C) |
| 119.0 | a. 5.27 dd | 2 | | 119.0 | a. 5.25 dd(1.8, | 2 | |
| | (1.8, 16.3) | | | | 16.8) | | |
| | b. 5.15 dd | 2 | | | b. 5.14 dd | 2 | |
| | (1.9, 10.2) | | | | (1.9, 10.2) | | |
| 136.3 | 6.39 m | 1a, 1b | | 136.3 | 6.39 m | 1a, 1b | |
| 132.9 | 6.29 dd | 4 | | 132.9 | 6.26 dd | 4 | |
| | (2.9, 10.8) | | | | (2.9, 9.8) | | |
| 132.6 | 5.83 dd | 5 | | 133.0 | 5.80 dd | 3, 5 | 2 |
| | (6.8, 15.0) | | | | (7.2, 15.1) | | |
| 63.1 | 4.77 dt | 6a, 6b | 4 | 60.2 | 4.44 dt | 6a, 6b | 4 |
| | (7.0, 6.7) | | | | (7.0, 7.1) | | |
| 132.2 | a. 3.49 dd | 5, 6b | | 132.2 | a. 3.23 dd | 5, 6b | |
| | (7.3, 11.0) | | | | (7.4, 11.1) | | |
| | b. 3.09 dd | 5, 6a | | | b. 2.83 dd | 5, 6a | |
| | (6.1, 11.0) | | | | (6.9, 11.1) | | |
| nd | — | | | 167.2 | — | | |
| 86.2 | 5.86 s | | 9 | 87.8 | 6.25 s | | 9, 7 |
| 185.5 | — | | | 184.5 | — | | |
| 136.4 | — | | | 131.6 | — | | |
| 117.1 | 6.99 dd | 12 | 13 | 116.7 | 6.90 dd | 12 | 15, 13 |
| | (8.2, 2.2) | | | | (8.3, 2.1) | | |
| 114.1 | 6.64 d | 11 | 10 | 114.1 | 6.65 d | 11 | 10 |
| | (8.0) | | | | (8.1) | | |
| 147.4 | — | | | 146.7 | — | | |
| Nd | — | | | Nd | — | | |
| 113.7 | 7.17 d | 11, 13 | 11 | 113.5 | 7.08 d | 11 | 11, 13 |
| | (2.3) | | | | (2.1) | | |

TABLE 14
HR masses [M + H]⁺ of compounds 26, and 31-52.

| Cpd. No. | formula | found | expected | deviation |
|---|---|---|---|---|
| 31 | C₁₅H₁₅NO₃S | 290.084 | 290.084 | 0.000 |
| 26 | C₁₅H₁₆N₂O₃S | 305.095 | 305.095 | 0.000 |
| 32 | C₁₅H₁₄NO₂S | 274.090 | 274.090 | 0.000 |
| 33 | C₁₅H₁₆N₂O₂S | 289.100 | 289.101 | −3.459 |
| 34 | C₁₅H₁₅NO₂S | 274.089 | 274.090 | −3.648 |
| 35 | C₁₅H₁₆N₂OS | 273.105 | 273.106 | −3.66 |
| 36 | C₁₅H₁₆N₂OS | 273.105 | 273.106 | −3.66 |
| 37 | C₁₅H₁₅NO₄S | 306.079 | 306.079 | 0.000 |
| 38 | C₁₅H₁₅NO₃S | 290.084 | 290.084 | 0.000 |
| 39 | C₁₅H₁₆N₂O₂S | 289.100 | 289.101 | −3.458 |
| 40 | C₁₆H₁₈N₂O₂S | 303.116 | 303.116 | 0.000 |
| 41 | C₁₅H₁₅FN₂O₃S | 323.086 | 323.086 | 0.000 |
| 42 | C₁₅H₁₅FN₂O₂S | 307.092 | 307.091 | 3.256 |
| 43 | C₁₅H₁₄FNO₃S | 308.075 | 308.075 | 0.000 |
| 44 | C₁₅H₁₄FNO₂S | 292.080 | 292.080 | 0.000 |
| 45 | C₂₅H₃₂N₂O₃S | 441.220 | 441.220 | 0.000 |
| 46 | C₂₅H₃₂N₂O₄S | 459.231 | 459.231 | 0.000 |
| 47 | C₂₅H₃₂N₂O₂S | 425.225 | 425.226 | −2.351 |
| 48 | C₂₅H₃₄N₂O₃S | 443.236 | 443.236 | 0.000 |
| 49 | C₁₅H₁₅NO₃S | 290.085 | 290.084 | 3.447 |
| 50 | C₁₅H₁₅NO₄S | 306.079 | 306.079 | 0.000 |
| 51 | C₁₅H₁₆N₂O₃S | 305.095 | 305.095 | 0.000 |
| 52 | C₁₅H₁₆N₂O₄S | 321.090 | 321.090 | 0.000 |

### Example 8: Liquid Culture and Handling for Electro Transformation and Biparental Conjugation

Depending on the strain, a conjugation and selection process in liquid can also be advantageous. Overnight cultures of the recipient and donor strains were inoculated according to growth requirements. The next day, the donor strain was diluted 1:5 and the recipient 1:2 followed by further 2 hours of incubation. Then, 200 μl of the donor was mixed with 800 μl of the recipient in a 2 ml reaction tube. The suspension is subsequently pelleted, and washed twice with 1 ml of growth medium LB with counterselection agent, i.e. 50 μg/ml ALA. Finally, 300 μl of LB containing 50 μg/ml ALA is left over the pellet and incubated overnight at 30 degrees without agitation. The following day, the cell pellet was washed and resuspended in 1 ml LB. The entire cell mass was then added to selection plates containing positive selection agent, i.e. 50 μg/ml Km, and without ALA in order to select only for mutated recipient strains and counter selecting for donor strains.

### Example 9: High-Throughput Conjugation

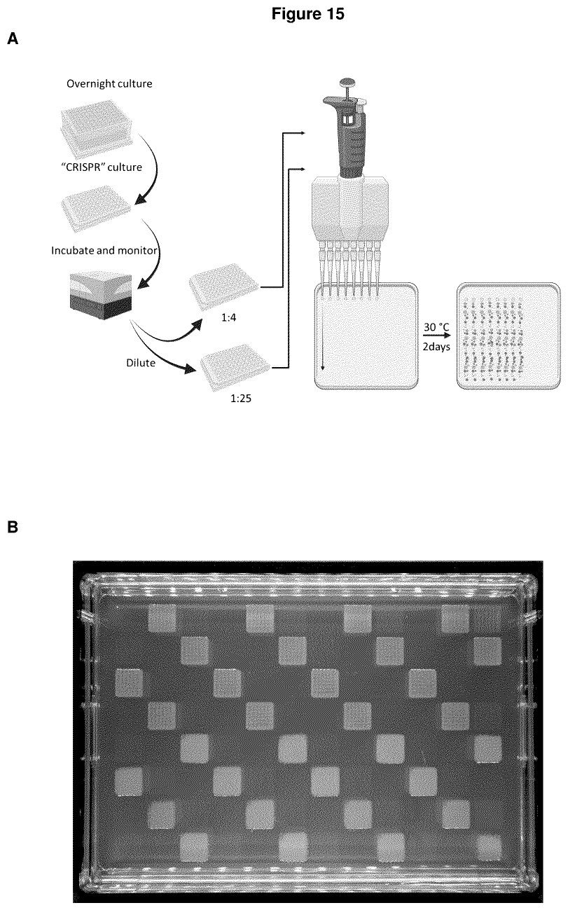

For high-throughput conjugation of pAR20 plasmids into recipient strains, the robot Rotor+system from Singer was used. Overnight cultures of donor strains were grown 10 ml LB containing 50 μg/ml Km and 50 μg/ml ALA acid in 96 well deep well plates. The culture was pelleted the next day and washed twice with ddH₂O. Subsequently, the pellet was resuspended in 100 μl LB and the suspension was spotted robotically in 7×7 patches to a LB agar plate containing 50 μg/ml ALA (FIG. 15B). The same procedure was used for the recipient spotted on top of donor strains. After overnight incubation at 30° C., patches were scraped through the Singer ROTOR+ benchtop robot and transferred to microtiter plates containing 100 μl LB with 25 μg/ml Km. After further incubation overnight, the suspension was spotted through the ROTOR+ onto LB agar containing 25 μg/ml Km. Colonies were visible after 1-2 days depending on the strain.

### Example 10: Culture of Strains after CRISPR/Cpf1 Assisted Gene Editing

For fluorescent reporter insertion/BGC replacement, or genome editing such as GR deletion or promoter exchange or BGC replacement, a strain carrying a specific pAR20 vector was inoculated as a 5 ml overnight culture in a 50 ml Erlenmeyer flask. The next day, this culture was diluted to a final OD of 0.5 in 10 ml LB containing 25 μg/ml Km. The culture was then incubated at 30° C. until an OD of 0.8-1.0 was reached. After addition of AHT to induce expression of the Cpf1 protein, the culture was further incubated at 25° C. for 1h. Subsequently, L-arabinose (0.4%) was added and incubated for another 3 h at the same temperature. Finally, 50 μl of the cell suspension was plated on a selection plate with Km and arabinose to induce expression of lambda red proteins. Edited colonies were visible after 2 days.

### Example 11: Screening in High-Throughput Format of Mutated Bacteria

This Example describes selection in high-throughput format (FIG. 15A), i.e. in 96-well microplates or higher, of bacteria, which have been mutated by experiments of CRISPR/Cpf1 mediated genetic replacement, or deletion, and can be adapted to random mutagenesis, or transposon mutagenesis generated mutants.

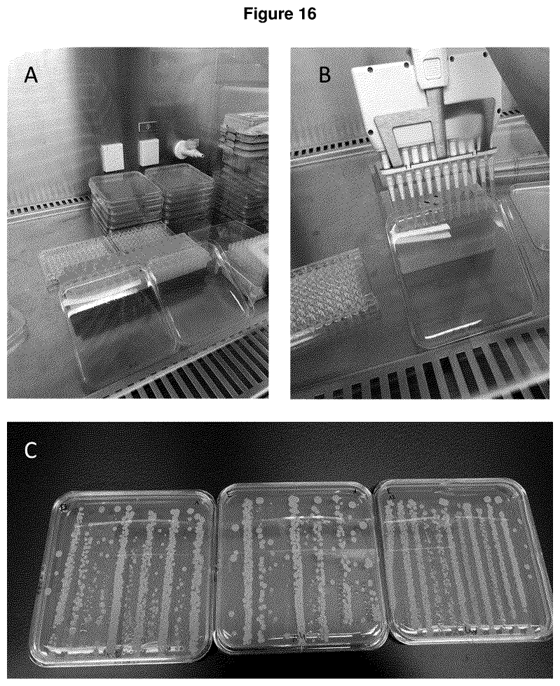

For high-throughput genome editing, the strains carrying gene-specific CRISPR/Cpf1-pAR20 were inoculated in a 1 ml overnight culture in 96 deep well plates. The next day, the cultures were diluted 1:25 in 100 μl fresh LB containing 25 μg/ml Km in 96-well microtiter plates (e.g. Greiner Flat bottom plates). Growth was monitored by incubation in a Tecan microtiter plate reader (Tecan Spark). The absorbance at 595 nm was measured every 15 minutes while the plate was incubated at 220 rpm at 30° C. between measurements. Once the absorbance doubled relative to the initial value, Cpf1 expression inducer AHT was added, the incubation temperature lowered to 25° C. and the plate was then incubated at 220 rpm. After another hour of incubation, L-arabinose was added and plates were incubated for another 3 hours. After the incubation period, two dilutions (1:4 and 1:20) of the original cultures were prepared in microtiter plates. From these dilutions, 20 μl each was spotted onto the top of a rectangular agar plate (containing 25 μg/ml Km and 0.4% arabinose) using a 12-channel multichannel pipette. By placing the plate vertically, the spot was “spread” downwards in a strip and thereby indirectly diluted (FIG. 16A,B,C). Individual colonies were selected after 2 days of growth at 30° C. in the dark and verified by colony PCR.

### Example 12: Activation of the Xenoamicin (Xab) BGC in Xenorhabdus doucetiae ΔcyaA

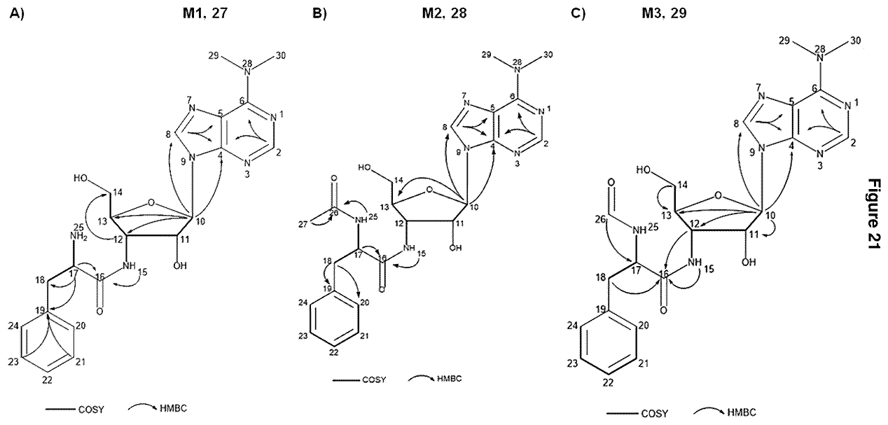

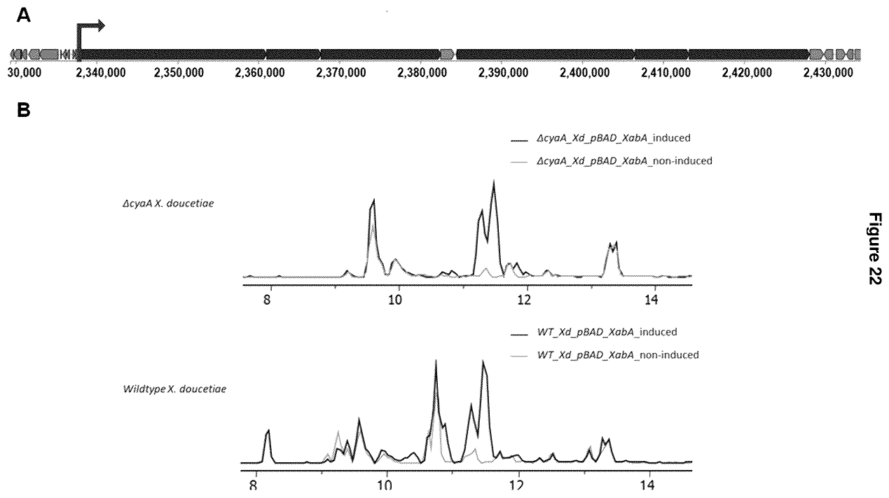

To test the utility of the global regulator cyaA discovered by the above described method, cyaA was deleted in Xenorhabdus doucetiae based on pAR20 targeted gene deletion. CyaA specific HA-L and R (SEQ ID NOs 187-188) were used as synthetic dsDNA fragments for CRISPR/Cas12 mediated genome editing for this approach. This was followed by replacing the native promotor upstream of the known BGC related to the biosynthesis of Xenoamicin A (30, FIG. 22A) in the cyaA deletion mutant and the wild type. This was achieved by the promoter exchange method based on pAR20 using the vanillic acid inducible promoter (SEQ ID NO 131) and the xabA specific HA-L and R SEQ ID NOs 189-190. Production was compared between the cyaA mutant and the wild type (FIG. 22B).

### Example 13: Construction of Multi-Producers of Natural Product Classes with Desired Bioactivities

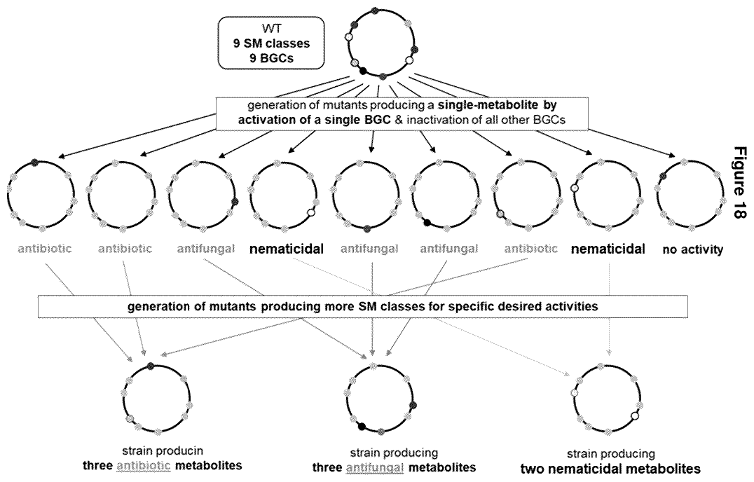

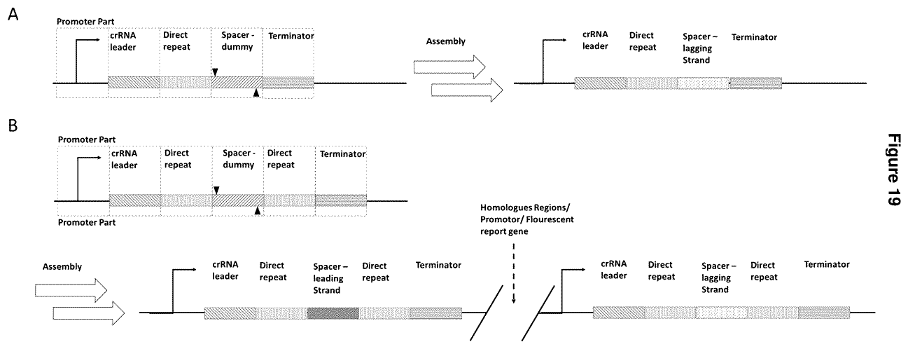

Following the identification of BGCs with desired bioactivities as described in Examples 3-7 (see above), the activatable BGCs can be combined following the inventive methods for promoter exchange and refactoring (see Examples 5-7) in a single strain, leading to strains producing exclusively and only metabolites with desired bioactivities, like antibiotic, antifungal, nematicidal (or any other) activity (FIG. 18). These bacterial strains can be used to generate extracts or compound mixtures with superior bioactivity than the individual metabolites alone and can be applied in medicine, animal health and agriculture.

Such a multi-producer strain (or an extract derived thereof) has the advantage of being easier to handle compared to culture of several mono-producing strains or handling of many extracts of individual compounds to be combined.

### Example 14: Activation of the Silent BGC dpr (Deoxy-Puromycin) in Xenorhabdus nematophila

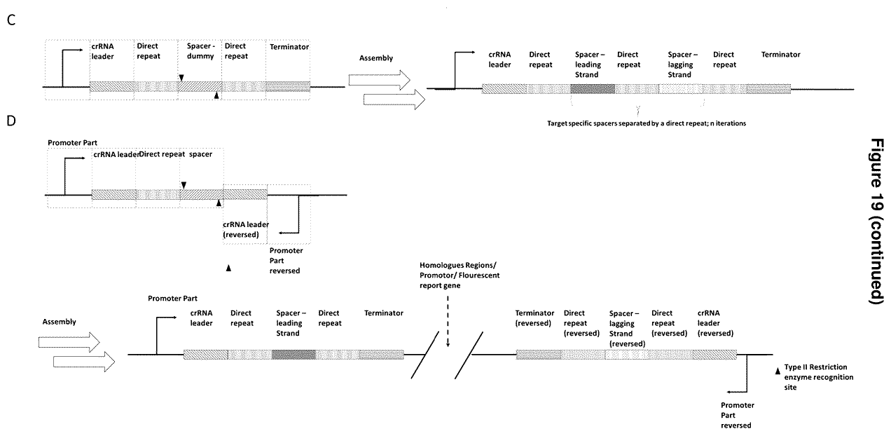

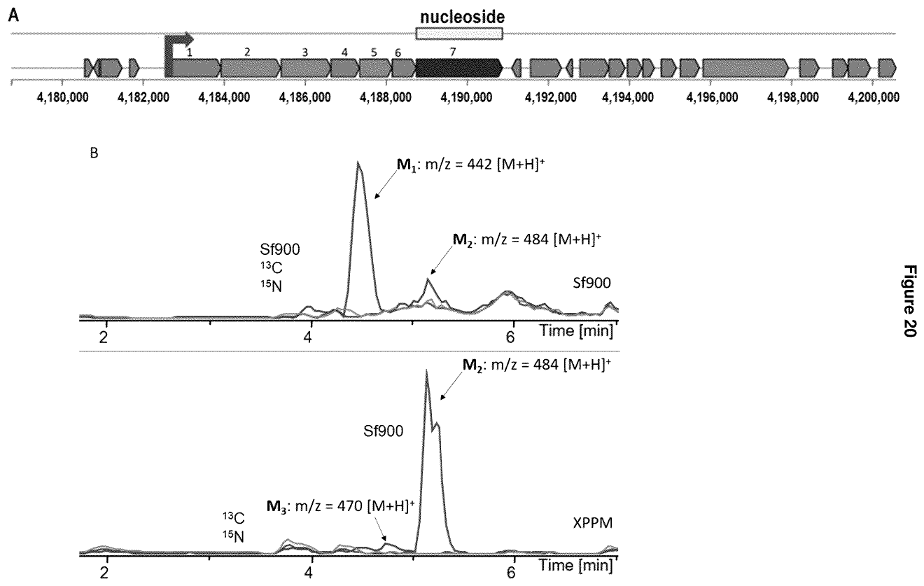

To further confirm the method developed by the inventor, a promoter exchange was performed in a global regulator cyaA and or hfq deficient mutant of Xenorhabdus nematophila upstream of the silent BGC dpr (FIG. 20A). Deletion of the GRs were done with the pAR20 system, with the: Δhfq specific HA-L and R having SEQ ID NOs 181-182 or ΔcyaA specific HA-L and R having SEQ ID NO 183.184. The activation of the dprA BGC by insertion of the Pvan promoter (SEQ ID NO 131) upstream of dprA was done together with the dprA specific HA-L and R having SEQ ID NO 185-186, also based on the pAR20 editing system.

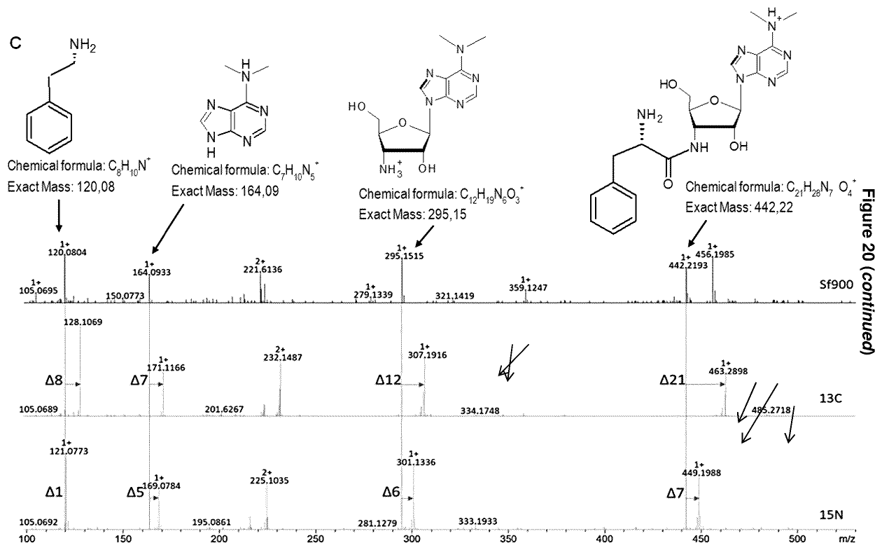

The activation of BGC led to the production of different derivatives in different culture media in Sf900: M1 (m/z=442 [M+H]⁺), M2 (m/z=484 [M+H]⁺) and in XPPM: M2 (m/z=484 [M+H]⁺), M3 (m/z=470 [M+H]⁺) (FIG. 20B). In Sf900, M1 was the major product and M2 as a byproduct. M3 was not detectable at all. When cultured in XPPM medium M2 was the main product and M3 was present as a byproduct. M1 was not produced. In order to determine a molecular formula, cultivation was carried out in ¹³C and ¹⁵N medium (ISOGRO®-13C Powder—Growth Medium; ISOGRO®-15N Powder-Growth Medium). The MS/MS spectra are shown in FIG. 20C. Using the data from FIG. 20C, mass shifts could be assigned to several mass signals. The altered masses result from the incorporation of heavy ¹³C/¹⁵N isotopes into the respective molecule, whereby the magnitude of the shift correlates with the number of isotopes incorporated. In this way, the molecular formula of M1 was determined as C₂₁H₂₇N₇O₄. According to the feeding experiments, the structures for the masses M1 to M3 could be confirmed by NMR and named as follows: Demethoxypuromycin (M1, 27), N-acetyl-demethoxypuromycin (M2, 28) and N-formyl-demethoxypuromycin (M3, 29) (FIG. 21A-C, Table 15 below).

TABLE 15

M₁ (441 Da)

| | | |
|---|---|---|
| Position | δ_{H} (mult., J) | δ_{C} (mult., J) |

Adenin

| | | |
|---|---|---|
| 2 | 8.24 (s) | 152.3, CH |
| 4 | — | 150.1, C |
| 5 | — | 120.1, C |
| 6 | — | 154.7, C |
| 8 | 8.45(s) | 138.3, CH |

Ribose

| | | |
|---|---|---|
| 10 | 5.99 (d, 2.7) | 89.8, CH |
| 11 | 4.51 (dd, 5.8, 2.8) | 73.5, CH |
| 12 | 4.47 (m) | 50.8, CH |
| 13 | 3.92 (m) | 83.8, CH |
| 14 | 3.67 (dd, 12.2, 2.2) | 61.3, CH₂ |
| | 3.45 (dd, 12.2, 4.1) | |

Phe

| | | |
|---|---|---|
| 16 | — | 171.2, C |
| 17 | 3.75 (ddd, 6.9) | 55.6, CH |
| 18 | 3.02 (dd, 13.5, 6.0) | 38.6, CH₂ |
| | 2.78 (dd, 13.5, 8.0) | |
| 19 | — | 137.9, C |
| 20 | 7.25 (m) | 129.8, CH |
| 21 | 7.30 (m) | 128.7, CH |
| 22 | 7.21 (m) | 126.9, CH |
| 23 | 7.30 (m) | 128.7, CH |
| 24 | 7.25 (m) | 129.8, CH |
| 25 | 8.35 (s) | — |

M₂ (483 Da)

| | | |
|---|---|---|
| Position | δ_{H} (mult., J) | δ_{C} (mult., J) |

Adenin

| | | |
|---|---|---|
| 2 | 8.24 (s) | 152.3, CH |
| 4 | — | 150.1, C |
| 5 | — | 120.1, C |
| 6 | — | 154.7, C |
| 8 | 8.45(s) | 138.3, CH |

Ribose

| | | |
|---|---|---|
| 10 | 6.01 (d, 3.1) | 89.8, CH |
| 11 | 4.52 (dd, 5.9, 3.1) | 73.5, CH |
| 12 | 4.48 (m) | 50.8, CH |
| 13 | 3.94 (m) | 83.8, CH |
| 14 | 3.67 (dd, 12.2, 2.1) | 61.3, CH₂ |
| | 3.46 (dd, 12.2, 4.0) | |
| 15 | 8.22 (d, 7.8) | — |

Phe

| | | |
|---|---|---|
| 16 | — | 171.2, C |
| 17 | 4.66 (ddd, 8.2, 7.2, 4.0) | 54.4, CH |
| 18 | 2.99 (dd, 13.6, 5.3) | 38.6, CH₂ |
| | 2.79 (dd, 13.6, 9.4) | |
| 19 | — | 138.3, C |
| 20 | 7.27 (m) | 129.7, CH |
| 21 | 7.27 (m) | 128.5, CH |
| 22 | 7.19 (m) | 126.7, CH |
| 23 | 7.27 (m) | 128.5, CH |
| 24 | 7.27 (m) | 129.7, CH |
| 25 | 8.17 (d, 8.4) | — |
| 26 | — | 169.6, C |
| 27 | 1.77 (s) | 22.9, CH₃ |

M₃ (469 Da)

| | | |
|---|---|---|
| Position | δ_{H} (mult., J) | δ_{C} (mult., J) |

Adenin

| | | |
|---|---|---|
| 2 | 8.24 (s) | 152.3, CH |
| 4 | — | 150.1, C |
| 5 | — | 120.1, C |
| 6 | — | 154.7, C |
| 8 | 8.45(s) | 138.3, CH |

Ribose

| | | |
|---|---|---|
| 10 | 6.00 (d, 3.0) | 89.8, CH |
| 11 | 5.19 (dd, 5.1) | 73.5, CH |
| 12 | 4.49 (m) | 50.8, CH |
| 13 | 3.94 (m) | 83.8, CH |
| 14 | 3.67 (d, 10.9) | 61.3, CH₂ |
| | 3.47 (m) | |
| 15 | 8.29 (m) | — |

Phe

| | | |
|---|---|---|
| 16 | — | 171.2, C |
| 17 | 4.66 (ddd, 5.3, 8.9) | 52.7, CH |
| 18 | 2.99 (dd, 13.7, 5.2) | 38.6, CH₂ |
| | 2.79 (dd, 13.7, 9.2) | |
| 19 | — | 137.9, C |
| 20 | 7.28 (m) | 129.7, CH |
| 21 | 7.28 (m) | 128.5, CH |
| 22 | 7.20 (m) | 126.8, CH |
| 23 | 7.28 (m) | 128.5, CH |
| 24 | 7.28 (m) | 129.7, CH |
| 25 | 6.08 (d, 3.9) | — |
| 26 | 7.93 (d, 0.9) | 161.2, C |

What is claimed is:

1.-21. (canceled)

22. A method to elicit production of a secondary metabolite by a bacterium, wherein the method comprises the following steps:
i) deleting a global regulator (GR) gene in said bacterium, and
ii) optionally activating a biosynthetic gene cluster (BGC) involved in production of said secondary metabolite in said bacterium;
or wherein the method comprises the following steps:
i′) essentially activating a biosynthetic gene cluster (BGC) involved in production of said secondary metabolite in said bacterium, and
ii′) optionally deleting a GR gene in said bacterium;
and the method further comprises:
iii) expressing in said bacterium at least one positive selectable marker gene and at least one negative selectable marker gene;
wherein deleting a GR gene of steps i) or ii′) comprises introducing in said bacterium at least one GR specific siRNA, or a group of sequences comprising a Cas gene, at least one GR specific crRNA array, a pair of GR specific homology regions left and right; and/or
wherein activating a BGC of steps ii) or i′) comprises introducing in said bacterium a group of sequences comprising a Cas gene, at least one BGC specific crRNA array, a pair of BGC specific homology regions left and right, a promoter system, and optionally an enhancer sequence.

23. The method according to claim 22, wherein step ii) or step i′) comprises activating at least 2 BGCs in said bacterium.

24. The method according to claim 22, wherein the GR gene is cyaA gene.

25. The method according claim 22, wherein said BGC is selected from the group comprising puromycin, madumycin II, xenoamicin, rupshomycin, rupshomycin derivatives, safracin, safracin derivatives.

26. The method according to claim 22, wherein the GR gene is cyaA gene and said BGC is xenoamicin.

27. The method of claim 22, wherein the step i)-iii) are performed using an automated liquid handling robotics, and wherein said robotics enables high-throughput manipulation of liquid added to or removed from cultures comprising the bacteria.

28. A method for screening for a global regulator (GR) gene in a bacterium, the method comprising:
a) replacing a gene sequence of a first constitutively expressed biosynthetic gene cluster (BGC) with a first fluorescent reporter gene;
b) replacing a gene sequence of a second constitutively expressed BGC with a second fluorescent reporter gene, wherein the first and second fluorescent reporters emit two non-overlapping fluorescent signals;
c) obtaining a bacterium indicator strain emitting the first and the second fluorescent signals;
d) performing random mutagenesis in said bacterium indicator strain to generate random mutations;
e) screening and identifying non-fluorescent mutant bacteria that do not emit the first and the second fluorescent signal,
f) isolating said non-fluorescent mutant bacteria,
g) performing whole genome sequencing of said non-fluorescent mutant bacterium to map the mutations generated in step d);
h) identifying the mutations generated in a GR gene, wherein said GR gene regulates the activity of the first constitutively expressed BGC and of the second constitutively expressed BGC in said bacterium;
wherein the replacing of a gene sequence of a BGC at step a) and b) does not disrupt a regulatory element of said BGC;
wherein in step a) and b) the reading frame is conserved;
wherein said GR gene regulates production of a target secondary metabolite.

29. The method of claim 28, wherein step e) is performed by fluorescence activated cell sorting, fluorescence spectroscopy, stereo microscopy, or fluorescence imaging.

30. The method of claim 28, further comprising after step h), the following step i):
i) screening for production of secondary metabolites regulated by said GR gene by HPLC/mass spectrometry analysis.

31. The method of claim 28, wherein performing random mutagenesis of step d) comprises chemical random mutagenesis, or UV-mediated random mutagenesis, or error-prone PCR or transposon mutagenesis.

32. The method of claim 28, wherein said BGC of step a) and/or b) encodes an enzyme responsible for production of secondary metabolites, wherein the enzyme is selected from the group comprising non-ribosomal peptide synthetase, terpene synthase/cyclase, polyketide synthase, ribosomally produced peptide (RiPP).

33. The method of claim 28, wherein the secondary metabolite is an antibiotic, or an anti-cancer drug, or an immune suppressive drug and is preferably selected from the group comprising Puromycin, Madumycin II, Xenoamicin, rupshomycin, rupshomycin derivatives, safracin, safracin derivatives.

34. The method of claim 28, wherein the bacterium is not Streptomyces.

35. The method of claim 28, wherein the bacterium is selected from the group comprising Xenorhabdus, Photorhabdus, Pseudomonas, Serratia, Vibrio, myxobacteria, cyanobacteria, Bacillus, and Paenibacillus.

36. The method of claim 28, wherein the steps a) and b) are performed introducing in said bacterium a Cas gene, a first and second constitutively expressed BGC specific crRNA arrays, a first and second fluorescent reporter gene, and a first and second pair of BGC specific homology regions left and right.

37. A compound of general formula (I) 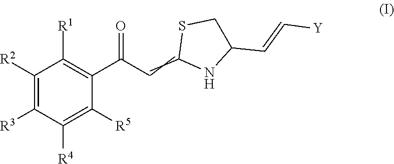
wherein
Y represents —CH═CH₂ 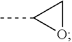
or
R¹, R², R³, R⁴, and R⁵ are independently of each other selected from —H, —NH₂, —NHR⁶, —F, —OH, —OCH₃, —OCH₂CH₃, and —OCH₂CH₂CH₃;
or R² and R³ or R³ and R⁴ together with the carbon atom to which they are bound to form one of the following rings: 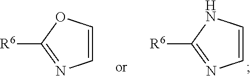
wherein R⁶ represents —H, —CH₃, —CH₂CH₂CH₃, —CH(CH₃)₂, —CH₂(CH₂)₂CH₃, —CH₂(CH₂)₃CH₃, —CH₂(CH₂)₄CH₃, —CH₂(CH₂)₅CH₃, —CH₂(CH₂)₆CH₃, —CH₂(CH₂)₇CH₃, —CH₂(CH₂)₈CH₃, —CH₂(CH₂)₉CH₃, —CH₂(CH₂)₁₀CH₃, —CH₂(CH₂)₁₁CH₃, —CH₂(CH₂)₁₂CH₃, —CH₂CH(CH₃)₂, —C(CH₃)₃, —CH₂CH═CH₂, —CH₂CH═CH(CH₃), —CH₂CH═C(CH₃)₂, or —CH₂CH═CHCH₂CH₃;
with the proviso that when Y represents —CH═CH₂, R¹, R², R⁴, and R⁵ cannot be —H, and R³ cannot be —OH simultaneously.

38. The compound according to claim 37 having the following structure 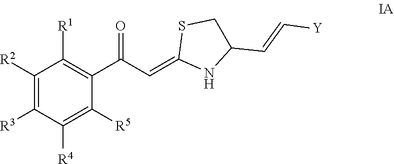
wherein Y, and R¹-R⁵ have the meanings as defined in claim 16.

39. The compound according to claim 37 having the following structure 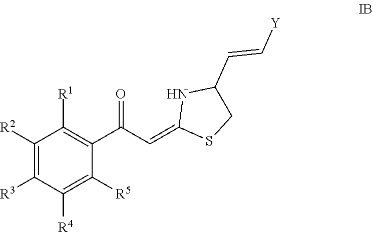
wherein Y, and R¹-R⁵ have the meanings as defined in claim 16.

40. The compound according to claim 37 selected from the group consisting of:

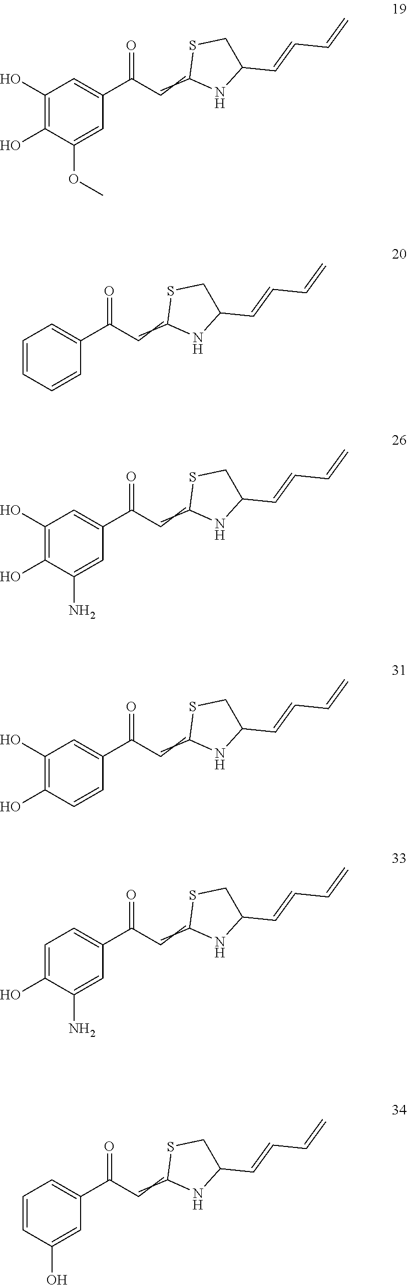 [SMILES: C=C/C=C/C1CSC(=CC(=O)c2cc(O)c(O)c(OC)c2)N1 ; C=C/C=C/C1CSC(=CC(=O)c2ccccc2)N1 ; C=C/C=C/C1CSC(=CC(=O)c2cc(N)c(O)c(O)c2)N1 ; C=C/C=C/C1CSC(=CC(=O)c2ccc(O)c(O)c2)N1 ; C=C/C=C/C1CSC(=CC(=O)c2ccc(O)c(N)c2)N1 ; C=C/C=C/C1CSC(=CC(=O)c2cccc(O)c2)N1]

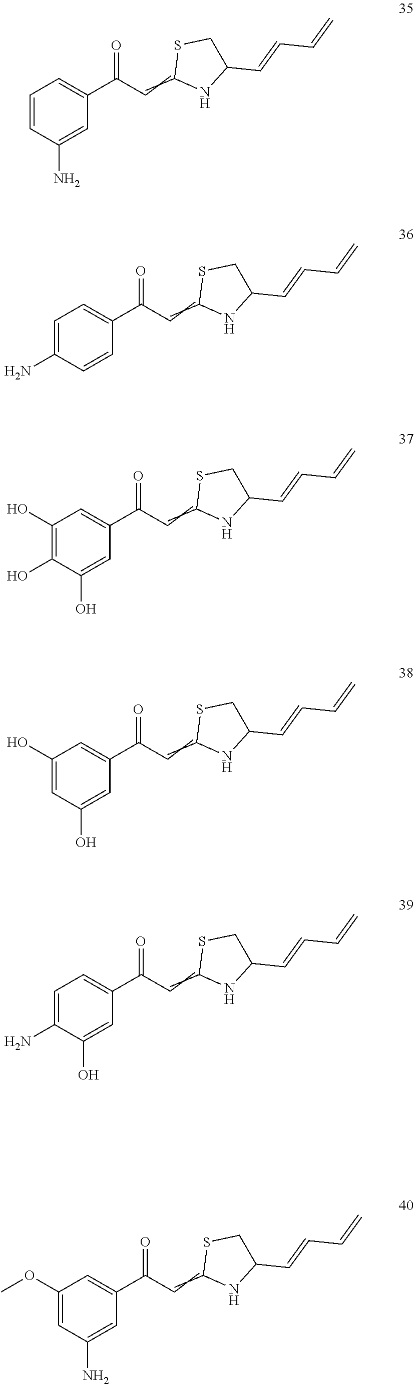

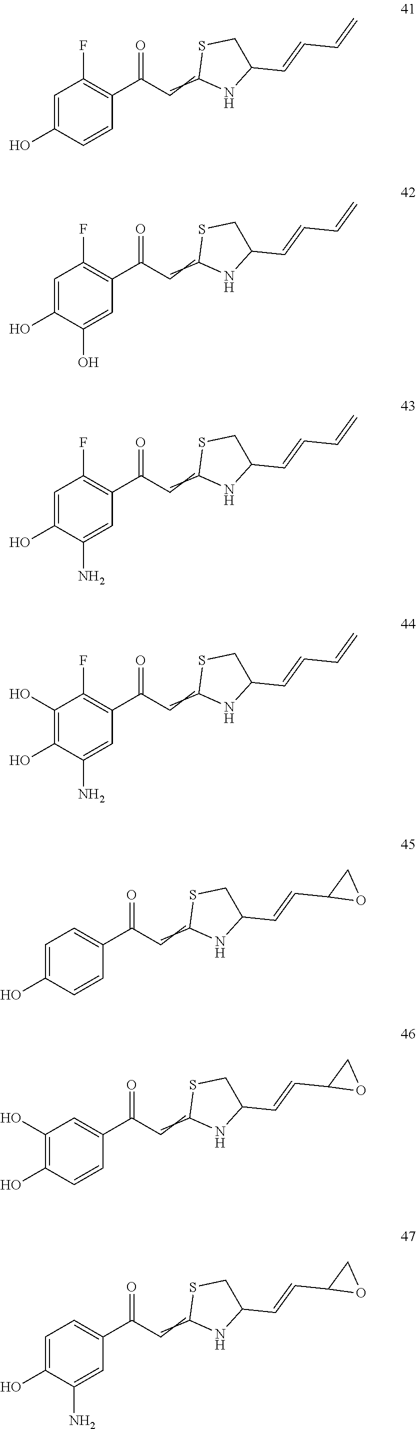

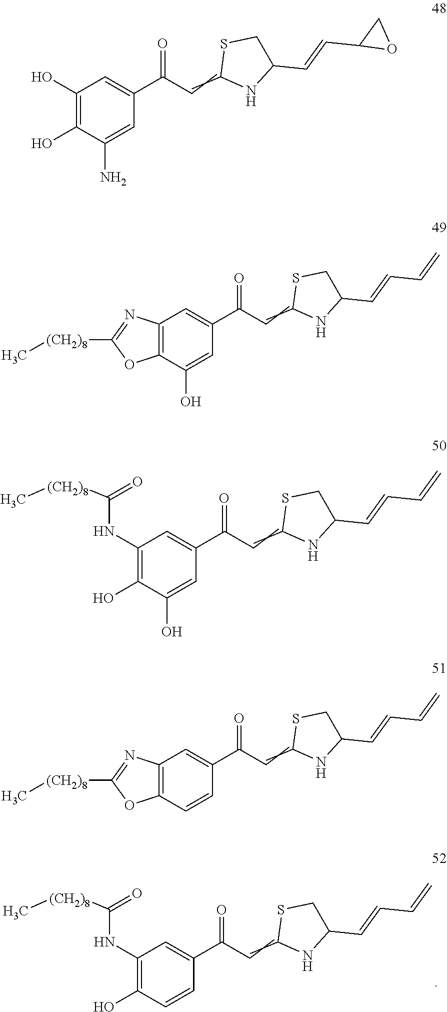

41. The compound according to claim 40, wherein the compound is a (2E)-thiazolidine isomer.

42. The compound according to claim 40, wherein the compound is a (2Z)-thiazolidine isomer.


SEQUENCE LISTING (212 sequences)

SEQ ID NO: 1 - DNA, 18 bp, synthetic construct
  source 1..18: mol_type = other DNA
  misc_feature 1..18: note = primer AR533
tgaatcccatagggcagg

SEQ ID NO: 2 - DNA, 22 bp, synthetic construct
  source 1..22: mol_type = other DNA
  misc_feature 1..22: note = primer AR534
gatctatcaacaggagtccaag

SEQ ID NO: 3 - DNA, 41 bp, synthetic construct
  source 1..41: mol_type = other DNA
  misc_feature 1..41: note = primer PCO45
gatcctctagagtcgacctgaaagtgtttcagttgtttttg

SEQ ID NO: 4 - DNA, 51 bp, synthetic construct
  source 1..51: mol_type = other DNA
  misc_feature 1..51: note = primer PC O46
caaaaagacaaatatcaatagtcatcgctgcttcagcgtttcaatatagag

SEQ ID NO: 5 - DNA, 47 bp, synthetic construct
  source 1..47: mol_type = other DNA
  misc_feature 1..47: note = primer PC047
ttgtatctctatattgaaacgctgcactattgatatttgtctttttg

SEQ ID NO: 6 - DNA, 51 bp, synthetic construct
  source 1..51: mol_type = other DNA
  misc_feature 1..51: note = PCO48
gtggaattcccgggagagctcagatcttatacggccccaagagaaatcaca

SEQ ID NO: 7 - DNA, 24 bp, synthetic construct
  source 1..24: mol_type = other DNA
  misc_feature 1..24: note = primer PCO49
cagagccagttccaactcctttac

SEQ ID NO: 8 - DNA, 24 bp, synthetic construct
  source 1..24: mol_type = other DNA
  misc_feature 1..24: note = primer PC O50
gccaagaccgtgcattttggagaa

SEQ ID NO: 9 - DNA, 45 bp, synthetic construct
  source 1..45: mol_type = other DNA
  misc_feature 1..45: note = primer PC O69
gatcctctagagtcgacctgcaggtatttctcaggcttttcagcc

SEQ ID NO: 10 - DNA, 44 bp, synthetic construct
  source 1..44: mol_type = other DNA
  misc_feature 1..44: note = PC O70
ggtaaatcagtaatgtgattcgatataaaggtacaagacgtctc

SEQ ID NO: 11 - DNA, 44 bp, synthetic construct
  source 1..44: mol_type = other DNA
  misc_feature 1..44: note = primer PC O71
ttcgatataaaggtacaatcacattactgatttaccccgcgatg

SEQ ID NO: 12 - DNA, 51 bp, synthetic construct
  source 1..51: mol_type = other DNA
  misc_feature 1..51: note = primer PC O72
gtggaattcccgggagagctcagatctcctatctgccaaacgacagatcag

SEQ ID NO: 13 - DNA, 22 bp, synthetic construct
  source 1..22: mol_type = other DNA
  misc_feature 1..22: note = primer PC O73
ccacatagctaaagggctttgg

SEQ ID NO: 14 - DNA, 24 bp, synthetic construct
  source 1..24: mol_type = other DNA
  misc_feature 1..24: note = primer PC O74
catatccggatcataaggtgcttc

SEQ ID NO: 15 - DNA, 46 bp, synthetic construct
  source 1..46: mol_type = other DNA
  misc_feature 1..46: note = primer ODA1
tttgggctaacaggaggctagcatatgcctatgtcatgcaatggta

SEQ ID NO: 16 - DNA, 46 bp, synthetic construct
  source 1..46: mol_type = other DNA
  misc_feature 1..46: note = ODA2
ccgtttaaacatttaaatctgcagatgatgacaacagaactgccag

SEQ ID NO: 17 - DNA, 20 bp, synthetic construct
  source 1..20: mol_type = other DNA
  misc_feature 1..20: note = primer ODA3
tgtgagtgtaaataggcatg

SEQ ID NO: 18 - DNA, 28 bp, synthetic construct
  source 1..28: mol_type = other DNA
  misc_feature 1..28: note = primer VPpCEP-fw
gctatgccatagcatttttatccataag

SEQ ID NO: 19 - DNA, 20 bp, synthetic construct
  source 1..20: mol_type = other DNA
  misc_feature 1..20: note = VPpDS132-rv
acatgtggaattgtgagcgg

SEQ ID NO: 20 - DNA, 31 bp, synthetic construct
  source 1..31: mol_type = other DNA
  misc_feature 1..31: note = AR426
ggctacggtctcacgaaaactcacgttaagg

SEQ ID NO: 21 - DNA, 38 bp, synthetic construct
  source 1..38: mol_type = other DNA
  misc_feature 1..38: note = AR427
ggctacggtctcaaggtatcaccgttacatatcaaagg

SEQ ID NO: 22 - DNA, 37 bp, synthetic construct
  source 1..37: mol_type = other DNA
  misc_feature 1..37: note = AR428
ggctacggtctcgacctggaagatgccaggaagatac

SEQ ID NO: 23 - DNA, 42 bp, synthetic construct
  source 1..42: mol_type = other DNA
  misc_feature 1..42: note = primer AR429
ggctacggtctctttcgcactagtaacaacttatatcgtatg

SEQ ID NO: 24 - DNA, 18 bp, synthetic construct
  source 1..18: mol_type = other DNA
  misc_feature 1..18: note = AR430
gaattctctagagtcgac

SEQ ID NO: 25 - DNA, 19 bp, synthetic construct
  source 1..19: mol_type = other DNA
  misc_feature 1..19: note = primer AR431
ggatccagcatatgcggtg

SEQ ID NO: 26 - DNA, 37 bp, synthetic construct
  source 1..37: mol_type = other DNA
  misc_feature 1..37: note = AR432
cacaccgcatatgctggatcccaatcgcgcgggtttg

SEQ ID NO: 27 - DNA, 42 bp, synthetic construct
  source 1..42: mol_type = other DNA
  misc_feature 1..42: note = primer AR433
ctgcaggtcgactctagagaattctttcgctcctgccctatg

SEQ ID NO: 28 - DNA, 49 bp, synthetic construct
  source 1..49: mol_type = other DNA
  misc_feature 1..49: note = AR434
gcttagatctattaccctgttatccctacttgacataagcctgttcggt

SEQ ID NO: 29 - DNA, 41 bp, synthetic construct
  source 1..41: mol_type = other DNA
  misc_feature 1..41: note = primer AR435
cttcggaataggaacttcatgagctcggccgggaagccgat

SEQ ID NO: 30 - DNA, 24 bp, synthetic construct
  source 1..24: mol_type = other DNA
  misc_feature 1..24: note = primer AR436
gctcatgaagttcctattccgaag

SEQ ID NO: 31 - DNA, 18 bp, synthetic construct
  source 1..18: mol_type = other DNA
  misc_feature 1..18: note = primer AR437
cgtgatggcttccatgtc

SEQ ID NO: 32 - DNA, 19 bp, synthetic construct
  source 1..19: mol_type = other DNA
  misc_feature 1..19: note = primer AR_seq_07
ctattggaatgcggatagg

SEQ ID NO: 33 - DNA, 19 bp, synthetic construct
  source 1..19: mol_type = other DNA
  misc_feature 1..19: note = primer AR_seq_08
cggaataaagtaagtcagg

SEQ ID NO: 34 - DNA, 20 bp, synthetic construct
  source 1..20: mol_type = other DNA
  misc_feature 1..20: note = primer AR_seq_11
acgcaaaatgtagaagaaag

SEQ ID NO: 35 - DNA, 17 bp, synthetic construct
  source 1..17: mol_type = other DNA
  misc_feature 1..17: note = primer AR_seq_12
caggcgctagaatttcc

SEQ ID NO: 36 - DNA, 19 bp, synthetic construct
  source 1..19: mol_type = other DNA
  misc_feature 1..19: note = primer AR_seq_13
ctattggaatgcggatagg

SEQ ID NO: 37 - DNA, 22 bp, synthetic construct
  source 1..22: mol_type = other DNA
  misc_feature 1..22: note = primer AR_seq_14
gacgggtataagcatgattatc

SEQ ID NO: 38 - DNA, 20 bp, synthetic construct
  source 1..20: mol_type = other DNA
  misc_feature 1..20: note = primer AR_seq_17
tttgctgcttcttttcgccc

SEQ ID NO: 39 - DNA, 20 bp, synthetic construct
  source 1..20: mol_type = other DNA
  misc_feature 1..20: note = primer AR_seq_18
gaatatggccagcttgcagc

SEQ ID NO: 40 - DNA, 20 bp, synthetic construct
  source 1..20: mol_type = other DNA
  misc_feature 1..20: note = primer AR_seq_21
tttcacccggcaagaaaggt

SEQ ID NO: 41 - DNA, 20 bp, synthetic construct
  source 1..20: mol_type = other DNA
  misc_feature 1..20: note = primer AR_seq_22
agggacttcttcaagctggc

SEQ ID NO: 42 - DNA, 20 bp, synthetic construct
  source 1..20: mol_type = other DNA
  misc_feature 1..20: note = primer AR_seq_29
agaagatcctgccagcccta

SEQ ID NO: 43 - DNA, 20 bp, synthetic construct
  source 1..20: mol_type = other DNA
  misc_feature 1..20: note = primer AR_seq_30
tcgggtcgtgagttctttcg

SEQ ID NO: 44 - DNA, 20 bp, synthetic construct
  source 1..20: mol_type = other DNA
  misc_feature 1..20: note = primer AR_seq_31
actggaacgagcgctgtaaa

SEQ ID NO: 45 - DNA, 20 bp, synthetic construct
  source 1..20: mol_type = other DNA
  misc_feature 1..20: note = primer AR_seq_32
ggcaattgtgttccgtgacc

SEQ ID NO: 46 - DNA, 20 bp, synthetic construct
  source 1..20: mol_type = other DNA
  misc_feature 1..20: note = primer AR_seq_33
tggtgctttggtgggttctt

SEQ ID NO: 47 - DNA, 20 bp, synthetic construct
  source 1..20: mol_type = other DNA
  misc_feature 1..20: note = primer AR_seq_34
gcagtctgaaatccccgaca

SEQ ID NO: 48 - DNA, 20 bp, synthetic construct
  source 1..20: mol_type = other DNA
  misc_feature 1..20: note = primer AR_seq_35
ccatcagattcctgcccgtt

SEQ ID NO: 49 - DNA, 20 bp, synthetic construct
  source 1..20: mol_type = other DNA
  misc_feature 1..20: note = primer AR_seq_36
ggcaattgtgttccgtgacc

SEQ ID NO: 50 - DNA, 20 bp, synthetic construct
  source 1..20: mol_type = other DNA
  misc_feature 1..20: note = primer AR_seq_37
ggaacaggtggccgagttaa

SEQ ID NO: 51 - DNA, 20 bp, synthetic construct
  source 1..20: mol_type = other DNA
  misc_feature 1..20: note = primer AR_seq_38
cgcccgcatcaattgtagtg

SEQ ID NO: 52 - DNA, 20 bp, synthetic construct
  source 1..20: mol_type = other DNA
  misc_feature 1..20: note = primer AR_seq_76
tgccagccctaacaaacgat

SEQ ID NO: 53 - DNA, 20 bp, synthetic construct
  source 1..20: mol_type = other DNA
  misc_feature 1..20: note = primer AR_seq_77
ttgtggtgcacagcgaattg

SEQ ID NO: 54 - DNA, 20 bp, synthetic construct
  source 1..20: mol_type = other DNA
  misc_feature 1..20: note = primer AR_seq_84
ctacccggagttatggcgac

SEQ ID NO: 55 - DNA, 20 bp, synthetic construct
  source 1..20: mol_type = other DNA
  misc_feature 1..20: note = primer AR_seq_85
cctgagtcgccgtactgatc

SEQ ID NO: 56 - DNA, 20 bp, synthetic construct
  source 1..20: mol_type = other DNA
  misc_feature 1..20: note = primer AR577
aaacgtgcatgggtttctgc

SEQ ID NO: 57 - DNA, 59 bp, Xenorhabdus indica
  source 1..59: mol_type = other DNA
  misc_feature 1..59: note = primer AR606
tgtttaatactagagaaagaggggaaatactagatgaatactggtataactcatcgctg

SEQ ID NO: 58 - DNA, 40 bp, Xenorhabdus indica
  source 1..40: mol_type = other DNA
  misc_feature 1..40: note = primer AR607
ataacatcaattgccattactatccccttagaaaacccca

SEQ ID NO: 59 - DNA, 42 bp, Xenorhabdus indica
  source 1..42: mol_type = other DNA
  misc_feature 1..42: note = primer AR608
aggggatagtaatggcaattgatgttattaatgtgactacgc

SEQ ID NO: 60 - DNA, 48 bp, Xenorhabdus indica
  source 1..48: mol_type = other DNA
  misc_feature 1..48: note = primer AR609
tcgccagggttttcccagtcacgacttagacttcttcgataaactcac

SEQ ID NO: 61 - DNA, 55 bp, Xenorhabdus indica
  source 1..55: mol_type = other DNA
  misc_feature 1..55: note = primer AR620
tgtttaatactagagaaagaggggaaatactagatgcaacagcataacattgcgt

SEQ ID NO: 62 - DNA, 49 bp, Xenorhabdus indica
  source 1..49: mol_type = other DNA
  misc_feature 1..49: note = primer AR621
tcgccagggttttcccagtcacgacttaatagatatcgatcaattgcct

SEQ ID NO: 63 - DNA, 57 bp, Xenorhabdus indica
  source 1..57: mol_type = other DNA
  misc_feature 1..57: note = primer AR616
tgtttaatactagagaaagaggggaaatactagatgatcaatattatcaagcgcgct

SEQ ID NO: 64 - DNA, 50 bp, Xenorhabdus indica
  source 1..50: mol_type = other DNA
  misc_feature 1..50: note = primer AR617
tcgccagggttttcccagtcacgacttattttgctaataaagccttagca

SEQ ID NO: 65 - DNA, 56 bp, Xenorhabdus indica
  source 1..56: mol_type = other DNA
  misc_feature 1..56: note = primer AR604
tgtttaatactagagaaagaggggaaatactagatgaaaattgttgattgcccagc

SEQ ID NO: 66 - DNA, 50 bp, Xenorhabdus indica
  source 1..50: mol_type = other DNA
  misc_feature 1..50: note = primer AR605
tcgccagggttttcccagtcacgacttattctacccctataaataactct

SEQ ID NO: 67 - DNA, 31 bp, Photorhabdus sp.
  source 1..31: mol_type = other DNA
gaagatatctatgacattgcgataaaacaaa

SEQ ID NO: 68 - DNA, 31 bp, Photorhabdus sp.
  source 1..31: mol_type = other DNA
gcagtttcaggcgcaatttcactaatattgc

SEQ ID NO: 69 - DNA, 31 bp, Photorhabdus sp.
  source 1..31: mol_type = other DNA
tatgaattccataacccggaagtatggatga

SEQ ID NO: 70 - DNA, 31 bp, Photorhabdus sp.
  source 1..31: mol_type = other DNA
tatccttagggagttcgtttcttcaaaatat

SEQ ID NO: 71 - DNA, 31 bp, Photorhabdus sp.
  source 1..31: mol_type = other DNA
ttgtcagaggctgaacgtacactgctattgg

SEQ ID NO: 72 - DNA, 31 bp, Photorhabdus sp.
  source 1..31: mol_type = other DNA
ttaccgatgttgcgctacagtccgtcacaat

SEQ ID NO: 73 - DNA, 31 bp, Photorhabdus sp.
  source 1..31: mol_type = other DNA
attgatgcctgatgtgtgcccgtttttattt

SEQ ID NO: 74 - DNA, 31 bp, Photorhabdus sp.
  source 1..31: mol_type = other DNA
aaaactgtcattctcaacagtcatcacaaat

SEQ ID NO: 75 - DNA, 31 bp, Xenorhabdus nematophila
  source 1..31: mol_type = other DNA
agactgatgatgtggcaatgagtgacttggc

SEQ ID NO: 76 - DNA, 31 bp, Xenorhabdus nematophila
  source 1..31: mol_type = other DNA
gctatctgggacgaattgatgatcagataaa

SEQ ID NO: 77 - DNA, 31 bp, Xenorhabdus nematophila
  source 1..31: mol_type = other DNA
attaatataaattcgatgttatcggaacagt

SEQ ID NO: 78 - DNA, 31 bp, Xenorhabdus nematophila
  source 1..31: mol_type = other DNA
gtgaagagcagacactacctatgcttgatgc

SEQ ID NO: 79 - DNA, 31 bp, Xenorhabdus nematophila
  source 1..31: mol_type = other DNA
acgcctcattgataatttgcacggccttttt

SEQ ID NO: 80 - DNA, 31 bp, Xenorhabdus nematophila
  source 1..31: mol_type = other DNA
actatatggatgctcagaatctctatgatta

SEQ ID NO: 81 - DNA, 31 bp, Xenorhabdus nematophila
  source 1..31: mol_type = other DNA
ataaaataaaagggtaataaaatatgtatga

SEQ ID NO: 82 - DNA, 31 bp, Xenorhabdus nematophila
  source 1..31: mol_type = other DNA
cctttagattatttttgtgttgtttttcccc

SEQ ID NO: 83 - DNA, 31 bp, Xenorhabdus nematophila
  source 1..31: mol_type = other DNA
aggtatgcccaacgcccgtatcaaagaggat

SEQ ID NO: 84 - DNA, 31 bp, Xenorhabdus nematophila
  source 1..31: mol_type = other DNA
caatatcgtatcatgctgggtattctccagc

SEQ ID NO: 85 - DNA, 31 bp, Photorhabdus sp.
  source 1..31: mol_type = other DNA
actacggagatattcattttattattatata

SEQ ID NO: 86 - DNA, 31 bp, Photorhabdus sp.
  source 1..31: mol_type = other DNA
gaggcagattaatacccttaccaattacatt

SEQ ID NO: 87 - DNA, 31 bp, Photorhabdus sp.
  source 1..31: mol_type = other DNA
atacgatgtcatcgtaattcaccctaataaa

SEQ ID NO: 88 - DNA, 31 bp, Photorhabdus sp.
  source 1..31: mol_type = other DNA
aattaattacgatatcttgggaaaaaacagt

SEQ ID NO: 89 - DNA, 31 bp, Xenorhabdus nematophila
  source 1..31: mol_type = other DNA
attcctatatcttcaagttaattttggaaaa

SEQ ID NO: 90 - DNA, 31 bp, Xenorhabdus nematophila
  source 1..31: mol_type = other DNA
atgaagatactgtcattatctatttaaccaa

SEQ ID NO: 91 - DNA, 31 bp, Xenorhabdus nematophila
  source 1..31: mol_type = other DNA
aggtgttggttaaatagataatgacagtatc

SEQ ID NO: 92 - DNA, 31 bp, Xenorhabdus nematophila
  source 1..31: mol_type = other DNA
acaataaaataaataaatcaatgattaaatt

SEQ ID NO: 93 - DNA, 31 bp, Xenorhabdus nematophila
  source 1..31: mol_type = other DNA
cggagaatgatgtcggcaacgatgcgggtca

SEQ ID NO: 94 - DNA, 31 bp, Xenorhabdus nematophila
  source 1..31: mol_type = other DNA
tacaggattggtcgatcactcatgaggaaat

SEQ ID NO: 95 - DNA, 31 bp, Photorhabdus luminescens
  source 1..31: mol_type = other DNA
gttgatgaacacttctctttagacgatatgc

SEQ ID NO: 96 - DNA, 30 bp, Photorhabdus luminescens
  source 1..30: mol_type = other DNA
cacaaccgtcagatgcatgaatgctcctgt

SEQ ID NO: 97 - DNA, 31 bp, Photorhabdus luminescens
  source 1..31: mol_type = other DNA
ttcatacaggttcaccaaaaacgtgggcacg

SEQ ID NO: 98 - DNA, 31 bp, Photorhabdus luminescens
  source 1..31: mol_type = other DNA
aggcgattcattactaataatagtgacggtt

SEQ ID NO: 99 - DNA, 31 bp, Photorhabdus luminescens
  source 1..31: mol_type = other DNA
tgattttgataaacataacagaatattagat

SEQ ID NO: 100 - DNA, 31 bp, Photorhabdus luminescens
  source 1..31: mol_type = other DNA
tctgtagtttgataaatgaatacggtgaata

SEQ ID NO: 101 - DNA, 31 bp, Xenorhabdus nematophila
  source 1..31: mol_type = other DNA
ggtttgactatatcagtcgtcctgatttatg

SEQ ID NO: 102 - DNA, 32 bp, Xenorhabdus nematophila
  source 1..32: mol_type = other DNA
atcgcatcggctataccaaacggaatagaggc

SEQ ID NO: 103 - DNA, 31 bp, Xenorhabdus nematophila
  source 1..31: mol_type = other DNA
gattagaacaagatgtactttatcgggtcgg

SEQ ID NO: 104 - DNA, 31 bp, Xenorhabdus nematophila
  source 1..31: mol_type = other DNA
tgcctctgacaacatcggtaaatcagcaggt

SEQ ID NO: 105 - DNA, 31 bp, Xenorhabdus sp.
  source 1..31: mol_type = other DNA
tctcccgatgtcaaattctatcgatcacaaa

SEQ ID NO: 106 - DNA, 31 bp, Xenorhabdus sp.
  source 1..31: mol_type = other DNA
tgtgaaaaaataaccagaataaattcatatt

SEQ ID NO: 107 - DNA, 31 bp, Xenorhabdus sp.
  source 1..31: mol_type = other DNA
agcacccataatggccgcgccaattccacgg

SEQ ID NO: 108 - DNA, 31 bp, Xenorhabdus sp.
  source 1..31: mol_type = other DNA
ctaatgcaggtgaattgcaacaattgttcat

SEQ ID NO: 109 - DNA, 31 bp, Xenorhabdus indica
  source 1..31: mol_type = other DNA
aaattaattgtatccattgagttactgtaac

SEQ ID NO: 110 - DNA, 31 bp, Xenorhabdus indica
  source 1..31: mol_type = other DNA
cttattggctaattgatagctgagtaaccat

SEQ ID NO: 111 - DNA, 31 bp, Xenorhabdus indica
  source 1..31: mol_type = other DNA
aaatgtgcagagaatttataataaaaataat

SEQ ID NO: 112 - DNA, 31 bp, Xenorhabdus indica
  source 1..31: mol_type = other DNA
ctcaggagcaatcctgacctaataaggccct

SEQ ID NO: 113 - DNA, 31 bp, Xenorhabdus indica
  source 1..31: mol_type = genomic DNA
gccaccatgcaggccaggttaatcctcgctt

SEQ ID NO: 114 - DNA, 31 bp, Xenorhabdus indica
  source 1..31: mol_type = other DNA
cctgaactcattcacgccaatcgactgacta

SEQ ID NO: 115 - DNA, 31 bp, Xenorhabdus indica
  source 1..31: mol_type = other DNA
aatcttatcggtagcatcccccagcagcgtt

SEQ ID NO: 116 - DNA, 31 bp, Xenorhabdus indica
  source 1..31: mol_type = other DNA
ctccggcaatcacgtcaaacagcccgtttgc

SEQ ID NO: 117 - DNA, 31 bp, Xenorhabdus indica
  source 1..31: mol_type = other DNA
acgtcgtaatgcgttcaggaatggatcttgc

SEQ ID NO: 118 - DNA, 31 bp, Xenorhabdus indica
  source 1..31: mol_type = other DNA
taaacacgccatctctactgttgtgccttct

SEQ ID NO: 119 - DNA, 243 bp, synthetic construct
  source 1..243: mol_type = other DNA
  misc_feature 1..35: note = PJ23119
  misc_feature 36..65: note = crRNA leader
  misc_feature 66..101: note = Direct Repeat
  misc_feature 102..149: note = Spacer Dummy with BsaI site
  misc_feature 150..185: note = Direct Repeat
  misc_feature 186..243: note = terminator
ttgacagctagctcagtcctaggtataatactagttcgagattttcaggagctaaggaagctaaagtctaagaactttaaataatttctactgttgtagatagagaccgtagccttaggccaattaccaacttggtggctacggtctctgtctaagaactttaaataatttctactgttgtagatgagaagtcatttaataaggccactggctcaccttcgggtgggcctttctgcgcaattg

SEQ ID NO: 120 - DNA, 163 bp, synthetic construct
  source 1..163: mol_type = other DNA
  misc_feature 1..17: note = BsaI site
  misc_feature 17^18: note = insertion site of selected target sequence A
  misc_feature 18..53: note = direct repeat
  misc_feature 54..111: note = terminator
  misc_feature 112..133: note = placeholder
  misc_feature 133^134: note = insertion site of homologues region left
  misc_feature 133..145: note = GS-linker sequence I
  misc_feature 146..163: note = BsaI site
gcctacggtctcaagatgtctaagaactttaaataatttctactgttgtagatgagaagtcatttaataaggccactggctcaccttcgggtgggcctttctgcgcaattgttaggccaattaccaacttggtggatccgctggcatctctgagaccgtagcc

SEQ ID NO: 121 - DNA, 181 bp, synthetic construct
  source 1..181: mol_type = other DNA
  misc_feature 1..35: note = Adapter with BsaI site
  misc_feature 35^36: note = Insertion site for selected target sequence
  misc_feature 38..71: note = direct repeat
  misc_feature 72..129: note = TERMINATOR
  misc_feature 130..151: note = placeholder
  misc_feature 151^152: note = Insertion site for homologues region
  misc_feature 152..181: note = Adapter with BsaI site
aaccggcgtattaagttgggctacggtctcgagatgtctaagaactttaaataatttctactgttgtagatgagaagtcatttaataaggccactggctcaccttcgggtgggcctttctgcgcaattgttaggccaattaccaacttggtggtatctgcgctccactagagaccgtagcc

SEQ ID NO: 122 - DNA, 150 bp, synthetic construct
  source 1..150: mol_type = other DNA
  misc_feature 1..17: note = Adapter with BsaI site
  misc_feature 17^18: note = Insertion site for selected target sequence
  misc_feature 18..53: note = direct repeat
  misc_feature 54..111: note = TERMINATOR
  misc_feature 112..134: note = placeholder
  misc_feature 134^135: note = Insertion site for homologues region
  misc_feature 135..150: note = Adapter with BsaI site
ggctacggtctctagatgtctaagaactttaaataatttctactgttgtagatgagaagtcatttaataaggccactggctcaccttcgggtgggcctttctgcgcaattgttaggccaattaccaacttggtcactagagaccgtagcc

SEQ ID NO: 123 - DNA, 308 bp, synthetic construct
  source 1..308: mol_type = other DNA
  misc_feature 1..17: note = Adapter with BsaI site
  misc_feature 17^18: note = Insertion site for selected target sequence
  misc_feature 18..53: note = direct repeat
  misc_feature 54..111: note = TERMINATOR
  misc_feature 112..133: note = placeholder
  misc_feature 133^134: note = Insertion site for homologues region
  misc_feature 135..291: note = proD-UTR(reversed)
  misc_feature 292..308: note = Adapter with BsaI site
ggctacggtctcaagatgtctaagaactttaaataatttctactgttgtagatgagaagtcatttaataaggccactggctcaccttcgggtgggcctttctgcgcaattgttaggccaattaccaacttggtatgataacctcctcaaagttaaacaaaattatttgtagagggaaaccgttgtcgtctccctgaatatattatacgagccttatgcatgcccgtaaagttatccagcaaccactcatagacctagggcagcagatagggacgacgtggtgttagctgtgcactagagaccgtagcc

SEQ ID NO: 124 - DNA, 57 bp, synthetic construct
  source 1..57: mol_type = other DNA
  misc_feature 1..17: note = BsaI site
  misc_feature 18..39: note = GS-linker sequence II
  misc_feature 39^40: note = insertion site of fluorescent reporter gene coding sequence
  misc_feature 40..57: note = BsaI site
ggctacggtctcatctccgctgctggttctggcgaattcgcttaagagaccgtagcc

SEQ ID NO: 125 - DNA, 157 bp, synthetic construct
  source 1..157: mol_type = other DNA
  misc_feature 1..17: note = Adapter with BsaI site
  misc_feature 17^18: note = insertion site of homologues region
  misc_feature 18..39: note = placeholder
  misc_feature 40..74: note = PJ23119
  misc_feature 75..104: note = crRNA leader
  misc_feature 105..140: note = direct repeat
  misc_feature 140^141: note = insertion site selected TS
  misc_feature 141..157: note = adapter with BsaI site
gcctacggtctctcttattaggccaattaccaacttggtttgacagctagctcagtcctaggtataatactagttcgagattttcaggagctaaggaagctaaagtctaagaactttaaataatttctactgttgtagatgtctcgagaccgtagcc

SEQ ID NO: 126 - DNA, 201 bp, synthetic construct
  source 1..201: mol_type = other DNA
  misc_feature 1..41: note = Adapter with BsaI site
  misc_feature 41^42: note = insertion site HR
  misc_feature 42..63: note = placeholder
  misc_feature 99..128: note = crRNA leader
  misc_feature 64..98: note = Pj23119
  misc_feature 129..137: note = direct repeat
  misc_feature 137^138: note = insertion site selected TS
  misc_feature 139..201: note = adapter with BsaI site
caccacaggtctcgcactggtatctgcgctccactaaagccttaggccaattaccaacttggtttgacagctagctcagtcctaggtataatactagttcgagattttcaggagctaaggaagctaaagtctaagaactttaaataatttctactgttgtagatgtctcgagaccgtagcccttccaagaatccgtctagt

SEQ ID NO: 127 - DNA, 156 bp, synthetic construct
  source 1..156: mol_type = other DNA
  misc_feature 1..16: note = adapter with BsaI site
  misc_feature 16^17: note = Insertion site for start codon-ATG- and HR
  misc_feature 17..38: note = placeholder
  misc_feature 39..73: note = Pj23119
  misc_feature 74..103: note = crRNA leader
  misc_feature 104..139: note = direct repeat
  misc_feature 139^140: note = Insertion site of selected TS
  misc_feature 140..156: note = adapter with BsaI site
gactacggtctcctccttaggccaattaccaacttggtttgacagctagctcagtcctaggtataatactagttcgagattttcaggagctaaggaagctaaagtctaagaactttaaataatttctactgttgtagatgtctcgagaccgtagcc

SEQ ID NO: 128 - DNA, 188 bp, synthetic construct
  source 1..188: mol_type = other DNA
  misc_feature 1..17: note = adapter with BsaI site
  misc_feature 172..188: note = adapter with BsaI site
  misc_feature 18..48: note = T7 g10 translational enhancer and RBS
  misc_feature 48^49: note = insertion site for start codon-ATG and HR
  misc_feature 49..70: note = placeholder
  misc_feature 71..105: note = PJ23119
  misc_feature 106..135: note = crRNA-leader
  misc_feature 136..171: note = direct repeat
  misc_feature 171^172: note = insertion site for selected TS
gactacggtctcctccactttaaaaaaaaaaaagaaggagaataatctttaggccaattaccaacttggtttgacagctagctcagtcctaggtataatactagttcgagattttcaggagctaaggaagctaaagtctaagaactttaaataatttctactgttgtagatgtctggagaccgtagcc

SEQ ID NO: 129 - DNA, 3903 bp, synthetic construct
  source 1..3903: mol_type = other DNA
atgtctatctaccaggaattcgttaacaaatactctctgtctaaaaccctgcgtttcgaactgatcccgcagggtaaaaccctggaaaacatcaaagctcgtggtctgatcctggacgacgaaaaacgtgctaaagactacaaaaaagctaaacagatcatcgacaaataccaccagttcttcatcgaagaaatcctctcgtctgtctgcatcagcgaagacctgctgcagaactactcggacgtgtacttcaaactaaaaaaatctgacgacgacaacctgcagaaagacttcaaatctgctaaagacaccatcaaaaaacagatctctgaatacatcaaagactctgaaaaattcaaaaacctgttcaaccagaacctgatcgacgctaaaaaaggtcaggaatctgacctgatcctgtggctgaaacagtctaaagacaacggtatcgaactgttcaaagctaactctgacatcaccgacatcgacgaagctctggaaatcatcaaatctttcaaaggttggaccacctacttcaaaggtttccacgaaaaccgtaaaaacgtttactcttctaacgacatcccgacctctatcatctaccgtatcgttgacgacaacctgccgaaattcctggaaaacaaagctaaatacgaatctctgaaagacaaagctccggaagctatcaactacgaacagatcaaaaaagacctggctgaagaactgaccttcgacatcgactacaaaacctctgaagttaaccagcgtgttttctctctggacgaagttttcgaaatcgctaacttcaacaactacctgaaccagtctggtatcaccaaattcaacaccatcatcggtggtaaattcgttaacggtgaaaacaccaaacgtaaaggtatcaacgaatacatcaacctgtactctcagcagatcaacgacaaaaccctgaaaaaatacaaaatgtctgttctgttcaaacagatcctgtctgacaccgaatctaaatctttcgttatcgacaaactggaagacgactctgacgttgttaccaccatgcagtctttctacgaacagatcgctgctttcaaaaccgttgaagaaaaatctatcaaagaaaccctgtctctgctgttcgacgacctgaaagctcagaaactggacctgtctaaaatctacttcaaaaacgacaaatctctgaccgacctctctcagcaggtattcgacgactacagcgttatcggtaccgctgttctggaatacatcacccagcagatcgctccgaaaaacctggacaacccgtctaaaaaagaacaggaactgatcgctaaaaaaaccgaaaaagctaaatacctgtctctggaaaccatcaaactggctctggaagaattcaacaaacaccgtgacatcgacaaacagtgccgtttcgaagaaatcctggctaacttcgctgctatcccgatgatcttcgacgaaatcgctcagaacaaagacaacctggctcagatctctatcaaataccagaaccagggtaaaaaagacctgctgcaggcttctgctgaagacgacgttaaagctatcaaagacctgctggaccagaccaacaacctgctgcacaaactgaaaatcttccacatctctcagtctgaagacaaagctaacatcctggacaaagacgaacacttctacctggttttcgaagaatgctacttcgaactggctaacatcgttccgctgtacaacaaaatccgtaactacatcacccagaaaccgtactctgacgaaaaattcaaactgaacttcgaaaactctaccctggctaacggttgggacaaaaacaaagaaccggacaacaccgctatcctgttcatcaaagacgacaaatactacctgggtgttatgaacaaaaaaaacaacaaaatcttcgacgacaaagctatcaaagaaaacaaaggtgaaggttacaaaaaaatcgtttacaaactgctgccgggtgctaacaaaatgctgccgaaagttttcttctctgctaaatctatcaaattctacaacccgtctgaagacatcctgcgtatccgtaaccactctacccacaccaaaaacggttctccgcagaaaggttacgaaaaattcgaattcaacatcgaagactgccgtaaattcatcgacttctacaaacagtctatctctaaacacccggaatggaaagacttcggtttccgtttctctgacacccagcgttacaactctatcgacgaattctaccgtgaagttgaaaaccagggttacaaactgaccttcgaaaacatctctgaatcttacatcgactctgttgttaaccagggtaaactgtacctgttccagatctacaacaaagacttctctgcttactctaaaggtcgtccgaacctgcacaccctgtactggaaagctctgttcgacgaacgtaacctgcaggacgttgtttacaaactgaacggtgaagctgaactgttctaccgtaaacagtctatcccgaaaaaaatcacccacccggctaaagaagctatcgctaacaaaaacaaagacaacccgaaaaaagaatctgttttcgaatacgacctgatcaaagacaaacgtttcaccgaagacaaattcttcttccactgcccgatcaccatcaacttcaaatcttctggtgctaacaaattcaacgacgaaatcaacctgctgctgaaagaaaaagctaacgacgttcacatcctgtctatcgaccgtggtgaacgtcacctggcttactacaccctggttgacggtaaaggtaacatcatcaaacaggacaccttcaacatcatcggtaacgaccgtatgaaaaccaactaccacgacaaactggctgctatcgaaaaagaccgtgactctgctcgtaaagactggaaaaaaatcaacaacatcaaagaaatgaaagaaggttacctgtctcaggttgttcacgaaatcgctaaactggttatagaatacaatgcgatagttgtatttgaagacctgaacttcggcttcaaacgtggtcgtttcaaagttgaaaaacaggtttaccagaaactggaaaaaatgctgatcgaaaaactgaactacctggttttcaaagacaacgaattcgacaaaaccggtggtgttctgcgtgcttaccagctgaccgctccgttcgaaaccttcaaaaaaatgggtaaacagaccggtatcatctactacgttccggctggtttcacctctaaaatctgccctgttactggcttcgttaaccagctctacccgaaatacgaatctgtttctaaatctcaggaattcttctctaaattcgacaaaatctgctacaacctggacaaaggttacttcgaattctctttcgactacaaaaacttcggtgacaaagctgctaaaggtaaatggaccatcgcttctttcggttctcgtctgatcaacttccgtaactctgacaaaaaccacaactgggacacccgtgaagtttacccgaccaaagaactggaaaaactgctgaaagactactctatcgaatacggtcacggtgaatgcatcaaagctgctatctgcggtgaatctgacaaaaaattcttcgctaaactgacctctgttctgaacaccatcctgcagatgcgtaactctaaaaccggtaccgaactggactacctgatctctccggttgctgacgttaacggtaacttcttcgactctcgtcaggctccgaaaaacatgccgcaggacgctgacgctaacggtgcttaccacatcggtctgaaaggtctgatgctgctgggtcgtatcaaaaacaaccaggaaggtaaaaaactgaacctggttatcaaaaacgaagaatacttcgaattcgttcagaaccgtaacaactaa

SEQ ID NO: 130 - DNA, 711 bp, synthetic construct
  source 1..711: mol_type = other DNA
atggtgagcaagggcgaggaggataacatggcctctctcccagcgacacatgagttacacatctttggctccatcaacggtgtggactttgacatggtgggtcagggcaccggcaatccaaatgatggttatgaggagttaaacctgaagtccaccaagggtgacctccagttctccccctggattctggtccctcatatcgggtatggcttccatcagtacctgccctaccctgacgggatgtcgcctttccaggccgccatggtagatggctccggctaccaagtccatcgcacaatgcagtttgaagatggtgcctcccttactgttaactaccgctacacctacgagggaagccacatcaaaggagaggcccaggtgaaggggactggtttccctgctgacggtcctgtgatgaccaactcgctgaccgctgcggactggtgcaggtcgaagaagacttaccccaacgacaaaaccatcatcagtacctttaagtggagttacaccactggaaatggcaagcgctaccggagcactgcgcggaccacctacacctttgccaagccaatggcggctaactatctgaagaaccagccgatgtacgtgttccgtaagacggagctcaagcactccaagaccgagctcaacttcaaggagtggcaaaaggcctttaccgatgtgatgggcatggacgagctgtacaagtaa

SEQ ID NO: 131 - DNA, 1123 bp, synthetic construct
  source 1..1123: mol_type = other DNA
  misc_feature 1..17: note = BsaI site
  misc_feature 18..69: note = terminator
  misc_feature 70..813: note = VanR coding sequence
  misc_feature 814..843: note = van2 RBS
  misc_feature 844..877: note = promoter J23100
  misc_feature 878..942: note = terminator reverse
  misc_feature 943..1112: note = fragment with PVanCC, RiboJ, B0064 RBS
  misc_feature 1113..1123: note = BsaI site
ggtctcacactgcacctttttgttatcaataaaaaaggccccccgatttgggaggcctttttagttagaggatccttatcaatctgcacgaattgaccatgctgcacccagcggtgcgcctgcgcttgctgctgcttcaaaaacttttgcattacgaattgctgccagtgcatgatcacgcataatacgttctgcaccttcggcatcaccacagctaactgcatccagaactgcctgatgctgacgatgtgctgccagcagatgttcatattcggcagacaggtccatcagatccagggccagtgcaccggctgctgcaaacggttcaaaaccattacgtgccagtgcgctttcaactgcaccattacctgctgcgctaaccagggtatcatgaaatgcctgattatatgcggcataacgatccagatcttcaccattcaggcgaccggctgcaaacagtgcttcaccttctgcaatcagtacaacaaaacgtgcatgggtttctgcggtcataccacgttctgccagacgacgtgctgcaaaaccttccagaacaccacgaacttcaattgcatcacgaatctgatcgctgctaacaccacgggctgcataaccacgtgcacccagacgaacaaccagaccttcttgttccagtgaacgcagtgcgatacgaaccggcatacggctaacacccagtgctgctgcggtcggaatttctgcaatacgttcaccacttttgatttcaccgcttgcaatcattttacgcagtgccatcataacacgctgacccggtttaatacgaggcatgtccattattcacccccctgaattgactctcttccgctagcactgtacctaggactgagctagccgtcaacattggaccaaaacgaaaaaaggcccccctttcgggaggcctcttttctggaatttggtaccgaggcttaacgatcgttggctgattggatccaattgacagctagctcagtcctaggtaccattggatccaatagctgtcaccggatgtgctttccggtctgatgagtccgtgaggacgaaacagcctctacaaataattttgtttaatactagagaaagaggggaaatactagtccaggagacc

SEQ ID NO: 132 - DNA, 1162 bp, synthetic construct
  source 1..1162: mol_type = other DNA
  misc_feature 1..41: note = Adapter with BsaI site
  misc_feature 1126..1162: note = Adapter with BsaI site
  misc_feature 42..93: note = terminator reverse
  misc_feature 104..837: note = vanR-AM CDS (reverse)
  misc_feature 838..901: note = van2 RBS and PJ23100
  misc_feature 906..966: note = terminator (reverse)
  misc_feature 986..1035: note = PvanCC
  misc_feature 1036..1110: note = RiboJ
  misc_feature 1111..1125: note = T7-g10 translational enhancer part
aaccggcgtattaagttgggctacggtctcacactgcacctttttgttatcaataaaaaaggccccccgatttgggaggcctttttagttagaggatccttatcaatctgcacgaattgaccatgctgcacccagcggtgcgcctgcgcttgctgctgcttcaaaaacttttgcattacgaattgctgccagtgcatgatcacgcataatacgttctgcaccttcggcatcaccacagctaactgcatccagaactgcctgatgctgacgatgtgctgccagcagatgttcatattcggcagacaggtccatcagatccagggccagtgcaccggctgctgcaaacggttcaaaaccattacgtgccagtgcgctttcaactgcaccattacctgctgcgctaaccagggtatcatgaaatgcctgattatatgcggcataacgatccagatcttcaccattcaggcgaccggctgcaaacagtgcttcaccttctgcaatcagtacaacaaaacgtgcatgggtttctgcggtcataccacgttctgccagacgacgtgctgcaaaaccttccagaacaccacgaacttcaattgcatcacgaatctgatcgctgctaacaccacgggctgcataaccacgtgcacccagacgaacaaccagaccttcttgttccagtgaacgcagtgcgatacgaaccggcatacggctaacacccagtgctgctgcggtcggaatttctgcaatacgttcaccacttttgatttcaccgcttgcaatcattttacgcagtgccatcataacacgctgacccggtttaatacgaggcatgtccattattcacccccctgaattgactctcttccgctagcactgtacctaggactgagctagccgtcaacattggaccaaaacgaaaaaaggcccccctttcgggaggcctcttttctggaatttggtaccgaggcttaacgatcgttggctgattggatccaattgacagctagctcagtcctaggtaccattggatccaatagctgtcaccggatgtgctttccggtctgatgagtccgtgaggacgaaacagcctctacaaataattttgtttaaaataattttgtttaactttcgagaccgtagcccttccaagaatccgtctagt

SEQ ID NO: 133 - DNA, 158 bp, synthetic construct
  source 1..158: mol_type = other DNA
cacagctaacaccacgtcgtccctatctgctgccctaggtctatgagtggttgctggataactttacgggcatgcataaggctcgtataatatattcagggagacgacaacggtttccctctacaaataattttgtttaactttgaggaggttatcat

SEQ ID NO: 134 - DNA, 306 bp, synthetic construct
  source 1..306: mol_type = other DNA
  misc_feature 1..41: note = spacer with BsaI site
  misc_feature 42..93: note = terminator (reverse)
  misc_feature 113..168: note = Ptac
  misc_feature 169..243: note = RiboJ
  misc_feature 252..263: note = B0064 RBS
  misc_feature 264..306: note = spacer with BsaI site
aaccggcgtattaagttgggctacggtctcacactgcacctttttgttatcaataaaaaaggccccccgatttgggaggcctttttagttagagcttaacgatcgttggctgtgttgacaattaatcatcggctcgtataatgtgtggaattgtgagcgctcacaattagctgtcaccggatgtgctttccggtctgatgagtccgtgaggacgaaacagcctctacaaataattttgtttaatactagagaaagaggggaaatactagtccaggagaccgtagcccttccaagaatccgtctagt

SEQ ID NO: 135 - DNA, 46 bp, synthetic construct
  source 1..46: mol_type = other DNA
  misc_feature 1..20: note = enhancer
  misc_feature 21..31: note = Poly-A
  misc_feature 32..39: note = RBS
aataattttgtttaactttaaaaaaaaaaaagaaggagaataatct

SEQ ID NO: 136 - DNA, 1729 bp, synthetic construct
  source 1..1729: mol_type = other DNA
  misc_feature 1..41: note = spacer with BsaI site
  misc_feature 42..296: note = terminator - reverse
  misc_feature 306..1388: note = lacI-AM CDS (reverse)
  misc_feature 1389..1451: note = ara1 RBS and PJ23100
  misc_feature 1456..1516: note = terminator (reverse)
  misc_feature 1536..1591: note = Ptac
  misc_feature 1592..1666: note = RiboJ
  misc_feature 1675..1686: note = B0064 RBS
  misc_feature 1687..1729: note = spacer with BsaI site
aaccggcgtattaagttgggctacggtctcacactgcacctcagataaaatatttgctcatgagcccgaagtggcgagcccgatcttccccatcggtgatgtcggcgatataggcgccagcaaccgcacctgtggcgccggtgatgccggccacgatgcgtctggcgtagaggatctgctcatgtttgacagcttatcatcgatgcataatgtgcctgtcaaatggacgaagcagggattctgcaaaccctatgctactccctcgagccgtcaattgtctgattcgttaccaattaggatccttatcactgcccgctttccagtcgggaaacctgtcgtgccagctgcattaatgaatcggccaacgcgcggggagaggcggtttgcgtattgggcgccagggtggtttttcttttcaccagtgagactggcaacagctgattgcccttcaccgcctggccctgagagagttgcagcaagcggtccacgctggtttgccccagcaggcgaaaatcctgtttgatggtggttaacggcgggatataacatgagctatcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcctcatgggagtaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccaccatcgccgcttccactttttcccgcgttttcgcagaaacgtggctggcctggttcaccacgcgggaaacggtcatataagagacaccggcatactctgcgacatcgtataacgttactggtttcatattcaccaccctgaattgactctcttccgctagcactgtacctaggactgagctagccgtcaacattggaccaaaacgaaaaaaggcccccctttcgggaggcctcttttctggaatttggtaccgaggcttaacgatcgttggctgtgttgacaattaatcatcggctcgtataatgtgtggaattgtgagcgctcacaattagctgtcaccggatgtgctttccggtctgatgagtccgtgaggacgaaacagcctctacaaataattttgtttaatactagagaaagaggggaaatactagtccaggagaccgtagcccttccaagaatccgtctagt

SEQ ID NO: 137 - DNA, 715 bp, synthetic construct
  source 1..715: mol_type = other DNA
aaccggcgtattaagttgggctacggtctcgagatgaagatatctatgacattgcgataaaacaaagtctaagaactttaaataatttctactgttgtagatgagaagtcatttaataaggccactggctcaccttcgggtgggcctttctgcgcaattgttaggccaattaccaacttggtattggtgcgggcaaataatggcgcttgctctttaatttgctgcttcttttcgccctcatcacctaatattccacagaaaccaataactaatttatcataagcaaaaggattatctaaaaggtgctgtaaatctattcctctacctcgacgaatgggatcttgatttccttgtaatcgaccgtcatattgcgaccaatctgtataataaccaaaaactttaggtttatttcgtgtgtcatatttattatagactggtaatgcgattcttcccgatgtgtaactgaaatttattgtttcagttgatggattgaaattatctatctgataagactgttcactaaaagaatctgtctggattatttttgacataaatcacctcgactagatatcagtttatttttcgatgaacactccaattgctttatttacgtggtaacataattacattctagtttgtattcttaggaaaacaaataattgttatttgtttgattgttaatgatggtatctgcgctccactagagaccgtagcc

SEQ ID NO: 138 - DNA, 738 bp, synthetic construct
  source 1..738: mol_type = other DNA
caccacaggtctcgcactggtatctgcgctccactaaagcctttcaagatagatcgcggcggcaaaggagcgaatccccgggagcatagcaaactatgtgaccggggtgagtgagtgcagccaacaaagaggcaacttgaaagataacgggtatactgatggtagaaacagtacgagtacacaagaggttttgtatgatcatttttgtgacaggagcaacttcaggttttggtgaatcaattgccagaaaattcattcaaaacggagctaaagttattgcgactggacgccgtaaagaacggttagaaaaattaaaagctgagctgggtgataacttttactttgttcaacttgatgtcactgatcgggccgctatcaaccaggttgtagagcaattacccgaatcactccgccatatcgatattttagtcaataatgccggtttagcgcttggattagaacccgctcatctggctaatttggatgactgggaaataatgattgataccaatgtcaaagggctcgtcaatatgacccgtgctggatcccacaccgcatatgctggatccttgacagctagctcagtcctaggtataatactagttcgagattttcaggagctaaggaagctaaagtctaagaactttaaataatttctactgttgtagatgcagtttcaggcgcaatttcactaatattgcgtctcgagaccgtagcccttccaagaatccgtctagt

SEQ ID NO: 139 - DNA, 712 bp, synthetic construct
  source 1..712: mol_type = other DNA
aaccggcgtattaagttgggctacggtctcgagatcagcttgttgaactttagccaatccacctaagtctaagaactttaaataatttctactgttgtagatgagaagtcatttaataaggccactggctcaccttcgggtgggcctttctgcgcaattgttaggccaattaccaacttggtatttttacccaaggataagcatcagatgttcctgcaccatctggctcggtgcctctattccaccatttagcttcccaattttgattgttccaactcactctatcgccggccttatatgccctatttgcatcccagacagggtaatcactccctgtatcgggatgaatgggatcaaccggttcaacattaacacccttgaagtagaatggtgtcatatctattttttccgtgattaactcacaaccaagcccttccctcgccgcattgaccaataaaccacgatcttgatcggcggtccatgtaaatattcctcccagattcttttctagtgcatattgtgctttagatttaacggaacgaggggtatcaagagaaataaacaactgactatccttattatagagataatcagcgtctgctatttcatcggtatataagtcaaaattatttcttcccctcttttcttccaaatccagataattatatatcgtgtcgtaccaggtatctgcgctccactagagaccgtagcc

SEQ ID NO: 140 - DNA, 716 bp, synthetic construct
  source 1..716: mol_type = other DNA
ggctacggtctcttccatgaaagctaaagatgttcagccaaccattattattaataaaaatggccttatctctttggaagatatctatgacattgcgataaaacaaaaaaaagtagaaatatcaacggagatcactgaacttttgacgcatggtcgtgaaaaattagaggaaaaattaaattcaggagaggttatatatggaatcaatacaggatttggagggaatgccaatttagttgtgccatttgagaaaatcgcagagcatcagcaaaatctgttaacttttctttctgctggtactggggactatatgtccaaaccttgtattaaagcgtcacaatttactatgttactttctgtttgcaaaggttggtctgcaaccagaccaattgtcgctcaagcaattgttgatcatattaatcatgacattgttcctctggttcctcgctatggctcagtgggtgcaagcggtgatttaattcctttatcttatattgcacgagcattatgtggtatcggcggatcccacaccgcatatgctggatccttgacagctagctcagtcctaggtataatactagttcgagattttcaggagctaaggaagctaaagtctaagaactttaaataatttctactgttgtagatatctccatatataagaatggtaataatatttgtctcgagaccgtagcccttccaagaatccgtctagt

SEQ ID NO: 141 - DNA, 713 bp, synthetic construct
  source 1..713: mol_type = other DNA
aaccggcgtattaagttgggctacggtctcgagatttgtcagaggctgaacgtacactgctattgggtctaagaactttaaataatttctactgttgtagatgagaagtcatttaataaggccactggctcaccttcgggtgggcctttctgcgcaattgttaggccaattaccaacttggttcgcgacggcaagggagcgaatccccgggagcatagataacgatgtgaccggggtgagtgagtgcagccaacaaagaggcaacttgaaagataacgggtataaatccatttttatttagttatggaattcagcgtcagccaataaaaaattgcacaaaatcaaaggcgcttattaaaaaaataggaaaaaaatggaatatcttttgccggttcacttggtgaatcgtttattccgttatcaggaaaataattgtcgaagaattgctaattatgaggctattaaccgggctattacctccagcgtccaagagaatgtgctgtctgaggattttaattgatgcctgatgtgtgcccgtttttatttcttggggggaaggaagaagggcaaaacatttgtgatgactgttgagaatgacagttttcaaataactaacagattcattgaggtagtaagtagatgaaagatagcatggctaaaaaggaaattatctttgatgctggggtatctgcgctccactagagaccgtagcc

SEQ ID NO: 142 - DNA, 737 bp, synthetic construct
  source 1..737: mol_type = other DNA
caccacaggtctcgcactggtatctgcgctccactaaagccactcttcgctacagttaacctcggtgccgggggatcattttagtctgatggaagatagtgaaaataaaactattttagctaagaccttaagcatggcgctggcaatgaattgtgaagcggaggcgctgtaatgtcagtgatattgtgggatgaaaagctatatttaaacaaagatagttaaaaatcaatttgtccggttaaaagtttttataatagccggacaatcttactgaatataggcaccgaatgttaatcacttcggtgccttatttttttatcaaatcctacacctgatgctagtggagaaaccctatttttcagcattgtctacgcttgtaaacaatgtttacacttttaaacaactcttacacttttaaacaattattacacttttaaattaatttaaacactcttaatgcttcaatgtcgaatataatgggagttattttctgtcatttacacacttataatcctatgaggtttaaaataatgatgattatggatcccacaccgcatatgctggatccttgacagctagctcagtcctaggtataatactagttcgagattttcaggagctaaggaagctaaagtctaagaactttaaataatttctactgttgtagatttaccgatgttgcgctacagtccgtcacaatgtctcgagaccgtagcccttccaagaatccgtctagt

SEQ ID NO: 143 - DNA, 712 bp, synthetic construct
  source 1..712: mol_type = other DNA
aaccggcgtattaagttgggctacggtctcgagatattgatgcctgatgtgtgcccgtttttatttgtctaagaactttaaataatttctactgttgtagatgagaagtcatttaataaggccactggctcaccttcgggtgggcctttctgcgcaattgttaggccaattaccaacttggtcatttttcagagaattatcaaagtaaattcggtgttaattggggagcaggaggtttagtttgtctcgataattatttaatttttcgtgaaataattcacatatgatattaattcaaaatggacagaatacagcagggatttaatactcatgccagtaaatatattcaatatacccaatagatttcaagatggatcgcgacggcaagggagcgaatccccgggagcatagataacgatgtgaccggggtgagtgagtgcagccaacaaagaggcaacttgaaagataacgggtataaatccatttttatttagttatggaattcagcgtcagccaataaaaaattgcacaaaatcaaaggcgcttattaaaaaaataggaaaaaaatggaatatcttttgccggttcacttggtgaatcgtttattccgttatcaggaaaataattgtcgaagaattgctaattatgaggctattaaccgggctattacctccagcgtccaggtatctgcgctccactagagaccgtagcc

SEQ ID NO: 144 - DNA, 715 bp, synthetic construct
  source 1..715: mol_type = other DNA
ggctacggtctcttccatgaaagatagcatggctaaaaaggaaattatctttgatgctggattagtgcaagtactgtcaaacaaggtcggtgacgacatatctgttcagaaacttaatttgttgtcagaggctgaacgtacactgctattggaaacttggaatgccactgaaaccgtctatcctgaccagttaggtatccatcagttgtttgaacgacaagtggagaaaaccccgactgcgacggcgttaatcgcaggggataagaccctgagttatgcggaattgaatgcttgtgctaaccgactgactcatcaactgattaagcaggggattggcccgaatgatcatgtggcaatcctgttagagcgatctatcgaattggtggtagctcaattggctattctcaaggcgggggcggtttatgtccctatcgataccagcgtgccggacgagagaaaaaagtggttgataaacgatggcttcgccaaattgttgctcaccggcgcacaagcgaatatcggatcccacaccgcatatgctggatccttgacagctagctcagtcctaggtataatactagttcgagattttcaggagctaaggaagctaaagtctaagaactttaaataatttctactgttgtagataaataactaacagattcattgaggtagtaagtctcgagaccgtagcccttccaagaatccgtctagt

SEQ ID NO: 145 - DNA, 662 bp, synthetic construct
  source 1..662: mol_type = other DNA
aaccggcgtattaagttgggctacggtctcgagatagactgatgatgtggcaatgagtgacttggcgtctaagaactttaaataatttctactgttgtagatgagaagtcatttaataaggccactggctcaccttcgggtgggcctttctgcgcaattgttaggccaattaccaacttggttatttaatcgatttcaatatggaacacgacagcaagaaaatgacaaacttacaatatttataacgaaagtatggaaataattgaattttgctgactcgatttatgaattactttttacgtgtttttgaaacatggccatttcattaatataaattcgatgttatcggaacagtattgggcattttttacgattattacaatgaaactccttaaaccttgatagtgaaattgcatcaagcataggtagtgtctgctcttcaccaaaacgagtatttaaaccatgaagaagacgatttttgagttgtcaagatcacagcaagctgttttcaaaatggaagcgtttcatctgactggctgtccgttttatttgggtgttgcagtgcgtttaaatggcagtattcctctggatgggctcgtgcgggcagccgaaaccgtccgggactcaatggaggtatctgcgctccactagagaccgtagcc

SEQ ID NO: 146 - DNA, 742 bp, synthetic construct
  source 1..742: mol_type = other DNA
caccacaggtctcgcactggtatctgcgctccactaaagccgtgcgggcaatctggcaggaagtccttgaaaatgaaagggtggggacggaaacgaatttctttgaagccggtggcaactcgataaagataattacattgcaatccctgttgcagcaggcattcaatcgtgaaattcgggtcatcaccttatttgaacatccgacgattgcttcctttacaaactggattaaacagggccaattgtcatgtggacaggtgagtgaaacggaattgacatccacatcggataaagtgtcttcccaggcacatttacagcgctcgttccagtcaggcaaaagcagattagcggacagacgacgcaaatccacagggaattaaggagagctaacttaacggataaagccggactcttccggacgggttggatgaggctggcttttgatggaggagcattaacgaagagagtctgaaatatatacccaattgatttcaagatgcagtgcggcggcaagggaatgacaaattcgtcgggaacgaatttgccggatcccacaccgcatatgctggatccttgacagctagctcagtcctaggtataatactagttcgagattttcaggagctaaggaagctaaagtctaagaactttaaataatttctactgttgtagatgctatctgggacgaattgatgatcagataaagtctcgagaccgtagcccttccaagaatccgtctagt

SEQ ID NO: 147 - DNA, 712 bp, synthetic construct
  source 1..712: mol_type = other DNA
aaccggcgtattaagttgggctacggtctcgagatattaatataaattcgatgttatcggaacagtgtctaagaactttaaataatttctactgttgtagatgagaagtcatttaataaggccactggctcaccttcgggtgggcctttctgcgcaattgttaggccaattaccaacttggtgtaatggagaacgtataaagggaatttatattttataataataaatgctatcaactttaggattattagggaaatggtttctcatcaatataatttcactagtggaagggtaggtttttattttttttggagtattgtcaaaatttatttcatatcaaatatgattagtcttattctatatggtctatcttattctaatataatcttagtaaataaggttgtatttgtctttaatgcagttacgtctcgtaacgcttcattttttgtgttcttctttcttaaaactatattgttttttattgcactcattcattttaatcgagaaattatctgtatttatgattgtttatctttttacttgtatgatgcaagtggaaatctattaattatggcaccaacttaatttatattctaatgtcaataaaaatcattatataaatatatgggagtaatgagttttttgtgaaaagcaattaacccataggttatattgattttttggtatctgcgctccactagagaccgtagcc

SEQ ID NO: 148 - DNA, 713 bp, synthetic construct
  source 1..713: mol_type = other DNA
ggctacggtctcttccatgaagaagacgatttttgagttgtcaagatcacagcaagctgttttcaaaatggaagcgtttcatctgactggctgtccgttttatttgggtgttgcagtgcgtttaaatggcagtattcctctggatgggctcgtgcgggcagccgaaaccgtccgggactcaatggatatatttcacatcggttttgagactgatgatgtggcaatgagtgacttggcaacacattggcatggtatccgccggctcacctcccattctgaagtaatccaagtcgatttcagcaactacccggactctaaacaggcttttgagagttggtcagatcgccaaatgttgataaaagaagatttgtcattgacgccggtgcggatttttgccgtgcattttcaacaggggcaaacaggatggtttattaaggcgcatcatgccgcgatggatggtgaggggttttcagtactgatagaacatctgatgaaggctctggagaaagaagagccgggatcccacaccgcatatgctggatccttgacagctagctcagtcctaggtataatactagttcgagattttcaggagctaaggaagctaaagtctaagaactttaaataatttctactgttgtagatgtgaagagcagacactacctatgcttgatgcgtctcgagaccgtagcccttccaagaatccgtctagt

SEQ ID NO: 149 - DNA, 692 bp, synthetic construct
  source 1..692: mol_type = other DNA
aaccggcgtattaagttgggtctcgagatacgcctcattgataatttgcacggcctttttgtctaagaactttaaataatttctactgttgtagatgagaagtcatttaataaggccactggctcaccttcgggtgggcctttctgcgcaattgttaggccaattaccaacttggtatatccggttcatgatattactgcgaaattacgtgagaagagcgtggctgttcgtttcggaattcatcccgttgcagggcgcttgccgggtcatatgaatgtgttgcttgctgaagctaaagtaccctacgacatcgttctggaaatggatgaaatcaatgatgatttttctgagacagacaccgttttagttattggtgccaatgatacagtaaacccggcagctcaagaagatcctgccagccctattgccggaatgccagtattggaagtgtggaaagcccaaaacgttattgtctttaaacgttcaatgaatacaggttatgccggggtacaaaacccattattctttaaagaaaatactcagatgttgtttggtgatgctaaagatagtgtagactcaatccttcgggctttataacgagtagcattatcttaatcacagagagcttcatgttattacatgatatatgaagctctttttttactgccatgacaccactagagaccgtagcc

SEQ ID NO: 150 - DNA, 710 bp, synthetic construct
  source 1..710: mol_type = other DNA
aggtctcgcactggccgttaatgaataaaaagtcaggcctcatgaatgaggccaattattgttaatcgtagtcaatttactggcgtaattttacttgggatattaatgagttcaatatcaggtttatattccgaatctatcaaaatataatcacagccatattcattgcacattttgaaattgtattcattttcttcaatcgttgattcaagtgtacaatccgaatcatctaaccgagattcaatggcatttgcatatttttttaacatccgaaaagtgattttcaatatactctttagtcatcacaagacagtgaaattggatttgcctcagatagttatctgtgaaagcatctttccagtcaaaaggaatgtaacttccttcaataacaagattctgattattctcaattgctgtcttgatgatctccttaacaatcggccaaagatacgattcaagtttcttatcagattcaagtttcttatcatcttccggggttataccaaagccctatggatcccacaccgcatatgctggatccttgacagctagctcagtcctaggtataatactagttcgagattttcaggagctaaggaagctaaagtctaagaactttaaataatttctactgttgtagatactatatggatgctcagaatctctatgattagtctcgagaccgtagcccttccaagaatccgtctagt

SEQ ID NO: 151 - DNA, 708 bp, synthetic construct
  source 1..708: mol_type = other DNA
aaccggcgtattaagttgggctacggtctcgagatataaaataaaagggtaataaaatatgtatgagtctaagaactttaaataatttctactgttgtagatgagaagtcatttaataaggccactggctcaccttcgggtgggcctttctgcgcaattgttaggccaattaccaacttggtacatccgtgattattactccgggctatggtatggccgtcgctcaggcgcaatatccggttcatgatattactgcgaaattacgtgagaagagcgtggctgttcgtttcggaattcatcccgttgcagggcgcttgccgggtcatatgaatgtgttgcttgctgaagctaaagtaccctacgacatcgttctggaaatggatgaaatcaatgatgatttttctgagacagacaccgttttagttattggtgccaatgatacagtaaacccggcagctcaagaagatcctgccagccctattgccggaatgccagtattggaagtgtggaaagcccaaaacgttattgtctttaaacgttcaatgaatacaggttatgccggggtacaaaacccattattctttaaagaaaatactcagatgttgtttggtgatgctaaagatagtgtagactcaatccttcgggctttataacgagtagcattatcttaatcacagaggtatctgcgctccactagagaccgtagcc

SEQ ID NO: 152 - DNA, 713 bp, synthetic construct
  source 1..713: mol_type = other DNA
ggctacggtctcttccatgaaaaaggccgtgcaaattatcaatgaggcgttaaatcagggaattaccctgtttgttgttgataatcaattacaatatgaaaccaaccgcgacagcataccgccagaattgctcagtgaatggaaaagccataagcaggagttgatcgattttctgaatcgaattgatgcacaagaggacgttcaaactcacccgctacaggatattcaacgtaatggcaacgttgcacattacccactttcatatgctcagcaacgtttatggtttattgaccaattgaccgaaggcagcacccagtacaactgtgtcggggatttcagactgcgtgagccacttaatatcaagtttttcgaagcagccctgaaagggatcattgagcgtcatgaagcattgcgaactcattttaaaactataggggatgaacctcgtcaggttattgccactcactacgatttgcctgtgaaataccacgatttgtcagccttttctgatgttgagggatcccacaccgcatatgctggatccttgacagctagctcagtcctaggtataatactagttcgagattttcaggagctaaggaagctaaagtctaagaactttaaataatttctactgttgtagatcctttagattatttttgtgttgtttttccccgtctcgagaccgtagcccttccaagaatccgtctagt

SEQ ID NO: 153 - DNA, 693 bp, synthetic construct
  source 1..693: mol_type = other DNA
gcctacggtctcaagatcaatatcgtatcatgctgggtattctccagcgtctaagaactttaaataatttctactgttgtagatgagaagtcatttaataaggccactggctcaccttcgggtgggcctttctgcgcaattgttaggccaattaccaacttggtgtatcatatgaaaaaggccgtgcaaattatcaatgaggcgttaaatcagggaattaccctgtttgttgttgataatcaattacaatatgaaaccaaccgcgacagcataccgccagaattgctcagtgaatggaaaagccataagcaggagttgatcgattttctgaatcgaattgatgcacaagaggacgttcaaactcacccgctacaggatattcaacgtaatggcaacgttgcacattacccactttcatatgctcagcaacgtttatggtttattgaccaattgaccgaaggcagcacccagtacaactgtgtcggggatttcagactgcgtgagccacttaatatcaagtttttcgaagcagccctgaaagggatcattgagcgtcatgaagcattgcgaactcattttaaaactataggggatgaacctcgtcaggttattgccactcactacgatttgcctgtgaaataccacgatttgtcagccttttctggatccgctggcatctctgagaccgtagcc

SEQ ID NO: 154 - DNA, 804 bp, synthetic construct
  source 1..804: mol_type = other DNA
gcctacggtctctcttaaaccgccgaataacagagaacacggtcacaagatcgcagattttagtgataagtggaatgccatgtggctgatcagcatgaacacggtcacaagatctagtgtgcgaggcgttcttacattctggtgaatcacaacatatcgccacatcaccacaagctgatcgttctcatattgacagccatcaaacaataggactgtttttgagtgacctgatagttaaatcaactatcagtaaagagatgagcctttatgaaatcattcagcaagtcagtgattttatttataaagcgctggaaaattcagatatccatatggctgcggtgattgataacgtaggggaaaaggtgagagataatatatttaatattgtctttaactatatggatgctcagaatctctatgattatgcagataattctgatagtggaaaaatatccctgccaagcctcgatatagaattgataaatattgacccgactccttttcaatcactggggataaacttcatcaacataccacaaaatatcatgtgcgagttaagctataacccagagctgtatactgaagaaactgccaataatcttgcaagatattatggacgactgctggaggatcccacaccgcatatgctggatccttgacagctagctcagtcctaggtataatactagttcgagattttcaggagctaaggaagctaaagtctaagaactttaaataatttctactgttgtagataggtatgcccaacgcccgtatcaaagaggatgtctcgagaccgtagcc

SEQ ID NO: 155 - DNA, 712 bp, synthetic construct
  source 1..712: mol_type = other DNA
aaccggcgtattaagttgggctacggtctcgagatgaggcagattaatacccttaccaattacattgtctaagaactttaaataatttctactgttgtagatgagaagtcatttaataaggccactggctcaccttcgggtgggcctttctgcgcaattgttaggccaattaccaacttggtatttatctcgctaaacccggtgattttttgtttcttcatttgattacctcgttgtttgcaatgaaaaatatcgcttcacgtttaccggctgaggaaagtaaaaatattattaaatgttactgggttggcatgttgtgcattctgttttcaacaactgatttatcgaagcctgccaaattttcggcgcttaatcagacttattcattcaggcaagacgaaatgacttatcctgtctgggaacaggagtggtcgcatattattgccagggcattagaggaagaggaagaacataatccgaaactggtttatgtgatgaatcaattgtggaaagaacagggtttaactatttatagggcagcatccagtcaatttacaagtactcctttgcttcctccttctttcgaagaagcaccaattgaataatattggcatatttaaagaataatgaattaattgattgataattccatatcgtgatgagatgaattttatcacgaaacggtatctgcgctccactagagaccgtagcc

SEQ ID NO: 156 - DNA, 716 bp, synthetic construct
  source 1..716: mol_type = other DNA
ggctacggtctcttccatgttagaaaataatattacacaatgtgactcaatcaatgatgtttatcttaaagaagaagcaataacattgatggatatgcttgagagtcaacttaagcaccaggcagatggatatgttgttattgatcaagaagaatctctcagttacgctgatttctatttgagggtgaaagagatagggtattgtctgtcagaaattagctcaaagaattcggtgggtattgggcttttttgtgatccttctatagatttaatttgtggtgcatggggtattttgtcagcggataaagcttatttgccgttatcgcctgactatccaactgaacgcctcaaatatatgatagaagattctggtattgatgtgatttttacgcaatcgcacttaaaagcacagctacaggacattgcaccaaaatcagtattaattatgacaccagaagatgtcgctctgacgataaaaacacgaacaatagaagatattctgggcacagttcaagttcctggatcccacaccgcatatgctggatccttgacagctagctcagtcctaggtataatactagttcgagattttcaggagctaaggaagctaaagtctaagaactttaaataatttctactgttgtagatactacggagatattcattttattattatatagtctcgagaccgtagcccttccaagaatccgtctagt

SEQ ID NO: 157 - DNA, 712 bp, synthetic construct
  source 1..712: mol_type = other DNA
aaccggcgtattaagttgggctacggtctcgagataattaattacgatatcttgggaaaaaacagtgtctaagaactttaaataatttctactgttgtagatgagaagtcatttaataaggccactggctcaccttcgggtgggcctttctgcgcaattgttaggccaattaccaacttggttttatatgtaaattttttttgccaaattaaaaataaagacccgagttgacattgtatactttgcgttctattttttatttaattttagtgaacatatttttattattggactgcttatatacccaatggatttcaagatgcatcgcgacggaaagggggtgagtaagtacagccaacaaagaggcaacttgaaagatgacgggtatatattgtttcacccggcaagaaaggtaatactattgaggtcattaattccctcaaaaaacagttgctataatatagtaggtttttttaatagcctatttatttaaatttagctctcttacttatccagccatatcctttactatctgatttgtcagaaattgattgattgactttgtagatcgacaaaaagcatggcagtgttgttctatgctaaatcgagacattcttatttataggtgtttttttaaatcattaaatatatatagatttcaagatgcatcgcggcggcaaggggtatctgcgctccactagagaccgtagcc

SEQ ID NO: 158 - DNA, 715 bp, synthetic construct
  source 1..715: mol_type = other DNA
ggctacggtctcttccatgggctggaatatatttattaacgcaccagatagttaccatcttacttccacacatatccgcaacagcttgcaccaacaaggttttgccactttcaatgccaccgatcttgacctttctgacagtgaaaaaatcgatcttatctctctgtgtgaattatcgaagtcactgccgttagatcgatttggtgaaggtggtcgtcaccgcagttattgtgagggagtgtggcaccgtgaaacagaaactattgattggaaaacaggtcatcagcaatctgatggtagtatagagattgattatcatcagggaagtgaatatcaacctgaatttggtggtgtggtacgaaaattcctgcgtatgcctgatgagatacttaataaagggctacttaataaactcatttggcacgatctgagtctaaccggcatggcagaacattattcccgtttgctatgtggtgtacatctgatccgaatgcaggcactaccgggtaaaccggcaaaggatcccacaccgcatatgctggatccttgacagctagctcagtcctaggtataatactagttcgagattttcaggagctaaggaagctaaagtctaagaactttaaataatttctactgttgtagatatacgatgtcatcgtaattcaccctaataaagtctcgagaccgtagcccttccaagaatccgtctagt

SEQ ID NO: 159 - DNA, 679 bp, synthetic construct
  source 1..679: mol_type = other DNA
gcctacggtctccagataggtgttggttaaatagataatgacagtatcgtctaagaactttaaataatttctactgttgtagatgagaagtcatttaataaggccactggctcaccttcgggtgggcctttctgcgcaattgttaggccaattaccaacttggtcaggaaaacacttcgtcactttttccatgtcacagcacaggaactggctacccggctgactgataattttcagattttaattcctatatcttcaagttaattttggaaaatacaatcaaatgctatctatttttctatgttgttatgtgattttaaattgatttttattttgttttttattctataatgctcacgattaaatgatttgtatttcaaccatatttaaattattacaaggttaactgtatagttatgtataaatataaccaagcatataaagtaattaaaaaggttattttggagataaactctgacttaactcaagatatcatcactccagatgtaagcatgaaggatctcaacttaagttctatcgatagaatggaggttgttgttgaatcaatgatgtatttgaaaataaaagttcctaatcaagaaatagttaaggctaataatattggtgatcttgctcatatattcgcaaagtatataaactaacactggagaccgtagcc

SEQ ID NO: 160 - DNA, 694 bp, synthetic construct
  source 1..694: mol_type = other DNA
gcctacggtctcgtccaatggaaaaggtagtcattacaggtgtaggaattgaaacggccgttgcatctgattataaggagttcagtcaggctttacattatgggatagcacctgagtttatccgccatcaggtggatgagattgaatattttgctgccaagttgccgcccgatagattgaagcggttaaaagctcagttttcagcatcactccggtgtcatttacggacagcccctgaatacatttattgggcaattaaagttgcaaagcaagcagttgaaatgtcatgtcttacaccggatgagctaacacgttgttgccttgttattgcaggaggagagtttgatgcccactatcgttacaccaattttatgctttatcagcaagagcctgaatatatcaagcccagttatgctcttgctcatatggatattgatgtattaggggcaatttcagaggtatttcctgtcatccaggatggctttattatcaacggtgcttcagcaagcggaaatattggatcccacaccgcatatgctggatccttgacagctagctcagtcctaggtataatactagttcgagattttcaggagctaaggaagctaaagtctaagaactttaaataatttctactgttgtagatacaataaaataaataaatcaatgattaaattgtctagagaccgtagcc

SEQ ID NO: 161 - DNA, 682 bp, synthetic construct
  source 1..682: mol_type = other DNA
gactacggtctcaagatcggagaatgatgtcggcaacgatgcgggtcagtctaagaactttaaataatttctactgttgtagatgagaagtcatttaataaggccactggctcaccttcgggtgggcctttctgcgcaattgttaggccaattaccaacttggtctgattatctgctccccattatccgggaaaaactcaaccgtgacagtccatttgtttttttcggccatagcatgggggcattggttgcttaccagttgacccagcgtctgatggagaagaaagaaagattacctctctggttgggaatatccgggcgtgctgaacccggatttgtatcgttgcaaaagcgccatttgttacctgatccccttttacaacaggaactaacgcgtttgggcggtatgccaccttcccttgagcgctacccggagttatggcgacaattacttcctctgttccgccatgatttaggtgttattgaaaattggcgcgaaaacccggctcaccgtgcgttacctgtccctgtgtctactttcagtgcatggcatgaccctattgttgcacctgatcagatgaaagactggcagaaagtctgtcttaagcccatcacatgccatgtcttccaaggggatcatttttattttctgggtcaataatccccactagagaccgtagcc

SEQ ID NO: 162 - DNA, 693 bp, synthetic construct
  source 1..693: mol_type = other DNA
gactacggtctcttccaatggatacggtggtagaacatgatgttttatctataagcccttcttcacaagcctattctcttgattggatggcgctattacgcacgatgcagctttcacgggaaattgataaacggtcagcattatttacccgtcagggacgtgcttggttccatatgtctgcggccggtcacgagggtctggcagtattggcacaattgctgacaccgtctgacctgattttcccccattatcgcgatcgcgcattggtgctggcgagaggaatgtctactgaggcgatggcgcgagagttgatggcaaaagcggattcccattcagggggacgcaatatgaccaatcatttttgtgatcatgcaaacggcatattttccattgccagccctaccgcttcgcaatgtcttcctgcaacgggtgcagcgtgggcagcgaagctggaaaaacagccccgcctgattgtctgtggaattggcgatgcctctacacggcagggagaattttacggatcccacaccgcatatgctggatccttgacagctagctcagtcctaggtataatactagttcgagattttcaggagctaaggaagctaaagtctaagaactttaaataatttctactgttgtagattacaggattggtcgatcactcatgaggaaatgtctggagaccgtagc

SEQ ID NO: 163 - DNA, 692 bp, synthetic construct
  source 1..692: mol_type = other DNA
ggctacggtctcaagatgttgatgaacacttctctttagacgatatgcgtctaagaactttaaataatttctactgttgtagatgagaagtcatttaataaggccactggctcaccttcgggtgggcctttctgcgcaattgttaggccaattaccaacttggtctaatgacaggaaaaagacagccattccctaacttaccgcgggcgggtgtatcggaggaagacgttaaagatgctacacgttttggtgatgcattgttggcagcgctcaaaacggataaagagaaagttgatgctccaatgttgaaaggtatgggagcggcggttgtcgaccaagcttttattttcggtgaacgtgcggcacatcgaggatttagattttggtctgggttgatttgccgtgtgggcaaacgagggatatgggtaagacgctctttattagcgttgtttgtaacctatttgatattgatggtactggttgtttttcctatttcagtggttgtgcgccgtttgctttccccattactaaaagaacgtcttaatgaattacagaaatactatgaacaaccttctggttctgcccgctatgatgataaggagataaataagtaaatgagtaacaacgtttatatcactaaagtttccgcgtttatgccgggaggatccgctggcatctctgagaccgtagcc

SEQ ID NO: 164 - DNA, 809 bp, synthetic construct
  source 1..809: mol_type = other DNA
ggctacggtctctcttaaaccgccgaataacagagaacacacgatcttcacagcagattttagtgataagtggaatgccatgtggctgatcagcatgtgtgaagatcgtgtgtttagtgtgcgaggcgttctttgtgcctttttttgtttttgattatttgaacagatctgcactgatagtggaggttccaccactggattgccattcacgggtaatatgatagtacttagcacctttagccgcggcacgtttagccacttcataagaaacatcaggcatgctgttgtagtaacctgaaaaggtgatggaatcaaaaggaaccatctgagcagcgctgactttatttaattcttggatttttgtgccatcaggaagagttacagtgtagcgtttgcctttagaatactgggtttcaaagaagcggccaacagaattactggttgaagttgaagaggctaaaccaggaatttctactttcttagctgattcgccaccagcagccaatgcaatacgtccagaatcggaatctgccgggataacggcttcatcagcctgaatttggcgttttggtgcatcatctttataaacataagctgtcaccgtctgattaccgccatcattgattgcaacctgggatcccacaccgcatatgctggatccttgacagctagctcagtcctaggtataatactagttcgagattttcaggagctaaggaagctaaagtctaagaactttaaataatttctactgttgtagatcacaaccgtcagatgcatgaatgctcctgtgtctcgagaccgtagcc

SEQ ID NO: 165 - DNA, 689 bp, synthetic construct
  source 1..689: mol_type = other DNA
ggctacggtctcaagataggcgattcattactaataatagtgacggttgtctaagaactttaaataatttctactgttgtagatgagaagtcatttaataaggccactggctcaccttcgggtgggcctttctgcgcaattgttaggccaattaccaacttggtatgaaaataacaggcatagccggattacttccttcccgattcgtctctaatcaagaggttattgatttagttgaatattattcaaaagaaattttccatggtgatttgcccaagacattgaagctaatcggaaaaatgcttaataaagcaggtataaattcccggtattggttggcagataacgagaaacctatggaactcattgaggccgcatttaatcatgccttagcacaagccaatatcattaaggaagatatcgatttactgatctattccagtgttgcccgcggcttcatagaaccagctaacagtacttttgtctcaaaggcactagggctaacttgtaggaactacgatgttgtagacgcctgtaacggttgggtggcagctatggatattatcaatagcaaaatgaaagctggtgaaatacgccatgccgctattgttaatatggagtttattatgtcccaaaatggaccaatttttccaaagactggatccgctggcatctctgagaccgtagcc

SEQ ID NO: 166 - DNA, 791 bp, synthetic construct
  source 1..791: mol_type = other DNA
ggctacggtctctcttaaaccgccgaataacagagaacacattcattcctccgcagattttagtgataagtggaatgccatgtggctgatcagcatgggaggaatgaatgtgtttagtgtgcgaggcgttcttaaaatttgcttataatctgccatccactcaaagtcagattagtgaatacttatttacacaggatatataccctatggatttcaagatgcatcgcggcggcaaaggagtgaatccccgggagcatagataactatgtgaccggggtgagcgagtgcagccaacaaagaggcaacttgaaagatgacgggtatagtgtcatagaattagacaggcgctcaagaaaactgaaaacgcctgtcaatcttagtagtaactttactgattaatcccagttcagaataactttaccagactgccctgaacgcatgatgtcaaagcctttctggaaatcatctatagagaaccggtgggtgataatgagtgtcagatccaaaccagattgaattaatgtagccattttataccaggtttcaaacatctcacggccataaatacctttgataaacaaccccttaaaaatcacctgattccaatcaattgccaggatcccacaccgcatatgctggatccttgacagctagctcagtcctaggtataatactagttcgagattttcaggagctaaggaagctaaagtctaagaactttaaataatttctactgttgtagatttcatacaggttcaccaaaaacgtgggcacgtctcgagaccgtagcc

SEQ ID NO: 167 - DNA, 711 bp, synthetic construct
  source 1..711: mol_type = other DNA
  misc_feature 537..694: note = proD-UTR
ggctacggtctcaagattgtgaaaaaataaccagaataaattcatattgtctaagaactttaaataatttctactgttgtagatgagaagtcatttaataaggccactggctcaccttcgggtgggcctttctgcgcaattgttaggccaattaccaacttggtcgaacaaacgatcaaatcaatactgcctgatgggagggccagttcctctgccattcccacaaggaatctggcgaatggcctgcgtttgatcgcttcatccaccataccttgggctggatcgatgccaatgcagttggcctgaggccatcgtatatgtaagtgctgcaacaatgcgcctgttccacaaccaatatccaatatactacgcggcaaagatgcccaatccatcttcttcaagatgctcaaacattcctcatgtgccaggttcaatttatcgccatatttttcttcataggtaggtgctattcgatcgaaagtttcaataaaaccaggtccttttttcaccctctttgttttctcagaatttaacatatgataacctcctcaaagttaaacaaaattatttgtagagggaaaccgttgtcgtctccctgaatatattatacgagccttatgcatgcccgtaaagttatccagcaaccactcatagacctagggcagcagatagggacgacgtggtgttagctgtgcactagagaccgtagcc

SEQ ID NO: 168 - DNA, 718 bp, synthetic construct
  source 1..718: mol_type = other DNA
  misc_feature 14..44: note = T7g10 Translational Enhancer + RBS
ggctacggtctcgctttaaaaaaaaaaaagaaggagaataatctatgataagtaaaaaacgggccactccacgacaagaagtgttatgtctacaacaagcaattgatccctatttaccaggtttaattcttgaaatcgtttttcgcattgatggttccctttccgggcaacaattgaaatcgcgcattgacgctatagtcaggcggcatgcatcgctcaggcagcgttttgaatttcaggatggaaaatattgggttgtggaagcaccaccggaagcatcagggtactgtcatattcgtttcctggatgaaatccctgattcatcattatctgaaattctgctccccgatactcgcgaatttatgggggtcgaatcagaacgattatttaaggcagaaatcattgaaacaggaaatcatgatttatatttagtggtcagggtgcatcacgccattgcagatctctggtctgttggcttgctgatagaaaacttaaatgatgccgatcctgaattcctccagaaaaaaagtgattctctgccaggatcccacaccgcatatgctggatccttgacagctagctcagtcctaggtataatactagttcgagattttcaggagctaaggaagctaaagtctaagaactttaaataatttctactgttgtagattctcccgatgtcaaattctatcgatcacaaagtctggagaccgtagcc

SEQ ID NO: 169 - DNA, 663 bp, synthetic construct
  source 1..663: mol_type = other DNA
ggctacggtctccagatagcacccataatggccgcgccaattccacgggtctaagaactttaaataatttctactgttgtagatgagaagtcatttaataaggccactggctcaccttcgggtgggcctttctgcgcaattgttaggccaattaccaacttggtgcctcagacatagaagacacaaatgcaaataaaactgctgccatgatggacttatatatgctgtctctttttggtatttctggcggtaaggaacgcacggaaaaagaatttagagaattactcggacaagctaattttaccgttaaagagatcacccaattacccagtggcaacggcattatatatgccacacccaagtgacgcatggcgccaataaactcgtaacactaataaaccagtaacaccataatgcgaatatacaatgacacatcttactttatagattataaagaacacggcctaatcgacctgataatttagagcctaagggaataggctttataaaactaatttaataatgttattaaattggctgaaattaattaatgaaactttttacttgtcaaacagggttaattagccaaagttttaattgtaatgtgtaactatgaggaaaaatgatgtcaaagctcatttattaacactagagaccgtagcc

SEQ ID NO: 170 - DNA, 592 bp, synthetic construct
  source 1..592: mol_type = other DNA
ggctacggtctcatccaatgactcgtatcattttgagcctgattttagtcgttgttcttggtttatctgtatatcaaggtgtcagcatggtacaaccgacaacgatgaccaccttaccaactaaagaaacaggatatttaaacgtacaaaaagcgctcagtatcattgcagccaaaccacatcctacagtatccacacagcaagaacagatccgccgctatttactcacagaaattcaagcaatgggataccatgttatcgaacaaccgttcctgttcactatcgatgaacttgttgctcgtcaggaaaaaatctactcacaacttgatgaataccaacgccgcgcatttgatgcagaactagcacgagtcggtgccaaaacttttgaagaagaagtgcgcatccgctcagggctaggatcccacaccgcatatgctggatccttgacagctagctcagtcctaggtataatactagttcgagattttcaggagctaaggaagctaaagtctaagaactttaaataatttctactgttgtagatctaatgcaggtgaattgcaacaattgttcatgtctcgagaccgtagcc

SEQ ID NO: 171 - DNA, 681 bp, synthetic construct
  source 1..681: mol_type = other DNA
aaccggcgtattaagttgggctacggtctcgagataaattaattgtatccattgagttactgtaacgtctaagaactttaaataatttctactgttgtagatgagaagtcatttaataaggccactggctcaccttcgggtgggcctttctgcgcaattgttaggccaattaccaacttggtggggcgttgttggggggattactcagtatgccgatgttcgatgcggtacgccaggctctgtcgatgatgcaggcggtgatcctgatggcgctgtatatcttaatcccgttgatcctgctgtttgcggcgtatgagtttaagaccgtcttcacgctgacgtttgtgatttttgcgctgaatttcctgactttctggtgggaaaccgcccgctggctggacagctatttgctggaagccctgtacggctcagacacccacagcctgtttaatctggcgggcattcagaatacctcggacgatttaattatggagctggtaatggggacgctgtttatcgtcctgccgatggtctggctgggggcattagcgtgggccggtgtacgcatggacgatatggcaggcatgatgagtcagggtgtagggcaggtcaggtaatcaccgggatgttcggacagctggtgatgcagaaggtatctgcgctccactagagaccgtagcc

SEQ ID NO: 172 - DNA, 713 bp, synthetic construct
  source 1..713: mol_type = other DNA
ggctacggtctcttccatgaatactggtataactcatcgctggtcgaagtttgttaataaaaaaaatggtcgctcgttaattattccaatagatcatggtttaactagtgggccaatggcgggaataaataacttctctgccgttgagagctggataaatagctcacatattgatggtatcgtcgttcacccaggcttcttatatcaccttgttaagcataatttgctactcggtacagctgtgtgtttacaaattaatggtaatagtcacctagcccaagacgcaagttttaagccactgctgtcaaatgcagatcttgctatgcagttaggtgccgatgctattagcttagatttaccctttagtcagcataatacagcccagaatttgatccaactcggtcaggttatgcaacacaccataccccgtggtataccagtattagtgatgttcagtttaattgagtcggatttgagtgataaagagtacgtgacacgtcaccgaaaatatatccgaggatcccacaccgcatatgctggatccttgacagctagctcagtcctaggtataatactagttcgagattttcaggagctaaggaagctaaagtctaagaactttaaataatttctactgttgtagatcttattggctaattgatagctgagtaaccatgtctcgagaccgtagcccttccaagaatccgtctagt

SEQ ID NO: 173 - DNA, 695 bp, synthetic construct
  source 1..695: mol_type = other DNA
aaccggcgtattaagttgggtctcgagataaatgtgcagagaatttataataaaaataatgtctaagaactttaaataatttctactgttgtagatgagaagtcatttaataaggccactggctcaccttcgggtgggcctttctgcgcaattgttaggccaattaccaacttggtgcggcgaatgccattattggctaagttacggcagcatacgagcgctaactgaatcaattaggagataaaagagtgcagattcaaacttttcacgcagaaagtaattggcaaggaaccctggctaccggcaagggaatgttaaatgtggggagcttagcgagcattatttcagttccgcaggatttaggtggattagggaaaggaaccaacccagaagatttaattgtcggcgcttcgacttcttgctttattattacattgagtgctatttttcaatttaataaaatagagtatgaaaatattagtgttaatagtgaagctgatttttccattactccaatgggacctaaattaaaagaaattagacattttgtttctattcaatcctctcatgatttaccgcgtgaaactgtggataaatatattaaacaagctgaaaaagggtgtatgattagtattgcactaagcggtaatgtagaggttatagctcagctaaattaaccactagagaccgtagcc

SEQ ID NO: 174 - DNA, 835 bp, synthetic construct
  source 1..835: mol_type = other DNA
  misc_feature 15..89: note = RiboJ51
  misc_feature 90..138: note = T7g10 Translational Enhancer + RBS
aggtctcgcactggagtagtcaccggctgtgcttgccggtctgatgagcctgtgaaggcgaaactacctctacaaataattttgtttaaagaaataattttgtttaactttaaaaaaaaaaaagaaggagaataatctatgtccagtgagttgcataataatattattttagagaatggattgaatgcgataaataaagggctttccttgcctgcgttatttgaaaaagtaactcagatattctctcgggaagtggcaatttattatggtaataaggcacaaaataaagcgacatataatgaaattgctttgcttatccaaaaagtcattgtaaaaattgagcaggcaggattacctcctggatcgcatattggagtgaattgtaataaatcccccctattatttgccagcatgtttgcgatttggcactgcggatacgtttacaccccgctagaacccacttggcctgcggattatcttcgtcggttagcggaatatgccgatgtgctcttgattcttcacgatacggaagatggcaaagtgcctgcttgggcagcgaattccacggtgctatccgcccatgacattgaggctgttagccagtttcaatatcaacccttaaatgagggaaatatttctggatcccacaccgcatatgctggatccttgacagctagctcagtcctaggtataatactagttcgagattttcaggagctaaggaagctaaagtctaagaactttaaataatttctactgttgtagatctcaggagcaatcctgacctaataaggccctgtctcgagaccgtagcccttccaagaatccgtctagt

SEQ ID NO: 175 - DNA, 673 bp, synthetic construct
  source 1..673: mol_type = other DNA
ggctacggtctctagatgccaccatgcaggccaggttaatcctcgcttgtctaagaactttaaataatttctactgttgtagatgagaagtcatttaataaggccactggctcaccttcgggtgggcctttctgcgcaattgttaggccaattaccaacttggttatctgaatgagatgatgtccgtctcgctgttaccttggttgcctgaaatccatgtgccgactcactgtgttttcggtgatttggataggattatcaatccgcgtcattcacacgatttcaaccagggtattcaggatgcgcgtctgacctgtatccccggttcagctcatttcccttatcttactcatgacacgcagtttattcctgtgttggaggcatttgtggatgagcatgaacagcagtgggttgttgttgcagagacacgtagggaggttagtgatgcaacagcataacattgcgtctgatttacaggattcctggtcactggtgggcgcgaaaggtgcttggtccggcgagcgttacaaggccagcctgcgggatcggagacaggtttggtatgacggtgaccgtattgaagtgacggaacatcctgacttcgtcagcatgttaaatcacctggctgccctgtacgatctgcaatatcagccggaactgggagaccgtagcc

SEQ ID NO: 176 - DNA, 694 bp, synthetic construct
  source 1..694: mol_type = other DNA
ggctacggtctccactgcgtgcctgggaaatggtagcggatgcctttggtagccgacaggatatttatgagttatggaatcgaggcgatgttgtacgcaaccgtatccatctctataaaacgcttggcgcaagagaagagattgaaaaccatattaggcaattgatcgatatctattaaatgaatattgggtgttgctaacgcggcctttagggctgcgtctaaataatattttaaaatatagtgtaggagttcgttatggataataagtcatctgtattgccattctctatttggagtttggcgattccgatatttatcgatatgttattatcattttcgatattttttgttgattcgttatttttgagtcatgtttcaacgacgatggctgcgtcggttggcgctattattcccatctttgttatttttgtcctgttatttatgatgatggctcagggagcaattaacgtaggtgggcaaattattggttctggtaaattagaaaagcttgatcttggatcccacaccgcatatgctggatccttgacagctagctcagtcctaggtataatactagttcgagattttcaggagctaaggaagctaaagtctaagaactttaaataatttctactgttgtagatcctgaactcattcacgccaatcgactgactagtctggagaccgtagcc

SEQ ID NO: 177 - DNA, 678 bp, synthetic construct
  source 1..678: mol_type = other DNA
ggctacggtctctagataatcttatcggtagcatcccccagcagcgttgtctaagaactttaaataatttctactgttgtagatgagaagtcatttaataaggccactggctcaccttcgggtgggcctttctgcgcaattgttaggccaattaccaacttggtgaggagtatctcgggcagtttcactttaacccaccgtcaatccccgtgattgctaatgcaaccagccagccttatagagcaacaagtataacggagaacttagtcagacaactgactgagcctgttcattggcagtcatgcattgaatttgtttaccggcaggggcccatgctctttatcgaattaggggcaaaaacggttctatctaaatttatgggctcactctttgatgcttattcatcactgtctgacgatatttaggaaatttttatgatcaatattatcaagcgcgcttctattccagaaattaactccatttttattgcagggaaagagcacaaaatcggcgcagtgcagcctttagcgcatagcgctatttttcgtcattttagaaaagataacgaaccacttagcattagttttacacgtcttgaaaagggtgaaacacatcaggtgcatcagcacagtgtagatagtatggttatcgttatggctgaactgggagaccgtagcc

SEQ ID NO: 178 - DNA, 662 bp, synthetic construct
  source 1..662: mol_type = other DNA
ggctacggtctccactgcgtccaagatgggaagatagagttatttataggggtagaataattatgatgcatttattagggactgtccatttcattagtacatgaaggactgtttgctctgaaatgccagcagtctctcctttattactcaacaaccaccgactaaagtcggtggttgttgagttaatttaaacttaagattgagataaagaacctattacaattcagtgtcatattgccgtttgataatttttatctgttttggtgaaacagatatttaattgaactgaaaatatccggtaaaaaataaagaacaaggtaaaacaaaaagttgaccgggattgatgcaatcattaacctatttcttgttatttcagtgatcccatcgcctgcaaggatcacccatcgggtggcgaaacaaatctatggtcttccccgtttttgcaacactcattttgatgtatggttggcttgcttaaatctatccggatcccacaccgcatatgctggatccttgacagctagctcagtcctaggtataatactagttcgagattttcaggagctaaggaagctaaagtctaagaactttaaataatttctactgttgtagatctccggcaatcacgtcaaacagcccgtttgcgtctggagaccgtagcc

SEQ ID NO: 179 - DNA, 615 bp, synthetic construct
  source 1..615: mol_type = other DNA
aaccggcgtattaagttgggctacggtctcgagatacgtcgtaatgcgttcaggaatggatcttgcgtctaagaactttaaataatttctactgttgtagatgagaagtcatttaataaggccactggctcaccttcgggtgggcctttctgcgcaattgttaggccaattaccaacttggtgtctggagaagtattgccttatcgtgttcatcagtttgcgattgcgcctgcaagccgtgaagttctgcatcagcgcattgctgcccgttttgaacaaatgatcaaatcagggtttgaagatgaagttaaagcgctttatgctcgtaacgatttgcatacagatttaccttccattcgttgtgttggttatcgtcagatgtggtcttatctcgcaggcgaaatctctcatgatgagatggtttatcgtggtatctgtgcgactcgccagctagcgaagcgtcagatcacatggctcaggagttgggacaatgtgacttggttggacagtgatcaacctgaacaagcactaaatacagtcatgcaggttattagtacataaatttattgattgtgtacaattgatggtatctgcgctccactagagaccgtagcc

SEQ ID NO: 180 - DNA, 743 bp, synthetic construct
  source 1..743: mol_type = other DNA
caccacaggtctcgcactggtatctgcgctccactaaagccatcagtgatagtgcctgatgtaaataaaaacataggtagggagactttacctatgtttttcctatggattttactcagcctgaggggttgcaccgttgtttgatcgttatgaaggcggagaactagctgttctggtgcatgtttttttctcacaggaaaaagatacggaaaatctcggtgaattcgaatcattggtgacttccgcaggtgtttctcctgtgcagatagtgacaggtagccgtaaggctccgcatccgaaatattttgtcggggaaggcaaagcagaagaaattgctgaagcagtcaaaaacagtggcgcagatgtggtgttgtttaatcatacactcagtcctgctcaggaacgtaatcttgaacgcctgtgtcaatgccgtgttgttgatcgcacgggtgtgattctggatatttttgcccagcgggcaagaacgcatgaaggtaagttacaggtagaacttgcacaattgcgtcatttatccacccgtttagttggatcccacaccgcatatgctggatccttgacagctagctcagtcctaggtataatactagttcgagattttcaggagctaaggaagctaaagtctaagaactttaaataatttctactgttgtagattaaacacgccatctctactgttgtgccttctgtctcgagaccgtagcccttccaagaatccgtctagt

SEQ ID NO: 181 - DNA, 682 bp, synthetic construct
  source 1..682: mol_type = other DNA
ggctacggtctcgagatcaagatccattcctgaatgctttacggcgtggtctaagaactttaaataatttctactgttgtagatgagaagtcatttaataaggccactggctcaccttcgggtgggcctttctgcgcaattgttaggccaattaccaacttggttgaattgactgaaacggctggagaaatattgccttatcatgttcatcaatttgcgatcgctcctgcaagccgtgaagttttgcatcagcggatcgctgcccgttttgaacaaatgatcaaatcagggtttgaagatgaagtgaaagcactttatgctcgtcgtgatttgcatacagatttaccttccattcgttgtgtcggttatcgtcagatgtggtcttacctcgcaggcgaaatatctcatgatgagatggtttatcgtggtatttgtgcgacacgccagctggcgaaacgtcagatcacgtggctcaggagttgggacaatgtgacttggttggatagcgatcaacctaaacaagtacttaatacggtcatacaggttattggtacataaattcattgattgtgtacaattgatcagtacaaagtgtcatttttgagtagttaatttttcgaaccaaaaggttcttagttaaaacaacaaaataaggaaaacatagagcagagagaccgtagcc

SEQ ID NO: 182 - DNA, 695 bp, synthetic construct
  source 1..695: mol_type = other DNA
ggctacggtctctgcagcaaatcagtgaccgtacctgacataaataaaaacataggtagggagactttacctatgttttttcctatggatttttattaagcctgaggggttgcgccgttgtttgatcgttatgaaggcggagaattagctgttctggtgcatgttttcttctcacaggaaaaagatacggaaaatctcagtgaatttgaatcattggtgacttccgctggtgtttctcctgtgcagattgtaacgggaagccggaaggcacctcatccaaaatattttgtcggtgagggcaaagcagaggaaattgccgaagctgttaaaaatagcggcgcagatgtggtgctgtttaaccataccctcagccctgctcaagaacgtaatcttgaacgcttgtgccaatgccgtgttgttgatcgtaccggggttattctggatatttttgcccagcgggcaagaacacacgaaggcaagctgcaagttgaattggctcagttgcgtcatctatcgactggatcccacaccgcatatgctggatccttgacagctagctcagtcctaggtataatactagttcgagattttcaggagctaaggaagctaaagtctaagaactttaaataatttctactgttgtagatctgttgtgtagccgggttagaacccgcgtgagtctagagaccgtagcc

SEQ ID NO: 183 - DNA, 683 bp, synthetic construct
  source 1..683: mol_type = other DNA
ggctacggtctcgagattcgtaccgcggtctgcatggcgggtaagcgtgtctaagaactttaaataatttctactgttgtagatgagaagtcatttaataaggccactggctcaccttcgggtgggcctttctgcgcaattgttaggccaattaccaacttggtaacaaggggctaacacgatcgtgggcactggaacagatatcgggcgaaactcttgtatctctatattgaaacgctgaagcagcgactggatgcaattaaccaacttcggctagagcgcgcaactagctcaatgagtgccgcttttaagcaagtttatagtttaattccagtattattacatcatcatcatcccatgatgccaggttatatcaaaaataatactcctcatggagtttgtttttttgctcccgatgaatcacaacagttttggattaatgatctggaaggccggctttctggtgtcttgccatatgcgcctaatggcgaattgccgattacgggaatttattcaatgggcagcacgtcttcaatagggcaaagttgttcctctgacattgatatatgggtttgtcaccaatcctggttggataatggcgaaaaagcgttattggaacaaaaatgtaccttgataggacaatgggctgcttcactcggtattgaagcagagagaccgtagcc

SEQ ID NO: 184 - DNA, 711 bp, synthetic construct
  source 1..711: mol_type = other DNA
ggctacggtctctgcagcacaaaaattggaaaatgcggtggaattctatggcgccatttccaacaccaaactacatggcctgcccgttaaaattgataccaaagaaaaacatttgccgccagtgatagacgggtttgcttgtgagggcattatccagttttttttcgaaaacattgagaagaaccaggggttcaatatctatatcctcgatgagtcaaaccgggtggaaatttattcacattgtgaaggcagtaaagaggaattggtgcgggatatcagccgcttttactcttcttcgcacgatcgttttacttacggttccagctttatcaatttcaacttaccgcagttctatcaaattattcagaaaggcgaacgtactcatgtcgttccttttgtggatgggacgtttccatcggatgatatggaagccaacgagacaagactcaatattgatagttgtcttgaagaggcctgccgaccccatgacccttaccgttctttgtatcactattaattgatattcggttttcccggatcccacaccgcatatgctggatccttgacagctagctcagtcctaggtataatactagttcgagattttcaggagctaaggaagctaaagtctaagaactttaaataatttctactgttgtagatcccaggatggtcttcagcgcttccagcactggtctagagaccgtagcc

SEQ ID NO: 185 - DNA, 681 bp, synthetic construct
  source 1..681: mol_type = other DNA
ggctacggtctcaagatgtgaacaccgaaaacagctccgaggaagaacgtctaagaactttaaataatttctactgttgtagatgagaagtcatttaataaggccactggctcaccttcgggtgggcctttctgcgcaattgttaggccaattaccaacttggtgactatctgcatggccgtggttctcttatcagtggattggagaaagcactggaaggccgtgaggcgggtgaacgcttcgatgtgagcgtagaagcaaacgatgcttacggtcagtatgatgataatctggttcagcgtgcaccaaaagatgtgtttgttggtgttgaggagcttgaagtgggcatgcgttttctggctgatacggatcaaggtcctatcccggttgaaatcacggcaattgaaggtgatgaagttgtcgttgatgctaaccacatgctggcgggtcaaaatctgaaattcaatgttgaaatcgtagcgattcgtgaagcaactgaagaagagttggctcatggccatgttcatggtcagggtggttgtggtgatcatcaccatgagcacgatgaacatggatgctgcggcggtagctgccactaatctgagatcattgactaactaacctggattctggataagaaattgacgggaagcctgttctaggacactagagaccgtagcc

SEQ ID NO: 186 - DNA, 1386 bp, synthetic construct
  source 1..1386: mol_type = other DNA
gactacggtctcctccatgaacagcactactaaaaatatatgtataaaatccactttgcctttttctaaaagctctctatgtcttcttgtcgcagtagcctctttattttttccagcctttgcaactgcaacaataaaggatacaaattcagtcataacaagcagtaaagtagataatgtactgcaacacattcagaaacttcaggaaatatcagataataatggaggtaatcgtgaagcaggaagcaatggacacgctgaatcagtacaatacttttccgagcagcttcgcaatgcaggtttgaaagtctggaccgagaaattcgaatttctatatagcaaagccgaacatgcacaattgatgaacaacggaaaaatgctggatctcgttcccgcaatgttcagttcatcgacacctgggcctattagtgcccctccacacttcatcggtggaaacggatgcgaaaaacaacagtatctcgatgtgcctcgtggatcaatcgtcatcatacaagagggatcccacaccgcatatgctggatccttgacagctagctcagtcctaggtataatactagttcgagattttcaggagctaaggaagctaaagtctaagaactttaaataatttctactgttgtagataatgaaatatgacatgcagccgatattccacgtctcgagaccgtagccgactacggtctcctccatgaacagcactactaaaaatatatgtataaaatccactttgcctttttctaaaagctctctatgtcttcttgtcgcagtagcctctttattttttccagcctttgcaactgcaacaataaaggatacaaattcagtcataacaagcagtaaagtagataatgtactgcaacacattcagaaacttcaggaaatatcagataataatggaggtaatcgtgaagcaggaagcaatggacacgctgaatcagtacaatacttttccgagcagcttcgcaatgcaggtttgaaagtctggaccgagaaattcgaatttctatatagcaaagccgaacatgcacaattgatgaacaacggaaaaatgctggatctcgttcccgcaatgttcagttcatcgacacctgggcctattagtgcccctccacacttcatcggtggaaacggatgcgaaaaacaacagtatctcgatgtgcctcgtggatcaatcgtcatcatacaagagggatcccacaccgcatatgctggatccttgacagctagctcagtcctaggtataatactagttcgagattttcaggagctaaggaagctaaagtctaagaactttaaataatttctactgttgtagataatgaaatatgacatgcagccgatattccacgtctcgagaccgtagcc

SEQ ID NO: 187 - DNA, 684 bp, synthetic construct
  source 1..684: mol_type = other DNA
ggctacggtctcgagatatgggtgacgctttcaagcaagtttatagttgtctaagaactttaaataatttctactgttgtagatgagaagtcatttaataaggccactggctcaccttcgggtgggcctttctgcgcaattgttaggccaattaccaacttggttaatattttatttgaatggagaaaataaacaaaaagattgacttaaaagcaaattaacatttttgtttagcgtgatgataaaaagcggtgcaaatatttaacaatagaaaaataggtttagattaaatatttacggaagaatgatgtacttacaatgctttacatctacgatacgaaaaataatcaactatgatagttatgctaaaaaagcatattattgtgtctatttcgtgattaacttaatattttacaaagggttaattatgttttggctattgcaggtgaagagaaaagatggtattgcgtactttacgctttgactgcaaggtgttaaattgatcacgtttccgacaataagtttgtaaaaataattttaaataatcttgcaataccacgcgttatcggaaacaggggactactttgggcactggaactgatatcaggcaaaactcttgtatctctatattgaaacgctgaagcagcgactggatgcaattaaccaagcagagagaccgtagcc

SEQ ID NO: 188 - DNA, 698 bp, synthetic construct
  source 1..698: mol_type = other DNA
ggctacggtctctgcagcatcgttaataaaggtgagcgcacccatgtcaccccgtttatagaaggcgtgtatccggtggatgatatcgaagcgaatgacgcaaggctgcatgttgaggtgtgccttgataagacatgccgggatgatccttatcgttcgttgtatcactattgatatttgtctttttgatatttttcgttatcaatgacactaagcgcctgttttatcaggcgcttttggttaatataatgatttttactggattgccgtgaaaaatattcacgaaaattgaaatggctcaccgctttggtcagtgatcgcttgtgccagcatggcgataaaatcgtgaccactgcggtcacaaatccactgactatcctgataatcaaaatggtaaccgccatttttggttgccagccatatctgatggaaaggctcttggcggttgataatgattttgctgccgttttcaaaactcagtgtcatcacaccgccgttggtttcatagtcaatatccgaatcggatcccacaccgcatatgctggatccttgacagctagctcagtcctaggtataatactagttcgagattttcaggagctaaggaagctaaagtctaagaactttaaataatttctactgttgtagatatagaactgcggtaaattgaaattgacaaaagtctagagaccgtagcc

SEQ ID NO: 189 - DNA, 675 bp, synthetic construct
  source 1..675: mol_type = other DNA
ggctacggtctcaagattgtgcgctagcgtgatgtaattgaaatatctgtctaagaactttaaataatttctactgttgtagatgagaagtcatttaataaggccactggctcaccttcgggtgggcctttctgcgcaattgttaggccaattaccaacttggtatttcacctgccctcggcggtgctgtgggttttattggcgtcgagaaaatccgcgagattgcgcttcgggccatcaataaacgtttaggagataaacaatgaccagagggatacgtaaccacaatcccggcaatattcgccacggggataaatggcagggtttgcgtgatattcagaccgataaatcattttgtcagtttatcgcaccagaatatggcatacgagcaatgctgaaaattctgcgtaattatgaacgcaaatatggatataacacgatccgccagtttatttcccgctgggcaccgcccaatgaaaataataccgagagttatattgcttatgtgtgcaaatccgttggcgtgcctgacagcgctgtcatcgatgtagataatatcgccaccatgagccgactggttaaggccattatccaaatggaaaacggccgacagccttattctgatgccgtcatcaaacgggcatttgaattattataacactagagaccgtagcc

SEQ ID NO: 190 - DNA, 690 bp, synthetic construct
  source 1..690: mol_type = other DNA
tacggtctcttccatgcctatgtcatgcaatggtattaataacgatagtattaacaacgatagtattaacaacagtactcacaaccgagatccacttccgccagcggatccttctgcctctggtaaattctctctcagttctactcagcaagtcgtttggctcgatcaatacctccgtcctgatagcacttgttacaacctgggttcagtcgttctgattgagggaaagttggatgaagccctgttaatacacgcctttaaaaccgttgtttatcgtcatgatgcgttgcgtttacggctcatcagtactcaggaattaccattgcaagaactcacggacacaagcaatgtattcgtgacaattcgtgatttttcagactatgcagacgcagaagaacaggcgaaacaatatatccatgctgcttttagctgtccgtttgatttaaacggtgaactatggcgttttgagttgttacgcgtgagtgataatcgttggtactggcagttctgttgtggatcccacaccgcatatgctggatccttgacagctagctcagtcctaggtataatactagttcgagattttcaggagctaaggaagctaaagtctaagaactttaaataatttctactgttgtagatattcattagccaagtgcaccagccttacgaggtctggagaccgtagcc

SEQ ID NO: 191 - DNA, 31 bp, Xenorhabdus nematophila
  source 1..31: mol_type = other DNA
caagatccattcctgaatgctttacggcgtg

SEQ ID NO: 192 - DNA, 31 bp, Xenorhabdus nematophila
  source 1..31: mol_type = other DNA
ctgttgtgtagccgggttagaacccgcgtga

SEQ ID NO: 193 - DNA, 31 bp, Xenorhabdus nematophila; identical to SEQ ID NO: 192
  source 1..31: mol_type = other DNA

SEQ ID NO: 194 - DNA, 31 bp, Xenorhabdus nematophila
  source 1..31: mol_type = other DNA
cccaggatggtcttcagcgcttccagcactg

SEQ ID NO: 195 - DNA, 31 bp, Xenorhabdus nematophila
  source 1..31: mol_type = other DNA
gtgaacaccgaaaacagctccgaggaagaac

SEQ ID NO: 196 - DNA, 31 bp, Xenorhabdus nematophila
  source 1..31: mol_type = other DNA
aatgaaatatgacatgcagccgatattccac

SEQ ID NO: 197 - DNA, 31 bp, Xenorhabdus doucetiae
  source 1..31: mol_type = other DNA
atgggtgacgctttcaagcaagtttatagtt

SEQ ID NO: 198 - DNA, 31 bp, Xenorhabdus doucetiae
  source 1..31: mol_type = other DNA
atagaactgcggtaaattgaaattgacaaaa

SEQ ID NO: 199 - DNA, 31 bp, Xenorhabdus doucetiae
  source 1..31: mol_type = other DNA
tgtgcgctagcgtgatgtaattgaaatatct

SEQ ID NO: 200 - DNA, 31 bp, Xenorhabdus doucetiae
  source 1..31: mol_type = other DNA
attcattagccaagtgcaccagccttacgag

SEQ ID NO: 201 - DNA, 26 bp, Xenorhabdus nematophila
  source 1..26: mol_type = other DNA
  misc_feature 1..26: note = AR_seq_86
acctaaacaagtacttaatacggtca

SEQ ID NO: 202 - DNA, 20 bp, Xenorhabdus nematophila
  source 1..20: mol_type = other DNA
  misc_feature 1..20: note = AR_seq_87
cccgcctttttgacgttcaa

SEQ ID NO: 203 - DNA, 20 bp, Xenorhabdus nematophila
  source 1..20: mol_type = other DNA
  misc_feature 1..20: note = AR_seq_88
aatgtcaccacgtgtcacca

SEQ ID NO: 204 - DNA, 24 bp, Xenorhabdus nematophila
  source 1..24: mol_type = other DNA
  misc_feature 1..24: note = AR_seq_89
acgatggagatgcagatattgact

SEQ ID NO: 205 - DNA, 20 bp, Xenorhabdus nematophila
  source 1..20: mol_type = other DNA
  misc_feature 1..20: note = AR_seq_90
cacccagacgaacaaccaga

SEQ ID NO: 206 - DNA, 21 bp, Xenorhabdus nematophila
  source 1..21: mol_type = other DNA
  misc_feature 1..21: note = AR_seq_91
gtgatcatcagaacgtccagc

SEQ ID NO: 207 - DNA, 20 bp, Xenorhabdus doucetiae
  source 1..20: mol_type = other DNA
  misc_feature 1..20: note = AR_seq_92
cacatattgcgcttgccaca

SEQ ID NO: 208 - DNA, 20 bp, Xenorhabdus doucetiae
  source 1..20: mol_type = other DNA
  misc_feature 1..20: note = AR_seq_93
agctaggccctgaaaagcaa

SEQ ID NO: 209 - DNA, 20 bp, Xenorhabdus doucetiae
  source 1..20: mol_type = other DNA
  misc_feature 1..20: note = AR_seq_94
cagcgggctggaactatttg

SEQ ID NO: 210 - DNA, 21 bp, Xenorhabdus doucetiae
  source 1..21: mol_type = other DNA
  misc_feature 1..21: note = AR_seq_95
ttcagcgatgagtcctaacgt

SEQ ID NO: 211 - DNA, 771 bp, synthetic construct
  source 1..771: mol_type = other DNA; note = clxD (codon optimized)
atgattatcgacgcacacgcacatattacggcgaagaattacggaagcgtcgaggcgctgattgagcggtacgacgaaatcggtataagcaagggcgtcctggtcccaggcggtatgatagacgttcgaatgatggataaggtcgtctctggggagaaaccgatagctactaagcccatacccaactacttggtcaaggatgcaatgaataaatatccggatagagttttcggtttcttctgcgtagaccccaatgatggtgaggtttccatcaacgagttcacggatgctgttaagacacaggggttctgcggcctgaaactcgcacctattgtacatcggttctcactgaaatcaaatatcgtgaaggagttggcccgcacatgcagtgaattagaagtgccttgcttctttcataccacagacttcgagggctcgtttacaaaggattgccacgaccttattaaagattgtcctaacactaagtttatcctgggtcacatgggtttcggcttttcggactccctgtcaatcgagtatgctaagaaatttgagaacttatacctggagacatcatgcgcatcaacgtccatagttcgccaagccatacagactttaggggacactaaggtaattttcggatctgagtaccctatggacgacccgttctatgctttaagtaagattacttgcctgaagctgagcgagaaatctttcgaaaatgtcacttgtaataatattctcaaccttttacaaaagcgtggctgataa

SEQ ID NO: 212 - DNA, 1173 bp, synthetic construct
  source 1..1173: mol_type = other DNA; note = styA (Bacillus) *T1-->A
atgaaaaaacgtatttgtattattggtagtggaacggcagggttaaatttagcctatgccttaagagaaaattttgaagttacaattttgcatcatcgatctgctgaagatataaagaatggaagaattatgtctactcaaattcaacccaaaaaaactattagaaatcattttgatttggagaagtggaatgatcaaacacttattcaaaatcttcatatgaatattggtaatgaaaatttatttgtagcaaattttaaggaatctgcattgtcagttgatcaacgtttttatttttttcatgagatgaaggcactttctaaacaggatgtctctttcgatttaaataaggtttcaaataaggaaataaaagaaataataaacgattttgatttggtcattgattgttcaggaaaacaggggcctattttcccatttcctattgaggaagatttatccccatttgacaaacctcaaaggaaatgtattgttggttactttttaggggtcgaaaggaaaaaacctattggtgtcaatgttactattctaccagaattaggtcaaataattgaaatacctgcaattactgagtatggtcctgttacaatattttacatttcagtaataccgaaaagcagattagatgtatttaaagggattaaaaatgctgatgagtttactaataaaatgaaagaggtagtccaagagtttttccctgaaatccagagtcgtatggatgagaaggaatttagattatgtgatgaaaaagggtttcttcaaattgcttttacccctgtaattcgtaagccatatttaacaattaaagataaactagtcattggttgtggagatagtgtttctttaaatgacccgattacagggcaaggttgcaatgtaatagcttattgtgtagagcaattagctcaaacacttaatgaacacaaggaatctcaatgggacgaaagaattggagaagtttattgggataagataaatcaatttgtaaaagaagtatcagaatggacaaatgctatgatgcaaccattaccggaacatgttattcaattattaatggctggctcaactaatccagaggtggctgatcaaattgcagaatggtttgtcaatcctcgaactgcatatgaagcatttttttctgttaaagccctatag